SEQUENCE LISTING (175 sequences)

SEQ ID NO: 1 - protein, 365 aa, synthetic construct
  UNSURE 1..365: note = HsFUT1
MWLRSHRQLCLAFLLVCVLSVIFFLHIHQDSFPHGLGLSILCPDRRLVTPPVAIFCLPGTAMGPNASSSCPQHPASLSGTWTVYPNGRFGNQMGQYATLLALAQLNGRRAFILPAMHAALAPVFRITLPVLAPEVDSRTPWRELQLHDWMSEEYADLRDPFLKLSGFPCSWTFFHHLREQIRREFTLHDHLREEAQSVLGQLRLGRTGDRPRTFVGVHVRRGDYLQVMPQRWKGVVGDSAYLRQAMDWFRARHEAPVFVVTSNGMEWCKENIDTSQGDVTFAGDGQEATPWKDFALLTQCNHTIMTIGTFGFWAAYLAGGDTVYLANFTLPDSEFLKIFKPEAAFLPEWVGINADLSPLWTLAKP

SEQ ID NO: 2 - protein, 343 aa, synthetic construct
  UNSURE 1..343: note = HsFUT2
MLVVQMPFSFPMAHFILFVFTVSTIFHVQQRLAKIQAMWELPVQIPVLASTSKALGPSQLRGMWTINAIGRLGNQMGEYATLYALAKMNGRPAFIPAQMHSTLAPIFRITLPVLHSATASRIPWQNYHLNDWMEEEYRHIPGEYVRFTGYPCSWTFYHHLRQEILQEFTLHDHVREEAQKFLRGLQVNGSRPGTFVGVHVRRGDYVHVMPKVWKGVVADRRYLQQALDWFRARYSSLIFVVTSNGMAWCRENIDTSHGDVVFAGDGIEGSPAKDFALLTQCNHTIMTIGTFGIWAAYLTGGDTIYLANYTLPDSPFLKIFKPEAAFLPEWTGIAADLSPLLKH

SEQ ID NO: 3 - protein, 361 aa, synthetic construct
  UNSURE 1..361: note = HsFUT3
MDPLGAAKPQWPWRRCLAALLFQLLVAVCFFSYLRVSRDDATGSPRAPSGSSRQDTTPTRPTLLILLWTWPFHIPVALSRCSEMVPGTADCHITADRKVYPQADTVIVHHWDIMSNPKSRLPPSPRPQGQRWIWFNLEPPPNCQHLEALDRYFNLTMSYRSDSDIFTPYGWLEPWSGQPAHPPLNLSAKTELVAWAVSNWKPDSARVRYYQSLQAHLKVDVYGRSHKPLPKGTMMETLSRYKFYLAFENSLHPDYITEKLWRNALEAWAVPVVLGPSRSNYERFLPPDAFIHVDDFQSPKDLARYLQELDKDHARYLSYFRWRETLRPRSFSWALDFCKACWKLQQESRYQTVRSIAAWFT

SEQ ID NO: 4 - protein, 530 aa, synthetic construct
  UNSURE 1..530: note = HsFUT4
MRRLWGAARKPSGAGWEKEWAEAPQEAPGAWSGRLGPGRSGRKGRAVPGWASWPAHLALAARPARHLGGAGQGPRPLHSGTAPFHSRASGERQRRLEPQLQHESRCRSSTPADAWRAEAALPVRAMGAPWGSPTAAAGGRRGWRRGRGLPWTVCVLAAAGLTCTALITYACWGQLPPLPWASPTPSRPVGVLLWWEPFGGRDSAPRPPPDCRLRFNISGCRLLTDRASYGEAQAVLFHHRDLVKGPPDWPPPWGIQAHTAEEVDLRVLDYEEAAAAAEALATSSPRPPGQRWVWMNFESPSHSPGLRSLASNLFNWTLSYRADSDVFVPYGYLYPRSHPGDPPSGLAPPLSRKQGLVAWVVSHWDERQARVRYYHQLSQHVTVDVFGRGGPGQPVPEIGLLHTVARYKFYLAFENSQHLDYITEKLWRNALLAGAVPVVLGPDRANYERFVPRGAFIHVDDFPSASSLASYLLFLDRNPAVYRRYFHWRRSYAVHITSFWDEPWCRVCQAVQRAGDRPKSIRNLASWFER

SEQ ID NO: 5 - protein, 374 aa, synthetic construct
  UNSURE 1..374: note = HsFUT5
MDPLGPAKPQWLWRRCLAGLLFQLLVAVCFFSYLRVSRDDATGSPRPGLMAVEPVTGAPNGSRCQDSMATPAHPTLLILLWTWPFNTPVALPRCSEMVPGAADCNITADSSVYPQADAVIVHHWDIMYNPSANLPPPTRPQGQRWIWFSMESPSNCRHLEALDGYFNLTMSYRSDSDIFTPYGWLEPWSGQPAHPPLNLSAKTELVAWAVSNWKPDSARVRYYQSLQAHLKVDVYGRSHKPLPKGTMMETLSRYKFYLAFENSLHPDYITEKLWRNALEAWAVPVVLGPSRSNYERFLPPDAFIHVDDFQSPKDLARYLQELDKDHARYLSYFRWRETLRPRSFSWALAFCKACWKLQQESRYQTVRSIAAWFT

SEQ ID NO: 6 - protein, 359 aa, synthetic construct
  UNSURE 1..359: note = HsFUT6
MDPLGPAKPQWSWRCCLTTLLFQLLMAVCFFSYLRVSQDDPTVYPNGSRFPDSTGTPAHSIPLILLWTWPFNKPIALPRCSEMVPGTADCNITADRKVYPQADAVIVHHREVMYNPSAQLPRSPRRQGQRWIWFSMESPSHCWQLKAMDGYFNLTMSYRSDSDIFTPYGWLEPWSGQPAHPPLNLSAKTELVAWAVSNWGPNSARVRYYQSLQAHLKVDVYGRSHKPLPQGTMMETLSRYKFYLAFENSLHPDYITEKLWRNALEAWAVPVVLGPSRSNYERFLPPDAFIHVDDFQSPKDLARYLQELDKDHARYLSYFRWRETLRPRSFSWALAFCKACWKLQEESRYQTRGIAAWFT

SEQ ID NO: 7 - protein, 342 aa, synthetic construct
  UNSURE 1..342: note = HsFUT7
MNNAGHGPTRRLRGLGVLAGVALLAALWLLWLLGSAPRGTPAPQPTITILVWHWPFTDQPPELPSDTCTRYGIARCHLSANRSLLASADAVVFHHRELQTRRSHLPLAQRPRGQPWVWASMESPSHTHGLSHLRGIFNWVLSYRRDSDIFVPYGRLEPHWGPSPPLPAKSRVAAWVVSNFQERQLRARLYRQLAPHLRVDVFGRANGRPLCASCLVPTVAQYRFYLSFENSQHRDYITEKFWRNALVAGTVPVVLGPPRATYEAFVPADAFVHVDDFGSARELAAFLTGMNESRYQRFFAWRDRLRVRLFTDWRERFCAICDRYPHLPRSQVYEDLEGWFQA

SEQ ID NO: 8 - protein, 575 aa, synthetic construct
  UNSURE 1..575: note = HsFUT8
MRPWTGSWRWIMLILFAWGTLLFYIGGHLVRDNDHPDHSSRELSKILAKLERLKQQNEDLRRMAESLRIPEGPIDQGPAIGRVRVLEEQLVKAKEQIENYKKQTRNGLGKDHEILRRRIENGAKELWFFLQSELKKLKNLEGNELQRHADEFLLDLGHHERSIMTDLYYLSQTDGAGDWREKEAKDLTELVQRRITYLQNPKDCSKAKKLVCNINKGCGYGCQLHHVVYCFMIAYGTQRTLILESQNWRYATGGWETVFRPVSETCTDRSGISTGHWSGEVKDKNVQVVELPIVDSLHPRPPYLPLAVPEDLADRLVRVHGDPAVWWVSQFVKYLIRPQPWLEKEIEEATKKLGFKHPVIGVHVRRTDKVGTEAAFHPIEEYMVHVEEHFQLLARRMQVDKKRVYLATDDPSLLKEAKTKYPNYEFISDNSISWSAGLHNRYTENSLRGVILDIHFLSQADFLVCTFSSQVCRVAYEIMQTLHPDASANFHSLDDIYYFGGQNAHNQIAIYAHQPRTADEIPMEPGDIIGVAGNHWDGYSKGVNRKLGRTGLYPSYKVREKIETVKYPTYPEAEK

SEQ ID NO: 9 - protein, 359 aa, synthetic construct
  UNSURE 1..359: note = HsFUT9
MTSTSKGILRPFLIVCIILGCFMACLLIYIKPTNSWIFSPMESASSVLKMKNFFSTKTDYFNETTILVWVWPFGQTFDLTSCQAMFNIQGCHLTTDRSLYNKSHAVLIHHRDISWDLTNLPQQARPPFQKWIWMNLESPTHTPQKSGIEHLFNLTLTYRRDSDIQVPYGFLTVSTNPFVFEVPSKEKLVCWVVSNWNPEHARVKYYNELSKSIEIHTYGQAFGEYVNDKNLIPTISTCKFYLSFENSIHKDYITEKLYNAFLAGSVPVVLGPSRENYENYIPADSFIHVEDYNSPSELAKYLKEVDKNNKLYLSYFNWRKDFTVNLPRFWESHACLACDHVKRHQEYKSVGNLEKWFWN

SEQ ID NO: 10 - protein, 479 aa, synthetic construct
  UNSURE 1..479: note = HsFUT10
MVRIQRRKLLASCLCVTATVFLLVTLQVMVELGKFERKEFKSSSLQDGHTKMEEAPTHLNSFLKKEGLTFNRKRKWELDSYPIMLWWSPLTGETGRLGQCGADACFFTINRTYLHHHMTKAFLFYGTDFNIDSLPLPRKAHHDWAVFHEESPKNNYKLFHKPVITLFNYTATFSRHSHLPLTTQYLESIEVLKSLRYLVPLQSKNKLRKRLAPLVYVQSDCDPPSDRDSYVRELMTYIEVDSYGECLRNKDLPQQLKNPASMDADGFYRIIAQYKFILAFENAVCDDYITEKFWRPLKLGVVPVYYGSPSITDWLPSNKSAILVSEFSHPRELASYIRRLDSDDRLYEAYVEWKLKGEISNQRLLTALRERKWGVQDVNQDNYIDAFECMVCTKVWANIRLQEKGLPPKRWEAEDTHLSCPEPTVFAFSPLRTPPLSSLREMWISSFEQSKKEAQALRWLVDRNQNFSSQEFWGLVFKD

SEQ ID NO: 11 - protein, 492 aa, synthetic construct
  UNSURE 1..492: note = HsFUT11
MAAGPIRVVLVLLGVLSVCAASGHGSVAEREAGGEAEWAEPWDGAVFRPPSALGAVGVTRSSGTPRPGREEAGDLPVLLWWSPGLFPHFPGDSERIECARGACVASRNRRALRDSRTRALLFYGTDFRASAAPLPRLAHQSWALLHEESPLNNFLLSHGPGIRLFNLTSTFSRHSDYPLSLQWLPGTAYLRRPVPPPMERAEWRRRGYAPLLYLQSHCDVPADRDRYVRELMRHIPVDSYGKCLQNRELPTARLQDTATATTEDPELLAFLSRYKFHLALENAICNDYMTEKLWRPMHLGAVPVYRGSPSVRDWMPNNHSVILIDDFESPQKLAEFIDFLDKNDEEYMKYLAYKQPGGITNQFLLDSLKHREWGVNDPLLPNYLNGFECFVCDYELARLDAEKAHAASPGDSPVFEPHIAQPSHMDCPVPTPGFGNVEEIPENDSWKEMWLQDYWQGLDQGEALTAMIHNNETEQTKFWDYLHEIFMKRQHL

SEQ ID NO: 12 - protein, 388 aa, synthetic construct
  UNSURE 1..388: note = HsPOFUT1
MGAAAWARPLSVSFLLLLLPLPGMPAGSWDPAGYLLYCPCMGRFGNQADHFLGSLAFAKLLNRTLAVPPWIEYQHHKPPFTNLHVSYQKYFKLEPLQAYHRVISLEDFMEKLAPTHWPPEKRVAYCFEVAAQRSPDKKTCPMKEGNPFGPFWDQFHVSFNKSELFTGISFSASYREQWSQRFSPKEHPVLALPGAPAQFPVLEEHRPLQKYMVWSDEMVKTGEAQIHAHLVRPYVGIHLRIGSDWKNACAMLKDGTAGSHFMASPQCVGYSRSTAAPLTMTMCLPDLKEIQRAVKLWVRSLDAQSVYVATDSESYVPELQQLFKGKVKVVSLKPEVAQVDLYILGQADHFIGNCVSSFTAFVKRERDLQGRPSSFFGMDRPPKLRDEF

SEQ ID NO: 13 - protein, 340 aa, synthetic construct
  UNSURE 1..340: note = HsST3Gal1
MVTLRKRTLKVLTFLVLFIFLTSFFLNYSHTMVATTWFPKQMVLELSENLKRLIKHRPCTCTHCIGQRKLSAWFDERFNQTMQPLLTAQNALLEDDTYRWWLRLQREKKPNNLNDTIKELFRVVPGNVDPMLEKRSVGCRRCAVVGNSGNLRESSYGPEIDSHDFVLRMNKAPTAGFEADVGTKTTHHLVYPESFRELGDNVSMILVPFKTIDLEWVVSAITTGTISHTYIPVPAKIRVKQDKILIYHPAFIKYVFDNWLQGHGRYPSTGILSVIFSMHVCDEVDLYGFGADSKGNWHHYWENNPSAGAFRKTGVHDADFESNVTATLASINKIRIFKGR

SEQ ID NO: 14 - protein, 375 aa, synthetic construct
  UNSURE 1..375: note = HsST3Gal3
MGLLVFVRNLLLALCLFLVLGFLYYSAWKLHLLQWEEDSNSVVLSFDSAGQTLGSEYDRLGFLLNLDSKLPAELATKYANFSEGACKPGYASALMTAIFPRFSKPAPMFLDDSFRKWARIREFVPPFGIKGQDNLIKAILSVTKEYRLTPALDSLRCRRCIIVGNGGVLANKSLGSRIDDYDIVVRLNSAPVKGFEKDVGSKTTLRITYPEGAMQRPEQYERDSLFVLAGFKWQDFKWLKYIVYKERVSASDGFWKSVATRVPKEPPEIRILNPYFIQEAAFTLIGLPFNNGLMGRGNIPTLGSVAVTMALHGCDEVAVAGFGYDMSTPNAPLHYYETVRMAAIKESWTHNIQREKEFLRKLVKARVITDLSSGI

SEQ ID NO: 15 - protein, 333 aa, synthetic construct
  UNSURE 1..333: note = HsST3Gal4
MVSKSRWKLLAMLALVLVVMVWYSISREDRYIELFYFPIPEKKEPCLQGEAESKASKLFGNYSRDQPIFLRLEDYFWVKTPSAYELPYGTKGSEDLLLRVLAITSSSIPKNIQSLRCRRCVVVGNGHRLRNSSLGDAINKYDVVIRLNNAPVAGYEGDVGSKTTMRLFYPESAHFDPKVENNPDTLLVLVAFKAMDFHWIETILSDKKRVRKGFWKQPPLIWDVNPKQIRILNPFFMEIAADKLLSLPMQQPRKIKQKPTTGLLAITLALHLCDLVHIAGFGYPDAYNKKQTIHYYEQITLKSMAGSGHNVSQEALAIKRMLEMGAIKNLTSF

SEQ ID NO: 16 - protein, 331 aa, synthetic construct
  UNSURE 1..331: note = HsST3Gal6
MRGYLVAIFLSAVFLYYVLHCILWGTNVYWVAPVEMKRRNKIQPCLSKPAFASLLRFHQFHPFLCAADFRKIASLYGSDKFDLPYGMRTSAEYFRLALSKLQSCDLFDEFDNIPCKKCVVVGNGGVLKNKTLGEKIDSYDVIIRMNNGPVLGHEEEVGRRTTFRLFYPESVFSDPIHNDPNTTVILTAFKPHDLRWLLELLMGDKINTNGFWKKPALNLIYKPYQIRILDPFIIRTAAYELLHFPKVFPKNQKPKHPTTGIIAITLAFYICHEVHLAGFKYNFSDLKSPLHYYGNATMSLMNKNAYHNVTAEQLFLKDIIEKNLVINLTQD

SEQ ID NO: 17 - protein, 406 aa, synthetic construct
  UNSURE 1..406: note = HsST6Gal1
MIHTNLKKKFSCCVLVFLLFAVICVWKEKKKGSYYDSFKLQTKEFQVLKSLGKLAMGSDSQSVSSSSTQDPHRGRQTLGSLRGLAKAKPEASFQVWNKDSSSKNLIPRLQKIWKNYLSMNKYKVSYKGPGPGIKFSAEALRCHLRDHVNVSMVEVTDFPFNTSEWEGYLPKESIRTKAGPWGRCAVVSSAGSLKSSQLGREIDDHDAVLRFNGAPTANFQQDVGTKTTIRLMNSQLVTTEKRFLKDSLYNEGILIVWDPSVYHSDIPKWYQNPDYNFFNNYKTYRKLHPNQPFYILKPQMPWELWDILQEISPEEIQPNPPSSGMLGIIIMMTLCDQVDIYEFLPSKRKTDVCYYYQKFFDSACTMGAYHPLLYEKNLVKHLNQGTDEDIYLLGKATLPGFRTIHC

SEQ ID NO: 18 - protein, 600 aa, synthetic construct
  UNSURE 1..600: note = HsST6GalNAc1
MRSCLWRCRHLSQGVQWSLLLAVLVFFLFALPSFIKEPQTKPSRHQRTENIKERSLQSLAKPKSQAPTRARRTTIYAEPVPENNALNTQTQPKAHTTGDRGKEANQAPPEEQDKVPHTAQRAAWKSPEKEKTMVNTLSPRGQDAGMASGRTEAQSWKSQDTKTTQGNGGQTRKLTASRTVSEKHQGKAATTAKTLIPKSQHRMLAPTGAVSTRTRQKGVTTAVIPPKEKKPQATPPPAPFQSPTTQRNQRLKAANFKSEPRWDFEEKYSFEIGGLQTTCPDSVKIKASKSLWLQKLFLPNLTLFLDSRHFNQSEWDRLEHFAPPFGFMELNYSLVQKVVTRFPPVPQQQLLLASLPAGSLRCITCAVVGNGGILNNSHMGQEIDSHDYVFRLSGALIKGYEQDVGTRTSFYGFTAFSLTQSLLILGNRGFKNVPLGKDVRYLHFLEGTRDYEWLEALLMNQTVMSKNLFWFRHRPQEAFREALHMDRYLLLHPDFLRYMKNRFLRSKTLDGAHWRIYRPTTGALLLLTALQLCDQVSAYGFITEGHERFSDHYYDTSWKRLIFYINHDFKLEREVWKRLHDEGIIRLYQRPGPGTAKAKN

SEQ ID NO: 19 - protein, 374 aa, synthetic construct
  UNSURE 1..374: note = HsST6GalNAc2
MGLPRGSFFWLLLLLTAACSGLLFALYFSAVQRYPGPAAGARDTTSFEAFFQSKASNSWTGKGQACRHLLHLAIQRHPHFRGLFNLSIPVLLWGDLFTPALWDRLSQHKAPYGWRGLSHQVIASTLSLLNGSESAKLFAPPRDTPPKCIRCAVVGNGGILNGSRQGPNIDAHDYVFRLNGAVIKGFERDVGTKTSFYGFTVNTMKNSLVSYWNLGFTSVPQGQDLQYIFIPSDIRDYVMLRSAILGVPVPEGLDKGDRPHAYFGPEASASKFKLLHPDFISYLTERFLKSKLINTHFGDLYMPSTGALMLLTALHTCDQVSAYGFITSNYWKFSDHYFERKMKPLIFYANHDLSLEAALWRDLHKAGILQLYQR

SEQ ID NO: 20 - protein, 302 aa, synthetic construct
  UNSURE 1..302: note = HsST6GalNAc4
MKAPGRLVLIILCSVVFSAVYILLCCWAGLPLCLATCLDHHFPTGSRPTVPGPLHFSGYSSVPDGKPLVREPCRSCAVVSSSGQMLGSGLGAEIDSAECVFRMNQAPTVGFEADVGQRSTLRVVSHTSVPLLLRNYSHYFQKARDTLYMVWGQGRHMDRVLGGRTYRTLLQLTRMYPGLQVYTFTERMMAYCDQIFQDETGKNRRQSGSFLSTGWFTMILALELCEEIVVYGMVSDSYCREKSHPSVPYHYFEKGRLDECQMYLAHEQAPRSAHRFITEKAVFSRWAKKRPIVFAHPSWRTE

SEQ ID NO: 21 - protein, 356 aa, synthetic construct
  UNSURE 1..356: note = HsST8Sia1
MSPCGRARRQTSRGAMAVLAWKFPRTRLPMGASALCVVVLCWLYIFPVYRLPNEKEIVQGVLQQGTAWRRNQTAARAFRKQMEDCCDPAHLFAMTKMNSPMGKSMWYDGEFLYSFTIDNSTYSLFPQATPFQLPLKKCAVVGNGGILKKSGCGRQIDEANFVMRCNLPPLSSEYTKDVGSKSQLVTANPSIIRQRFQNLLWSRKTFVDNMKIYNHSYIYMPAFSMKTGTEPSLRVYYTLSDVGANQTVLFANPNFLRSIGKFWKSRGIHAKRLSTGLFLVSAALGLCEEVAIYGFWPFSVNMHEQPISHHYYDNVLPFSGFHAMPEEFLQLWYLHKIGALRMQLDPCEDTSLQPTS

SEQ ID NO: 22 - protein, 375 aa, synthetic construct
  UNSURE 1..375: note = HsST8Sia2
MQLQFRSWMLAALTLLVVFLIFADISEIEEEIGNSGGRGTIRSAVNSLHSKSNRAEVVINGSSSPAVVDRSNESIKHNIQPASSKWRHNQTLSLRIRKQILKFLDAEKDISVLKGTLKPGDIIHYIFDRDSTMNVSQNLYELLPRTSPLKNKHFGTCAIVGNSGVLLNSGCGQEIDAHSFVIRCNLAPVQEYARDVGLKTDLVTMNPSVIQRAFEDLVNATWREKLLQRLHSLNGSILWIPAFMARGGKERVEWVNELILKHHVNVRTAYPSLRLLHAVRGYWLTNKVHIKRPTTGLLMYTLATRFCKQIYLYGFWPFPLDQNQNPVKYHYYDSLKYGYTSQASPHTMPLEFKALKSLHEQGALKLTVGQCDGAT

SEQ ID NO: 23 - protein, 380 aa, synthetic construct
  UNSURE 1..380: note = HsST8Sia3
MRNCKMARVASVLGLVMLSVALLILSLISYVSLKKENIFTTPKYASPGAPRMYMFHAGFRSQFALKFLDPSFVPITNSLTQELQEKPSKWKFNRTAFLHQRQEILQHVDVIKNFSLTKNSVRIGQLMHYDYSSHKYVFSISNNFRSLLPDVSPIMNKHYNICAVVGNSGILTGSQCGQEIDKSDFVFRCNFAPTEAFQRDVGRKTNLTTFNPSILEKYYNNLLTIQDRNNFFLSLKKLDGAILWIPAFFFHTSATVTRTLVDFFVEHRGQLKVQLAWPGNIMQHVNRYWKNKHLSPKRLSTGILMYTLASAICEEIHLYGFWPFGFDPNTREDLPYHYYDKKGTKFTTKWQESHQLPAEFQLLYRMHGEGLTKLTLSHCA

SEQ ID NO: 24 - protein, 359 aa, synthetic construct
  UNSURE 1..359: note = HsST8Sia4
MRSIRKRWTICTISLLLIFYKTKEIARTEEHQETQLIGDGELSLSRSLVNSSDKIIRKAGSSIFQHNVEGWKINSSLVLEIRKNILRFLDAERDVSVVKSSFKPGDVIHYVLDRRRTLNISHDLHSLLPEVSPMKNRRFKTCAVVGNSGILLDSECGKEIDSHNFVIRCNLAPVVEFAADVGTKSDFITMNPSVVQRAFGGFRNESDREKFVHRLSMLNDSVLWIPAFMVKGGEKHVEWVNALILKNKLKVRTAYPSLRLIHAVRGYWLTNKVPIKRPSTGLLMYTLATRFCDEIHLYGFWPFPKDLNGKAVKYHYYDDLKYRYFSNASPHRMPLEFKTLNVLHNRGALKLTTGKCVKQ

SEQ ID NO: 25 - protein, 559 aa, synthetic construct
  UNSURE 1..559: note = HsGalNAcT1
MRKFAYCKVVLATSLIWVLLDMFLLLYFSECNKCDEKKERGLPAGDVLEPVQKPHEGPGEMGKPVVIPKEDQEKMKEMFKINQFNLMASEMIALNRSLPDVRLEGCKTKVYPDNLPTTSVVIVFHNEAWSTLLRTVHSVINRSPRHMIEEIVLVDDASERDFLKRPLESYVKKLKVPVHVIRMEQRSGLIRARLKGAAVSKGQVITFLDAHCECTVGWLEPLLARIKHDRRTVVCPIIDVISDDTFEYMAGSDMTYGGFNWKLNFRWYPVPQREMDRRKGDRTLPVRTPTMAGGLFSIDRDYFQEIGTYDAGMDIWGGENLEISFRIWQCGGTLEIVTCSHVGHVFRKATPYTFPGGTGQIINKNNRRLAEVWMDEFKNFFYIISPGVTKVDYGDISSRVGLRHKLQCKPFSWYLENIYPDSQIPRHYFSLGEIRNVETNQCLDNMARKENEKVGIFNCHGMGGNQVFSYTANKEIRTDDLCLDVSKLNGPVTMLKCHHLKGNQLWEYDPVKLTLQHVNSNQCLDKATEEDSQVPSIRDCNGSRSQQWLLRNVTLPEIF

SEQ ID NO: 26 - protein, 571 aa, synthetic construct
  UNSURE 1..571: note = HsGalNAcT2
MRRRSRMLLCFAFLWVLGIAYYMYSGGGSALAGGAGGGAGRKEDWNEIDPIKKKDLHHSNGEEKAQSMETLPPGKVRWPDFNQEAYVGGTMVRSGQDPYARNKFNQVESDKLRMDRAIPDTRHDQCQRKQWRVDLPATSVVITFHNEARSALLRTVVSVLKKSPPHLIKEIILVDDYSNDPEDGALLGKIEKVRVLRNDRREGLMRSRVRGADAAQAKVLTFLDSHCECNEHWLEPLLERVAEDRTRVVSPIIDVINMDNFQYVGASADLKGGFDWNLVFKWDYMTPEQRRSRQGNPVAPIKTPMIAGGLFVMDKFYFEELGKYDMMMDVWGGENLEISFRVWQCGGSLEIIPCSRVGHVFRKQHPYTFPGGSGTVFARNTRRAAEVWMDEYKNFYYAAVPSARNVPYGNIQSRLELRKKLSCKPFKWYLENVYPELRVPDHQDIAFGALQQGTNCLDTLGHFADGVVGVYECHNAGGNQEWALTKEKSVKHMDLCLTVVDRAPGSLIKLQGCRENDSRQKWEQIEGNSKLRHVGSNLCLDSRTAKSGGLSVEVCGPALSQQWKFTLNLQQ

SEQ ID NO: 27 - protein, 633 aa, synthetic construct
  UNSURE 1..633: note = HsGalNAcT3
MAHLKRLVKLHIKRHYHKKFWKLGAVIFFFIIVLVLMQREVSVQYSKEESRMERNMKNKNKMLDLMLEAVNNIKDAMPKMQIGAPVRQNIDAGERPCLQGYYTAAELKPVLDRPPQDSNAPGASGKAFKTTNLSVEEQKEKERGEAKHCFNAFASDRISLHRDLGPDTRPPECIEQKFKRCPPLPTTSVIIVFHNEAWSTLLRTVHSVLYSSPAILLKEIILVDDASVDEYLHDKLDEYVKQFSIVKIVRQRERKGLITARLLGATVATAETLTFLDAHCECFYGWLEPLLARIAENYTAVVSPDIASIDLNTFEFNKPSPYGSNHNRGNFDWSLSFGWESLPDHEKQRRKDETYPIKTPTFAGGLFSISKEYFEYIGSYDEEMEIWGGENIEMSFRVWQCGGQLEIMPCSVVGHVFRSKSPHSFPKGTQVIARNQVRLAEVWMDEYKEIFYRRNTDAAKIVKQKAFGDLSKRFEIKHRLQCKNFTWYLNNIYPEVYVPDLNPVISGYIKSVGQPLCLDVGENNQGGKPLIMYTCHGLGGNQYFEYSAQHEIRHNIQKELCLHAAQGLVQLKACTYKGHKTVVTGEQIWEIQKDQLLYNPFLKMCLSANGEHPSLVSCNPSDPLQKWILSQND

SEQ ID NO: 28 - protein, 578 aa, synthetic construct
  UNSURE 1..578: note = HsGalNAcT4
MAVRWTWAGKSCLLLAFLTVAYIFVELLVSTFHASAGAGRARELGSRRLSDLQKNTEDLSRPLYKKPPADSRALGEWGKASKLQLNEDELKQQEELIERYAINIYLSDRISLHRHIEDKRMYECKSQKFNYRTLPTTSVIIAFYNEAWSTLLRTIHSVLETSPAVLLKEIILVDDLSDRVYLKTQLETYISNLDRVRLIRTNKREGLVRARLIGATFATGDVLTFLDCHCECNSGWLEPLLERIGRDETAVVCPVIDTIDWNTFEFYMQIGEPMIGGFDWRLTFQWHSVPKQERDRRISRIDPIRSPTMAGGLFAVSKKYFQYLGTYDTGMEVWGGENLELSFRVWQCGGKLEIHPCSHVGHVFPKRAPYARPNFLQNTARAAEVWMDEYKEHFYNRNPPARKEAYGDISERKLLRERLRCKSFDWYLKNVFPNLHVPEDRPGWHGAIRSRGISSECLDYNSPDNNPTGANLSLFGCHGQGGNQFFEYTSNKEIRFNSVTELCAEVPEQKNYVGMQNCPKDGFPVPANIIWHFKEDGTIFHPHSGLCLSAYRTPEGRPDVQMRTCDALDKNQIWSFEK

SEQ ID NO: 29 - protein, 940 aa, synthetic construct
  UNSURE 1..940: note = HsGalNAcT5
MNRIRKFFRGSGRVLAFIFVASVIWLLFDMAALRLSFSEINTRVIKEDIVRRERIGFRVQPDQGKIFYSSIKEMKPPLRGHGKGAWGKENVRKTEESVLKVEVDLDQTQRERKMQNALGRGKVVPLWHPAHLQTLPVTPNKQKTDGRGTKPEASSHQGTPKQTTAQGAPKTSFIAAKGTQVVKISVHMGRVSLKQEPRKSHSPSSDTSKLAAERDLNVTISLSTDRPKQRSQAVANERAHPASTAVPKSGEAMALNKTKTQSKEVNANKHKANTSLPFPKFTVNSNRLRKQSINETPLGSLSKDDGARGAHGKKLNFSESHLVIITKEEEQKADPKEVSNSKTKTIFPKVLGKSQSKHISRNRSEMSSSSLAPHRVPLSQTNHALTGGLEPAKINITAKAPSTEYNQSHIKALLPEDSGTHQVLRIDVTLSPRDPKAPGQFGRPVVVPHGKEKEAERRWKEGNFNVYLSDLIPVDRAIEDTRPAGCAEQLVHNNLPTTSVIMCFVDEVWSTLLRSVHSVINRSPPHLIKEILLVDDFSTKDYLKDNLDKYMSQFPKVRILRLKERHGLIRARLAGAQNATGDVLTFLDSHVECNVGWLEPLLERVYLSRKKVACPVIEVINDKDMSYMTVDNFQRGIFVWPMNFGWRTIPPDVIAKNRIKETDTIRCPVMAGGLFSIDKSYFFELGTYDPGLDVWGGENMELSFKVWMCGGEIEIIPCSRVGHIFRNDNPYSFPKDRMKTVERNLVRVAEVWLDEYKELFYGHGDHLIDQGLDVGNLTQQRELRKKLKCKSFKWYLENVFPDLRAPIVRASGVLINVALGKCISIENTTVILEDCDGSKELQQFNYTWLRLIKCGEWCIAPIPDKGAVRLHPCDNRNKGLKWLHKSTSVFHPELVNHIVFENNQQLLCLEGNFSQKILKVAACDPVKPYQKWKFEKYYEA

SEQ ID NO: 30 - protein, 622 aa, synthetic construct
  UNSURE 1..622: note = HsGalNAcT6
MRLLRRRHMPLRLAMVGCAFVLFLFLLHRDVSSREEATEKPWLKSLVSRKDHVLDLMLEAMNNLRDSMPKLQIRAPEAQQTLFSINQSCLPGFYTPAELKPFWERPPQDPNAPGADGKAFQKSKWTPLETQEKEEGYKKHCFNAFASDRISLQRSLGPDTRPPECVDQKFRRCPPLATTSVIIVFHNEAWSTLLRTVYSVLHTTPAILLKEIILVDDASTEEHLKEKLEQYVKQLQVVRVVRQEERKGLITARLLGASVAQAEVLTFLDAHCECFHGWLEPLLARIAEDKTVVVSPDIVTIDLNTFEFAKPVQRGRVHSRGNFDWSLTFGWETLPPHEKQRRKDETYPIKSPTFAGGLFSISKSYFEHIGTYDNQMEIWGGENVEMSFRVWQCGGQLEIIPCSVVGHVFRTKSPHTFPKGTSVIARNQVRLAEVWMDSYKKIFYRRNLQAAKMAQEKSFGDISERLQLREQLHCHNFSWYLHNVYPEMFVPDLTPTFYGAIKNLGTNQCLDVGENNRGGKPLIMYSCHGLGGNQYFEYTTQRDLRHNIAKQLCLHVSKGALGLGSCHFTGKNSQVPKDEEWELAQDQLIRNSGSGTCLTSQDKKPAMAPCNPSDPHQLWLFV

SEQ ID NO: 31 - protein, 657 aa, synthetic construct
  UNSURE 1..657: note = HsGalNAcT7
MRLKIGFILRSLLVVGSFLGLVVLWSSLTPRPDDPSPLSRMREDRDVNDPMPNRGGNGLAPGEDRFKPVVPWPHVEGVEVDLESIRRINKAKNEQEHHAGGDSQKDIMQRQYLTFKPQTFTYHDPVLRPGILGNFEPKEPEPPGVVGGPGEKAKPLVLGPEFKQAIQASIKEFGFNMVASDMISLDRSVNDLRQEECKYWHYDENLLTSSVVIVFHNEGWSTLMRTVHSVIKRTPRKYLAEIVLIDDFSNKEHLKEKLDEYIKLWNGLVKVFRNERREGLIQARSIGAQKAKLGQVLIYLDAHCEVAVNWYAPLVAPISKDRTICTVPLIDVINGNTYEIIPQGGGDEDGYARGAWDWSMLWKRVPLTPQEKRLRKTKTEPYRSPAMAGGLFAIEREFFFELGLYDPGLQIWGGENFEISYKIWQCGGKLLFVPCSRVGHIYRLEGWQGNPPPIYVGSSPTLKNYVRVVEVWWDEYKDYFYASRPESQALPYGDISELKKFREDHNCKSFKWFMEEIAYDITSHYPLPPKNVDWGEIRGFETAYCIDSMGKTNGGFVELGPCHRMGGNQLFRINEANQLMQYDQCLTKGADGSKVMITHCNLNEFKEWQYFKNLHRFTHIPSGKCLDRSEVLHQVFISNCDSSKTTQKWEMNNIHSV

SEQ ID NO: 32 - protein, 637 aa, synthetic construct
  UNSURE 1..637: note = HsGalNAcT8
MMFWRKLPKALFIGLTLAIAVNLLLVFSSKGTLQNLFTGGLHRELPLHLNKRYGAVIKRLSHLEVELQDLKESMKLALRQQENVNSTLKRAKDEVRPLLKAMETKVNETKKHKTQMKLFPHSQLFRQWGEDLSEAQQKAAQDLFRKFGYNAYLSNQLPLNRTIPDTRDYRCLRKTYPSQLPSLSVILIFVNEALSIIQRAITSIINRTPSRLLKEIILVDDFSSNGELKVHLDEKIKLYNQKYPGLLKIIRHPERKGLAQARNTGWEAATADVVAILDAHIEVNVGWAEPILARIQEDRTVIVSPVFDNIRFDTFKLDKYELAVDGFNWELWCRYDALPQAWIDLHDVTAPVKSPSIMGILAANRHFLGEIGSLDGGMLIYGGENVELSLRVWQCGGKVEILPCSRIAHLERHHKPYALDLTAALKRNALRVAEIWMDEHKHMVYLAWNIPLQNSGIDFGDVSSRMALREKLKCKTFDWYLKNVYPLLKPLHTIVGYGRMKNLLDENVCLDQGPVPGNTPIMYYCHEFSSQNVYYHLTGELYVGQLIAEASASDRCLTDPGKAEKPTLEPCSKAAKNRLHIYWDFKPGGAVINRDTKRCLEMKKDLLGSHVLVLQTCSTQVWEIQHTVRDWGQTNSQ

SEQ ID NO: 33 - protein, 603 aa, synthetic construct
  UNSURE 1..603: note = HsGalNAcT9
MAVARKIRTLLTVNILVFVGIVLFSVYCRLQGRSQELVRIVSGDRRVRSRHAKVGTLGDREAILQRLDHLEEVVYNQLNGLAKPIGLVEGPGGLGQGGLAATLRDDGQEAEGKYEEYGYNAQLSDRISLDRSIPDYRPRKCRQMSYAQDLPQVSVVFIFVNEALSVILRSVHSVVNHTPSQLLKEVILVDDNSDNVELKFNLDQYVNKRYPGLVKIVRNSRREGLIRARLQGWKAATAPVVGFFDAHVEFNTGWAEPALSRIREDRRRIVLPAIDNIKYSTFEVQQYANAAHGYNWGLRCMYIIPPQDWLDRGDESAPIRTPAMIGCSFVVDREYFGDIGLLDPGMEVYGGENVELGMRVWQCGGSMEVLPCSRVAHIERTRKPYNNDIDYYAKRNALRAAEVWMDDFKSHVYMAWNIPMSNPGVDFGDVSERLALRQRLKCRSFKWYLENVYPEMRVYNNTLTYGEVRNSKASAYCLDQGAEDGDRAILYPCHGMSSQLVRYSADGLLQLGPLGSTAFLPDSKCLVDDGTGRMPTLKKCEDVARPTQRLWDFTQSGPIVSRATGRCLEVEMSKDANFGLRLVVQRCSGQKWMIRNWIKHARH

SEQ ID NO: 34 - protein, 603 aa, synthetic construct
  UNSURE 1..603: note = HsGalNAcT10
MRRKEKRLLQAVALVLAALVLLPNVGLWALYRERQPDGTPGGSGAAVAPAAGQGSHSRQKKTFFLGDGQKLKDWHDKEAIRRDAQRVGNGEQGRPYPMTDAERVDQAYRENGFNIYVSDKISLNRSLPDIRHPNCNSKRYLETLPNTSIIIPFHNEGWSSLLRTVHSVLNRSPPELVAEIVLVDDFSDREHLKKPLEDYMALFPSVRILRTKKREGLIRTRMLGASVATGDVITFLDSHCEANVNWLPPLLDRIARNRKTIVCPMIDVIDHDDFRYETQAGDAMRGAFDWEMYYKRIPIPPELQKADPSDPFESPVMAGGLFAVDRKWFWELGGYDPGLEIWGGEQYEISFKVWMCGGRMEDIPCSRVGHIYRKYVPYKVPAGVSLARNLKRVAEVWMDEYAEYIYQRRPEYRHLSAGDVAVQKKLRSSLNCKSFKWFMTKIAWDLPKFYPPVEPPAAAWGEIRNVGTGLCADTKHGALGSPLRLEGCVRGRGEAAWNNMQVFTFTWREDIRPGDPQHTKKFCFDAISHTSPVTLYDCHSMKGNQLWKYRKDKTLYHPVSGSCMDCSESDHRIFMNTCNPSSLTQQWLFEHTNSTVLEKFNRN

SEQ ID NO: 35 - protein, 331 aa, synthetic construct
  UNSURE 1..331: note = HsB3GALNT1
MASALWTVLPSRMSLRSLKWSLLLLSLLSFFVMWYLSLPHYNVIERVNWMYFYEYEPIYRQDFHFTLREHSNCSHQNPFLVILVTSHPSDVKARQAIRVTWGEKKSWWGYEVLTFFLLGQEAEKEDKMLALSLEDEHLLYGDIIRQDFLDTYNNLTLKTIMAFRWVTEFCPNAKYVMKTDTDVFINTGNLVKYLLNLNHSEKFFTGYPLIDNYSYRGFYQKTHISYQEYPFKVFPPYCSGLGYIMSRDLVPRIYEMMGHVKPIKFEDVYVGICLNLLKVNIHIPEDTNLFFLYRIHLDVCQLRRVIAAHGFSSKEIITFWQVMLRNTTCHY

SEQ ID NO: 36 - protein, 533 aa, synthetic construct
  UNSURE 1..533: note = HsB4GALNT1
MWLGRRALCALVLLLACASLGLLYASTRDAPGLRLPLAPWAPPQSPRRPELPDLAPEPRYAHIPVRIKEQVVGLLAWNNCSCESSGGGLPLPFQKQVRAIDLTKAFDPAELRAASATREQEFQAFLSRSQSPADQLLIAPANSPLQYPLQGVEVQPLRSILVPGLSLQAASGQEVYQVNLTASLGTWDVAGEVTGVTLTGEGQADLTLVSPGLDQLNRQLQLVTYSSRSYQTNTADTVRFSTEGHEAAFTIRIRHPPNPRLYPPGSLPQGAQYNISALVTIATKTFLRYDRLRALITSIRRFYPTVTVVIADDSDKPERVSGPYVEHYLMPFGKGWFAGRNLAVSQVTTKYVLWVDDDFVFTARTRLERLVDVLERTPLDLVGGAVREISGFATTYRQLLSVEPGAPGLGNCLRQRRGFHHELVGFPGCVVTDGVVNFFLARTDKVREVGFDPRLSRVAHLEFFLDGLGSLRVGSCSDVVVDHASKLKLPWTSRDAGAETYARYRYPGSLDESQMAKHRLLFFKHRLQCMTSQ

SEQ ID NO: 37 - protein, 354 aa, synthetic construct
  UNSURE 1..354: note = HsA-ABO
MAEVLRTLAGKPKCHALRPMILFLIMLVLVLFGYGVLSPRSLMPGSLERGFCMAVREPDHLQRVSLPRMVYPQPKVLTPCRKDVLVVTPWLAPIVWEGTFNIDILNEQFRLQNTTIGLTVFAIKKYVAFLKLFLETAEKHFMVGHRVHYYVFTDQPAAVPRVTLGTGRQLSVLEVRAYKRWQDVSMRRMEMISDFCERRFLSEVDYLVCVDVDMEFRDHVGVEILTPLFGTLHPGFYGSSREAFTYERRPQSQAYIPKDEGDFYYLGGFFGGSVQEVQRLTRACHQAMMVDQANGIEAVWHDESHLNKYLLRHKPTKVLSPEYLWDQQLLGWPAVLRKLRFTAVPKNHQAVRNP

SEQ ID NO: 38 - protein, 353 aa, synthetic construct
  UNSURE 1..353: note = HsA4GALT
MSKPPDLLLRLLRGAPRQRVCTLFIIGFKFTFFVSIMIYWHVVGEPKEKGQLYNLPAEIPCPTLTPPTPPSHGPTPGNIFFLETSDRTNPNFLFMCSVESAARTHPESHVLVLMKGLPGGNASLPRHLGISLLSCFPNVQMLPLDLRELFRDTPLADWYAAVQGRWEPYLLPVLSDASRIALMWKFGGIYLDTDFIVLKNLRNLTNVLGTQSRYVLNGAFLAFERRHEFMALCMRDFVDHYNGWIWGHQGPQLLTRVFKKWCSIRSLAESRACRGVTTLPPEAFYPIPWQDWKKYFEDINPEELPRLLSATYAVHVWNKKSQGTRFEATSRALLAQLHARYCPTTHEAMKMYL

SEQ ID NO: 39 - protein, 326 aa, synthetic construct
  UNSURE 1..326: note = HsB3GalT1
MASKVSCLYVLTVVCWASALWYLSITRPTSSYTGSKPFSHLTVARKNFTFGNIRTRPINPHSFEFLINEPNKCEKNIPFLVILISTTHKEFDARQAIRETWGDENNFKGIKIATLFLLGKNADPVLNQMVEQESQIFHDIIVEDFIDSYHNLTLKTLMGMRWVATFCSKAKYVMKTDSDIFVNMDNLIYKLLKPSTKPRRRYFTGYVINGGPIRDVRSKWYMPRDLYPDSNYPPFCSGTGYIFSADVAELIYKTSLHTRLLHLEDVYVGLCLRKLGIHPFQNSGFNHWKMAYSLCRYRRVITVHQISPEEMHRIWNDMSSKKHLRC

SEQ ID NO: 40 - protein, 422 aa, synthetic construct
  UNSURE 1..422: note = HsB3GalT2
MLQWRRRHCCFAKMTWNAKRSLFRTHLIGVLSLVFLFAMFLFFNHHDWLPGRAGFKENPVTYTFRGFRSTKSETNHSSLRNIWKETVPQTLRPQTATNSNNTDLSPQGVTGLENTLSANGSIYNEKGTGHPNSYHFKYIINEPEKCQEKSPFLILLIAAEPGQIEARRAIRQTWGNESLAPGIQITRIFLLGLSIKLNGYLQRAILEESRQYHDIIQQEYLDTYYNLTIKTLMGMNWVATYCPHIPYVMKTDSDMFVNTEYLINKLLKPDLPPRHNYFTGYLMRGYAPNRNKDSKWYMPPDLYPSERYPVFCSGTGYVFSGDLAEKIFKVSLGIRRLHLEDVYVGICLAKLRIDPVPPPNEFVFNHWRVSYSSCKYSHLITSHQFQPSELIKYWNHLQQNKHNACANAAKEKAGRYRHRKLH

SEQ ID NO: 41 - protein, 398 aa, synthetic construct
  UNSURE 1..398: note = HsB4GalT1
MRLREPLLSGSAAMPGASLQRACRLLVAVCALHLGVTLVYYLAGRDLSRLPQLVGVSTPLQGGSNSAAAIGQSSGELRTGGARPPPPLGASSQPRPGGDSSPVVDSGPGPASNLTSVPVPHTTALSLPACPEESPLLVGPMLIEFNMPVDLELVAKQNPNVKMGGRYAPRDCVSPHKVAIIIPFRNRQEHLKYWLYYLHPVLQRQQLDYGIYVINQAGDTIFNRAKLLNVGFQEALKDYDYTCFVFSDVDLIPMNDHNAYRCFSQPRHISVAMDKFGFSLPYVQYFGGVSALSKQQFLTINGFPNNYWGWGGEDDDIFNRLVFRGMSISRPNAVVGRCRMIRHSRDKKNEPNPQRFDRIAHTKETMLSDGLNSLTYQVLDVQRYPLYTQITVDIGTPS

SEQ ID NO: 42 - protein, 372 aa, synthetic construct
  UNSURE 1..372: note = HsB4GalT2
MSRLLGGTLERVCKAVLLLCLLHFLVAVILYFDVYAQHLAFFSRFSARGPAHALHPAASSSSSSSNCSRPNATASSSGLPEVPSALPGPTAPTLPPCPDSPPGLVGRLLIEFTSPMPLERVQRENPGVLMGGRYTPPDCTPAQTVAVIIPFRHREHHLRYWLHYLHPILRRQRLRYGVYVINQHGEDTFNRAKLLNVGFLEALKEDAAYDCFIFSDVDLVPMDDRNLYRCGDQPRHFAIAMDKFGFRLPYAGYFGGVSGLSKAQFLRINGFPNEYWGWGGEDDDIFNRISLTGMKISRPDIRIGRYRMIKHDRDKHNEPNPQRFTKIQNTKLTMKRDGIGSVRYQVLEVSRQPLFTNITVDIGRPPSWPPRG

SEQ ID NO: 43 - protein, 393 aa, synthetic construct
  UNSURE 1..393: note = HsB4GalT3
MLRRLLERPCTLALLVGSQLAVMMYLSLGGFRSLSALFGRDQGPTFDYSHPRDVYSNLSHLPGAPGGPPAPQGLPYCPERSPLLVGPVSVSFSPVPSLAEIVERNPRVEPGGRYRPAGCEPRSRTAIIVPHRAREHHLRLLLYHLHPFLQRQQLAYGIYVIHQAGNGTFNRAKLLNVGVREALRDEEWDCLFLHDVDLLPENDHNLYVCDPRGPRHVAVAMNKFGYSLPYPQYFGGVSALTPDQYLKMNGFPNEYWGWGGEDDDIATRVRLAGMKISRPPTSVGHYKMVKHRGDKGNEENPHRFDLLVRTQNSWTQDGMNSLTYQLLARELGPLYTNITADIGTDPRGPRAPSGPRYPPGSSQAFRQEMLQRRPPARPGPLSTANHTALRGSH

SEQ ID NO: 44 - protein, 344 aa, synthetic construct
  UNSURE 1..344: note = HsB4GalT4
MGFNLTFHLSYKFRLLLLLTLCLTVVGWATSNYFVGAIQEIPKAKEFMANFHKTLILGKGKTLTNEASTKKVELDNCPSVSPYLRGQSKLIFKPDLTLEEVQAENPKVSRGRYRPQECKALQRVAILVPHRNREKHLMYLLEHLHPFLQRQQLDYGIYVIHQAEGKKFNRAKLLNVGYLEALKEENWDCFIFHDVDLVPENDFNLYKCEEHPKHLVVGRNSTGYRLRYSGYFGGVTALSREQFFKVNGFSNNYWGWGGEDDDLRLRVELQRMKISRPLPEVGKYTMVFHTRDKGNEVNAERMKLLHQVSRVWRTDGLSSCSYKLVSVEHNPLYINITVDFWFGA

SEQ ID NO: 45 - protein, 388 aa, synthetic construct
  UNSURE 1..388: note = HsB4GalT5
MRARRGLLRLPRRSLLAALFFFSLSSSLLYFVYVAPGIVNTYLFMMQAQGILIRDNVRTIGAQVYEQVLRSAYAKRNSSVNDSDYPLDLNHSETFLQTTTFLPEDFTYFANHTCPERLPSMKGPIDINMSEIGMDYIHELFSKDPTIKLGGHWKPSDCMPRWKVAILIPFRNRHEHLPVLFRHLLPMLQRQRLQFAFYVVEQVGTQPFNRAMLFNVGFQEAMKDLDWDCLIFHDVDHIPESDRNYYGCGQMPRHFATKLDKYMYLLPYTEFFGGVSGLTVEQFRKINGFPNAFWGWGGEDDDLWNRVQNAGYSVSRPEGDTGKYKSIPHHHRGEVQFLGRYALLRKSKERQGLDGLNNLNYFANITYDALYKNITVNLTPELAQVNEY

SEQ ID NO: 46 - protein, 382 aa, synthetic construct
  UNSURE 1..382: note = HsB4GalT6
MSVLRRMMRVSNRSLLAFIFFFSLSSSCLYFIYVAPGIANTYLFMVQARGIMLRENVKTIGHMIRLYTNKNSTLNGTDYPEGNNSSDYLVQTTTYLPENFTYSPYLPCPEKLPYMRGFLNVNVSEVSFDEIHQLFSKDLDIEPGGHWRPKDCKPRWKVAVLIPFRNRHEHLPIFFLHLIPMLQKQRLEFAFYVIEQTGTQPFNRAMLFNVGFKEAMKDSVWDCVIFHDVDHLPENDRNYYGCGEMPRHFAAKLDKYMYILPYKEFFGGVSGLTVEQFRKINGFPNAFWGWGGEDDDLWNRVHYAGYNVTRPEGDLGKYKSIPHHHRGEVQFLGRYKLLRYSKERQYIDGLNNLIYRPKILVDRLYTNISVNLMPELAPIEDY

SEQ ID NO: 47 - protein, 354 aa, synthetic construct; identical to SEQ ID NO: 37
  UNSURE 1..354: note = HsB-ABO

SEQ ID NO: 48 - protein, 541 aa, synthetic construct
  UNSURE 1..541: note = HsUGT8
MKSYTPYFILLWSAVGIAKAAKIIIVPPIMFESHMYIFKTLASALHERGHHTVFLLSEGRDIAPSNHYSLQRYPGIFNSTTSDAFLQSKMRNIFSGRLTAIELFDILDHYTKNCDLMVGNHALIQGLKKEKFDLLLVDPNDMCGFVIAHLLGVKYAVFSTGLWYPAEVGAPAPLAYVPEFNSLLTDRMNLLQRMKNTGVYLISRLGVSFLVLPKYERIMQKYNLLPEKSMYDLVHGSSLWMLCTDVALEFPRPTLPNVVYVGGILTKPASPLPEDLQRWVNGANEHGFVLVSFGAGVKYLSEDIANKLAGALGRLPQKVIWRFSGPKPKNLGNNTKLIEWLPQNDLLGHSKIKAFLSHGGLNSIFETIYHGVPVVGIPLFGDHYDTMTRVQAKGMGILLEWKTVTEKELYEALVKVINNPSYRQRAQKLSEIHKDQPGHPVNRTIYWIDYIIRHNGAHHLRAAVHQISFCQYFLLDIAFVLLLGAALLYFLLSWVTKFIYRKIKSLWSRNKHSTVNGHYHNGILNGKYKRNGHIKHEKKVK

SEQ ID NO: 49 - protein, 363 aa, synthetic construct
  UNSURE 1..363: note = HsC1GLT
MASKSWLNFLTFLCGSAIGFLLCSQLFSILLGEKVDTQPNVLHNDPHARHSDDNGQNHLEGQMNFNADSSQHKDENTDIAENLYQKVRILCWVMTGPQNLEKKAKHVKATWAQRCNKVLFMSSEENKDFPAVGLKTKEGRDQLYWKTIKAFQYVHEHYLEDADWFLKADDDTYVILDNLRWLLSKYDPEEPIYFGRRFKPYVKQGYMSGGAGYVLSKEALKRFVDAFKTDKCTHSSSIEDLALGRCMEIMNVEAGDSRDTIGKETFHPFVPEHHLIKGYLPRTFWYWNYNYYPPVEGPGCCSDLAVSFHYVDSTTMYELEYLVYHLRPYGYLYRYQPTLPERILKEISQANKNEDTKVKLGNP

SEQ ID NO: 50 - protein, 318 aa, synthetic construct
  UNSURE 1..318: note = HsCOSMC
MLSESSSFLKGVMLGSIFCALITMLGHIRIGHGNRMHHHEHHHLQAPNKEDILKISEDERMELSKSFRVYCIILVKPKDVSLWAAVKETWTKHCDKAEFFSSENVKVFESINMDTNDMWLMMRKAYKYAFDKYRDQYNWFFLARPTTFAIIENLKYFLLKKDPSQPFYLGHTIKSGDLEYVGMEGGIVLSVESMKRLNSLLNIPEKCPEQGGMIWKISEDKQLAVCLKYAGVFAENAEDADGKDVFNTKSVGLSIKEAMTYHPNQVVEGCCSDMAVTFNGLTPNQMHVMMYGVYRLRAFGHIFNDALVFLPPNGSDND

SEQ ID NO: 51 - protein, 464 aa, synthetic construct
  UNSURE 1..464: note = HsAlg1
MAASCLVLLALCLLLPLLLLGGWKRWRRGRAARHVVAVVLGDVGRSPRMQYHALSLAMHGFSVTLLGFCNSKPHDELLQNNRIQIVGLTELQSLAVGPRVFQYGVKVVLQAMYLLWKLMWREPGAYIFLQNPPGLPSIAVCWFVGCLCGSKLVIDWHNYGYSIMGLVHGPNHPLVLLAKWYEKFFGRLSHLNLCVTNAMREDLADNWHIRAVTVYDKPASFFKETPLDLQHRLFMKLGSMHSPFRARSEPEDPVTERSAFTERDAGSGLVTRLRERPALLVSSTSWTEDEDFSILLAALEKFEQLTLDGHNLPSLVCVITGKGPLREYYSRLIHQKHFQHIQVCTPWLEAEDYPLLLGSADLGVCLHTSSSGLDLPMKVVDMFGCCLPVCAVNFKCLHELVKHEENGLVFEDSEELAAQLQMLFSNFPDPAGKLNQFRKNLRESQQLRWDESWVQTVLPLVMDT

SEQ ID NO: 52 - protein, 416 aa, synthetic construct
  UNSURE 1..416: note = HsAlg2
MAEEQGRERDSVPKPSVLFLHPDLGVGGAERLVLDAALALQARGCSVKIWTAHYDPGHCFAESRELPVRCAGDWLPRGLGWGGRGAAVCAYVRMVFLALYVLFLADEEFDVVVCDQVSACIPVFRLARRRKKILFYCHFPDLLLTKRDSFLKRLYRAPIDWIEEYTTGMADCILVNSQFTAAVFKETFKSLSHIDPDVLYPSLNVTSFDSVVPEKLDDLVPKGKKFLLLSINRYERKKNLTLALEALVQLRGRLTSQDWERVHLIVAGGYDERVLENVEHYQELKKMVQQSDLGQYVTFLRSFSDKQKISLLHSCTCVLYTPSNEHFGIVPLEAMYMQCPVIAVNSGGPLESIDHSVTGFLCEPDPVHFSEAIEKFIREPSLKATMGLAGRARVKEKFSPEAFTEQLYRYVTKLLV

SEQ ID NO: 53 - protein, 438 aa, synthetic construct
  UNSURE 1..438: note = HsAlg3
MAAGLRKRGRSGSAAQAEGLCKQWLQRAWQERRLLLREPRYTLLVAACLCLAEVGITFWVIHRVAYTEIDWKAYMAEVEGVINGTYDYTQLQGDTGPLVYPAGFVYIFMGLYYATSRGTDIRMAQNIFAVLYLATLLLVFLIYHQTCKVPPFVFFFMCCASYRVHSIFVLRLFNDPVAMVLLFLSINLLLAQRWGWGCCFFSLAVSVKMNVLLFAPGLLFLLLTQFGFRGALPKLGICAGLQVVLGLPFLLENPSGYLSRSFDLGRQFLFHWTVNWRFLPEALFLHRAFHLALLTAHLTLLLLFALCRWHRTGESILSLLRDPSKRKVPPQPLTPNQIVSTLFTSNFIGICFSRSLHYQFYVWYFHTLPYLLWAMPARWLTHLLRLLVLGLIELSWNTYPSTSCSSAALHICHAVILLQLWLGPQPFPKSTQHSKKAH

SEQ ID NO: 54 - protein, 492 aa, synthetic construct
  UNSURE 1..492: note = HsAlg11
MAAGERSWCLCKLLRFFYSLFFPGLIVCGTLCVCLVIVLWGIRLLLQRKKKLVSTSKNGKNQMVIAFFHPYCNAGGGGERVLWCALRALQKKYPEAVYVVYTGDVNVNGQQILEGAFRRFNIRLIHPVQFVFLRKRYLVEDSLYPHFTLLGQSLGSIFLGWEALMQCVPDVYIDSMGYAFTLPLFKYIGGCQVGSYVHYPTISTDMLSVVKNQNIGFNNAAFITRNPFLSKVKLIYYYLFAFIYGLVGSCSDVVMVNSSWTLNHILSLWKVGNCTNIVYPPCDVQTFLDIPLHEKKMTPGHLLVSVGQFRPEKNHPLQIRAFAKLLNKKMVESPPSLKLVLIGGCRNKDDELRVNQLRRLSEDLGVQEYVEFKINIPFDELKNYLSEATIGLHTMWNEHFGIGVVECMAAGTIILAHNSGGPKLDIVVPHEGDITGFLAESEEDYAETIAHILSMSAEKRLQIRKSARASVSRFSDQEFEVTFLSSVEKLFK

SEQ ID NO: 55 - protein, 488 aa, synthetic construct
  UNSURE 1..488: note = HsAlg12
MAGKGSSGRRPLLLGLLVAVATVHLVICPYTKVEESFNLQATHDLLYHWQDLEQYDHLEFPGVVPRTFLGPVVIAVFSSPAVYVLSLLEMSKFYSQLIVRGVLGLGVIFGLWTLQKEVRRHFGAMVATMFCWVTAMQFHLMFYCTRTLPNVLALPVVLLALAAWLRHEWARFIWLSAFAIIVFRVELCLFLGLLLLLALGNRKVSVVRALRHAVPAGILCLGLTVAVDSYFWRQLTWPEGKVLWYNTVLNKSSNWGTSPLLWYFYSALPRGLGCSLLFIPLGLVDRRTHAPTVLALGFMALYSLLPHKELRFIIYAFPMLNITAARGCSYLLNNYKKSWLYKAGSLLVIGHLVVNAAYSATALYVSHFNYPGGVAMQRLHQLVPPQTDVLLHIDVAAAQTGVSRFLQVNSAWRYDKREDVQPGTGMLAYTHILMEAAPGLLALYRDTHRVLASVVGTTGVSLNLTQLPPFNVHLQTKLVLLERLPRPS

SEQ ID NO: 56 - protein, 165 aa, synthetic construct
  UNSURE 1..165: note = HsAlg13
MKCVFVTVGTTSFDDLIACVSAPDSLQKIESLGYNRLILQIGRGTVVPEPFSTESFTLDVYRYKDSLKEDIQKADLVISHAGAGSCLETLEKGKPLVVVINEKLMNNHQLELAKQLHKEGHLFYCTCSTLPGLLQSMDLSTLKCYPPGQPEKFSAFLDKVVGLQK

SEQ ID NO: 57 - protein, 216 aa, synthetic construct
  UNSURE 1..216: note = HsAlg14
MVCVLVLAAAAGAVAVFLILRIWVVLRSMDVTPRESLSILVVAGSGGHTTEILRLLGSLSNAYSPRHYVIADTDEMSANKINSFELDRADRDPSNMYTKYYIHRIPRSREVQQSWPSTVFTTLHSMWLSFPLIHRVKPDLVLCNGPGTCVPICVSALLLGILGIKKVIIVYVESICRVETLSMSGKILFHLSDYFIVQWPALKEKYPKSVYLGRIV

SEQ ID NO: 58 - protein, 260 aa, synthetic construct
  UNSURE 1..260: note = HsDPM1
MASLEVSRSPRRSRRELEVRSPRQNKYSVLLPTYNERENLPLIVWLLVKSFSESGINYEIIIIDDGSPDGTRDVAEQLEKIYGSDRILLRPREKKLGLGTAYIHGMKHATGNYIIIMDADLSHHPKFIPEFIRKQKEGNFDIVSGTRYKGNGGVYGWDLKRKIISRGANFLTQILLRPGASDLTGSFRLYRKEVLEKLIEKCVSKGYVFQMEMIVRARQLNYTIGEVPISFVDRVYGESKLGGNEIVSFLKGLLTLFATT

SEQ ID NO: 59 - protein, 423 aa, synthetic construct [sequence omitted]

SEQ ID NO: 60 - protein, 554 aa, synthetic construct
  UNSURE 1..554: note = HsPIGB
MRRPLSKCGMEPGGGDASLTLHGLQNRSHGKIKLRKRKSTLYFNTQEKSARRRGDLLGENIYLLLFTIALRILNCFLVQTSFVPDEYWQSLEVSHHMVFNYGYLTWEWTERLRSYTYPLIFASIYKILHLLGKDSVQLLIWIPRLAQALLSAVADVRLYSLMKQLENQEVARWVFFCQLCSWFTWYCCTRTLTNTMETVLTIIALFYYPLEGSKSMNSVKYSSLVALAFIIRPTAVILWTPLLFRHFCQEPRKLDLILHHFLPVGFVTLSLSLMIDRIFFGQWTLVQFNFLKFNVLQNWGTFYGSHPWHWYFSQGFPVILGTHLPFFIHGCYLAPKRYRILLVTVLWTLLVYSMLSHKEFRFIYPVLPFCMVFCGYSLTHLKTWKKPALSFLFLSNLFLALYTGLVHQRGTLDVMSHIQKVCYNNPNKSSASIFIMMPCHSTPYYSHVHCPLPMRFLQCPPDLTGKSHYLDEADVFYLNPLNWLHREFHDDASLPTHLITFSILEEEISAFLISSNYKRTAVFFHTHLPEGRIGSHIYVYERKLKGKFNMKMKF

SEQ ID NO: 61 - protein, 579 aa, synthetic construct
  UNSURE 1..579: note = HsPIGZ
MQICGSSVASVAAGTSFQVLGPVCWQQLDLKMAVRVLWGGLSLLRVLWCLLPQTGYVHPDEFFQSPEVMAEDILGVQAARPWEFYPSSSCRSVLFPLLISGSTFWLLRLWEELGPWPGLVSGYALLVGPRLLLTALSFALDGAVYHLAPPMGADRWNALALLSGSYVTLVFYTRTFSNTIEGLLFTWLLVLVSSHVTWGPTRKEPAPGPRWRSWLLGGIVAAGFFNRPTFLAFAVVPLYLWGTRGATNPGLKSLTREALVLLPGAALTAAVFVATDSWYFSSPATSRNLVLTPVNFLHYNLNPQNLARHGTHARLTHLAVNGFLLFGVLHAQALQAAWQRLQVGLQASAQMGLLRALGARSLLSSPRSYLLLLYFMPLALLSAFSHQEARFLIPLLVPLVLLCSPQTQPVPWKGTVVLFNALGALLFGCLHQGGLVPGLEYLEQVVHAPVLPSTPTHYTLLFTHTYMPPRHLLHLPGLGAPVEVVDMGGTEDWALCQTLKSFTRQPACQVAGGPWLCRLFVVTPGTTRRAVEKCSFPFKNETLLFPHLTLEDPPALSSLLSGAWRDHLSLHIVELGEET

SEQ ID NO: 62 - protein, 324 aa, synthetic construct
  UNSURE 1..324: note = HsAlg5
MAPLLLQLAVLGAALAAAALVLISIVAFTTATKMPALHRHEEEKFFLNAKGQKETLPSIWDSPTKQLSVVVPSYNEEKRLPVMMDEALSYLEKRQKRDPAFTYEVIVVDDGSKDQTSKVAFKYCQKYGSDKVRVITLVKNRGKGGAIRMGIFSSRGEKILMADADGATKFPDVEKLEKGLNDLQPWPNQMAIACGSRAHLEKESIAQRSYFRTLLMYGFHFLVWFLCVKGIRDTQCGFKLFTREAASRTFSSLHVERWAFDVELLYIAQFFKIPIAEIAVNWTEIEGSKLVPFWSWLQMGKDLLFIRLRYLTGAWRLEQTRKMN

SEQ ID NO: 63 - protein, 507 aa, synthetic construct
  UNSURE 1..507: note = HsAlg6
MEKWYLMTVVVLIGLTVRWTVSLNSYSGAGKPPMFGDYEAQRHWQEITFNLPVKQWYFNSSDNNLQYWGLDYPPLTAYHSLLCAYVAKFINPDWIALHTSRGYESQAHKLFMRTTVLIADLLIYIPAVVLYCCCLKEISTKKKIANALCILLYPGLILIDYGHFQYNSVSLGFALWGVLGISCDCDLLGSLAFCLAINYKQMELYHALPFFCFLLGKCFKKGLKGKGFVLLVKLACIVVASFVLCWLPFFTEREQTLQVLRRLFPVDRGLFEDKVANIWCSFNVFLKIKDILPRHIQLIMSFCSTFLSLLPACIKLILQPSSKGFKFTLVSCALSFFLFSFQVHEKSILLVSLPVCLVLSEIPFMSTWFLLVSTFSMLPLLLKDELLMPSVVTTMAFFIACVTSFSIFEKTSEEELQLKSFSISVRKYLPCFTFLSRIIQYLFLISVITMVLLTLMTVTLDPPQKLPDLFSVLVCFVSCLNFLFFLVYFNIIIMWDSKSGRNQKKIS

SEQ ID NO: 64 - protein, 526 aa, synthetic construct
  UNSURE 1..526: note = HsAlg8
MAALTIATGTGNWFSALALGVTLLKCLLIPTYHSTDFEVHRNWLAITHSLPISQWYYEATSEWTLDYPPFFAWFEYILSHVAKYFDQEMLNVHNLNYSSSRTLLFQRFSVIFMDVLFVYAVRECCKCIDGKKVGKELTEKPKFILSVLLLWNFGLLIVDHIHFQYNGFLFGLMLLSIARLFQKRHMEGAFLFAVLLHFKHIYLYVAPAYGVYLLRSYCFTANKPDGSIRWKSFSFVRVISLGLVVFLVSALSLGPFLALNQLPQVFSRLFPFKRGLCHAYWAPNFWALYNALDKVLSVIGLKLKFLDPNNIPKASMTSGLVQQFQHTVLPSVTPLATLICTLIAILPSIFCLWFKPQGPRGFLRCLTLCALSSFMFGWHVHEKAILLAILPMSLLSVGKAGDASIFLILTTTGHYSLFPLLFTAPELPIKILLMLLFTIYSISSLKTLFRKEKPLFNWMETFYLLGLGPLEVCCEFVFPFTSWKVKYPFIPLLLTSVYCAVGITYAWFKLYVSVLIDSAIGKTKKQ

SEQ ID NO: 65 - protein, 473 aa, synthetic construct
  UNSURE 1..473: note = HsAlg10
MAQLEGYCFSAALSCTFLVSCLLFSAFSRALREPYMDEIFHLPQAQRYCEGHFSLSQWDPMITTLPGLYLVSVGVVKPAIWIFAWSEHVVCSIGMLRFVNLLFSVGNFYLLYLLFHKVQPRNKAASSIQRVLSTLTLAVFPTLYFFNFLYYTEAGSMFFTLFAYLMCLYGNHKTSAFLGFCGFMFRQTNIIWAVFCAGNVIAQKLTEAWKTELQKKEDRLPPIKGPFAEFRKILQFLLAYSMSFKNLSMLFCLTWPYILLGFLFCAFVVVNGGIVIGDRSSHEACLHFPQLFYFFSFTLFFSFPHLLSPSKIKTFLSLVWKHGILFLVVTLVSVFLVWKFTYAHKYLLADNRHYTFYVWKRVFQRYAILKYLLVPAYIFAGWSIADSLKSKPIFWNLMFFICLFIVIVPQKLLEFRYFILPYVIYRLNITLPPTSRLVCELSCYAIVNFITFYIFLNKTFQWPNSQDIQRFMW

SEQ ID NO: 66 - protein, 394 aa, synthetic construct
  UNSURE 1..394: note = HsUGCG
MALLDLALEGMAVFGFVLFLVLWLMHFMAIIYTRLHLNKKATDKQPYSKLPGVSLLKPLKGVDPNLINNLETFFELDYPKYEVLLCVQDHDDPAIDVCKKLLGKYPNVDARLFIGGKKVGINPKINNLMPGYEVAKYDLIWICDSGIRVIPDTLTDMVNQMTEKVGLVHGLPYVADRQGFAATLEQVYFGTSHPRYYISANVTGFKCVTGMSCLMRKDVLDQAGGLIAFAQYIAEDYFMAKAIADRGWRFAMSTQVAMQNSGSYSISQFQSRMIRWTKLRINMLPATIICEPISECFVASLIIGWAAHHVFRWDIMVFFMCHCLAWFIFDYIQLRGVQGGTLCFSKLDYAVAWFIRESMTIYIFLSALWDPTISWRTGRYRLRCGGTAEEILDV

SEQ ID NO: 67 - protein, 498 aa, synthetic construct
  UNSURE 1..498: note = HsB3GLCT
MRPPACWWLLAPPALLALLTCSLAFGLASEDTKKEVKQSQDLEKSGISRKNDIDLKGIVFVIQSQSNSFHAKRAEQLKKSILKQAADLTQELPSVLLLHQLAKQEGAWTILPLLPHFSVTYSRNSSWIFFCEEETRIQIPKLLETLRRYDPSKEWFLGKALHDEEATIIHHYAFSENPTVFKYPDFAAGWALSIPLVNKLTKRLKSESLKSDFTIDLKHEIALYIWDKGGGPPLTPVPEFCTNDVDFYCATTFHSFLPLCRKPVKKKDIFVAVKTCKKFHGDRIPIVKQTWESQASLIEYYSDYTENSIPTVDLGIPNTDRGHCGKTFAILERFLNRSQDKTAWLVIVDDDTLISISRLQHLLSCYDSGEPVFLGERYGYGLGTGGYSYITGGGGMVFSREAVRRLLASKCRCYSNDAPDDMVLGMCFSGLGIPVTHSPLFHQARPVDYPKDYLSHQVPISFHKHWNIDPVKVYFTWLAPSDEDKARQETQKGFREEL

SEQ ID NO: 68 - protein, 350 aa, synthetic construct
  UNSURE 1..350: note = HsGLYG
MTDQAFVTLTTNDAYAKGALVLGSSLKQHRTTRRLVVLATPQVSDSMRKVLETVFDEVIMVDVLDSGDSAHLTLMKRPELGVTLTKLHCWSLTQYSKCVFMDADTLVLANIDDLFDREELSAAPDPGWPDCFNSGVFVYQPSVETYNQLLHLASEQGSFDGGDQGILNTFFSSWATTDIRKHLPFIYNLSSISIYSYLPAFKVFGASAKVVHFLGRVKPWNYTYDPKTKSVKSEAHDPNMTHPEFLILWWNIFTTNVLPLLQQFGLVKDTCSYVNVLSDLVYTLAFSCGFCRKEDVSGAISHLSLGEIPAMAQPFVSSEERKERWEQGQADYMGADSFDNIKRKLDTYLQ

SEQ ID NO: 69 - protein, 392 aa, synthetic construct
  UNSURE 1..392: note = HsPOGLUT1
MEWWASSPLRLWLLLFLLPSAQGRQKESGSKWKVFIDQINRSLENYEPCSSQNCSCYHGVIEEDLTPFRGGISRKMMAEVVRRKLGTHYQITKNRLYRENDCMFPSRCSGVEHFILEVIGRLPDMEMVINVRDYPQVPKWMEPAIPVFSFSKTSEYHDIMYPAWTFWEGGPAVWPIYPTGLGRWDLFREDLVRSAAQWPWKKKNSTAYFRGSRTSPERDPLILLSRKNPKLVDAEYTKNQAWKSMKDTLGKPAAKDVHLVDHCKYKYLFNFRGVAASFRFKHLFLCGSLVFHVGDEWLEFFYPQLKPWVHYIPVKTDLSNVQELLQFVKANDDVAQEIAERGSQFIRNHLQMDDITCYWENLLSEYSKFLSYNVTRRKGYDQIIPKMLKTEL

SEQ ID NO: 70 - protein, 445 aa, synthetic construct
  UNSURE 1..445: note = HsGnT1
MLKKQSAGLVLWGAILFVAWNALLLLFFWTRPAPGRPPSVSALDGDPASLTREVIRLAQDAEVELERQRGLLQQIGDALSSQRGRVPTAAPPAQPRVPVTPAPAVIPILVIACDRSTVRRCLDKLLHYRPSAELFPIIVSQDCGHEETAQAIASYGSAVTHIRQPDLSSIAVPPDHRKFQGYYKIARHYRWALGQVFRQFRFPAAVVVEDDLEVAPDFFEYFRATYPLLKADPSLWCVSAWNDNGKEQMVDASRPELLYRTDFFPGLGWLLLAELWAELEPKWPKAFWDDWMRRPEQRQGRACIRPEISRTMTFGRKGVSHGQFFDQHLKFIKLNQQFVHFTQLDLSYLQREAYDRDFLARVYGAPQLQVEKVRTNDRKELGEVRVQYTGRDSFKAFAKALGVMDDLKSGVPRAGYRGIVTFQFRGRRVHLAPPLTWEGYDPSWN

SEQ ID NO: 71 - protein, 447 aa, synthetic construct
  UNSURE 1..447: note = HsGnT2
MRFRIYKRKVLILTLVVAACGFVLWSSNGRQRKNEALAPPLLDAEPARGAGGRGGDHPSVAVGIRRVSNVSAASLVPAVPQPEADNLTLRYRSLVYQLNFDQTLRNVDKAGTWAPRELVLVVQVHNRPEYLRLLLDSLRKAQGIDNVLVIFSHDFWSTEINQLIAGVNFCPVLQVFFPFSIQLYPNEFPGSDPRDCPRDLPKNAALKLGCINAEYPDSFGHYREAKFSQTKHHWWWKLHFVWERVKILRDYAGLILFLEEDHYLAPDFYHVFKKMWKLKQQECPECDVLSLGTYSASRSFYGMADKVDVKTWKSTEHNMGLALTRNAYQKLIECTDTFCTYDDYNWDWTLQYLTVSCLPKFWKVLVPQIPRIFHAGDCGMHHKKTCRPSTQSAQIESLLNNNKQYMFPETLTISEKFTVVAISPPRKNGGWGDIRDHELCKSYRRLQ

SEQ ID NO: 72 - protein, 533 aa, synthetic construct
  UNSURE 1..533: note = HsGnT3
MKMRRYKLFLMFCMAGLCLISFLHFFKTLSYVTFPRELASLSPNLVSSFFWNNAPVTPQASPEPGGPDLLRTPLYSHSPLLQPLPPSKAAEELHRVDLVLPEDTTEYFVRTKAGGVCFKPGTKMLERPPPGRPEEKPEGANGSSARRPPRYLLSARERTGGRGARRKWVECVCLPGWHGPSCGVPTVVQYSNLPTKERLVPREVPRRVINAINVNHEFDLLDVRFHELGDVVDAFVVCESNFTAYGEPRPLKFREMLTNGTFEYIRHKVLYVFLDHFPPGGRQDGWIADDYLRTFLTQDGVSRLRNLRPDDVFIIDDADEIPARDGVLFLKLYDGWTEPFAFHMRKSLYGFFWKQPGTLEVVSGCTVDMLQAVYGLDGIRLRRRQYYTMPNFRQYENRTGHILVQWSLGSPLHFAGWHCSWCFTPEGIYFKLVSAQNGDFPRWGDYEDKRDLNYIRGLIRTGGWFDGTQQEYPPADPSEHMYAPKYLLKNYDRFHYLLDNPYQEPRSTAAGGWRHRGPEGRPPARGKLDEAEV

SEQ ID NO: 73 - protein, 535 aa, synthetic construct
  UNSURE 1..535: note = HsGnT4a
MRLRNGTVATALAFITSFLTLSWYTTWQNGKEKLIAYQREFLALKERLRIAEHRISQRSSELNTIVQQFKRVGAETNGSKDALNKFSDNTLKLLKELTSKKSLQVPSIYYHLPHLLKNEGSLQPAVQIGNGRTGVSIVMGIPTVKREVKSYLIETLHSLIDNLYPEEKLDCVIVVFIGETDIDYVHGVVANLEKEFSKEISSGLVEVISPPESYYPDLTNLKETFGDSKERVRWRTKQNLDYCFLMMYAQEKGIYYIQLEDDIIVKQNYFNTIKNFALQLSSEEWMILEFSQLGFIGKMFQAPDLTLIVEFIFMFYKEKPIDWLLDHILWVKVCNPEKDAKHCDRQKANLRIRFRPSLFQHVGLHSSLSGKIQKLTDKDYMKPLLLKIHVNPPAEVSTSLKVYQGHTLEKTYMGEDFFWAITPIAGDYILFKFDKPVNVESYLFHSGNQEHPGDILLNTTVEVLPFKSEGLEISKETKDKRLEDGYFRIGKFENGVAEGMVDPSLNPISAFRLSVIQNSAVWAILNEIHIKKATN

SEQ ID NO: 74 - protein, 428 aa, synthetic construct
  UNSURE 1..428: note = HsGCNT1
MLRTLLRRRLFSYPTKYYFMVLVLSLITFSVLRIHQKPEFVSVRHLELAGENPSSDINCTKVLQGDVNEIQKVKLEILTVKFKKRPRWTPDDYINMTSDCSSFIKRRKYIVEPLSKEEAEFPIAYSIVVHHKIEMLDRLLRAIYMPQNFYCIHVDTKSEDSYLAAVMGIASCFSNVFVASRLESVVYASWSRVQADLNCMKDLYAMSANWKYLINLCGMDFPIKTNLEIVRKLKLLMGENNLETERMPSHKEERWKKRYEVVNGKLTNTGTVKMLPPLETPLFSGSAYFVVSREYVGYVLQNEKIQKLMEWAQDTYSPDEYLWATIQRIPEVPGSLPASHKYDLSDMQAVARFVKWQYFEGDVSKGAPYPPCDGVHVRSVCIFGAGDLNWMLRKHHLFANKFDVDVDLFAIQCLDEHLRHKALETLKH

SEQ ID NO: 75 - protein, 402 aa, synthetic construct
  UNSURE 1..402: note = HsGCNT2
MMGSWKHCLFSASLISALIFVFVYNTELWENKRFLRAALSNASLLAEACHQIFEGKVFYPTENALKTTLDEATCYEYMVRSHYVTETLSEEEAGFPLAYTVTIHKDFGTFERLFRAIYMPQNVYCVHLDQKATDAFKGAVKQLLSCFPNAFLASKKESVVYGGISRLQADLNCLEDLVASEVPWKYVINTCGQDFPLKTNREIVQYLKGFKGKNITPGVLPPDHAVGRTKYVHQELLNHKNSYVIKTTKLKTPPPHDMVIYFGTAYVALTRDFANFVLQDQLALDLLSWSKDTYSPDEHFWVTLNRIPGVPGSMPNASWTGNLRAIKWSDMEDRHGGCHGHYVHGICIYGNGDLKWLVNSPSLFANKFELNTYPLTVECLELRHRERTLNQSETAIQPSWYF

SEQ ID NO: 76 - protein, 397 aa, synthetic construct
  UNSURE 1..397: note = HsB3GNT2
MSVGRRRIKLLGILMMANVFIYFIMEVSKSSSQEKNGKGEVIIPKEKFWKISTPPEAYWNREQEKLNRQYNPILSMLTNQTGEAGRLSNISHLNYCEPDLRVTSVVTGFNNLPDRFKDFLLYLRCRNYSLLIDQPDKCAKKPFLLLAIKSLTPHFARRQAIRESWGQESNAGNQTVVRVFLLGQTPPEDNHPDLSDMLKFESEKHQDILMWNYRDTFFNLSLKEVLFLRWVSTSCPDTEFVFKGDDDVFVNTHHILNYLNSLSKTKAKDLFIGDVIHNAGPHRDKKLKYYIPEVVYSGLYPPYAGGGGFLYSGHLALRLYHITDQVHLYPIDDVYTGMCLQKLGLVPEKHKGFRTFDIEEKNKNNICSYVDLMLVHSRKPQEMIDIWSQLQSAHLKC

SEQ ID NO: 77 - protein, 384 aa, synthetic construct
  UNSURE 1..384: note = HsB3GNT6
MAFPCRRSLTAKTLACLLVGVSFLALQQWFLQAPRSPREERSPQEETPEGPTDAPAADEPPSELVPGPPCVANASANATADFEQLPARIQDFLRYRHCRHFPLLWDAPAKCAGGRGVFLLLAVKSAPEHYERRELIRRTWGQERSYGGRPVRRLFLLGTPGPEDEARAERLAELVALEAREHGDVLQWAFADTFLNLTLKHLHLLDWLAARCPHARFLLSGDDDVFVHTANVVRFLQAQPPGRHLFSGQLMEGSVPIRDSWSKYFVPPQLFPGSAYPVYCSGGGFLLSGPTARALRAAARHTPLFPIDDAYMGMCLERAGLAPSGHEGIRPFGVQLPGAQQSSFDPCMYRELLLVHRFAPYEMLLMWKALHSPALSCDRGHRVS

SEQ ID NO: 78 - protein, 484 aa, synthetic construct
  UNSURE 1..484: note = HsPIGA
MACRGGAGNGHRASATLSRVSPGSLYTCRTRTHNICMVSDFFYPNMGGVESHIYQLSQCLIERGHKVIIVTHAYGNRKGIRYLTSGLKVYYLPLKVMYNQSTATTLFHSLPLLRYIFVRERVTIIHSHSSFSAMAHDALFHAKTMGLQTVFTDHSLFGFADVSSVLTNKLLTVSLCDTNHIICVSYTSKENTVLRAALNPEIVSVIPNAVDPTDFTPDPFRRHDSITIVVVSRLVYRKGIDLLSGIIPELCQKYPDLNFIIGGEGPKRIILEEVRERYQLHDRVRLLGALEHKDVRNVLVQGHIFLNTSLTEAFCMAIVEAASCGLQVVSTRVGGIPEVLPENLIILCEPSVKSLCEGLEKAIFQLKSGTLPAPENIHNIVKTFYTWRNVAERTEKVYDRVSVEAVLPMDKRLDRLISHCGPVTGYIFALLAVFNFLFLIFLRWMTPDSIIDVAIDATGPRGAWTNNYSHSKRGGENNEISETR

SEQ ID NO: 79 - protein, 393 aa, synthetic construct
  TRANSIT 1..393: note = HsXXLT1
MGLLRGGLPCARAMARLGAVRSHYCALLLAAALAVCAFYYLGSGRETFSSATKRLKEARAGAPAAPSPPALELARGSVAPAPGAKAKSLEGGGAGPVDYHLLMMFTKAEHNAALQAKARVALRSLLRLAKFEAHEVLNLHFVSEEASREVAKGLLRELLPPAAGFKCKVIFHDVAVLTDKLFPIVEAMQKHFSAGLGTYYSDSIFFLSVAMHQIMPKEILQIIQLDLDLKFKTNIRELFEEFDSFLPGAIIGIAREMQPVYRHTFWQFRHENPQTRVGGPPPEGLPGFNSGVMLLNLEAMRQSPLYSRLLEPAQVQQLADKYHFRGHLGDQDFFTMIGMEHPKLFHVLDCTWNRQLCTWWRDHGYSDVFEAYFRCEGHVKIYHGNCNTPIPED

SEQ ID NO: 80 - protein, 533 aa, synthetic construct
  UNSURE 1..533: note = HsUGT1A1
MAVESQGGRPLVLGLLLCVLGPVVSHAGKILLIPVDGSHWLSMLGAIQQLQQRGHEIVVLAPDASLYIRDGAFYTLKTYPVPFQREDVKESFVSLGHNVFENDSFLQRVIKTYKKIKKDSAMLLSGCSHLLHNKELMASLAESSFDVMLTDPFLPCSPIVAQYLSLPTVFFLHALPCSLEFEATQCPNPFSYVPRPLSSHSDHMTFLQRVKNMLIAFSQNFLCDVVYSPYATLASEFLQREVTVQDLLSSASVWLFRSDFVKDYPRPIMPNMVFVGGINCLHQNPLSQEFEAYINASGEHGIVVFSLGSMVSEIPEKKAMAIADALGKIPQTVLWRYTGTRPSNLANNTILVKWLPQNDLLGHPMTRAFITHAGSHGVYESICNGVPMVMMPLFGDQMDNAKRMETKGAGVTLNVLEMTSEDLENALKAVINDKSYKENIMRLSSLHKDRPVEPLDLAVFWVEFVMRHKGAPHLRPAAHDLTWYQYHSLDVIGFLLAVVLTVAFITFKCCAYGYRKCLGKKGRVKKAHKSKTH

SEQ ID NO: 81 - protein, 415 aa, synthetic construct
  UNSURE 1..415: note = HsB4GAT1
MQMSYAIRCAFYQLLLAALMLVAMLQLLYLSLLSGLHGQEEQDQYFEFFPPSPRSVDQVKAQLRTALASGGVLDASGDYRVYRGLLKTTMDPNDVILATHASVDNLLHLSGLLERWEGPLSVSVFAATKEEAQLATVLAYALSSHCPDMRARVAMHLVCPSRYEAAVPDPREPGEFALLRSCQEVFDKLARVAQPGINYALGTNVSYPNNLLRNLAREGANYALVIDVDMVPSEGLWRGLREMLDQSNQWGGTALVVPAFEIRRARRMPMNKNELVQLYQVGEVRPFYYGLCTPCQAPTNYSRWVNLPEESLLRPAYVVPWQDPWEPFYVAGGKVPTFDERFRQYGFNRISQACELHVAGFDFEVLNEGFLVHKGFKEALKFHPQKEAENQHNKILYRQFKQELKAKYPNSPRRC

SEQ ID NO: 82 - protein, 534 aa, synthetic construct
  UNSURE 1..534: note = HsUGT1A3
MATGLQVPLPWLATGLLLLLSVQPWAESGKVLVVPIDGSHWLSMREVLRELHARGHQAVVLTPEVNMHIKEENFFTLTTYAISWTQDEFDRHVLGHTQLYFETEHFLKKFFRSMAMLNNMSLVYHRSCVELLHNEALIRHLNATSFDVVLTDPVNLCAAVLAKYLSIPTVFFLRNIPCDLDFKGTQCPNPSSYIPRLLTTNSDHMTFMQRVKNMLYPLALSYICHAFSAPYASLASELFQREVSVVDILSHASVWLFRGDFVMDYPRPIMPNMVFIGGINCANRKPLSQEFEAYINASGEHGIVVFSLGSMVSEIPEKKAMAIADALGKIPQTVLWRYTGTRPSNLANNTILVKWLPQNDLLGHPMTRAFITHAGSHGVYESICNGVPMVMMPLFGDQMDNAKRMETKGAGVTLNVLEMTSEDLENALKAVINDKSYKENIMRLSSLHKDRPVEPLDLAVFWVEFVMRHKGAPHLRPAAHDLTWYQYHSLDVIGFLLAVVLTVAFITFKCCAYGYRKCLGKKGRVKKAHKSKTH

SEQ ID NO: 83 - protein, 291 aa, synthetic construct
  UNSURE 1..291: note = CjCsTII
MKNNIIIAGNGSSLKEIDYSRLPNDFDVFRCNQFYFEDKYYLGKKCKAVFYNSTLFFEQYYTLKHLIQNQEYETELIMCSNFNQAHLENENFLKNFYDYFPDAHLGYDFFKQLKDFNAYFKFHEIYFNQRITSGIYMCAVAIALGYKEIYLSGIDFYQNGSSYAFDTKQKNLLKLAPNFKNDNSHYIGHSKNTDIKALEFLEKTYKIKLYCLCPNSLLANFIELAPNLNSNFTIQEKNNYTKDILIPSKEAYKKFTKMTKTQEILKNNIYYKLTKDLLRLPSDIKHYFKGK

SEQ ID NO: 84 - protein, 495 aa, synthetic construct
  UNSURE 1..495: note = NmPolysiaT
MLKKIKKALFQPKKFFQDSMWLTTSPFYLTPPRNNLFVISNLGQLNQVQSLIKIQKLTNNLLVILYTSKNLKMPKLVHQSANKNLFESIYLFELPRSPNNITPKKLLYIYRSYKKILNIIQPAHLYMLSFTGHYSYLISIAKKKNITTHLIDEGTGTYAPLLESFSYHPTKLERYLIGNNLNIKGYIDHFDILHVPFPEYAKKIFNAKKYNRFFAHAGGISINNNIANLQKKYQISKNDYIFVSQRYPISDDLYYKSIVEILNSISLQIKGKIFIKLHPKEMGNNYVMSLFLNMVEINPRLVVINEPPFLIEPLIYLTNPKGIIGLASSSLIYTPLLSPSTQCLSIGELIINLIQKYSMVENTEMIQEHLEIIKKFNFINILNDLNGVISNPLFKTEETFETLLKSAEFAYKSKNYFQAIFYWQLASKNNITLLGHKALWYYNALYNVKQIYKMEYSDIFYIDNISVDFHSKDKLTWEKIKHYYYSADNRIGRDR

SEQ ID NO: 85 - protein, 302 aa, synthetic construct
  UNSURE 1..302: note = CjCgtB
MGFKISIILPTYNVEQYIARAIESCIKQTFKDIEIIVVDDCGNDNSINIAKEYSKKDKRIKIIHNEKNIGLLRARYEGVKVANSPYIMFLDPDDYLELNACEECIKILDEQDEVDLVFFNAIVESNVISYKKFDFNSGFYSKKEFVKKIIAEKNLYWTMWGKLIRKKLYLEAFASLRLEKDVKINMAEDVLLYYPMLSQAQKIAYMNCNLYHYVPNNNSICNTKNEVGVKNNIQDLQLVLNYLRQNYILNKYCSVLYVLIKYLLYIQIYKIKRTKLMVTLLAKINILTLKILFKYKKFLKQC

SEQ ID NO: 86 - protein, 273 aa, synthetic construct
  UNSURE 1..273: note = HpLgtB
MRVFIISLNQKVCDQFGLVFRDTTTLLHNINATHHKAQIFDAIYSKTFESELHPLVKKHLHPYFITQNIKDMGITTNLISRVSKFYYALKYHAKFMSFGELGCYASHYSLWEKCIELNEPICILEDDITLKEDFKEGLDFLEKHIQELGYARLMYLLYDANVKSEPLSHKNHEIQERVGIIKAYSHGVGTQGYVITPKIAKVFKKCSRKWVVPVDTIMDATFIHGVKNLVLQPFVIADDEQISTIARKEEPYSSKIALMRKLHFKYLKYWQFV

SEQ ID NO: 87 - protein, 275 aa, synthetic construct
  UNSURE 1..275: note = NmLgtB
MQNHVISLASAAERRAHIADTFGRHGIPFQFFDALMPSERLEQAMAELVPGLSAHPYLSGVEKACFMSHAVLWKQALDEGLPYITVFEDDVLLGEGAEKFLAEDAWLQERFDPDTAFIVRLETMFMHVLTSPSGVADYCGRAFPLLESEHWGTAGYIISRKAMRFFLDRFAALPPEGLHPVDLMMFSDFFDREGMPVCQLNPALCAQELHYAKFHDQNSALGSLIEHDRLLNRKQQRRDSPANTFKHRLIRALTKISREREKRRQRREQFIVPFQ

SEQ ID NO: 88 - protein, 279 aa, synthetic construct
  SIGNAL 1..279: note = NgLgtB
MQNHVISLASAAERRAHIAATFGSRGIPFQFFDALMPSERLERAMAELVPGLSAHPYLSGVEKACFMSHAVLWEQALDEGVPYIAVFEDDVLLGEGAEQFLAEDTWLQERFDPDSAFVVRLETMFMHVLTSPSGVADYGGRAFPLLESEHCGTAGYIISRKAMRFFLDRFAVLPPERLHPVDLMMFGNPDDREGMPVCQLNPALCAQELHYAKFHDQNSALGSLIEHDRRLNRKQQWRDSPANTFKHRLIRALTKIGREREKRRQRREQLIGKIIVPFQ

SEQ ID NO: 89 - protein, 297 aa, synthetic construct
  UNSURE 1..297: note = EcFUT
MSIIRLQGGLGNQLFQFSFGYALSKINGTPLYFDISHYAENDDHGGYRLNNLQIPEEYLQYYTPKINNIYKFLVRGSRLYPEIFLFLGFCNEFHAYGYDFEYIAQKWKSKKYIGYWQSEHFFHKHILDLKEFFIPKNVSEQANLLAAKILESQSSLSIHIRRGDYIKNKTATLTHGVCSLEYYKKALNKIRDLAMIRDVFIFSDDIFWCKENIETLLSKKYNIYYSEDLSQEEDLWLMSLANHHIIANSSFSWWGAYLGTSASQIVIYPTPWYDITPKNTYIPIVNHWINVDKHSSC

SEQ ID NO: 90 - protein, 367 aa, synthetic construct
  UNSURE 1..367: note = EcWecA
MNLLTVSTDLISIFLFTTLFLFFARKVAKKVGLVDKPNFRKRHQGLIPLVGGISVYAGICFTFGIVDYYIPHASLYLACAGVLVFIGALDDRFDISVKIRATIQAAVGIVMMVFGKLYLSSLGYIFGSWEMVLGPFGYFLTLFAVWAAINAFNMVDGIDGLLGGLSCVSFAAIGMILWFDGQTSLAIWCFAMIAAILPYIMLNLGILGRRYKVFMGDAGSTLIGFTVIWILLETTQGKTHPISPVTALWIIAIPLMDMVAIMYRRLRKGMSPFSPDRQHIHHLIMRAGFTSRQAFVLITLAAALLASIGVLAEYSHFVPEWVMLVLFLLAFFLYGYCIKRAWKVARFIKRVKRRLRRNRGGSPNLTK

SEQ ID NO: 91 - protein, 644 aa, synthetic construct
  UNSURE 1..644: note = LpSetA
MYKIYSYLGWRIDMKTENLPQAGQEAQIDKKIHFIWVGHIMPQKNIQVVSEWAEKNPGYETIIWVDKKIAPAKELDLFILDMKSKGITVKDINEEGVCRDSIRHELDQESPNYGMVSDMLRLNILAAEGGIYLDSDILCSAPFPDEIYAPFGFLLSPWSQGANNTLCNDIILCSKGNQIIQQLADAIEQSYIARDSFEFTHEYASMKETKGERIAKTLGVTGPGFLFHQLKKMGILNDKSEMEAIHWELQDQRYLIDGSVKEPDYFYVPQNNTNDASWVPSIKRPGIENMSFQERLENAVQLIAFDIQKTGLFNLDHYANELKVKQNSWCIAAETSPELKPDSYLLIRPRDKTGEWTLYYVDEDKKLNPVTLPVIKGAIKLSEVSDPLRKFHTLLSQVSDPVNPTAHELKQIGRALIELKPRQDEWHCKNKWSGAEEIAQELWQRITSNETLRAQIKQCFTQFESLKPRVAELGLTRASGAGTEVEAHESTVKEQEIISQNTVGEEGTKEKNSVQLASENSSDEKIKTAHDLIDEIIQDVIQLDGKLGLLGGNTRQLEDGRVINIPNGAAMIFDDYKKYKQGELTAESALESMIKIAKLSNQLNRHTFFNQRQPETGQFYKKVAAIDLQTTIAAEYDNNHGLRI

SEQ ID NO: 92 - protein, 492 aa, synthetic construct
  UNSURE 1..492: note = NmSynE
MLQKIRKALFHPKKFFQDSQWFATPLFSSFAPKSNLFIISTFAQLNQAHSLTKMQKLKNNLLVILYTTQNMKMPKLIQKSVDKELFSVTYMFELPRKPGIVSPKKFLYIQRGYKKLLKTIQPAHLYVMSFAGHYSSLLSLAKKMNITTHLVEEGTATYAPLLESFTYKPTKFEQRFVGNNLHQKGYFDKFDILHVAFPEYAKKIFNANEYHRFFAHSGGISTSQSIAKIQDKYRISQNDYIFVSQRYPVSDEVYYKTIVETLNQMSLRIEGKIFIKLHPKEMENKNIMSLFLNMVTINPRLVVINEPPFLIDPLIYLTTPKGIIGLTSTSIVYTPLLSPTTQCLSIGQIVIDSIHHTAQQENTALIEEHLEIVKQFDFIKILSSIEDGIDTNSFKTEETLEMLLKSAEYAYKNKNFYQAIFYWQLASNNDLSVLGYKSLWYYNALNKVKQNYKMKYLEINYIERISLYFNDKDKMIWQNIKNDFFKYSLCNQ

SEQ ID NO: 93 - protein, 449 aa, synthetic construct
  UNSURE 1..449: note = ScAlg1
MFLEIPRWLLALIILYLSIPLVVYYVIPYLFYGNKSTKKRIIIFVLGDVGHSPRICYHAISFSKLGWQVELCGYVEDTLPKIISSDPNITVHHMSNLKRKGGGTSVIFMVKKVLFQVLSIFKLLWELRGSDYILVQNPPSIPILPIAVLYKLTGCKLIIDWHNLAYSILQLKFKGNFYHPLVLISYMVEMIFSKFADYNLTVTEAMRKYLIQSFHLNPKRCAVLYDRPASQFQPLAGDISRQKALTTKAFIKDYIRDDFDTEKGDKIIVTSTSFTPDEDIGILLGALKIYENSYVKFDSSLPKILCFITGKGPLKEKYMKQVEEYDWKRCQIEFVWLSAEDYPKLLQLCDYGVSLHTSSSGLDLPMKILDMFGSGLPVIAMNYPVLDELVQHNVNGLKFVDRRELHESLIFAMKDADLYQKLKKNVTQEAENRWQSNWERTMRGLKLIH

SEQ ID NO: 94 - protein, 548 aa, synthetic construct
  UNSURE 1..548: note = ScAlg11
MGSAWTNYNFEEVKSHFGFKKYVVSSLVLVYGLIKVLTWIFRQWVYSSLNPFSKKSSLLNRAVASCGEKNVKVFGFFHPYCNAGGGGEKVLWKAVDITLRKDAKNVIVIYSGDFVNGENVTPENILNNVKAKFDYDLDSDRIFFISLKLRYLVDSSTWKHFTLIGQAIGSMILAFESIIQCPPDIWIDTMGYPFSYPIIARFLRRIPIVTYTHYPIMSKDMLNKLFKMPKKGIKVYGKILYWKVFMLIYQSIGSKIDIVITNSTWTNNHIKQIWQSNTCKIIYPPCSTEKLVDWKQKFGTAKGERLNQAIVLAQFRPEKRHKLIIESFATFLKNLPDSVSPIKLIMAGSTRSKQDENYVKSLQDWSENVLKIPKHLISFEKNLPFDKIEILLNKSTFGVNAMWNEHFGIAVVEYMASGLIPIVHASAGPLLDIVTPWDANGNIGKAPPQWELQKKYFAKLEDDGETTGFFFKEPSDPDYNTTKDPLRYPNLSDLFLQITKLDYDCLRVMGARNQQYSLYKFSDLKFDKDWENFVLNPICKLLEEEERG

SEQ ID NO: 95 - protein, 446 aa, synthetic construct
  UNSURE 1..446: note = NtGnTI
MRGNKFCCDFRYLLILAAVAFIYTQMRLFATQSEYADRLAAAIEAENHCTSQTRLLIDQISLQQGRIVALEEQMKRQDQECRQLRALVQDLESKGIKKLIGNVQMPVAAVVVMACNRADYLEKTIKSILKYQISVASKYPLFISQDGSHPDVRKLALSYDQLTYMQHLDFEPVHTERPGELIAYYKIARHYKWALDQLFYKHNFSRVIILEDDMEIAPDFFDFFEAGATLLDRDKSIMAISSWNDNGQMQFVQDPYALYRSDFFPGLGWMLSKSTWDELSPKWPKAYWDDWLRLKENHRGRQFIRPEVCRTYNFGEHGSSLGQFFKQYLEPIKLNDVQVDWKSMDLSYLLEDNYVKHFGDLVKKAKPIHGADAVLKAFNIDGDVRIQYRDQLDFENIARQFGIFEEWKDGVPRAAYKGIVVFRYQTSRRVFLVGHDSLQQLGIEDT

SEQ ID NO: 96 - protein, 429 aa, synthetic construct
  UNSURE 1..429: note = NtGnTII
MASCKKARIKDAAFRRLVSLALITVSGVVLLLILLRTNTVSNLINSYTNESSESFESIEEAKVSNTHLTRIPKLPLQNELSILLSKRNQMPPRNLNLYSKLAKDHVVIVLYVHNRPQYLQIVIDSLSRVEGISETLLIVSHDGYFEEMNKIVESIKFCQVKQIFAPYSPHIFSDSFPGVSPKDCKEKDDPVEKHCEGMPDQYGNHRSPKIVSLKHHWWWMMNTVWDGLEVTRHHSGHILFIEEDHFIYPNAYRNMQLLAELKSKKCPDCYAANLAPSEVKLRGEGWECLVAERMGNVGYAFNRTVWRKIHKKAAEFCSFDDYNWDITMWSTVYPSFGSPVYTLRGSRTSAVHFGKCGLHQGHNRNVACMDNGGVNIVVDEIDKVANIKPEWEVRKYEHQAGYKAGFKGWGGWGDVRDRELCMEFAKMYI

SEQ ID NO: 97 - protein, 368 aa, synthetic construct
  UNSURE 1..368: note = BtGGTA1
MNVKGKVILSMLVVSTVIVVFWEYIHSPEGSLFWINPSRNPEVGGSSIQKGWWLPRWFNNGYHEEDGDINEEKEQRNEDESKLKLSDWFNPFKRPEVVTMTKWKAPVVWEGTYNRAVLDNYYAKQKITVGLTVFAVGRYIEHYLEEFLTSANKHFMVGHPVIFYIMVDDVSRMPLIELGPLRSFKVFKIKPEKRWQDISMMRMKTIGEHIVAHIQHEVDFLFCMDVDQVFQDKFGVETLGESVAQLQAWWYKADPNDFTYERRKESAAYIPFGEGDFYYHAAIFGGTPTQVLNITQECFKGILKDKKNDIEAQWHDESHLNKYFLLNKPTKILSPEYCWDYHIGLPADIKLVKMSWQTKEYNVVRNNV

SEQ ID NO: 98 - protein, 394 aa, synthetic construct
  UNSURE 1..392: note = MmGGTA1
MITMLQDLHVNKISMSRSKSETSLPSSRSGSQEKIMNVKGKVILLMLIVSTVVVVFWEYVNRIPEVGENRWQKDWWFPSWFKNGTHSYQEDNVEGRREKGRNGDRIEEPQLWDWFNPKNRPDVLTVTPWKAPIVWEGTYDTALLEKYYATQKLTVGLTVFAVGKYIEHYLEDFLESADMYFMVGHRVIFYVMIDDTSRMPVVHLNPLHSLQVFEIRSEKRWQDISMMRMKTIGEHILAHIQHEVDFLFCMDVDQVFQDNFGVETLGQLVAQLQAWWYKASPEKFTYERRELSAAYIPFGEGDFYYHAAIFGGTPTHILNLTRECFKGILQDKKHDIEAQWHDESHLNKYFLFNKPTKILSPEYCWDYQIGLPSDIKSVKVAWQTKEYNLVRNNV

SEQ ID NO: 99 - protein, 371 aa, synthetic construct
  UNSURE 1..371: note = RnGGTA1
MVTMPQDLHVKVSMSRSKSETSLLSSRSGSQEKIMNVKGKIILSVLMVSTVLVVFWEYVNRTHSYQEEDIERAREKGRNGDSIVEPQLWDWFNPKNRPEVLTVTPWKAPIVWEGTYDTALLEKYYARQKITVGLTVFAVGKYIEHYLEDFLESANKYFMVGHRVIFYVMMDDTSRMPAVHLSPLHSLQVFEIRSEKRWQDISMMRMKTIGEHILDHIQHEVDFLFCMDVDQVFQDNFGVETLGQLVAQLQAWWYKASPDEFTYERRELSAAYIPFGEGDFYYHAAVFGGTPVHILNLTRECFKGILQDKKHDIEAQWHDESHLNKYFLFNKPTKILSPEYCWDYHIGLPSDIKNVKIAWQTKEYNLVRSNV

SEQ ID NO: 100 - protein, 402 aa, synthetic construct
  UNSURE 1..402: note = BtB4GalT1
MKFREPLLGGSAAMPGASLQRACRLLVAVCALHLGVTLVYYLAGRDLRRLPQLVGVHPPLQGSSHGAAAIGQPSGELRLRGVAPPPPLQNSSKPRSRAPSNLDAYSHPGPGPGPGSNLTSAPVPSTTTRSLTACPEESPLLVGPMLIEFNIPVDLKLVEQQNPKVKLGGRYTPMDCISPHKVAIIIPFRNRQEHLKYWLYYLHPILQRQQLDYGIYVINQAGESMFNRAKLLNVGFKEALKDYDYNCFVFSDVDLIPMNDHNTYRCFSQPRHISVAMDKFGFSLPYVQYFGGVSALSKQQFLSINGFPNNYWGWGGEDDDIYNRLAFRGMSVSRPNAVIGKCRMIRHSRDKKNEPNPQRFDRIAHTKETMLSDGLNSLTYMVLEVQRYPLYTKITVDIGTPS

SEQ ID NO: 101 - protein, 341 aa, synthetic construct
  UNSURE 1..341: note = HsFUT1
MHIHQDSFPHGLGLSILCPDRRLVTPPVAIFCLPGTAMGPNASSSCPQHPASLSGTWTVYPNGRFGNQMGQYATLLALAQLNGRRAFILPAMHAALAPVFRITLPVLAPEVDSRTPWRELQLHDWMSEEYADLRDPFLKLSGFPCSWTFFHHLREQIRREFTLHDHLREEAQSVLGQLRLGRTGDRPRTFVGVHVRRGDYLQVMPQRWKGVVGDSAYLRQAMDWFRARHEAPVFVVTSNGMEWCKENIDTSQGDVTFAGDGQEATPWKDFALLTQCNHTIMTIGTFGFWAAYLAGGDTVYLANFTLPDSEFLKIFKPEAAFLPEWVGINADLSPLWTLAKP

SEQ ID NO: 102 - protein, 315 aa, synthetic construct
  UNSURE 1..315: note = HsFUT2
MQRLAKIQAMWELPVQIPVLASTSKALGPSQLRGMWTINAIGRLGNQMGEYATLYALAKMNGRPAFIPAQMHSTLAPIFRITLPVLHSATASRIPWQNYHLNDWMEEEYRHIPGEYVRFTGYPCSWTFYHHLRQEILQEFTLHDHVREEAQKFLRGLQVNGSRPGTFVGVHVRRGDYVHVMPKVWKGVVADRRYLQQALDWFRARYSSLIFVVTSNGMAWCRENIDTSHGDVVFAGDGIEGSPAKDFALLTQCNHTIMTIGTFGIWAAYLTGGDTIYLANYTLPDSPFLKIFKPEAAFLPEWTGIAADLSPLLKH

SEQ ID NO: 103 - protein, 328 aa, synthetic construct
  UNSURE 1..328: note = HsFUT3
MRVSRDDATGSPRAPSGSSRQDTTPTRPTLLILLWTWPFHIPVALSRCSEMVPGTADCHITADRKVYPQADTVIVHHWDIMSNPKSRLPPSPRPQGQRWIWFNLEPPPNCQHLEALDRYFNLTMSYRSDSDIFTPYGWLEPWSGQPAHPPLNLSAKTELVAWAVSNWKPDSARVRYYQSLQAHLKVDVYGRSHKPLPKGTMMETLSRYKFYLAFENSLHPDYITEKLWRNALEAWAVPVVLGPSRSNYERFLPPDAFIHVDDFQSPKDLARYLQELDKDHARYLSYFRWRETLRPRSFSWALDFCKACWKLQQESRYQTVRSIAAWFT

SEQ ID NO: 104 - protein, 359 aa, synthetic construct
  UNSURE 1..359: note = HsFUT4
MGQLPPLPWASPTPSRPVGVLLWWEPFGGRDSAPRPPPDCRLRFNISGCRLLTDRASYGEAQAVLFHHRDLVKGPPDWPPPWGIQAHTAEEVDLRVLDYEEAAAAAEALATSSPRPPGQRWVWMNFESPSHSPGLRSLASNLFNWTLSYRADSDVFVPYGYLYPRSHPGDPPSGLAPPLSRKQGLVAWVVSHWDERQARVRYYHQLSQHVTVDVFGRGGPGQPVPEIGLLHTVARYKFYLAFENSQHLDYITEKLWRNALLAGAVPVVLGPDRANYERFVPRGAFIHVDDFPSASSLASYLLFLDRNPAVYRRYFHWRRSYAVHITSFWDEPWCRVCQAVQRAGDRPKSIRNLASWFER

SEQ ID NO: 105 - protein, 341 aa, synthetic construct
  UNSURE 1..341: note = HsFUT5
MRVSRDDATGSPRPGLMAVEPVTGAPNGSRCQDSMATPAHPTLLILLWTWPFNTPVALPRCSEMVPGAADCNITADSSVYPQADAVIVHHWDIMYNPSANLPPPTRPQGQRWIWFSMESPSNCRHLEALDGYFNLTMSYRSDSDIFTPYGWLEPWSGQPAHPPLNLSAKTELVAWAVSNWKPDSARVRYYQSLQAHLKVDVYGRSHKPLPKGTMMETLSRYKFYLAFENSLHPDYITEKLWRNALEAWAVPVVLGPSRSNYERFLPPDAFIHVDDFQSPKDLARYLQELDKDHARYLSYFRWRETLRPRSFSWALAFCKACWKLQQESRYQTVRSIAAWFT

SEQ ID NO: 106 - protein, 326 aa, synthetic construct
  UNSURE 1..326: note = HsFUT6
MRVSQDDPTVYPNGSRFPDSTGTPAHSIPLILLWTWPFNKPIALPRCSEMVPGTADCNITADRKVYPQADAVIVHHREVMYNPSAQLPRSPRRQGQRWIWFSMESPSHCWQLKAMDGYFNLTMSYRSDSDIFTPYGWLEPWSGQPAHPPLNLSAKTELVAWAVSNWGPNSARVRYYQSLQAHLKVDVYGRSHKPLPQGTMMETLSRYKFYLAFENSLHPDYITEKLWRNALEAWAVPVVLGPSRSNYERFLPPDAFIHVDDFQSPKDLARYLQELDKDHARYLSYFRWRETLRPRSFSWALAFCKACWKLQEESRYQTRGIAAWFT

SEQ ID NO: 107 - protein, 308 aa, synthetic construct
  UNSURE 1..308: note = HsFUT7
MAPRGTPAPQPTITILVWHWPFTDQPPELPSDTCTRYGIARCHLSANRSLLASADAVVFHHRELQTRRSHLPLAQRPRGQPWVWASMESPSHTHGLSHLRGIFNWVLSYRRDSDIFVPYGRLEPHWGPSPPLPAKSRVAAWVVSNFQERQLRARLYRQLAPHLRVDVFGRANGRPLCASCLVPTVAQYRFYLSFENSQHRDYITEKFWRNALVAGTVPVVLGPPRATYEAFVPADAFVHVDDFGSARELAAFLTGMNESRYQRFFAWRDRLRVRLFTDWRERFCAICDRYPHLPRSQVYEDLEGWFQA

SEQ ID NO: 108 - protein, 546 aa, synthetic construct
  UNSURE 1..546: note = HsFUT8
MRDNDHPDHSSRELSKILAKLERLKQQNEDLRRMAESLRIPEGPIDQGPAIGRVRVLEEQLVKAKEQIENYKKQTRNGLGKDHEILRRRIENGAKELWFFLQSELKKLKNLEGNELQRHADEFLLDLGHHERSIMTDLYYLSQTDGAGDWREKEAKDLTELVQRRITYLQNPKDCSKAKKLVCNINKGCGYGCQLHHVVYCFMIAYGTQRTLILESQNWRYATGGWETVFRPVSETCTDRSGISTGHWSGEVKDKNVQVVELPIVDSLHPRPPYLPLAVPEDLADRLVRVHGDPAVWWVSQFVKYLIRPQPWLEKEIEEATKKLGFKHPVIGVHVRRTDKVGTEAAFHPIEEYMVHVEEHFQLLARRMQVDKKRVYLATDDPSLLKEAKTKYPNYEFISDNSISWSAGLHNRYTENSLRGVILDIHFLSQADFLVCTFSSQVCRVAYEIMQTLHPDASANFHSLDDIYYFGGQNAHNQIAIYAHQPRTADEIPMEPGDIIGVAGNHWDGYSKGVNRKLGRTGLYPSYKVREKIETVKYPTYPEAEK

SEQ ID NO: 109 - protein, 328 aa, synthetic construct
  UNSURE 1..328: note = HsFUT9
MTNSWIFSPMESASSVLKMKNFFSTKTDYFNETTILVWVWPFGQTFDLTSCQAMFNIQGCHLTTDRSLYNKSHAVLIHHRDISWDLTNLPQQARPPFQKWIWMNLESPTHTPQKSGIEHLFNLTLTYRRDSDIQVPYGFLTVSTNPFVFEVPSKEKLVCWVVSNWNPEHARVKYYNELSKSIEIHTYGQAFGEYVNDKNLIPTISTCKFYLSFENSIHKDYITEKLYNAFLAGSVPVVLGPSRENYENYIPADSFIHVEDYNSPSELAKYLKEVDKNNKLYLSYFNWRKDFTVNLPRFWESHACLACDHVKRHQEYKSVGNLEKWFWN

SEQ ID NO: 110 - protein, 449 aa, synthetic construct
  UNSURE 1..449: note = HsFUT10
MLGKFERKEFKSSSLQDGHTKMEEAPTHLNSFLKKEGLTFNRKRKWELDSYPIMLWWSPLTGETGRLGQCGADACFFTINRTYLHHHMTKAFLFYGTDFNIDSLPLPRKAHHDWAVFHEESPKNNYKLFHKPVITLFNYTATFSRHSHLPLTTQYLESIEVLKSLRYLVPLQSKNKLRKRLAPLVYVQSDCDPPSDRDSYVRELMTYIEVDSYGECLRNKDLPQQLKNPASMDADGFYRIIAQYKFILAFENAVCDDYITEKFWRPLKLGVVPVYYGSPSITDWLPSNKSAILVSEFSHPRELASYIRRLDSDDRLYEAYVEWKLKGEISNQRLLTALRERKWGVQDVNQDNYIDAFECMVCTKVWANIRLQEKGLPPKRWEAEDTHLSCPEPTVFAFSPLRTPPLSSLREMWISSFEQSKKEAQALRWLVDRNQNFSSQEFWGLVFKD

SEQ ID NO: 111 - protein, 469 aa, synthetic construct
  UNSURE 1..469: note = HsFUT11
MGSVAEREAGGEAEWAEPWDGAVFRPPSALGAVGVTRSSGTPRPGREEAGDLPVLLWWSPGLFPHFPGDSERIECARGACVASRNRRALRDSRTRALLFYGTDFRASAAPLPRLAHQSWALLHEESPLNNFLLSHGPGIRLFNLTSTFSRHSDYPLSLQWLPGTAYLRRPVPPPMERAEWRRRGYAPLLYLQSHCDVPADRDRYVRELMRHIPVDSYGKCLQNRELPTARLQDTATATTEDPELLAFLSRYKFHLALENAICNDYMTEKLWRPMHLGAVPVYRGSPSVRDWMPNNHSVILIDDFESPQKLAEFIDFLDKNDEEYMKYLAYKQPGGITNQFLLDSLKHREWGVNDPLLPNYLNGFECFVCDYELARLDAEKAHAASPGDSPVFEPHIAQPSHMDCPVPTPGFGNVEEIPENDSWKEMWLQDYWQGLDQGEALTAMIHNNETEQTKFWDYLHEIFMKRQHL

SEQ ID NO: 112 - protein, 363 aa, synthetic construct
  UNSURE 1..363: note = HsPOFUT1
MGSWDPAGYLLYCPCMGRFGNQADHFLGSLAFAKLLNRTLAVPPWIEYQHHKPPFTNLHVSYQKYFKLEPLQAYHRVISLEDFMEKLAPTHWPPEKRVAYCFEVAAQRSPDKKTCPMKEGNPFGPFWDQFHVSFNKSELFTGISFSASYREQWSQRFSPKEHPVLALPGAPAQFPVLEEHRPLQKYMVWSDEMVKTGEAQIHAHLVRPYVGIHLRIGSDWKNACAMLKDGTAGSHFMASPQCVGYSRSTAAPLTMTMCLPDLKEIQRAVKLWVRSLDAQSVYVATDSESYVPELQQLFKGKVKVVSLKPEVAQVDLYILGQADHFIGNCVSSFTAFVKRERDLQGRPSSFFGMDRPPKLRDEF

SEQ ID NO: 113 - protein, 307 aa, synthetic construct
  UNSURE 1..307: note = HsST3Gal1
MTTWFPKQMVLELSENLKRLIKHRPCTCTHCIGQRKLSAWFDERFNQTMQPLLTAQNALLEDDTYRWWLRLQREKKPNNLNDTIKELFRVVPGNVDPMLEKRSVGCRRCAVVGNSGNLRESSYGPEIDSHDFVLRMNKAPTAGFEADVGTKTTHHLVYPESFRELGDNVSMILVPFKTIDLEWVVSAITTGTISHTYIPVPAKIRVKQDKILIYHPAFIKYVFDNWLQGHGRYPSTGILSVIFSMHVCDEVDLYGFGADSKGNWHHYWENNPSAGAFRKTGVHDADFESNVTATLASINKIRIFKGR

SEQ ID NO: 114 - protein, 348 aa, synthetic construct
  UNSURE 1..348: note = HsST3Gal3
MKLHLLQWEEDSNSVVLSFDSAGQTLGSEYDRLGFLLNLDSKLPAELATKYANFSEGACKPGYASALMTAIFPRFSKPAPMFLDDSFRKWARIREFVPPFGIKGQDNLIKAILSVTKEYRLTPALDSLRCRRCIIVGNGGVLANKSLGSRIDDYDIVVRLNSAPVKGFEKDVGSKTTLRITYPEGAMQRPEQYERDSLFVLAGFKWQDFKWLKYIVYKERVSASDGFWKSVATRVPKEPPEIRILNPYFIQEAAFTLIGLPFNNGLMGRGNIPTLGSVAVTMALHGCDEVAVAGFGYDMSTPNAPLHYYETVRMAAIKESWTHNIQREKEFLRKLVKARVITDLSSGI

SEQ ID NO: 115 - protein, 308 aa, synthetic construct
  UNSURE 1..308: note = HsST3Gal4
MREDRYIELFYFPIPEKKEPCLQGEAESKASKLFGNYSRDQPIFLRLEDYFWVKTPSAYELPYGTKGSEDLLLRVLAITSSSIPKNIQSLRCRRCVVVGNGHRLRNSSLGDAINKYDVVIRLNNAPVAGYEGDVGSKTTMRLFYPESAHFDPKVENNPDTLLVLVAFKAMDFHWIETILSDKKRVRKGFWKQPPLIWDVNPKQIRILNPFFMEIAADKLLSLPMQQPRKIKQKPTTGLLAITLALHLCDLVHIAGFGYPDAYNKKQTIHYYEQITLKSMAGSGHNVSQEALAIKRMLEMGAIKNLTSF

SEQ ID NO: 116 - protein, 307 aa, synthetic construct
  UNSURE 1..307: note = HsST3Gal6
MTNVYWVAPVEMKRRNKIQPCLSKPAFASLLRFHQFHPFLCAADFRKIASLYGSDKFDLPYGMRTSAEYFRLALSKLQSCDLFDEFDNIPCKKCVVVGNGGVLKNKTLGEKIDSYDVIIRMNNGPVLGHEEEVGRRTTFRLFYPESVFSDPIHNDPNTTVILTAFKPHDLRWLLELLMGDKINTNGFWKKPALNLIYKPYQIRILDPFIIRTAAYELLHFPKVFPKNQKPKHPTTGIIAITLAFYICHEVHLAGFKYNFSDLKSPLHYYGNATMSLMNKNAYHNVTAEQLFLKDIIEKNLVINLTQD

SEQ ID NO: 117 - protein, 381 aa, synthetic construct
  UNSURE 1..381: note = HsST6Gal1
MKEKKKGSYYDSFKLQTKEFQVLKSLGKLAMGSDSQSVSSSSTQDPHRGRQTLGSLRGLAKAKPEASFQVWNKDSSSKNLIPRLQKIWKNYLSMNKYKVSYKGPGPGIKFSAEALRCHLRDHVNVSMVEVTDFPFNTSEWEGYLPKESIRTKAGPWGRCAVVSSAGSLKSSQLGREIDDHDAVLRFNGAPTANFQQDVGTKTTIRLMNSQLVTTEKRFLKDSLYNEGILIVWDPSVYHSDIPKWYQNPDYNFFNNYKTYRKLHPNQPFYILKPQMPWELWDILQEISPEEIQPNPPSSGMLGIIIMMTLCDQVDIYEFLPSKRKTDVCYYYQKFFDSACTMGAYHPLLYEKNLVKHLNQGTDEDIYLLGKATLPGFRTIHC

SEQ ID NO: 118 - protein, 566 aa, synthetic construct
  UNSURE 1..566: note = HsST6GalNAc1
MKEPQTKPSRHQRTENIKERSLQSLAKPKSQAPTRARRTTIYAEPVPENNALNTQTQPKAHTTGDRGKEANQAPPEEQDKVPHTAQRAAWKSPEKEKTVVNTLSPRGQDAGMASGRTEAQSWKSQDTKTTQGNGGQTRKLTASRTVSEKHQGKAATTAKTLIPKSQHRMLVPTGAVSTRTRQKGVTTAVIPPKEKKPQATPPPAPFQSPTTQRNQRLKAANFKSEPRWDFEEKYSFEIGGLQTTCPDSVKIKASKSLWLQKLFLPNLTLFLDSRHFNQSEWDRLEHFAPPFGFMELNYSLVQKVVTRFPPVPQQQLLLASLPAGSLRCITCAVVGNGGILNNSHIGQEIDSHDYVFRLSGALIKGYEQDVGTRTSFYGFTAFSLTQSLLILGNRGFKNVPLGKDVRYLHFLEGTRDYEWLEALLMNQTVMSKNLFWFRHRPQEAFREALHMDRYLLLHPDFLRYMKNRFLRSKTLDGAHWRIYRPTTGALLLLTALQLCDQVSAYGFITEGHERFSDHYYDTSWKRLIFYINHDFKLEREVWKRLHDEGIIRLYQRPGPGTAKAKN

SEQ ID NO: 119 - protein, 347 aa, synthetic construct
  UNSURE 1..347: note = HsST6GalNAc2
MSAVQRYPGPAAGARDTTSFEAFFQSKASNSWTGKGQACRHLLHLAIQRHPHFRGLFNLSIPVLLWGDLFTPALWDRLSQHKAPYGWRGLSHQVIASTLSLLNGSESAKLFAPPRDTPPKCIRCAVVGNGGILNGSRQGPNIDAHDYVFRLNGAVIKGFERDVGTKTSFYGFTVNTMKNSLVSYWNLGFTSVPQGQDLQYIFIPSDIRDYVMLRSAILGVPVPEGLDKGDRPHAYFGPEASASKFKLLHPDFISYLTERFLKSKLINTHFGDLYMPSTGALMLLTALHTCDQVSAYGFITSNYWKFSDHYFERKMKPLIFYANHDLSLEAALWRDLHKAGILQLYQR

SEQ ID NO: 120 - protein, 276 aa, synthetic construct
  UNSURE 1..276: note = HsST6GalNAc4
MAGLPLCLATCLDHHFPTGSRPTVPGPLHFSGYSSVPDGKPLVREPCRSCAVVSSSGQMLGSGLGAEIDSAECVFRMNQAPTVGFEADVGQRSTLRVVSHTSVPLLLRNYSHYFQKARDTLYMVWGQGRHMDRVLGGRTYRTLLQLTRMYPGLQVYTFTERMMAYCDQIFQDETGKNRRQSGSFLSTGWFTMILALELCEEIVVYGMVSDSYCREKSHPSVPYHYFEKGRLDECQMYLAHEQAPRSAHRFITEKAVFSRWAKKRPIVFAHPSWRTE

SEQ ID NO: 121 - protein, 309 aa, synthetic construct
  UNSURE 1..309: note = HsST8Sia1
MYRLPNEKEIVQGVLQQGTAWRRNQTAARAFRKQMEDCCDPAHLFAMTKMNSPMGKSMWYDGEFLYSFTIDNSTYSLFPQATPFQLPLKKCAVVGNGGILKKSGCGRQIDEANFVMRCNLPPLSSEYTKDVGSKSQLVTANPSIIRQRFQNLLWSRKTFVDNMKIYNHSYIYMPAFSMKTGTEPSLRVYYTLSDVGANQTVLFANPNFLRSIGKFWKSRGIHAKRLSTGLFLVSAALGLCEEVAIYGFWPFSVNMHEQPISHHYYDNVLPFSGFHAMPEEFLQLWYLHKIGALRMQLDPCEDTSLQPTS

SEQ ID NO: 122 - protein, 353 aa, synthetic construct
  UNSURE 1..353: note = HsST8Sia2
MDISEIEEEIGNSGGRGTIRSAVNSLHSKSNRAEVVINGSSSPAVVDRSNESIKHNIQPASSKWRHNQTLSLRIRKQILKFLDAEKDISVLKGTLKPGDIIHYIFDRDSTMNVSQNLYELLPRTSPLKNKHFGTCAIVGNSGVLLNSGCGQEIDAHSFVIRCNLAPVQEYARDVGLKTDLVTMNPSVIQRAFEDLVNATWREKLLQRLHSLNGSILWIPAFMARGGKERVEWVNELILKHHVNVRTAYPSLRLLHAVRGYWLTNKVHIKRPTTGLLMYTLATRFCKQIYLYGFWPFPLDQNQNPVKYHYYDSLKYGYTSQASPHTMPLEFKALKSLHEQGALKLTVGQCDGAT

SEQ ID NO: 123 - protein, 348 aa, synthetic construct
  UNSURE 1..348: note = HsST8Sia3
MKKENIFTTPKYASPGAPRMYMFHAGFRSQFALKFLDPSFVPITNSLTQELQEKPSKWKFNRTAFLHQRQEILQHVDVIKNFSLTKNSVRIGQLMHYDYSSHKYVFSISNNFRSLLPDVSPIMNKHYNICAVVGNSGILTGSQCGQEIDKSDFVFRCNFAPTEAFQRDVGRKTNLTTFNPSILEKYYNNLLTIQDRNNFFLSLKKLDGAILWIPAFFFHTSATVTRTLVDFFVEHRGQLKVQLAWPGNIMQHVNRYWKNKHLSPKRLSTGILMYTLASAICEEIHLYGFWPFGFDPNTREDLPYHYYDKKGTKFTTKWQESHQLPAEFQLLYRMHGEGLTKLTLSHCA

SEQ ID NO: 124 - protein, 340 aa, synthetic construct
  UNSURE 1..340: note = HsST8Sia4
MKTKEIARTEEHQETQLIGDGELSLSRSLVNSSDKIIRKAGSSIFQHNVEGWKINSSLVLEIRKNILRFLDAERDVSVVKSSFKPGDVIHYVLDRRRTLNISHDLHSLLPEVSPMKNRRFKTCAVVGNSGILLDSECGKEIDSHNFVIRCNLAPVVEFAADVGTKSDFITMNPSVVQRAFGGFRNESDREKFVHRLSMLNDSVLWIPAFMVKGGEKHVEWVNALILKNKLKVRTAYPSLRLIHAVRGYWLTNKVPIKRPSTGLLMYTLATRFCDEIHLYGFWPFPKDLNGKAVKYHYYDDLKYRYFSNASPHRMPLEFKTLNVLHNRGALKLTTGKCVKQ

SEQ ID NO: 125 - protein, 532 aa, synthetic construct
  UNSURE 1..532: note = HsGalNAcT1
MSECNKCDEKKERGLPAGDVLEPVQKPHEGPGEMGKPVVIPKEDQEKMKEMFKINQFNLMASEMIALNRSLPDVRLEGCKTKVYPDNLPTTSVVIVFHNEAWSTLLRTVHSVINRSPRHMIEEIVLVDDASERDFLKRPLESYVKKLKVPVHVIRMEQRSGLIRARLKGAAVSKGQVITFLDAHCECTVGWLEPLLARIKHDRRTVVCPIIDVISDDTFEYMAGSDMTYGGFNWKLNFRWYPVPQREMDRRKGDRTLPVRTPTMAGGLFSIDRDYFQEIGTYDAGMDIWGGENLEISFRIWQCGGTLEIVTCSHVGHVFRKATPYTFPGGTGQIINKNNRRLAEVWMDEFKNFFYIISPGVTKVDYGDISSRVGLRHKLQCKPFSWYLENIYPDSQIPRHYFSLGEIRNVETNQCLDNMARKENEKVGIFNCHGMGGNQVFSYTANKEIRTDDLCLDVSKLNGPVTMLKCHHLKGNQLWEYDPVKLTLQHVNSNQCLDKATEEDSQVPSIRDCNGSRSQQWLLRNVTLPEIF

SEQ ID NO: 126 - protein, 548 aa, synthetic construct
  UNSURE 1..548: note = HsGalNAcT2
MSGGGSALAGGAGGGAGRKEDWNEIDPIKKKDLHHSNGEEKAQSMETLPPGKVRWPDFNQEAYVGGTMVRSGQDPYARNKFNQVESDKLRMDRAIPDTRHDQCQRKQWRVDLPATSVVITFHNEARSALLRTVVSVLKKSPPHLIKEIILVDDYSNDPEDGALLGKIEKVRVLRNDRREGLMRSRVRGADAAQAKVLTFLDSHCECNEHWLEPLLERVAEDRTRVVSPIIDVINMDNFQYVGASADLKGGFDWNLVFKWDYMTPEQRRSRQGNPVAPIKTPMIAGGLFVMDKFYFEELGKYDMMMDVWGGENLEISFRVWQCGGSLEIIPCSRVGHVFRKQHPYTFPGGSGTVFARNTRRAAEVWMDEYKNFYYAAVPSARNVPYGNIQSRLELRKKLSCKPFKWYLENVYPELRVPDHQDIAFGALQQGTNCLDTLGHFADGVVGVYECHNAGGNQEWALTKEKSVKHMDLCLTVVDRAPGSLIKLQGCRENDSRQKWEQIEGNSKLRHVGSNLCLDSRTAKSGGLSVEVCGPALSQQWKFTLNLQQ

SEQ ID NO: 127 - protein, 597 aa, synthetic construct [sequence omitted]

SEQ ID NO: 128 - protein, 544 aa, synthetic construct
  UNSURE 1..544: note = HsGalNAcT4
MAGAGRARELGSRRLSDLQKNTEDLSRPLYKKPPADSRALGEWGKASKLQLNEDELKQQEELIERYAINIYLSDRISLHRHIEDKRMYECKSQKFNYRTLPTTSVIIAFYNEAWSTLLRTIHSVLETSPAVLLKEIILVDDLSDRVYLKTQLETYISNLDRVRLIRTNKREGLVRARLIGATFATGDVLTFLDCHCECNSGWLEPLLERIGRDETAVVCPVIDTIDWNTFEFYMQIGEPMIGGFDWRLTFQWHSVPKQERDRRISRIDPIRSPTMAGGLFAVSKKYFQYLGTYDTGMEVWGGENLELSFRVWQCGGKLEIHPCSHVGHVFPKRAPYARPNFLQNTARAAEVWMDEYKEHFYNRNPPARKEAYGDISERKLLRERLRCKSFDWYLKNVFPNLHVPEDRPGWHGAIRSRGISSECLDYNSPDNNPTGANLSLFGCHGQGGNQFFEYTSNKEIRFNSVTELCAEVPEQKNYVGMQNCPKDGFPVPANIIWHFKEDGTIFHPHSGLCLSAYRTPEGRPDVQMRTCDALDKNQIWSFEK

SEQ ID NO: 129 - protein, 906 aa, synthetic construct
  UNSURE 1..906: note = HsGalNAcT5
MSFSEINTRVIKEDIVRRERIGFRVQPDQGKIFYSSIKEMKPPLRGHGKGAWGKENVRKTEESVLKVEVDLDQTQRERKMQNALGRGKVVPLWHPAHLQTLPVTPNKQKTDGRGTKPEASSHQGTPKQTTAQGAPKTSFIAAKGTQVVKISVHMGRVSLKQEPRKSHSPSSDTSKLAAERDLNVTISLSTDRPKQRSQAVANERAHPASTAVPKSGEAMALNKTKTQSKEVNANKHKANTSLPFPKFTVNSNRLRKQSINETPLGSLSKDDGARGAHGKKLNFSESHLVIITKEEEQKADPKEVSNSKTKTIFPKVLGKSQSKHISRNRSEMSSSSLAPHRVPLSQTNHALTGGLEPAKINITAKAPSTEYNQSHIKALLPEDSGTHQVLRIDVTLSPRDPKAPGQFGRPVVVPHGKEKEAERRWKEGNFNVYLSDLIPVDRAIEDTRPAGCAEQLVHNNLPTTSVIMCFVDEVWSTLLRSVHSVINRSPPHLIKEILLVDDFSTKDYLKDNLDKYMSQFPKVRILRLKERHGLIRARLAGAQNATGDVLTFLDSHVECNVGWLEPLLERVYLSRKKVACPVIEVINDKDMSYMTVDNFQRGIFVWPMNFGWRTIPPDVIAKNRIKETDTIRCPVMAGGLFSIDKSYFFELGTYDPGLDVWGGENMELSFKVWMCGGEIEIIPCSRVGHIFRNDNPYSFPKDRMKTVERNLVRVAEVWLDEYKELFYGHGDHLIDQGLDVGNLTQQRELRKKLKCKSFKWYLENVFPDLRAPIVRASGVLINVALGKCISIENTTVILEDCDGSKELQQFNYTWLRLIKCGEWCIAPIPDKGAVRLHPCDNRNKGLKWLHKSTSVFHPELVNHIVFENNQQLLCLEGNFSQKILKVAACDPVKPYQKWKFEKYYEA

SEQ ID NO: 130 - protein, 595 aa, synthetic construct
  UNSURE 1..595: note = HsGalNAcT6
MRDVSSREEATEKPWLKSLVSRKDHVLDLMLEAMNNLRDSMPKLQIRAPEAQQTLFSINQSCLPGFYTPAELKPFWERPPQDPNAPGADGKAFQKSKWTPLETQEKEEGYKKHCFNAFASDRISLQRSLGPDTRPPECVDQKFRRCPPLATTSVIIVFHNEAWSTLLRTVYSVLHTTPAILLKEIILVDDASTEEHLKEKLEQYVKQLQVVRVVRQEERKGLITARLLGASVAQAEVLTFLDAHCECFHGWLEPLLARIAEDKTVVVSPDIVTIDLNTFEFAKPVQRGRVHSRGNFDWSLTFGWETLPPHEKQRRKDETYPIKSPTFAGGLFSISKSYFEHIGTYDNQMEIWGGENVEMSFRVWQCGGQLEIIPCSVVGHVFRTKSPHTFPKGTSVIARNQVRLAEVWMDSYKKIFYRRNLQAAKMAQEKSFGDISERLQLREQLHCHNFSWYLHNVYPEMFVPDLTPTFYGAIKNLGTNQCLDVGENNRGGKPLIMYSCHGLGGNQYFEYTTQRDLRHNIAKQLCLHVSKGALGLGSCHFTGKNSQVPKDEEWELAQDQLIRNSGSGTCLTSQDKKPAMAPCNPSDPHQLWLFV

SEQ ID NO: 131 - protein, 629 aa, synthetic construct
  UNSURE 1..629: note = HsGalNAcT7
MPRPDDPSPLSRMREDRDVNDPMPNRGGNGLAPGEDRFKPVVPWPHVEGVEVDLESIRRINKAKNEQEHHAGGDSQKDIMQRQYLTFKPQTFTYHDPVLRPGILGNFEPKEPEPPGVVGGPGEKAKPLVLGPEFKQAIQASIKEFGFNMVASDMISLDRSVNDLRQEECKYWHYDENLLTSSVVIVFHNEGWSTLMRTVHSVIKRTPRKYLAEIVLIDDFSNKEHLKEKLDEYIKLWNGLVKVFRNERREGLIQARSIGAQKAKLGQVLIYLDAHCEVAVNWYAPLVAPISKDRTICTVPLIDVINGNTYEIIPQGGGDEDGYARGAWDWSMLWKRVPLTPQEKRLRKTKTEPYRSPAMAGGLFAIEREFFFELGLYDPGLQIWGGENFEISYKIWQCGGKLLFVPCSRVGHIYRLEGWQGNPPPIYVGSSPTLKNYVRVVEVWWDEYKDYFYASRPESQALPYGDISELKKFREDHNCKSFKWFMEEIAYDITSHYPLPPKNVDWGEIRGFETAYCIDSMGKTNGGFVELGPCHRMGGNQLFRINEANQLMQYDQCLTKGADGSKVMITHCNLNEFKEWQYFKNLHRFTHIPSGKCLDRSEVLHQVFISNCDSSKTTQKWEMNNIHSV

SEQ ID NO: 132 - protein, 609 aa, synthetic construct
  UNSURE 1..609: note = HsGalNAcT8
MKGTLQNLFTGGLHRELPLHLNKRYGAVIKRLSHLEVELQDLKESMKLALRQQENVNSTLKRAKDEVRPLLKAMETKVNETKKHKTQMKLFPHSQLFRQWGEDLSEAQQKAAQDLFRKFGYNAYLSNQLPLNRTIPDTRDYRCLRKTYPSQLPSLSVILIFVNEALSIIQRAITSIINRTPSRLLKEIILVDDFSSNGELKVHLDEKIKLYNQKYPGLLKIIRHPERKGLAQARNTGWEAATADVVAILDAHIEVNVGWAEPILARIQEDRTVIVSPVFDNIRFDTFKLDKYELAVDGFNWELWCRYDALPQAWIDLHDVTAPVKSPSIMGILAANRHFLGEIGSLDGGMLIYGGENVELSLRVWQCGGKVEILPCSRIAHLERHHKPYALDLTAALKRNALRVAEIWMDEHKHMVYLAWNIPLQNSGIDFGDVSSRMALREKLKCKTFDWYLKNVYPLLKPLHTIVGYGRMKNLLDENVCLDQGPVPGNTPIMYYCHEFSSQNVYYHLTGELYVGQLIAEASASDRCLTDPGKAEKPTLEPCSKAAKNRLHIYWDFKPGGAVINRDTKRCLEMKKDLLGSHVLVLQTCSTQVWEIQHTVRDWGQTNSQ

SEQ ID NO: 133 - protein, 575 aa, synthetic construct
  UNSURE 1..575: note = HsGalNAcT9
MLQGRSQELVRIVSGDRRVRSRHAKVGTLGDREAILQRLDHLEEVVYNQLNGLAKPIGLVEGPGGLGQGGLAATLRDDGQEAEGKYEEYGYNAQLSDRISLDRSIPDYRPRKCRQMSYAQDLPQVSVVFIFVNEALSVILRSVHSVVNHTPSQLLKEVILVDDNSDNVELKFNLDQYVNKRYPGLVKIVRNSRREGLIRARLQGWKAATAPVVGFFDAHVEFNTGWAEPALSRIREDRRRIVLPAIDNIKYSTFEVQQYANAAHGYNWGLRCMYIIPPQDWLDRGDESAPIRTPAMIGCSFVVDREYFGDIGLLDPGMEVYGGENVELGMRVWQCGGSMEVLPCSRVAHIERTRKPYNNDIDYYAKRNALRAAEVWMDDFKSHVYMAWNIPMSNPGVDFGDVSERLALRQRLKCRSFKWYLENVYPEMRVYNNTLTYGEVRNSKASAYCLDQGAEDGDRAILYPCHGMSSQLVRYSADGLLQLGPLGSTAFLPDSKCLVDDGTGRMPTLKKCEDVARPTQRLWDFTQSGPIVSRATGRCLEVEMSKDANFGLRLVVQRCSGQKWMIRNWIKHARH

SEQ ID NO: 134 - protein, 573 aa, synthetic construct
  UNSURE 1..573: note = HsGalNAcT10
MRERQPDGTPGGSGAAVAPAAGQGSHSRQKKTFFLGDGQKLKDWHDKEAIRRDAQRVGNGEQGRPYPMTDAERVDQAYRENGFNIYVSDKISLNRSLPDIRHPNCNSKRYLETLPNTSIIIPFHNEGWSSLLRTVHSVLNRSPPELVAEIVLVDDFSDREHLKKPLEDYMALFPSVRILRTKKREGLIRTRMLGASVATGDVITFLDSHCEANVNWLPPLLDRIARNRKTIVCPMIDVIDHDDFRYETQAGDAMRGAFDWEMYYKRIPIPPELQKADPSDPFESPVMAGGLFAVDRKWFWELGGYDPGLEIWGGEQYEISFKVWMCGGRMEDIPCSRVGHIYRKYVPYKVPAGVSLARNLKRVAEVWMDEYAEYIYQRRPEYRHLSAGDVAVQKKLRSSLNCKSFKWFMTKIAWDLPKFYPPVEPPAAAWGEIRNVGTGLCADTKHGALGSPLRLEGCVRGRGEAAWNNMQVFTFTWREDIRPGDPQHTKKFCFDAISHTSPVTLYDCHSMKGNQLWKYRKDKTLYHPVSGSCMDCSESDHRIFMNTCNPSSLTQQWLFEHTNSTVLEKFNRN

SEQ ID NO: 135 - protein, 289 aa, synthetic construct
  UNSURE 1..289: note = HsB3GALNT1
MIERVNWMYFYEYEPIYRQDFHFTLREHSNCSHQNPFLVILVTSHPSDVKARQAIRVTWGEKKSWWGYEVLTFFLLGQEAEKEDKMLALSLEDEHLLYGDIIRQDFLDTYNNLTLKTIMAFRWVTEFCPNAKYVMKTDTDVFINTGNLVKYLLNLNHSEKFFTGYPLIDNYSYRGFYQKTHISYQEYPFKVFPPYCSGLGYIMSRDLVPRIYEMMGHVKPIKFEDVYVGICLNLLKVNIHIPEDTNLFFLYRIHLDVCQLRRVIAAHGFSSKEIITFWQVMLRNTTCHY

SEQ ID NO: 136 - protein, 509 aa, synthetic construct
  UNSURE 1..509: note = HsB4GALNT1
MSTRDAPGLRLPLAPWAPPQSPRRPELPDLAPEPRYAHIPVRIKEQVVGLLAWNNCSCESSGGGLPLPFQKQVRAIDLTKAFDPAELRAASATREQEFQAFLSRSQSPADQLLIAPANSPLQYPLQGVEVQPLRSILVPGLSLQAASGQEVYQVNLTASLGTWDVAGEVTGVTLTGEGQADLTLVSPGLDQLNRQLQLVTYSSRSYQTNTADTVRFSTEGHEAAFTIRIRHPPNPRLYPPGSLPQGAQYNISALVTIATKTFLRYDRLRALITSIRRFYPTVTVVIADDSDKPERVSGPYVEHYLMPFGKGWFAGRNLAVSQVTTKYVLWVDDDFVFTARTRLERLVDVLERTPLDLVGGAVREISGFATTYRQLLSVEPGAPGLGNCLRQRRGFHHELVGFPGCVVTDGVVNFFLARTDKVREVGFDPRLSRVAHLEFFLDGLGSLRVGSCSDVVVDHASKLKLPWTSRDAGAETYARYRYPGSLDESQMAKHRLLFFKHRLQCMTSQ

SEQ ID NO: 137 - protein, 302 aa, synthetic construct [sequence omitted]

SEQ ID NO: 138 - protein, 311 aa, synthetic construct
  UNSURE 1..311: note = HsA4GALT
MGEPKEKGQLYNLPAEIPCPTLTPPTPPSHGPTPGNIFFLETSDRTNPNFLFMCSVESAARTHPESHVLVLMKGLPGGNASLPRHLGISLLSCFPNVQMLPLDLRELFRDTPLADWYAAVQGRWEPYLLPVLSDASRIALMWKFGGIYLDTDFIVLKNLRNLTNVLGTQSRYVLNGAFLAFERRHEFMALCMRDFVDHYNGWIWGHQGPQLLTRVFKKWCSIRSLAESRACRGVTTLPPEAFYPIPWQDWKKYFEDINPEELPRLLSATYAVHVWNKKSQGTRFEATSRALLAQLHARYCPTTHEAMKMYL

SEQ ID NO: 139 - protein, 301 aa, synthetic construct
  UNSURE 1..301: note = HsB3GalT1
MRPTSSYTGSKPFSHLTVARKNFTFGNIRTRPINPHSFEFLINEPNKCEKNIPFLVILISTTHKEFDARQAIRETWGDENNFKGIKIATLFLLGKNADPVLNQMVEQESQIFHDIIVEDFIDSYHNLTLKTLMGMRWVATFCSKAKYVMKTDSDIFVNMDNLIYKLLKPSTKPRRRYFTGYVINGGPIRDVRSKWYMPRDLYPDSNYPPFCSGTGYIFSADVAELIYKTSLHTRLLHLEDVYVGLCLRKLGIHPFQNSGFNHWKMAYSLCRYRRVITVHQISPEEMHRIWNDMSSKKHLRC

SEQ ID NO: 140 - protein, 378 aa, synthetic construct
  UNSURE 1..378: note = HsB3GalT2
MHDWLPGRAGFKENPVTYTFRGFRSTKSETNHSSLRNIWKETVPQTLRPQTATNSNNTDLSPQGVTGLENTLSANGSIYNEKGTGHPNSYHFKYIINEPEKCQEKSPFLILLIAAEPGQIEARRAIRQTWGNESLAPGIQITRIFLLGLSIKLNGYLQRAILEESRQYHDIIQQEYLDTYYNLTIKTLMGMNWVATYCPHIPYVMKTDSDMFVNTEYLINKLLKPDLPPRHNYFTGYLMRGYAPNRNKDSKWYMPPDLYPSERYPVFCSGTGYVFSGDLAEKIFKVSLGIRRLHLEDVYVGICLAKLRIDPVPPPNEFVFNHWRVSYSSCKYSHLITSHQFQPSELIKYWNHLQQNKHNACANAAKEKAGRYRHRKLH

SEQ ID NO: 141 - protein, 355 aa, synthetic construct
  UNSURE 1..355: note = HsB4GalT1
MRDLSRLPQLVGVSTPLQGGSNSAAAIGQSSGELRTGGARPPPPLGASSQPRPGGDSSPVVDSGPGPASNLTSVPVPHTTALSLPACPEESPLLVGPMLIEFNMPVDLELVAKQNPNVKMGGRYAPRDCVSPHKVAIIIPFRNRQEHLKYWLYYLHPVLQRQQLDYGIYVINQAGDTIFNRAKLLNVGFQEALKDYDYTCFVFSDVDLIPMNDHNAYRCFSQPRHISVAMDKFGFSLPYVQYFGGVSALSKQQFLTINGFPNNYWGWGGEDDDIFNRLVFRGMSISRPNAVVGRCRMIRHSRDKKNEPNPQRFDRIAHTKETMLSDGLNSLTYQVLDVQRYPLYTQITVDIGTPS

SEQ ID NO: 142 - protein, 337 aa, synthetic construct
  UNSURE 1..337: note = HsB4GalT2
MQHLAFFSRFSARGPAHALHPAASSSSSSSNCSRPNATASSSGLPEVPSALPGPTAPTLPPCPDSPPGLVGRLLIEFTSPMPLERVQRENPGVLMGGRYTPPDCTPAQTVAVIIPFRHREHHLRYWLHYLHPILRRQRLRYGVYVINQHGEDTFNRAKLLNVGFLEALKEDAAYDCFIFSDVDLVPMDDRNLYRCGDQPRHFAIAMDKFGFRLPYAGYFGGVSGLSKAQFLRINGFPNEYWGWGGEDDDIFNRISLTGMKISRPDIRIGRYRMIKHDRDKHNEPNPQRFTKIQNTKLTMKRDGIGSVRYQVLEVSRQPLFTNITVDIGRPPSWPPRG

SEQ ID NO: 143 - protein, 363 aa, synthetic construct
  UNSURE 1..363: note = HsB4GalT3
MRSLSALFGRDQGPTFDYSHPRDVYSNLSHLPGAPGGPPAPQGLPYCPERSPLLVGPVSVSFSPVPSLAEIVERNPRVEPGGRYRPAGCEPRSRTAIIVPHRAREHHLRLLLYHLHPFLQRQQLAYGIYVIHQAGNGTFNRAKLLNVGVREALRDEEWDCLFLHDVDLLPENDHNLYVCDPRGPRHVAVAMNKFGYSLPYPQYFGGVSALTPDQYLKMNGFPNEYWGWGGEDDDIATRVRLAGMKISRPPTSVGHYKMVKHRGDKGNEENPHRFDLLVRTQNSWTQDGMNSLTYQLLARELGPLYTNITADIGTDPRGPRAPSGPRYPPGSSQAFRQEMLQRRPPARPGPLSTANHTALRGSH

SEQ ID NO: 144 - protein, 307 aa, synthetic construct
  UNSURE 1..307: note = HsB4GalT4
MQEIPKAKEFMANFHKTLILGKGKTLTNEASTKKVELDNCPSVSPYLRGQSKLIFKPDLTLEEVQAENPKVSRGRYRPQECKALQRVAILVPHRNREKHLMYLLEHLHPFLQRQQLDYGIYVIHQAEGKKFNRAKLLNVGYLEALKEENWDCFIFHDVDLVPENDFNLYKCEEHPKHLVVGRNSTGYRLRYSGYFGGVTALSREQFFKVNGFSNNYWGWGGEDDDLRLRVELQRMKISRPLPEVGKYTMVFHTRDKGNEVNAERMKLLHQVSRVWRTDGLSSCSYKLVSVEHNPLYINITVDFWFGA

SEQ ID NO: 145 - protein, 354 aa, synthetic construct
  UNSURE 1..354: note = HsB4GalT5
MPGIVNTYLFMMQAQGILIRDNVRTIGAQVYEQVLRSAYAKRNSSVNDSDYPLDLNHSETFLQTTTFLPEDFTYFANHTCPERLPSMKGPIDINMSEIGMDYIHELFSKDPTIKLGGHWKPSDCMPRWKVAILIPFRNRHEHLPVLFRHLLPMLQRQRLQFAFYVVEQVGTQPFNRAMLFNVGFQEAMKDLDWDCLIFHDVDHIPESDRNYYGCGQMPRHFATKLDKYMYLLPYTEFFGGVSGLTVEQFRKINGFPNAFWGWGGEDDDLWNRVQNAGYSVSRPEGDTGKYKSIPHHHRGEVQFLGRYALLRKSKERQGLDGLNNLNYFANITYDALYKNITVNLTPELAQVNEY

SEQ ID NO: 146 - protein, 348 aa, synthetic construct
  UNSURE 1..348: note = HsB4GalT6
MPGIANTYLFMVQARGIMLRENVKTIGHMIRLYTNKNSTLNGTDYPEGNNSSDYLVQTTTYLPENFTYSPYLPCPEKLPYMRGFLNVNVSEVSFDEIHQLFSKDLDIEPGGHWRPKDCKPRWKVAVLIPFRNRHEHLPIFFLHLIPMLQKQRLEFAFYVIEQTGTQPFNRAMLFNVGFKEAMKDSVWDCVIFHDVDHLPENDRNYYGCGEMPRHFAAKLDKYMYILPYKEFFGGVSGLTVEQFRKINGFPNAFWGWGGEDDDLWNRVHYAGYNVTRPEGDLGKYKSIPHHHRGEVQFLGRYKLLRYSKERQYIDGLNNLIYRPKILVDRLYTNISVNLMPELAPIEDY

SEQ ID NO: 147 - protein, 302 aa, synthetic construct [sequence omitted]

SEQ ID NO: 148 - protein, 522 aa, synthetic construct
  UNSURE 1..522: note = HsUGT8
MAKIIIVPPIMFESHMYIFKTLASALHERGHHTVFLLSEGRDIAPSNHYSLQRYPGIFNSTTSDAFLQSKMRNIFSGRLTAIELFDILDHYTKNCDLMVGNHALIQGLKKEKFDLLLVDPNDMCGFVIAHLLGVKYAVFSTGLWYPAEVGAPAPLAYVPEFNSLLTDRMNLLQRMKNTGVYLISRLGVSFLVLPKYERIMQKYNLLPEKSMYDLVHGSSLWMLCTDVALEFPRPTLPNVVYVGGILTKPASPLPEDLQRWVNGANEHGFVLVSFGAGVKYLSEDIANKLAGALGRLPQKVIWRFSGPKPKNLGNNTKLIEWLPQNDLLGHSKIKAFLSHGGLNSIFETIYHGVPVVGIPLFGDHYDTMTRVQAKGMGILLEWKTVTEKELYEALVKVINNPSYRQRAQKLSEIHKDQPGHPVNRTIYWIDYIIRHNGAHHLRAAVHQISFCQYFLLDIAFVLLLGAALLYFLLSWVTKFIYRKIKSLWSRNKHSTVNGHYHNGILNGKYKRNGHIKHEKKVK

SEQ ID NO: 149 - protein, 335 aa, synthetic construct
  UNSURE 1..335: note = HsC1GLT
MLLGEKVDTQPNVLHNDPHARHSDDNGQNHLEGQMNFNADSSQHKDENTDIAENLYQKVRILCWVMTGPQNLEKKAKHVKATWAQRCNKVLFMSSEENKDFPAVGLKTKEGRDQLYWKTIKAFQYVHEHYLEDADWFLKADDDTYVILDNLRWLLSKYDPEEPIYFGRRFKPYVKQGYMSGGAGYVLSKEALKRFVDAFKTDKCTHSSSIEDLALGRCMEIMNVEAGDSRDTIGKETFHPFVPEHHLIKGYLPRTFWYWNYNYYPPVEGPGCCSDLAVSFHYVDSTTMYELEYLVYHLRPYGYLYRYQPTLPERILKEISQANKNEDTKVKLGNP

SEQ ID NO: 150 - protein, 442 aa, synthetic construct
  UNSURE 1..442: note = HsAlg1
MKRWRRGRAARHVVAVVLGDVGRSPRMQYHALSLAMHGFSVTLLGFCNSKPHDELLQNNRIQIVGLTELQSLAVGPRVFQYGVKVVLQAMYLLWKLMWREPGAYIFLQNPPGLPSIAVCWFVGCLCGSKLVIDWHNYGYSIMGLVHGPNHPLVLLAKWYEKFFGRLSHLNLCVTNAMREDLADNWHIRAVTVYDKPASFFKETPLDLQHRLFMKLGSMHSPFRARSEPEDPVTERSAFTERDAGSGLVTRLRERPALLVSSTSWTEDEDFSILLAALEKFEQLTLDGHNLPSLVCVITGKGPLREYYSRLIHQKHFQHIQVCTPWLEAEDYPLLLGSADLGVCLHTSSSGLDLPMKVVDMFGCCLPVCAVNFKCLHELVKHEENGLVFEDSEELAAQLQMLFSNFPDPAGKLNQFRKNLRESQQLRWDESWVQTVLPLVMDT

SEQ ID NO: 151 - protein, 453 aa, synthetic construct
  UNSURE 1..453: note = HsAlg11
MGIRLLLQRKKKLVSTSKNGKNQMVIAFFHPYCNAGGGGERVLWCALRALQKKYPEAVYVVYTGDVNVNGQQILEGAFRRFNIRLIHPVQFVFLRKRYLVEDSLYPHFTLLGQSLGSIFLGWEALMQCVPDVYIDSMGYAFTLPLFKYIGGCQVGSYVHYPTISTDMLSVVKNQNIGFNNAAFITRNPFLSKVKLIYYYLFAFIYGLVGSCSDVVMVNSSWTLNHILSLWKVGNCTNIVYPPCDVQTFLDIPLHEKKMTPGHLLVSVGQFRPEKNHPLQIRAFAKLLNKKMVESPPSLKLVLIGGCRNKDDELRVNQLRRLSEDLGVQEYVEFKINIPFDELKNYLSEATIGLHTMWNEHFGIGVVECMAAGTIILAHNSGGPKLDIVVPHEGDITGFLAESEEDYAETIAHILSMSAEKRLQIRKSARASVSRFSDQEFEVTFLSSVEKLFK

SEQ ID NO: 152 - protein, 193 aa, synthetic construct
  UNSURE 1..193: note = HsAlg14
MVLRSMDVTPRESLSILVVAGSGGHTTEILRLLGSLSNAYSPRHYVIADTDEMSANKINSFELDRADRDPSNMYTKYYIHRIPRSREVQQSWPSTVFTTLHSMWLSFPLIHRVKPDLVLCNGPGTCVPICVSALLLGILGIKKVIIVYVESICRVETLSMSGKILFHLSDYFIVQWPALKEKYPKSVYLGRIV

SEQ ID NO: 153 - protein, 297 aa, synthetic construct
  UNSURE 1..297: note = HsAlg5
MTTATKMPALHRHEEEKFFLNAKGQKETLPSIWDSPTKQLSVVVPSYNEEKRLPVMMDEALSYLEKRQKRDPAFTYEVIVVDDGSKDQTSKVAFKYCQKYGSDKVRVITLVKNRGKGGAIRMGIFSSRGEKILMADADGATKFPDVEKLEKGLNDLQPWPNQMAIACGSRAHLEKESIAQRSYFRTLLMYGFHFLVWFLCVKGIRDTQCGFKLFTREAASRTFSSLHVERWAFDVELLYIAQFFKIPIAEIAVNWTEIEGSKLVPFWSWLQMGKDLLFIRLRYLTGAWRLEQTRKMN

SEQ ID NO: 154 - protein, 472 aa, synthetic construct [sequence omitted]

SEQ ID NO: 155 - protein, 417 aa, synthetic construct
  UNSURE 1..417: note = HsGnT1
MTRPAPGRPPSVSALDGDPASLTREVIRLAQDAEVELERQRGLLQQIGDALSSQRGRVPTAAPPAQPRVPVTPAPAVIPILVIACDRSTVRRCLDKLLHYRPSAELFPIIVSQDCGHEETAQAIASYGSAVTHIRQPDLSSIAVPPDHRKFQGYYKIARHYRWALGQVFRQFRFPAAVVVEDDLEVAPDFFEYFRATYPLLKADPSLWCVSAWNDNGKEQMVDASRPELLYRTDFFPGLGWLLLAELWAELEPKWPKAFWDDWMRRPEQRQGRACIRPEISRTMTFGRKGVSHGQFFDQHLKFIKLNQQFVHFTQLDLSYLQREAYDRDFLARVYGAPQLQVEKVRTNDRKELGEVRVQYTGRDSFKAFAKALGVMDDLKSGVPRAGYRGIVTFQFRGRRVHLAPPLTWEGYDPSWN

SEQ ID NO: 156 - protein, 418 aa, synthetic construct
  UNSURE 1..418: note = HsGnT2
MQRKNEALAPPLLDAEPARGAGGRGGDHPSVAVGIRRVSNVSAASLVPAVPQPEADNLTLRYRSLVYQLNFDQTLRNVDKAGTWAPRELVLVVQVHNRPEYLRLLLDSLRKAQGIDNVLVIFSHDFWSTEINQLIAGVNFCPVLQVFFPFSIQLYPNEFPGSDPRDCPRDLPKNAALKLGCINAEYPDSFGHYREAKFSQTKHHWWWKLHFVWERVKILRDYAGLILFLEEDHYLAPDFYHVFKKMWKLKQQECPECDVLSLGTYSASRSFYGMADKVDVKTWKSTEHNMGLALTRNAYQKLIECTDTFCTYDDYNWDWTLQYLTVSCLPKFWKVLVPQIPRIFHAGDCGMHHKKTCRPSTQSAQIESLLNNNKQYMFPETLTISEKFTVVAISPPRKNGGWGDIRDHELCKSYRRLQ

SEQ ID NO: 157 - protein, 511 aa, synthetic construct
  UNSURE 1..511: note = HsGnT
MHFFKTLSYVTFPRELASLSPNLVSSFFWNNAPVTPQASPEPGGPDLLRTPLYSHSPLLQPLPPSKAAEELHRVDLVLPEDTTEYFVRTKAGGVCFKPGTKMLERPPPGRPEEKPEGANGSSARRPPRYLLSARERTGGRGARRKWVECVCLPGWHGPSCGVPTVVQYSNLPTKERLVPREVPRRVINAINVNHEFDLLDVRFHELGDVVDAFVVCESNFTAYGEPRPLKFREMLTNGTFEYIRHKVLYVFLDHFPPGGRQDGWIADDYLRTFLTQDGVSRLRNLRPDDVFIIDDADEIPARDGVLFLKLYDGWTEPFAFHMRKSLYGFFWKQPGTLEVVSGCTVDMLQAVYGLDGIRLRRRQYYTMPNFRQYENRTGHILVQWSLGSPLHFAGWHCSWCFTPEGIYFKLVSAQNGDFPRWGDYEDKRDLNYIRGLIRTGGWFDGTQQEYPPADPSEHMYAPKYLLKNYDRFHYLLDNPYQEPRSTAAGGWRHRGPEGRPPARGKLDEAEV

SEQ ID NO: 158 - protein, 509 aa, synthetic construct
  UNSURE 1..509: note = HsGnT4a
MQNGKEKLIAYQREFLALKERLRIAEHRISQRSSELNTIVQQFKRVGAETNGSKDALNKFSDNTLKLLKELTSKKSLQVPSIYYHLPHLLKNEGSLQPAVQIGNGRTGVSIVMGIPTVKREVKSYLIETLHSLIDNLYPEEKLDCVIVVFIGETDIDYVHGVVANLEKEFSKEISSGLVEVISPPESYYPDLTNLKETFGDSKERVRWRTKQNLDYCFLMMYAQEKGIYYIQLEDDIIVKQNYFNTIKNFALQLSSEEWMILEFSQLGFIGKMFQAPDLTLIVEFIFMFYKEKPIDWLLDHILWVKVCNPEKDAKHCDRQKANLRIRFRPSLFQHVGLHSSLSGKIQKLTDKDYMKPLLLKIHVNPPAEVSTSLKVYQGHTLEKTYMGEDFFWAITPIAGDYILFKFDKPVNVESYLFHSGNQEHPGDILLNTTVEVLPFKSEGLEISKETKDKRLEDGYFRIGKFENGVAEGMVDPSLNPISAFRLSVIQNSAVWAILNEIHIKKATN

SEQ ID NO: 159 - protein, 396 aa, synthetic construct
  UNSURE 1..396: note = HsGCNT1
MRIHQKPEFVSVRHLELAGENPSSDINCTKVLQGDVNEIQKVKLEILTVKFKKRPRWTPDDYINMTSDCSSFIKRRKYIVEPLSKEEAEFPIAYSIVVHHKIEMLDRLLRAIYMPQNFYCIHVDTKSEDSYLAAVMGIASCFSNVFVASRLESVVYASWSRVQADLNCMKDLYAMSANWKYLINLCGMDFPIKTNLEIVRKLKLLMGENNLETERMPSHKEERWKKRYEVVNGKLTNTGTVKMLPPLETPLFSGSAYFVVSREYVGYVLQNEKIQKLMEWAQDTYSPDEYLWATIQRIPEVPGSLPASHKYDLSDMQAVARFVKWQYFEGDVSKGAPYPPCDGVHVRSVCIFGAGDLNWMLRKHHLFANKFDVDVDLFAIQCLDEHLRHKALETLK

SEQ ID NO: 160 - protein, 380 aa, synthetic construct
  UNSURE 1..380: note = HsGCNT2
MYNTELWENKRFLRAALSNASLLAEACHQIFEGKVFYPTENALKTTLDEATCYEYMVRSHYVTETLSEEEAGFPLAYTVTIHKDFGTFERLFRAIYMPQNVYCVHLDQKATDAFKGAVKQLLSCFPNAFLASKKESVVYGGISRLQADLNCLEDLVASEVPWKYVINTCGQDFPLKTNREIVQYLKGFKGKNITPGVLPPDHAVGRTKYVHQELLNHKNSYVIKTTKLKTPPPHDMVIYFGTAYVALTRDFANFVLQDQLALDLLSWSKDTYSPDEHFWVTLNRIPGVPGSMPNASWTGNLRAIKWSDMEDRHGGCHGHYVHGICIYGNGDLKWLVNSPSLFANKFELNTYPLTVECLELRHRERTLNQSETAIQPSWYF

SEQ ID NO: 161 - protein, 370 aa, synthetic construct
  UNSURE 1..370: note = HsB3GNT2
MKSSSQEKNGKGEVIIPKEKFWKISTPPEAYWNREQEKLNRQYNPILSMLTNQTGEAGRLSNISHLNYCEPDLRVTSVVTGFNNLPDRFKDFLLYLRCRNYSLLIDQPDKCAKKPFLLLAIKSLTPHFARRQAIRESWGQESNAGNQTVVRVFLLGQTPPEDNHPDLSDMLKFESEKHQDILMWNYRDTFFNLSLKEVLFLRWVSTSCPDTEFVFKGDDDVFVNTHHILNYLNSLSKTKAKDLFIGDVIHNAGPHRDKKLKYYIPEVVYSGLYPPYAGGGGFLYSGHLALRLYHITDQVHLYPIDDVYTGMCLQKLGLVPEKHKGFRTFDIEEKNKNNICSYVDLMLVHSRKPQEMIDIWSQLQSAHLKC

SEQ ID NO: 162 - protein, 354 aa, synthetic construct
  UNSURE 1..354: note = HsB3GNT6
MQAPRSPREERSPQEETPEGPTDAPAADEPPSELVPGPPCVANASANATADFEQLPARIQDFLRYRHCRHFPLLWDAPAKCAGGRGVFLLLAVKSAPEHYERRELIRRTWGQERSYGGRPVRRLFLLGTPGPEDEARAERLAELVALEAREHGDVLQWAFADTFLNLTLKHLHLLDWLAARCPHARFLLSGDDDVFVHTANVVRFLQAQPPGRHLFSGQLMEGSVPIRDSWSKYFVPPQLFPGSAYPVYCSGGGFLLSGPTARALRAAARHTPLFPIDDAYMGMCLERAGLAPSGHEGIRPFGVQLPGAQQSSFDPCMYRELLLVHRFAPYEMLLMWKALHSPALSCDRGHRVS

SEQ ID NO: 163 - protein, 350 aa, synthetic construct
  UNSURE 1..350: note = HsXXLT1
MRETFSSATKRLKEARAGAPAAPSPPALELARGSVAPAPGAKAKSLEGGGAGPVDYHLLMMFTKAEHNAALQAKARVALRSLLRLAKFEAHEVLNLHFVSEEASREVAKGLLRELLPPAAGFKCKVIFHDVAVLTDKLFPIVEAMQKHFSAGLGTYYSDSIFFLSVAMHQIMPKEILQIIQLDLDLKFKTNIRELFEEFDSFLPGAIIGIAREMQPVYRHTFWQFRHENPQTRVGGPPPEGLPGFNSGVMLLNLEAMRQSPLYSRLLEPAQVQQLADKYHFRGHLGDQDFFTMIGMEHPKLFHVLDCTWNRQLCTWWRDHGYSDVFEAYFRCEGHVKIYHGNCNTPIPED

SEQ ID NO: 164 - protein, 509 aa, synthetic construct
  UNSURE 1..509: note = HsUGT1A1
MHAGKILLIPVDGSHWLSMLGAIQQLQQRGHEIVVLAPDASLYIRDGAFYTLKTYPVPFQREDVKESFVSLGHNVFENDSFLQRVIKTYKKIKKDSAMLLSGCSHLLHNKELMASLAESSFDVMLTDPFLPCSPIVAQYLSLPTVFFLHALPCSLEFEATQCPNPFSYVPRPLSSHSDHMTFLQRVKNMLIAFSQNFLCDVVYSPYATLASEFLQREVTVQDLLSSASVWLFRSDFVKDYPRPIMPNMVFVGGINCLHQNPLSQEFEAYINASGEHGIVVFSLGSMVSEIPEKKAMAIADALGKIPQTVLWRYTGTRPSNLANNTILVKWLPQNDLLGHPMTRAFITHAGSHGVYESICNGVPMVMMPLFGDQMDNAKRMETKGAGVTLNVLEMTSEDLENALKAVINDKSYKENIMRLSSLHKDRPVEPLDLAVFWVEFVMRHKGAPHLRPAAHDLTWYQYHSLDVIGFLLAVVLTVAFITFKCCAYGYRKCLGKKGRVKKAHKSKTH

SEQ ID NO: 165 - protein, 380 aa, synthetic construct
  UNSURE 1..380: note = HsB4GAT1
MHGQEEQDQYFEFFPPSPRSVDQVKAQLRTALASGGVLDASGDYRVYRGLLKTTMDPNDVILATHASVDNLLHLSGLLERWEGPLSVSVFAATKEEAQLATVLAYALSSHCPDMRARVAMHLVCPSRYEAAVPDPREPGEFALLRSCQEVFDKLARVAQPGINYALGTNVSYPNNLLRNLAREGANYALVIDVDMVPSEGLWRGLREMLDQSNQWGGTALVVPAFEIRRARRMPMNKNELVQLYQVGEVRPFYYGLCTPCQAPTNYSRWVNLPEESLLRPAYVVPWQDPWEPFYVAGGKVPTFDERFRQYGFNRISQACELHVAGFDFEVLNEGFLVHKGFKEALKFHPQKEAENQHNKILYRQFKQELKAKYPNSPRRC

SEQ ID NO: 166 - protein, 507 aa, synthetic construct
  UNSURE 1..507: note = HsUGT1A3
MGKVLVVPIDGSHWLSMREVLRELHARGHQAVVLTPEVNMHIKEENFFTLTTYAISWTQDEFDRHVLGHTQLYFETEHFLKKFFRSMAMLNNMSLVYHRSCVELLHNEALIRHLNATSFDVVLTDPVNLCAAVLAKYLSIPTVFFLRNIPCDLDFKGTQCPNPSSYIPRLLTTNSDHMTFMQRVKNMLYPLALSYICHAFSAPYASLASELFQREVSVVDILSHASVWLFRGDFVMDYPRPIMPNMVFIGGINCANRKPLSQEFEAYINASGEHGIVVFSLGSMVSEIPEKKAMAIADALGKIPQTVLWRYTGTRPSNLANNTILVKWLPQNDLLGHPMTRAFITHAGSHGVYESICNGVPMVMMPLFGDQMDNAKRMETKGAGVTLNVLEMTSEDLENALKAVINDKSYKENIMRLSSLHKDRPVEPLDLAVFWVEFVMRHKGAPHLRPAAHDLTWYQYHSLDVIGFLLAVVLTVAFITFKCCAYGYRKCLGKKGRVKKAHKSKTH

SEQ ID NO: 167 - protein, 418 aa, synthetic construct
  UNSURE 1..418: note = ScAlg1
MGNKSTKKRIIIFVLGDVGHSPRICYHAISFSKLGWQVELCGYVEDTLPKIISSDPNITVHHMSNLKRKGGGTSVIFMVKKVLFQVLSIFKLLWELRGSDYILVQNPPSIPILPIAVLYKLTGCKLIIDWHNLAYSILQLKFKGNFYHPLVLISYMVEMIFSKFADYNLTVTEAMRKYLIQSFHLNPKRCAVLYDRPASQFQPLAGDISRQKALTTKAFIKDYIRDDFDTEKGDKIIVTSTSFTPDEDIGILLGALKIYENSYVKFDSSLPKILCFITGKGPLKEKYMKQVEEYDWKRCQIEFVWLSAEDYPKLLQLCDYGVSLHTSSSGLDLPMKILDMFGSGLPVIAMNYPVLDELVQHNVNGLKFVDRRELHESLIFAMKDADLYQKLKKNVTQEAENRWQSNWERTMRGLKLIH

SEQ ID NO: 168 - protein, 504 aa, synthetic construct
  UNSURE 1..504: note = ScAlg11
MYSSLNPFSKKSSLLNRAVASCGEKNVKVFGFFHPYCNAGGGGEKVLWKAVDITLRKDAKNVIVIYSGDFVNGENVTPENILNNVKAKFDYDLDSDRIFFISLKLRYLVDSSTWKHFTLIGQAIGSMILAFESIIQCPPDIWIDTMGYPFSYPIIARFLRRIPIVTYTHYPIMSKDMLNKLFKMPKKGIKVYGKILYWKVFMLIYQSIGSKIDIVITNSTWTNNHIKQIWQSNTCKIIYPPCSTEKLVDWKQKFGTAKGERLNQAIVLAQFRPEKRHKLIIESFATFLKNLPDSVSPIKLIMAGSTRSKQDENYVKSLQDWSENVLKIPKHLISFEKNLPFDKIEILLNKSTFGVNAMWNEHFGIAVVEYMASGLIPIVHASAGPLLDIVTPWDANGNIGKAPPQWELQKKYFAKLEDDGETTGFFFKEPSDPDYNTTKDPLRYPNLSDLFLQITKLDYDCLRVMGARNQQYSLYKFSDLKFDKDWENFVLNPICKLLEEEERG

SEQ ID NO: 169 - protein, 417 aa, synthetic construct
  UNSURE 1..417: note = NtGnTI
ATQSEYADRLAAAIEAENHCTSQTRLLIDQISLQQGRIVALEEQMKRQDQECRQLRALVQDLESKGIKKLIGNVQMPVAAVVVMACNRADYLEKTIKSILKYQISVASKYPLFISQDGSHPDVRKLALSYDQLTYMQHLDFEPVHTERPGELIAYYKIARHYKWALDQLFYKHNFSRVIILEDDMEIAPDFFDFFEAGATLLDRDKSIMAISSWNDNGQMQFVQDPYALYRSDFFPGLGWMLSKSTWDELSPKWPKAYWDDWLRLKENHRGRQFIRPEVCRTYNFGEHGSSLGQFFKQYLEPIKLNDVQVDWKSMDLSYLLEDNYVKHFGDLVKKAKPIHGADAVLKAFNIDGDVRIQYRDQLDFENIARQFGIFEEWKDGVPRAAYKGIVVFRYQTSRRVFLVGHDSLQQLGIEDT

SEQ ID NO: 170 - protein, 394 aa, synthetic construct
  UNSURE 1..394: note = NtGnTII
RTNTVSNLINSYTNESSESFESIEEAKVSNTHLTRIPKLPLQNELSILLSKRNQMPPRNLNLYSKLAKDHVVIVLYVHNRPQYLQIVIDSLSRVEGISETLLIVSHDGYFEEMNKIVESIKFCQVKQIFAPYSPHIFSDSFPGVSPKDCKEKDDPVEKHCEGMPDQYGNHRSPKIVSLKHHWWWMMNTVWDGLEVTRHHSGHILFIEEDHFIYPNAYRNMQLLAELKSKKCPDCYAANLAPSEVKLRGEGWECLVAERMGNVGYAFNRTVWRKIHKKAAEFCSFDDYNWDITMWSTVYPSFGSPVYTLRGSRTSAVHFGKCGLHQGHNRNVACMDNGGVNIVVDEIDKVANIKPEWEVRKYEHQAGYKAGFKGWGGWGDVRDRELCMEFAKMYI

SEQ ID NO: 171 - protein, 346 aa, synthetic construct
  UNSURE 1..346: note = BtGGTA1
EYIHSPEGSLFWINPSRNPEVGGSSIQKGWWLPRWFNNGYHEEDGDINEEKEQRNEDESKLKLSDWFNPFKRPEVVTMTKWKAPVVWEGTYNRAVLDNYYAKQKITVGLTVFAVGRYIEHYLEEFLTSANKHFMVGHPVIFYIMVDDVSRMPLIELGPLRSFKVFKIKPEKRWQDISMMRMKTIGEHIVAHIQHEVDFLFCMDVDQVFQDKFGVETLGESVAQLQAWWYKADPNDFTYERRKESAAYIPFGEGDFYYHAAIFGGTPTQVLNITQECFKGILKDKKNDIEAQWHDESHLNKYFLLNKPTKILSPEYCWDYHIGLPADIKLVKMSWQTKEYNVVRNNV

SEQ ID NO: 172 - protein, 334 aa, synthetic construct
  UNSURE 1..334: note = MmGGTA1
NRIPEVGENRWQKDWWFPSWFKNGTHSYQEDNVEGRREKGRNGDRIEEPQLWDWFNPKNRPDVLTVTPWKAPIVWEGTYDTALLEKYYATQKLTVGLTVFAVGKYIEHYLEDFLESADMYFMVGHRVIFYVMIDDTSRMPVVHLNPLHSLQVFEIRSEKRWQDISMMRMKTIGEHILAHIQHEVDFLFCMDVDQVFQDNFGVETLGQLVAQLQAWWYKASPEKFTYERRELSAAYIPFGEGDFYYHAAIFGGTPTHILNLTRECFKGILQDKKHDIEAQWHDESHLNKYFLFNKPTKILSPEYCWDYQIGLPSDIKSVKVAWQTKEYNLVRNNV

SEQ ID NO: 173 - protein, 311 aa, synthetic construct
  UNSURE 1..311: note = RnGGTA1
RTHSYQEEDIERAREKGRNGDSIVEPQLWDWFNPKNRPEVLTVTPWKAPIVWEGTYDTALLEKYYARQKITVGLTVFAVGKYIEHYLEDFLESANKYFMVGHRVIFYVMMDDTSRMPAVHLSPLHSLQVFEIRSEKRWQDISMMRMKTIGEHILDHIQHEVDFLFCMDVDQVFQDNFGVETLGQLVAQLQAWWYKASPDEFTYERRELSAAYIPFGEGDFYYHAAVFGGTPVHILNLTRECFKGILQDKKHDIEAQWHDESHLNKYFLFNKPTKILSPEYCWDYHIGLPSDIKNVKIAWQTKEYNLVRSNV

SEQ ID NO: 174 - protein, 358 aa, synthetic construct
  UNSURE 1..358: note = BtB4GalT1
RDLRRLPQLVGVHPPLQGSSHGAAAIGQPSGELRLRGVAPPPPLQNSSKPRSRAPSNLDAYSHPGPGPGPGSNLTSAPVPSTTTRSLTACPEESPLLVGPMLIEFNIPVDLKLVEQQNPKVKLGGRYTPMDCISPHKVAIIIPFRNRQEHLKYWLYYLHPILQRQQLDYGIYVINQAGESMFNRAKLLNVGFKEALKDYDYNCFVFSDVDLIPMNDHNTYRCFSQPRHISVAMDKFGFSLPYVQYFGGVSALSKQQFLSINGFPNNYWGWGGEDDDIYNRLAFRGMSVSRPNAVIGKCRMIRHSRDKKNEPNPQRFDRIAHTKETMLSDGLNSLTYMVLEVQRYPLYTKITVDIGTPS

SEQ ID NO: 175 - protein, 14 aa, synthetic construct
  UNSURE 1..14: note = linker
AAAGGMPPAAAGGM

(19) United States
(12) Patent Application Publication
(10) Pub. No.: US 2025/0066746 A1
(43) Pub. Date: Feb. 27, 2025

(54) COMPOSITIONS AND METHODS FOR SOLUBILIZING GLYCOSYLTRANSFERASES

(71) Applicant: CORNELL UNIVERSITY, Ithaca, NY (US)

(72) Inventors: Matthew DELISA, Ithaca, NY (US); Thapakorn JAROENTOMEECHAI, Ithaca, NY (US); Dario MIZRACHI, Ithaca, NY (US)

(21) Appl. No.: 18/725,018

(22) PCT Filed: Jan. 6, 2023
(86) PCT No.: PCT/US2023/010330

§ 371 Date: Jun. 27, 2024

Related U.S. Application Data
(60) Provisional application No. 63/297,419, filed on Jan. 7, 2022.

(51) Int. Cl. C12N 9/10; C07K 14/775; C07K 16/00; C12N 15/62

(52) U.S. Cl. CPC C12N 9/1048; C07K 14/775; C07K 16/00; C12N 9/1051; C12N 15/62; C12Y 204/01101; C12Y 204/01143; C07K 2319/24

(57) ABSTRACT

The present disclosure relates to a nucleic acid construct having a chimeric nucleic acid molecule encoding a tripartite glycosyltransferase fusion protein. The chimeric nucleic acid molecule includes a first nucleic acid moiety encoding an amphipathic shield domain protein; a second nucleic acid moiety encoding a glycosyltransferase; and a third nucleic acid moiety encoding a water soluble expression decoy protein. The first nucleic acid moiety is coupled to the second nucleic acid moiety's 3′ end and the third nucleic acid moiety is coupled to the second nucleic acid moiety's 5′ end. The coupling may be direct or indirect. The present disclosure further relates to an expression vector, a host cell, and a tripartite glycosyltransferase fusion protein encoded by the nucleic acid construct. Also disclosed are methods of recombinantly producing a tripartite glycosyltransferase fusion protein in soluble form and methods of cell-free glycan remodeling.

This application claims the benefit of U.S. Provisional Patent Application Ser. No. 63/297,419, filed Jan. 7, 2022, which is hereby incorporated by reference in its entirety.

This invention was made with government support under HDTRA1-14-10052 awarded by Defense Threat Reduction Agency; CBET-1159581, CBET-1264701, CBET-1936823, and MCB 1413563 awarded by National Science Foundation; and 1R01GM137314, and 1R01GM127578 awarded by National Institutes of Health. The government has certain rights in the invention.

### FIELD

The present disclosure relates to compositions and methods for making and using water-soluble glycosyltransferase fusion proteins.

### BACKGROUND

Glycosylation—the process by which carbohydrate-based compounds known as glycans are covalently attached to acceptor molecules, typically proteins and lipids —is fundamental to all life (Varki, A., “Biological Roles of Glycans,” Glycobiology 27:3-49 (2017) and Varki, A. et al., “Essentials of Glycobiology,” (Cold Spring Harbor (NY) (2015)). Following conjugation to biomolecules, glycans add an additional layer of information and play important roles in numerous biological processes (Moremen et al., “Vertebrate Protein Glycosylation: Diversity, Synthesis and Function,” Nat. Rev. Mol. Cell Biol. 13:448-62 (2012)) including cell adhesion and signaling (Crocker, P. R., “Siglecs: Sialic-acid-binding Immunoglobulin-like Lectins in Cell-cell Interactions and Signaling,” Curr. Opin. Struct. Biol. 12:609-15 (2002) and Cummings, R. D., “Stuck on Sugars—How Carbohydrates Regulate Cell Adhesion, Recognition, and Signaling,” Glycoconj. J. 36:241-257 (2019)), cell growth and development (Haltiwanger and Lowe, “Role of Glycosylation in Development,” Annu. Rev. Biochem. 73:491-537 (2004)), and immune recognition/response (Zhou and Cobb, “Glycans in Immunologic Health and Disease,” Annu. Rev. Immunol. 39:511-536 (2021) and Rudd et al., “Glycosylation and the Immune System,” Science 291:2370-6 (2001)), among others. Moreover, structural remodeling of protein-linked glycans can improve therapeutic properties in a number of ways such as extending activity and stability both in vitro and in vivo (Sola and Griebenow, “Effects of Glycosylation on the Stability of Protein Pharmaceuticals,” J. Pharm. Sci. 98:1223-45 (2009) and Sinclair and Elliott, “Glycoengineering: The Effect of Glycosylation on the Properties of Therapeutic Proteins,” J. Pharm. Sci. 94:1626-35 (2005), modulating interactions with specific immune receptors (Rothman et al., “Antibody-dependent Cytotoxicity Mediated by Natural Killer Cells is Enhanced by Castanospermine-induced Alterations of IgG Glycosylation,” Molecular Immunology 26:1113-23 (1989)), and targeting to specific cells or tissues (Friedman et al., “A Comparison of the Pharmacological Properties of Carbohydrate Remodeled Recombinant and Placental-derived Beta-glucocerebrosidase: Implications for Clinical Efficacy in Treatment of Gaucher Disease,” Blood 93:2807-16 (1999)).

As appreciation for the biological roles and therapeutic potential of glycans continues to grow, so too does the need for reliable, user-friendly technologies that enable their synthesis and remodeling. However, quantitative preparation of structurally-defined glycans and glycoconjugates remains technically challenging and represents a critical technology gap that limits widespread access to this important biomolecule class (Transforming Glycoscience: A Roadmap for the Future (The National Academies Press) (2012)). A major reason for this difficulty is the lack of template encoding in glycan biosynthesis, which distances carbohydrate structure and function from gene sequence. Hence, unlike nucleic acids and proteins, glycans cannot be directly produced from recombinant DNA technology. Instead, glycan biosynthesis is controlled by the availability, abundance, and activities of glycoenzymes, in particular, glycosyltransferases (GTs) that catalyze formation of specific glycosidic linkages by transferring sugar molecules from donor substrates (e.g., nucleotide sugar or lipid-linked sugar) to hydroxyl groups of acceptor molecules (Lairson et al., “Glycosyltransferases: Structures, Functions, and Mechanisms,” Annu. Rev. Biochem. 77:521-55 (2008) and Taniguchi et al., “Handbook of Glycosyltransferases and Related Genes,” (Springer, Tokyo, Japan (2014)) and glycosyl hydrolases (GHs) that cleave glycan structures during oligosaccharide maturation (Davies and Henrissat, “Structures and Mechanisms of Glycosyl hydrolases,” Structure 3:853-9 (1995)).

GTs exhibit unique catalytic specificities for a wide range of sugar donors and acceptor substrates and generate products with distinct anomeric configurations, which helps to explain the vast structural diversity of “glycospace”. In mammals alone, it is estimated that there are approximately 7,000 oligosaccharide structures (Cummings, R. D., “The Repertoire of Glycan Determinants in the Human Glycome,” Mol. Biosyst. 5:1087-104 (2009)) whose generation involves more than 200 GTs (Moremen Et Al., “Expression System For Structural And Functional studies of Human Glycosylation Enzymes,” Nat. Chem. Biol. 14:156-162 (2018) from 45 different protein families that have been annotated in the carbohydrate-active enzymes (CAZy) database (Lombard et al., “The Carbohydrate-active Enzymes Database (CAZy) in 2013,” Nucleic Acids Res 42: D490-5 (2014)). Moreover, GTs are proficient at replicating the diversity of naturally occurring glycans and glycoconjugates in unnatural contexts, leading to their emergence as powerful synthetic tools for building complex glycomolecules in the laboratory. Much of the progress in this regard exploits sugar nucleotide-dependent GTs of mammalian and bacterial origin for synthesis of complex carbohydrates, glycoconjugates, and glycosylated natural products, which are generated by functionally reconstituting artificial networks of these glycoenzymes within model cellular systems (Clausen et al., “Glycosylation Engineering in Essentials of Glycobiology (eds. rd et al.) 713-728 (Cold Spring Harbor (NY) (2015); Natarajan et al., “Metabolic Engineering of Glycoprotein Biosynthesis in Bacteria,” Emerg. Top Life Sci. 2:419-432 (2018); Williams et al., “Metabolic Engineering of Capsular Polysaccharides,” Emerg. Top Life Sci. 2:337-348 (2018); and Pandey et al., “Metabolic Engineering of Glycosylated Polyketide Biosynthesis,” Emerg. Top Life Sci. 2:389-403 (2018)) or in cell-free, one-pot reaction systems (Na et al., “Recent Progress in Synthesis of Carbohydrates With Sugar Nucleotide-dependent Glycosyltransferases,” Curr. Opin. Chem. Biol. 61:81-95 (2021); Jaroentomeechai et al., “Cell-free Synthetic Glycobiology: Designing and Engineering Glycomolecules Outside of Living Cells,” Front. Chem. 8:645 (2020); and Kightlinger et al., “Synthetic Glycobiology: Parts, Systems, and Applications,” ACS Synth. Biol. 9:1534-1562 (2020)).

These developments notwithstanding, broad access to GTs for fundamental and applied research is bottlenecked by difficulties associated with their recombinant expression. A major reason for this difficulty is that many GTs catalyze reactions at membrane interfaces (e.g., between the cytoplasm and periplasm in Gram-negative bacteria or between the cytosol and endoplasmic reticulum (ER)/Golgi organelles within eukaryotes). As such, these enzymes are typically either secretory proteins or integral membrane proteins (IMPs) that need post-translational modifications (PTMs) (e.g., disulfide bonds, N-linked glycosylation) and/or specialized chaperones to achieve proper folding, membrane translocation/insertion, and function. Efforts to express GTs in the absence of required PTMs or chaperones, or in the presence of single- or multi-pass transmembrane domains (TMDs) or terminal signal peptides (e.g., N-terminal export signals, C-terminal retention signals), are often met with non-functional protein aggregates. This is particularly pronounced for expression of mammalian GTs in bacterial hosts, with successful reports often involving time- and labor-intensive searches for solubility-enhancing fusion partners and molecular chaperones, optimal host strains and culture conditions, and compatible detergents and denaturants for IMP solubilization and in vitro refolding from inclusion bodies, respectively (Skretas et al., “Expression of Active Human Sialyltransferase ST6GalNAcI in Escherichia coli,” Microb. Cell Fact 8:50 (2009); Rao et al., “Structural Insight Into mammalian Sialyltransferases,” Nat. Struct. Mol. Biol. 16:1186-8 (2009); and Ramakrishnan and Qasba, “Crystal Structure of Lactose Synthase Reveals a Large Conformational Change in its Catalytic Component, the Beta1,4-galactosyltransferase-I,” J. Mol. Biol. 310:205-18 (2001)).

For these reasons, functional expression of mammalian GTs in bacteria remains rare. Instead, eukaryotic cells remain the preferred host for producing recombinant glycoenzymes albeit with most studies involving small-scale expression of just one or a few GTs (Taniguchi et al., “Handbook of Glycosyltransferases and Related Genes,” (Springer, Tokyo, Japan (2014)). To date, there are only a few reports of larger-scale expression campaigns involving significant numbers of GTs: one such study describes expression of 51 human GTs as fusions to the yeast cell wall Pir proteins to enable immobilization on the surface of Saccharomyces cerevisiae (Shimma et al., “Construction of a Library of Human Glycosyltransferases Immobilized in the Cell Wall of Saccharomyces cerevisiae,” Appl. Environ. Microbiol. 72:7003-12 (2006)) while a second study describes expression of 339 human glycoenzymes as fusions to a solubility-enhancing GFP domain in either mammalian cells (HEK293) or baculovirus-infected insect cells (Moremen Et Al., “Expression System For Structural And Functional studies of Human Glycosylation Enzymes,” Nat. Chem. Biol. 14:156-162 (2018)). Interestingly, the authors of this latter study explore the potential of Escherichia coli for human glycoenzyme expression but report that all GTs expressed in this host accumulate as insoluble aggregates (Moremen Et Al., “Expression System For Structural And Functional studies of Human Glycosylation Enzymes,” Nat. Chem. Biol. 14:156-162 (2018)). Thus, the biosynthetic capacity and versatility of simple E. coli bacteria, one of the most important model organisms in biology and biotechnology (Blount, Z. D., “The Unexhausted Potential of E. coli,” Elife 4(2015)), is yet to be unlocked for functional expression of GTs on a large scale.

The present disclosure is directed to overcoming these and other deficiencies in the art.

### SUMMARY

A first aspect of the present disclosure relates to a nucleic acid construct. The nucleic acid construct includes a chimeric nucleic acid molecule encoding a tripartite glycosyltransferase fusion protein. The chimeric nucleic acid molecule includes a first nucleic acid moiety encoding an amphipathic shield domain protein; a second nucleic acid moiety encoding a glycosyltransferase; and a third nucleic acid moiety encoding a water soluble expression decoy protein. The first nucleic acid moiety is coupled to the second nucleic acid moiety's 3′ end and the third nucleic acid moiety is coupled to the second nucleic acid moiety's 5′ end. The coupling may be direct or indirect.

Another aspect of the present disclosure relates to an expression vector including the nucleic acid construct according to the present disclosure.

Another aspect of the present disclosure relates to a host cell comprising the nucleic acid construct of the present disclosure.

Another aspect of the present disclosure relates to a tripartite glycosyltransferase fusion protein produced by a host cell according to the present disclosure.

Another aspect of the present disclosure relates to a cell-free protein expression system. The cell-free protein expression system comprises a cell lysate or extract and a nucleic acid construct according to the present disclosure.

Another aspect of the present disclosure relates to a tripartite glycosyltransferase fusion protein produced by the cell-free expression system according to the present disclosure.

Another aspect of the present disclosure relates to a method of recombinantly producing a tripartite glycosyltransferase fusion protein in water soluble form. This method involves providing a host cell according to the present disclosure or a cell-free expression system according to the present disclosure. The method further involves culturing the host cell or using the cell-free expression system under conditions effective to express the tripartite glycosyltransferase fusion protein in a water soluble form within the host cell cytoplasm or the cell-free expression system.

Another aspect of the present disclosure relates to a tripartite glycosyltransferase fusion protein produced by the methods of recombinantly producing a tripartite glycosyltransferase fusion protein according to the present disclosure.

Another aspect of the present disclosure relates to a tripartite glycosyltransferase fusion protein comprising: an amino terminal water soluble expression decoy protein; a glycosyltransferase; and a carboxyl terminal amphipathic shield domain protein.

Another aspect of the present disclosure relates to a method of cell-free glycan remodeling. This method involves providing a glycan primer; providing one or more tripartite glycosyltransferase fusion protein(s) according to the present disclosure; and incubating the glycan primer with the one or more tripartite glycosyltransferase fusion protein(s) under conditions effective to transfer a glycosyl group to the glycan primer to produce a modified glycan structure.

The Examples of the present disclosure describe a generalizable workflow for efficient production of structurally diverse GTs using standard E. coli expression strains. At the heart of this workflow is a protein engineering method called SIMPLEx (solubilization of integral membrane proteins with high levels of expression) (Mizrachi et al., “Making Water-soluble Integral Membrane Proteins In Vivo Using an Amphipathic Protein Fusion Strategy,” Nat Commun 6:6826 (2015), which is hereby incorporated by reference in its entirety) that enables topological conversion of secretory and membrane-bound proteins into water-soluble variants. In the Examples of the present disclosure, this conversion is achieved for GTs by modifying their N-termini with a decoy protein that prevents membrane insertion and their C-termini with an amphipathic protein that effectively shields hydrophobic surfaces from the aqueous environment (FIG. 3A). Using this approach, soluble expression of nearly 100 GTs, including many of human origin, directly within the E. coli cytoplasm at titers in the 5-10 mg/L range is demonstrated. Importantly, this large-scale expression platform furnishes functional glycoenzymes that can subsequently be used to remodel the structures of diverse glycan donors, leading to the formation of a variety of important glycoforms including human complex-type N-glycans on the therapeutic monoclonal antibody (mAb) trastuzumab. It is anticipated that SIMPLEx-remodeled GTs will help to deepen the understanding of glycoenzymes from all kingdoms of life and accelerate the assembly of these enzymes into cell-based and cell-free systems that enable biosynthesis of important glycomolecules.

### BRIEF DESCRIPTION OF THE DRAWINGS

FIGS. 1A-1B are tables providing the strains, cell lines, and plasmids used in Examples 1-9. Table 1A provides strains, cell lines, and plasmids from various sources. Table 1B provides plasmids designed and evaluated in Examples 1-9. Reference 1: Dyson et al., “Production of Soluble Mammalian Proteins in Escherichia coli: Identification of Protein Features that Correlate with Successful Expression,” BMC Biotechnol. 4:32 (2004); Reference 2: Glasscock et al., “A Flow Cytometric Approach to Engineering Escherichia coli for Improved Eukaryotic Protein Glycosylation,” Metab. Eng. 47:488-495 (2018); Reference 3: Hamilton et al., “A Library of Chemically Defined Human N-Glycans Synthesized from Microbial Oligosaccharide Precursors,” Sci. Rep. 7: 15907 (2017); Reference 4: Mizrachi et al., “Making Water-Soluble Integral Membrane Proteins in vivo Using an Amphipathic Protein Fusion Strategy,” Nat. Commun. 6:6826 (2015); Reference 5: Stark et al., “BioBits Bright: A Fluorescent Synthetic Biology Education Kit,” Sci. Adv. 4:eaat5107 (2018); Reference 6: Dodev et al., “A Tool Kit for Rapid Cloning and Expression of Recombinant Antibodies,” Sci. Rep. 4:5885 (2014); and Reference 7: Feldman et al., “Engineering N-Linked Protein Glycosylation with Diverse O Antigen Lipopolysaccharide Structures in Escherichia coli,” Proc. Natl. Acad. Sci. USA 102:3016-21 (2005), each of which are hereby incorporated by reference in their entirety.

FIGS. 2A-2D are tables showing the results of experiments carried out with glycosyltransferase enzymes according to the present disclosure. FIG. 2A is a table providing the name, glycosyltransferase family, Uniprot ID (*which is hereby incorporated by reference in its entirety), protein structure/topology, and E. coli host strain of the glycosyltransferase enzymes used in Examples 1-9. FIG. 2B is a table providing the 3D structure availability (UNIPROT), SIMPLEX expression score, unfused expression score, pI—full length (ExPASy), MW-full length (ExPASy), solubility prediction-full length, pI-truncated (ExPASy), MW-truncated (ExPASy), solubility prediction-truncated, MW-SIMPLEx, pI-SIMPLEx, pI-SIMPLEx (ExPASy), and solubility prediction-SIMPLEx for the glycosyltransferase enzymes used in Examples 1-9. FIG. 2C is a table providing the full length sequence (FASTA) of the glycosyltransferase enzymes used in Examples 1-9 (SEQ ID NOs. 1-100). FIG. 2D is a table providing the truncated sequence (FASTA) of the glycosyltransferase enzymes used in Examples 1-9 (SEQ ID NOs: 101-174).

FIGS. 3A-3D demonstrate SIMPLEx-mediated expression of biologically-active HsST6Gal1. FIG. 3A is a schematic showing membrane topology of type II transmembrane proteins and molecular architecture of SIMPLEx constructs. Each construct consisted of N-terminal ΔspMBP and C-terminal ApoAI* that flanked HsST6Gal1. Intervening flexible linker (L) connects ΔspMBP and ApoAI* to the GT domains while the 6×His tag was placed at the C-terminus to facilitate detection and purification. HsST6Gal1 domain variants studied were wild-type (wt) HsST6Gal1 (top) and truncated Δ26HsST6Gal1 (bottom), in which the cytoplasmic tail (CT) and transmembrane domain (TMD) were removed. FIG. 3B shows immunoblot analysis of the soluble (S), detergent-solubilized (D), and insoluble (I) fractions prepared from E. coli SHuffle T7 Express lysY cells carrying plasmid pET28a(+) encoding each of the indicated constructs. An equivalent amount of total protein was loaded in each lane. Blots were probed with anti-polyhistidine antibody (αHis). Control blots were generated by probing with anti-GroEL antibody. Results are representative of three biological replicates. Molecular weight (M_{w}) markers are shown at left. FIG. 3C shows kinetic analysis of purified Sx-Δ26HsST6Gal1 and commercial human ST6Gal1 performed using asialofetuin as acceptor substrate and CMP-Neu5Ac as donor substrate. A standard phosphate curve was generated (see FIG. 9B) to convert the initial raw absorbance reading to the enzymatically released inorganic phosphate from CMP-Neu5Ac. Values for Vₘₐₓ and Kₘ values were determined using Prism 9. Data are the mean of three biological replicates+/−SEM. FIG. 2D shows functional characterization of sialyltransferase-mediated chemoenzymatic remodeling of protein-linked glycans using bioorthogonal click chemistry-based assay. Fluorescence (501/523 nm ex/em) measured in clarified lysates prepared from E. coli cells expressing: Sx-Δ26HsST6Gal1 (Sx-ST6), ΔspMBP-Δ26HsST6Gal1 (ΔspMBP-ST6), Δ26HsST6Gal1-ApoAI (ST6-ApoAI), or Δ26HsST6Gal1 (ST6), as indicated. Lysates from E. coli cells carrying empty pET28a(+) plasmid was used as a negative control (empty lysate). Fluorescence data, corresponding to the extent of chemoenzymatic modification, are the mean of three biological replicates (starting from freshly transformed cells)+/−SEM.

FIGS. 4A-4J demonstrate soluble expression of Sx-GT constructs in the E. coli cytoplasm. 98 GTs were evaluated for soluble, cytoplasmic expression in the SIMPLEx framework. Immunoblot analysis of soluble fractions derived from either BL21(DE3) or SHuffle T7 Express cells carrying plasmids for Sx-GT (top blot in each panel) or unfused GT (bottom blot in each panel) constructs. GTs were clustered according to origin and activity as follows: human glucosyltransferases (HsGlcTs) (FIG. 4A); human galactosyltransferases (HsGalTs) (FIG. 4B); human mannosyltransferases (HsManTs) (FIG. 4C); human N-acetylglucosaminyltransferases (HsGlcNAcTs) (FIG. 4D); human N-acetylgalactosaminyltransferases (HsGalNAcTs) (FIG. 4E); human fucosyltransferases (HsFucTs) (FIG. 4F); human sialyltransferases (HsSiaTs) (FIG. 4G); other human GTs (HsGTs) (FIG. 4H); eukaryotic GTs (EukGTs) (FIG. 4I); and bacterial GTs (BacGTs) (FIG. 4J). The expression strain and sequence for each GT including information about truncation of TMD domains are provided in FIG. 2. Graphical representations of monosaccharide substrates are presented according to symbol nomenclature for glycans (Symbol Nomenclature for Graphical Representation of Glycans, Glycobiology 25: 1323-1324 (2015), which is hereby incorporated by reference in its entirety). An equivalent amount of total protein was loaded in each lane and blots were probed with anti-polyhistidine antibody (αHis) to detect GTs. To confirm equivalent loading, the same samples were probed with anti-GroEL antibody (see FIGS. 10A-10J). Blots are representative of three biological replicates. Molecular weight (M_{w}) markers are indicated at left.

FIGS. 5A-5C demonstrate compatibility of SIMPLEx reformatting with diverse expression platforms. Immunoblot analysis of the soluble (S) and insoluble (I) fractions derived from: cell-free protein synthesis (CFPS) using crude S30 extract prepared from E. coli BL21(DE3) cells (FIG. 5A); and cell-based expression using S. cerevisiae strain SBY49 (FIG. 5B) or HEK 293T cells (FIG. 5C) as indicated. All three systems involved plasmids for expressing either Sx-Δ26HsST6Gal1 (Sx-ST6) or unfused Δ26HsST6Gal1 (ST6). Empty plasmid was used as a negative control (empty) in each case. Blots were probed with anti-polyhistidine (αHis) antibody to detect GT expression, with longer exposures (αHis-long) provided to better identify protein products with low expression. An equivalent amount of total protein was loaded in each lane and confirmed by probing blots with antibodies specific for GroEL, Tubulin, and GAPDH, which are housekeeping proteins in E. coli, yeast, and mammalian cells, respectively. Results are representative of three biological replicates. Molecular weight (M_{w}) markers are shown on the left.

FIGS. 6A-6B demonstrate cell-free construction of hybrid- and complex-type N-glycans using Sx-GTs. FIG. 6A is a schematic of bioenzymatic routes to hybrid- and complex-type N-glycan structures. Man₃GlcNAc₂ glycan (M3; glycan 1) derived from glycoengineered E. coli cells equipped with biosynthesis pathway for eukaryotic trimannosyl core N-glycan was used as primer for glycan construction. Subsequent cell-free glycan elaboration reactions yielded the following N-glycan structures: 2 G0-GlcNAc; 3 G0; 4 G2; 5 G2S; 6 G2S2; 7 G0F; 8 G2F; 9 G2S1F; and 10 G2S2F. Glycan naming follows shorthand notation for IgG glycans. For complete glycan list with chemical structures, see FIG. 23. Synthesis steps: (i) non-enzymatic acid hydrolysis; (ii) Sx-Δ29HsGnTI; (iii) Sx-Δ29HsGnTII; (iv) Sx-Δ30HsFucT8; (v) Sx-Δ44Hsβ4GalT1; and (vi, vii) Sx-Δ26HsST6Gal1. All Sx-GTs were produced using E. coli BL21(DE3) or its derivative SHuffle T7 Express lysY. FIG. 6B shows MALDI-TOF MS spectra of glycans 1-10, where glycan 1 served as primer that was used as starting material to generate enzymatically-derived product glycans 2-10.

FIGS. 7A-7B show remodeling of IgG-Fc N-glycans on trastuzumab using Sx-GTs. FIG. 7A is a schematic of bioenzymatic routes to hybrid- and complex-type N-glycan structures linked to asparagine 297 (N297) of the trastuzumab antibody. Trastuzumab bearing Man₅GlcNAc₂ glycan (M5; glycan 11) derived from glycoengineered HEK293F lacking GnTI activity was used as a glycan primer. Subsequent cell-free glycan remodeling reactions yielded the following N-glycan structures: 12 (M5+GlcNAc); 2 (G0-GlcNAc); 3 (G0); 4 (G2); and 6 (G2S2). Glycan notation follows IgG glycan short naming system. For complete glycan list with chemical structures, see FIG. 23. SIMPLEx-reformatted GTs and glycosidase for each synthesis step are provided above reaction arrow. FIG. 7B shows deconvoluted LC-MS spectra in 140-160 kDa range using intact antibody analysis of trastuzumab bearing glycan 11 as starting material and enzymatically-derived product glycans 2-4, 6, and 12. Structures of anticipated N-glycan products are provided in each spectrum. Full MS spectra (0-200 kDa) for all structures are provided in FIG. 20.

FIGS. 8A-8B demonstrates that SIMPLEx architecture promotes soluble expression of difficult-to-express proteins (DTEPs). FIG. 8A shows the results of immunoblot analysis of the soluble fraction prepared from E. coli cells expressing human-derived DTEPs as SIMPLEx fusions (left panel) or as unfused proteins (right panel). E. coli BL21(DE3) was used to express human transcription factors (GATA2, JUN, and FOS), human cyclin-dependent kinase 4 (CDK4), and human cyclin-dependent kinase inhibitor 2A (CDKN2A), while SHuffle T7 Express lysY strain was used to express human epidermal growth factor receptor tyrosine kinase domain (EGFRTK), human matrix metallopeptidase 1 (MMP1), and human proinsulin (ProIns). Human gene constructs were based on work of Dyson et all. Dashed line indicates cropped membrane to remove empty lane. FIG. 8B shows a Coomassie-stained SDS-PAGE (left) and immunoblot analysis (right) of whole cell lysates derived from E. coli SHuffle T7 Express lysY cells carrying plasmid pET28a(+) encoding each of the indicated constructs. For immunoblots in FIG. 8A, an equivalent amount of total protein was loaded in each lane. For SDS-PAGE gel and immunoblot in FIG. 8B, samples were normalized by culture OD₆₀₀ such that an equivalent number of cells were loaded in each lane. Immunoblots in FIGS. 8A-8B were probed with anti-polyhistidine antibody (αHis). Control blots were generated by probing membranes with anti-GroEL antibody. Results are representative of three biological replicates. Molecular weight (M_{w}) markers are shown on left of each blot/gel. Red arrows denote expression products.

FIGS. 9A-9B demonstrate the functional characterization of Sx-Δ26HsST6Gal1. FIG. 9A shows the results of Coomassie-stained SDS-PAGE gel analysis of fractions corresponding to purification of Sx-Δ26HsST6Gal1 by Ni-NTA chromatography. Gel is representative of three biological replicates. FIG. 9B shows the specific activity of sialyltransferase determined using malachite green phosphate reagents. Absorbance readings (OD₆₀₀) correspond to amount of released inorganic phosphate from CMP-Neu5Ac after glycosylation. Data are the mean of three biological replicates+/−SEM. FIG. 9C is a schematic of a bioorthogonal click chemistry-based ST assay. Purified human alpha-1 antitrypsin (A1AT) was treated with a2-3,6,8,9 neuraminidase (NA) to remove native sialic acid and used as substrate to evaluate Sx-Δ26HsST6Gal1-mediated installation of azido-Neu5Ac. Depicted glycans are representative glycoforms of native human A1AT. Azido (N3-) functional groups on Neu5Ac provide a chemical handle on A1AT for conjugation with carboxyrhodamine 110 (CR110) fluorophore or PEG4-biotin reporters via strain-promoted azide-alkyne cycloaddition (SPAAC) using dibenzocyclooctyne group (DBCO) as reactive alkyne. FIG. 9D shows representative SDS-PAGE and immunoblot of reaction products of in vitro ST assay. After labeling with CR110, reaction mixtures were separated on SDS-PAGE gel and fluorescence signal of labeled glycoproteins was measured at 501/523 nm λₑₓ/λₑₘ. Coomassie-stained gel served as loading control. Results are representative of three biological replicates. Molecular weight (Mw) markers are shown at left. FIG. 9E shows fluorescence corresponding to in vitro ST activity of purified Sx-Δ26HsST6Gal1 and commercial HsST6Gal1 as function of enzyme concentration. Data are the mean of three biological replicates+/−SEM. Inset is logarithmic representation of same data.

FIGS. 10A-10J show loading control blots for FIGS. 4A-4J. Control immunoblots corresponding to each panel in FIGS. 5A-5C that were generated by loading an identical amount of each sample and probing with anti-GroEL antibody. Immunoblot analysis of soluble fractions derived from either BL21(DE3) or SHuffle T7 Express cells carrying plasmids for Sx-GT (top blot in each panel) or unfused GT (bottom blot in each panel) constructs. GTs were clustered according to origin and activity as follows: human glucosyltransferases (HsGlcTs) (FIG. 10A); human galactosyltransferases (HsGalTs) (FIG. 10B); human mannosyltransferases (HsManTs) (FIG. 10C); human N-acetylglucosaminyltransferases (HsGlcNAcTs) (FIG. 10D); human N-acetylgalactosaminyltransferases (HsGalNAcTs) (FIG. 10E); human fucosyltransferases (HsFucTs) (FIG. 10F); human sialyltransferases (HsSiaTs) (FIG. 10G); other human GTs (HsGTs) (FIG. 10H); eukaryotic GTs (EukGTs) (FIG. 10I); and bacterial GTs (BacGTs) (FIG. 10J). Results are representative of three biological replicates. Molecular weight (M_{w}) markers are indicated at left.

FIGS. 11A-11I show subcellular fractionation analysis of SIMPLEx-reformatted GT expression. Western blot analysis of the soluble (S), detergent-solubilized (D), and insoluble (I) fractions prepared from either BL21(DE3) or SHuffle T7 Express cells carrying plasmid pET28a(+) encoding either Sx-GT (left blot in each panel) or GT (right blot in each panel) constructs corresponding to the following GTs: Δ29HsGnTI (FIG. 11A); A36HsFucT7 (FIG. 111B); Δ35HsST6 GalNAc1 (FIG. 11C); Δ40HsAlg11 (FIG. 11D); Δ30NtGnTI (FIG. 11E); CjCsTII (FIG. 11F); Hpβ4GalT1 (FIG. 11G); Ngβ4GalT1 (FIG. 11H); and Nmβ4GalT1 (FIG. 11I). An equivalent amount of total protein was loaded in each lane. Blots were probed with anti-polyhistidine antibody (αHis). Results are representative of three biological replicates. Molecular weight (M_{w}) markers are shown on the left.

FIG. 12 shows the cell density of E. coli cultures expressing GT enzymes. Representative data points for cultures expressing SIMPLEx-reformatted GT fusions (Sx-GT; blue) or unfused GTs (red). All data points for each GT were plotted on the same axis. Final cell density values were recorded as OD₆₀₀ readings taken after 16-18 hours of growth. Graph depicts three biological replicates for each construct.

FIGS. 13A-13C show yield quantification for select SIMPLEx-reformatted GTs. FIG. 13A shows the results of Coomassie-stained SDS-PAGE gel analysis of three representative Sx-GTs (Sx-Δ26hsST6Gal1, Sx-Δ29hsGnT1, Sx-Δ30hsFUT8) following expression and purification by Ni-NTA chromatography. An equivalent amount of total protein was loaded in each lane. Results are representative of three biological replicates. Molecular weight (M_{w}) marker is shown on the left. Red arrows denote full-length expression products. Soluble protein yields determined on a mass basis (mg/L) (FIG. 13B) and a molar basis (nM) (FIG. 13C). Each indicated protein was purified as in FIG. 13A using Ni-NTA chromatography from 1-L cultures of E. coli carrying each SIMPLEx-reformatted GT construct as indicated. E. coli cultures expressing unfused GT constructs served as controls. Yield values are representative of three biological replicates and error bars represent SEM. Red asterisks denote purified proteins depicted in FIG. 13A.

FIG. 14A-14B demonstrate the physicochemical properties of GTs that correlate with successful expression. FIG. 14A shows immunoblot analysis of SIMPLEx-reformatted GTs (Sx-GTs) and unfused GTs (GTs) and demonstrates expression score assignment. Blots were probed with anti-polyhistidine antibody (αHis) and results are representative of three biological replicates. Based on relative band intensities from immunoblot analysis, each GT as a SIMPLEx construct or unfused enzyme was categorized as non-expressor (score 0; grey circle), weak expressor (score 1; cyan circle), medium expressor (score 2; light blue circle), and strong expressor (score 3; dark blue circle). Bar graph summarizes total number of GTs for each category, comparing SIMPLEx and unfused formats as indicated. FIG. 14B shows scatter plots relating the following physicochemical properties: (i) protein molecular weight (Mw) excluding added mass from ΔspMBP and ApoAI* domains; (ii) protein isoelectric point (pI); and (iii) protein solubility score as calculated by Protein-Sol server. Individual data points colored according to their expressor score category. Plots were generated using R version 3.4.2 software. All data used for analysis are provided in FIG. 2.

FIGS. 15A-15B demonstrate the relationship between soluble expression, protein size and isoelectric point. FIG. 15A shows the expression scores for unfused GTs (GTs) or SIMPLEx-reformatted GTs (Sx-GTs) as a function of protein molecular weight to provide average expression score (E̅x̅) for small (<40 kDa), medium (40-60 kDa), and large (>60 kDa) proteins. Note that the added molecular weight from ΔspMBP and ApoAI* domains of the SIMPLEx construct was excluded from size classification. Graphs depict mean of expression scores determined in FIG. 14A+/−SEM. Statistical significance was determined by Welch's two-sided t-test (p<0.05 considered significant; ns, not significant), leading to the following p-values: 0.0423 for Sx-GTs<40 kDa vs. >60 kDa; 0.0467 for Sx-GTs 40-60 kDa vs. >60 kDa; and 0.0464 for GTs<40 kDa vs. 40-60 kDa. FIG. 15B is a scatter plot of protein isoelectric point (pI) of unfused and SIMPLEx-fused GTs. Data points were labeled according to change in expression score difference between SIMPLEx-fused and unfused GTs as indicated in legend. Diagonal line represents no change in pI due to SIMPLEx fusion. Arrows signify shift towards a more basic or more acidic protein following SIMPLEx fusion. Plots were generated using R version 3.4.2 software. All data used for analysis are provided in FIG. 2.

FIGS. 16A-16C shows MS analysis of sialylated N-glycans from cell-free remodeling. FIG. 16A is an HILIC-LC-MS chromatogram of cell-free reaction to install sialic acid on glycan 4 using Sx-Δ26HsST6Gal1. Schematic for stepwise conversion of glycan 4 to 5 and 6 is shown, with the preferred substrate for HsST6Gal1, the al-3Man branch, highlighted in green and the less-preferred α1-6Man branch highlighted in red. FIG. 16B shows an MS (left panel) and an MS/MS (right panel) spectra of the doubly charged glycan at m/z 966.8463. Positive ion MS/MS fragmentation pattern confirmed the identity of A2G2S1 product. FIG. 16C shows an MS (left panel) and an MS/MS (right panel) spectra of the doubly charged glycan at m/z 1112.3943. Positive ion MS/MS fragmentation pattern confirmed the identity of A2G2S2 product.

FIGS. 17A-17C show MS analysis of sialylated, core-fucosylated N-glycans from cell-free remodeling. FIG. 17A is an HILIC-LC-MS chromatogram of cell-free reaction to install sialic acid on glycan 8 using Sx-Δ26HsST6Gal1. Schematic for stepwise conversion of glycan 8 to 9 and 10 is shown, with the preferred substrate for HsST6Gal1, the al-3Man branch, highlighted in green and the less-preferred α1-6Man branch highlighted in red. FIG. 17B shows an MS (left panel) and an MS/MS (right panel) spectra of the doubly charged glycan at m/z 1039.8713. Positive ion MS/MS fragmentation pattern confirmed the identity of A2G2S1 product. FIG. 17C shows an MS (left panel) and an MS/MS (right panel) spectra of the doubly charged glycan at m/z 1185.4182. Positive ion MS/MS fragmentation pattern confirmed the identity of A2G2S2 product.

FIGS. 18A-18D demonstrates remodeling glycans on therapeutic glycoproteins with Sx-GTs. Cell-free reactions catalyzing: sialyltransferase reaction on N-glycan of neuraminidase (NA)-treated A1A1 using Sx-CjCstII (FIG. 18A); fucosyltransferase reaction on N-glycan of NA-treated A1AT using Sx-Δ36HsFucT7 (FIG. 18B); sialyltransferase reaction on O-glycan of NA-treated bovine submaxillary mucin using Sx-Δ34HsST3Gal1 (FIG. 18C); and N-acetylglucosaminyl transferase reaction on Man₃GlcNAc₂ N-glycan of MBP-glucagon fusion protein2 using Sx-Δ29HsGnT1 (FIG. 18D). In all cases, reactions were performed using nucleotide-activated sugars modified with azide group as indicated. Following Sx-GT-catalyzed reactions, glycoprotein products were labeled with either dibenzocyclooctyne-carboxyrhodamine 110 (DBCO-CR110) or DBCO-PEG₄-biotin reporters via SPACC reaction. In-gel fluorescence analysis was used to detect glycoproteins modified with fluorescent CR110 reporter while immunoblot analysis using streptavidin-HRP was used to detect glycoproteins modified with biotin reporter. Results are representative of three biological replicates. Molecular weight (M_{w}) markers are shown at left.

FIGS. 19A-19D demonstrate glycosidase sensitivity of N-glycans on trastuzumab. Deconvoluted MS analysis of intact trastuzumab derived from Expi293F™ GnTI-cells following incubation with: PBS (FIG. 19A); endoglycosidase S2 (FIG. 19B); endoglycosidase F1 (FIG. 19C); and endoglycosidase F3 (FIG. 19D). All reactions were carried out at 37° C. for 16 hours in 10-μL reaction volume. Full spectra in the range of 25-200 kDa are shown in the panels on the left. Red box indicates region between 140-160 kDa and the spectra for this mass range is provided in the panels on the right. Structures of anticipated N-glycoprotein products are provided within each spectrum.

FIG. 20 shows an MS spectra for trastuzumab glycoforms. Full MS spectra in the range of 0-200 kDa corresponding to each glycoform of trastuzumab detected in FIGS. 7A-7B. Structures of anticipated N-linked glycoprotein products are provided in each spectrum.

FIGS. 21A-21B demonstrate remodeling of IgG-Fc N-glycans on trastuzumab using Sx-GTs. FIG. 21A is an extended schematic of bioenzymatic routes to hybrid- and complex-type N-glycan structures linked to N297 of trastuzumab. Trastuzumab bearing Man₅GlcNAc₂ glycan (M5; glycan 11) derived from glycoengineered HEK293F lacking GnTI activity was used as a glycan primer. Subsequent cell-free glycan remodeling reactions yielded the following N-glycan structures: 1 (M3); 2 (G0-GlcNAc); 3 (G0); 4 (G2); 6 (G2S2); 7 (G0F); 12 (M5+GlcNAc); 13 (M5+GlcNAcGal); 14 (M3+GlcNAc); and 15 (G0F-GlcNAc). For complete glycan list with chemical structures, see FIG. 23. SIMPLEx-reformatted GTs and glycosidases for each synthesis step are provided above reaction arrow. FIG. 21B shows deconvoluted LC-MS spectra in 140-160 kDa range using intact antibody analysis of enzymatically-derived product glycans 1, 7, and 13-15. Structures of the anticipated N-glycoprotein products are provided in each spectrum. Asterisk indicates unidentified product.

FIGS. 22A-22B demonstrates remodeling of IgG-Fc N-glycans on trastuzumab using Sx-GTs. FIG. 22A is a schematic of bioenzymatic routes to unnatural N-glycan structures linked to N297 of trastuzumab. Trastuzumab bearing Man₅GlcNAc₂ glycan (M5; glycan 11) was used as a glycan primer to generate N-glycan 16 (M5-GlcNAz). Subsequent strain-promoted cycloaddition reaction was performed to install either DBCO-PEG4-Biotin or DBCO-carboxyrhodamine (CR110) to the azide group, yielding glycan 17 and glycan 18, respectively. FIG. 22B shows deconvoluted LC-MS spectra in 140-160 kDa range using intact antibody analysis of enzymatically-derived product glycans 16-18. Structures of the anticipated N-glycoprotein products are provided in each spectrum.

FIG. 23 is a table providing N-glycan structures produced in Examples 1-9.

### DETAILED DESCRIPTION

A first aspect of the present disclosure relates to a nucleic acid construct. The nucleic acid construct includes a chimeric nucleic acid molecule encoding a tripartite glycosyltransferase fusion protein. The chimeric nucleic acid molecule includes a first nucleic acid moiety encoding an amphipathic shield domain protein; a second nucleic acid moiety encoding a glycosyltransferase; and a third nucleic acid moiety encoding a water soluble expression decoy protein. The first nucleic acid moiety is coupled to the second nucleic acid moiety's 3′ end and the third nucleic acid moiety is coupled to the second nucleic acid moiety's 5′ end. The coupling may be direct or indirect.

Another aspect of the present disclosure relates to a tripartite glycosyltransferase fusion protein produced by the methods of recombinantly producing a tripartite glycosyltransferase fusion protein according to the present disclosure.

The nucleic acid molecules encoding the various polypeptide components of a tripartite glycosyltransferase fusion protein can be ligated together along with appropriate regulatory elements that provide for expression of the tripartite glycosyltransferase fusion protein. Typically, the nucleic acid construct encoding the chimeric protein can be inserted into any of the many available expression vectors and cell systems using reagents that are well known in the art and further described infra.

As used herein, “nucleic acid”, refers to a polymeric form of nucleotides of any length, either ribonucleotides or deoxynucleotides. Thus, this term includes, but is not limited to, single-, double-, or multi-stranded DNA or RNA, genomic DNA, cDNA, DNA-RNA hybrids, or a polymer comprising purine and pyrimidine bases or other natural, chemically or biochemically modified, non-natural, or derivatized nucleotide bases. The nucleic acid construct may be a synthetic nucleic acid construct. As used herein “synthetic” nucleic acid construct refers to a nucleic acid construct that is artificially produced and/or that does not exist in nature. As described in more detail herein, the nucleic acid constructs of the present disclosure are utilized to make water-soluble glycosyltransferases using an amphipathic protein fusion strategy. In particular, the nucleic acid constructs are part of a new strategy for the solubilization of glycosyltransferases based on the affinity for hydrophobic surfaces displayed by amphipathic proteins.

As used herein, the term “glycosyltransferase” (GT) includes an enzyme or fragment thereof which catalyzes the transfer of a donor glycosyl moiety from a glycosyl donor to an acceptor. Suitable glycosyl donors include, without limitation, CMP-sialic acid, GDP-fucose, GDP-mannose, UDP-glucose, UDP-galactose, UDP-xylose, UDP-N-acetylglucosamine, UDP-N-acetylgalactosamine, UDP-glucuronic acid, Dolichol-P-glucose, Dolichol-P-mannose, Dolichol-P-P-(glucose₃-mannose₉-GlcNAc₂), and undecaprenyl-P—P—N-acetylmuramic acid-pentapeptide-GlcNAc). Suitable acceptor moieties include, without limitation, oligosaccharides, monosaccharides, polypeptides, proteins, lipids such as ceramides, small organic molecules, and nucleic acid molecules such as DNA.

GTs may be classified as (i) single-pass transmembrane proteins with C-termini in the cytoplasm (type I transmembrane protein); (ii) single-pass transmembrane proteins with N-termini in the cytoplasm (type II transmembrane protein); (iii) multi-pass transmembrane proteins; (iv) secretory proteins with N-terminal signal peptides and C-terminal ER retention domains; and (v) cytosolic proteins. In some embodiments, the glycosyltransferase is selected from the group consisting of (i) a single-pass transmembrane protein with C-terminus in cytoplasm (type I transmembrane protein); (ii) a single-pass transmembrane protein with N-terminus in cytoplasm (type II transmembrane protein); (iii) a multi-pass transmembrane protein; and (iv) a secretory protein with N-terminal signal peptide and C-terminal ER retention domain.

In some embodiments, the glycosyltransferase is a full-length glycosyltransferase. Accordingly the second nucleic acid moiety encodes a full-length glycosyltransferase. In accordance with such embodiments, the second nucleic acid moiety comprises a full-length GT gene.

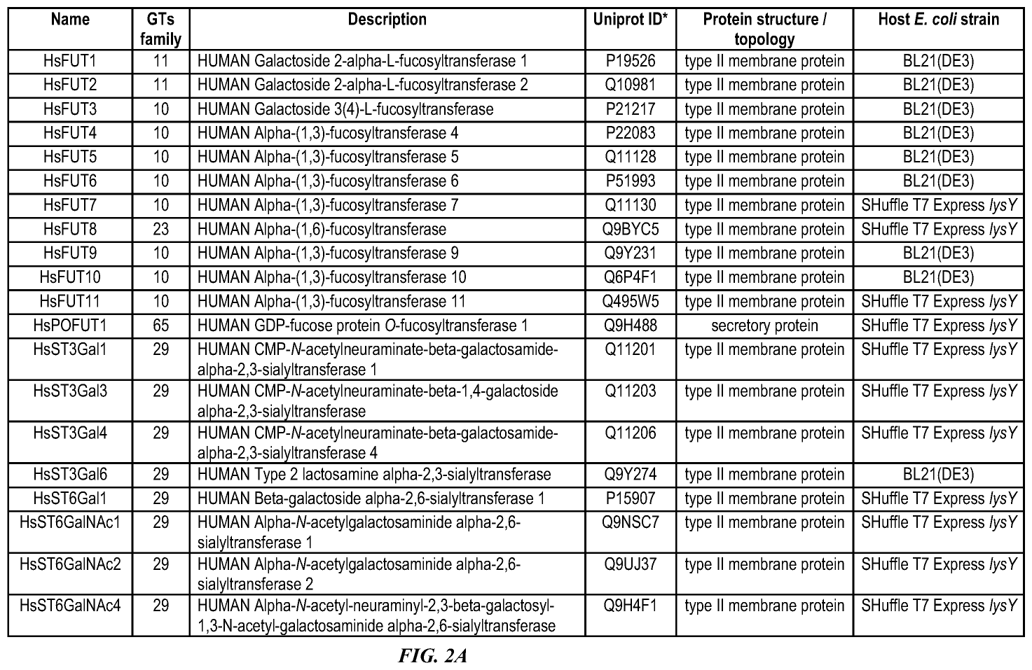

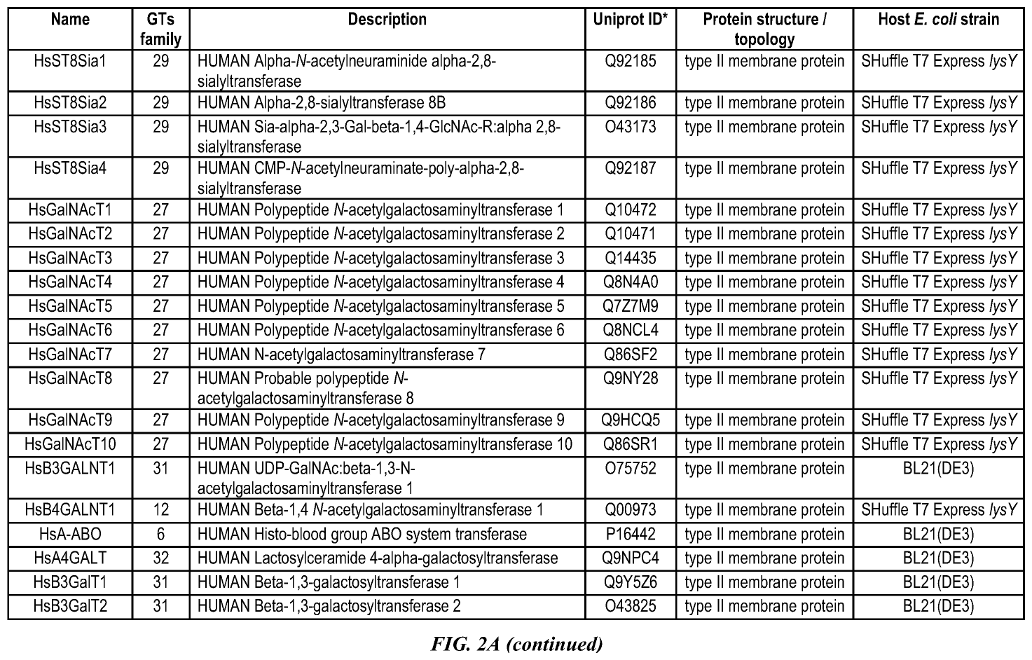

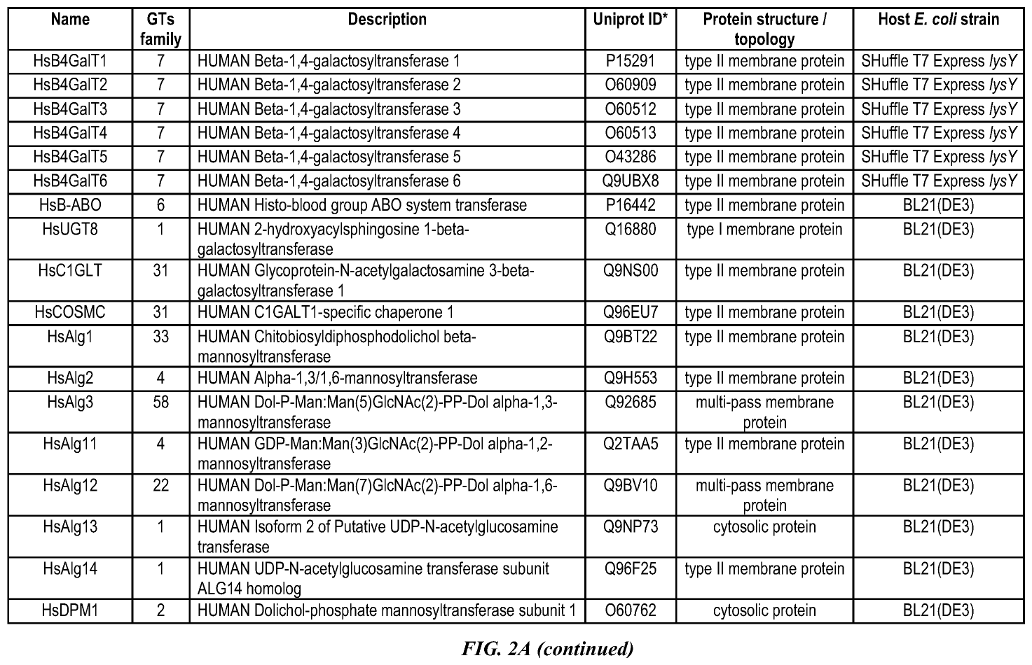

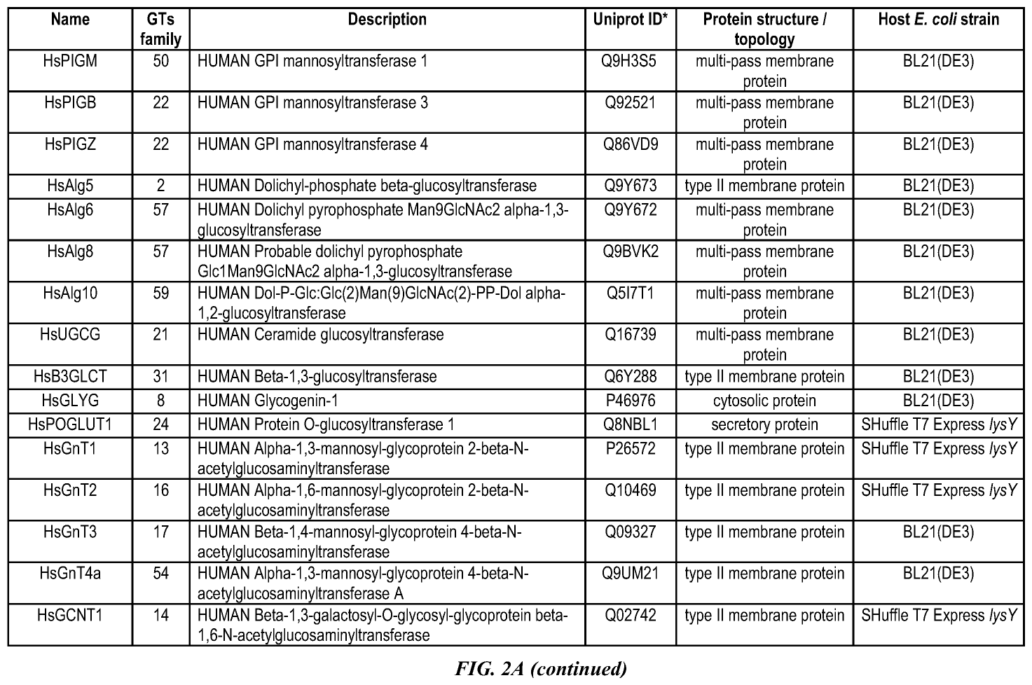

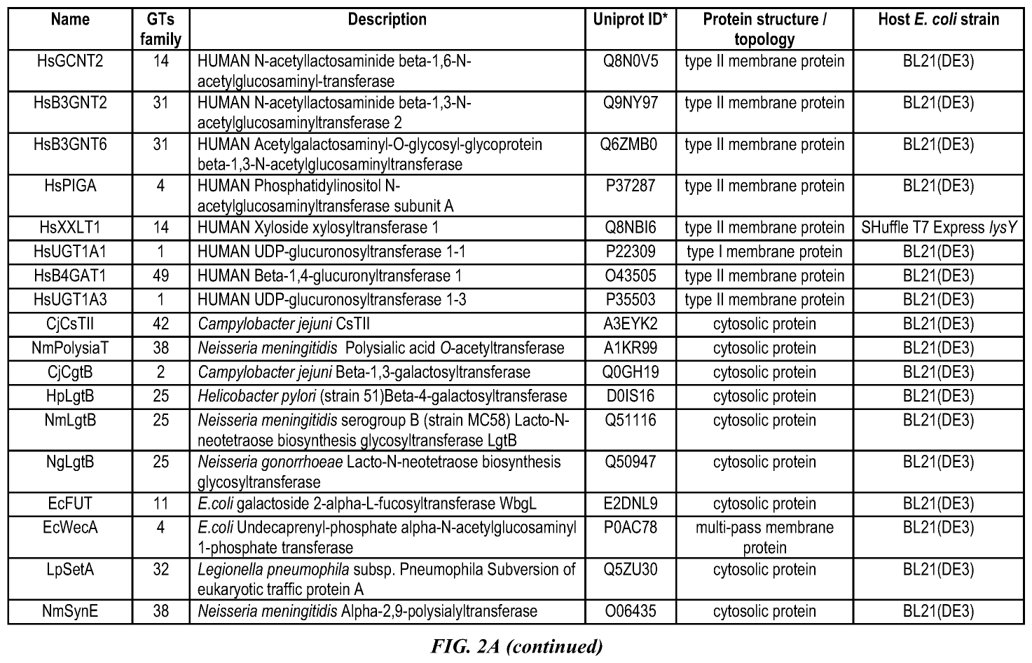

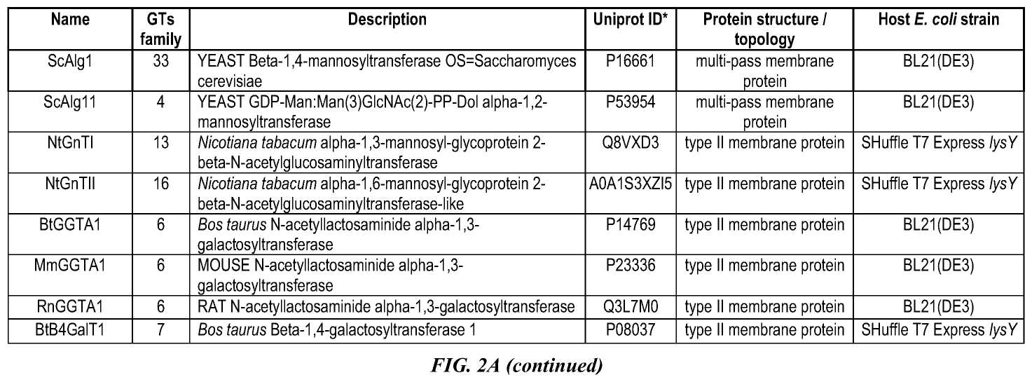

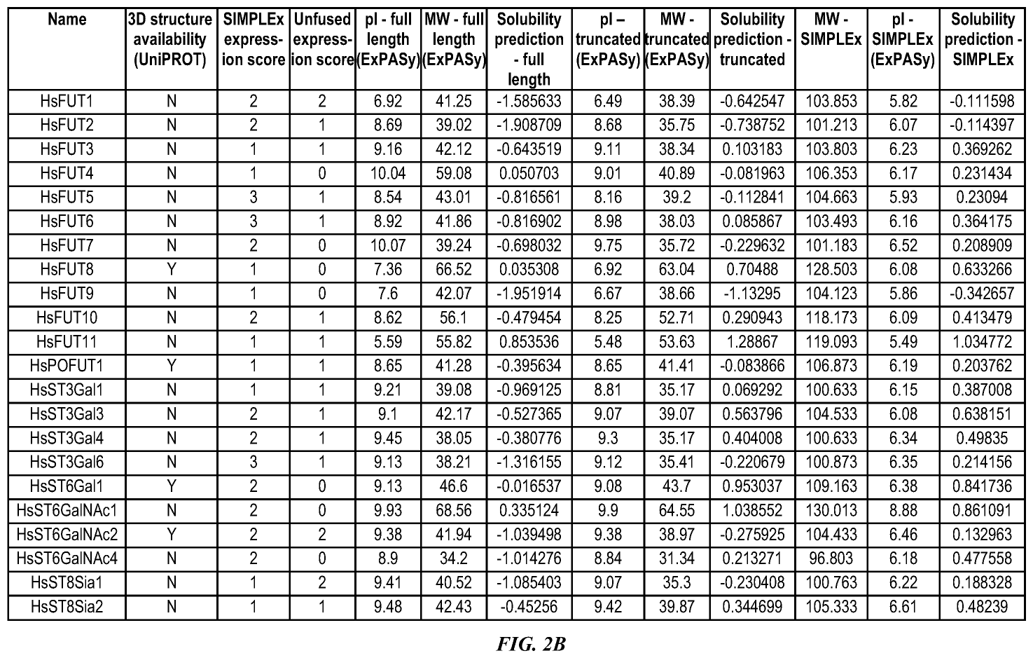

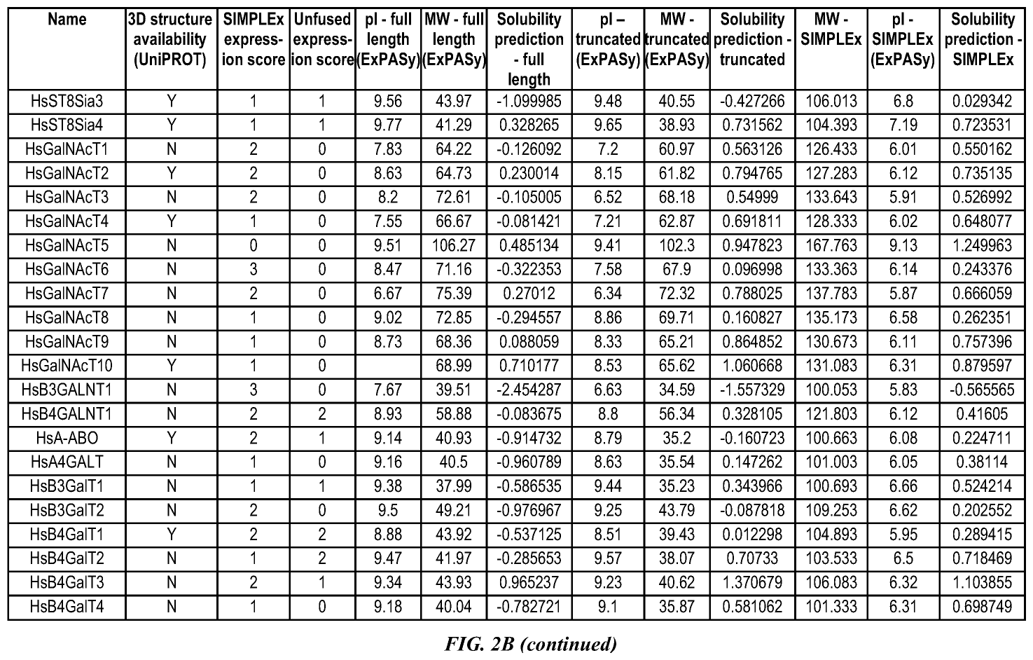

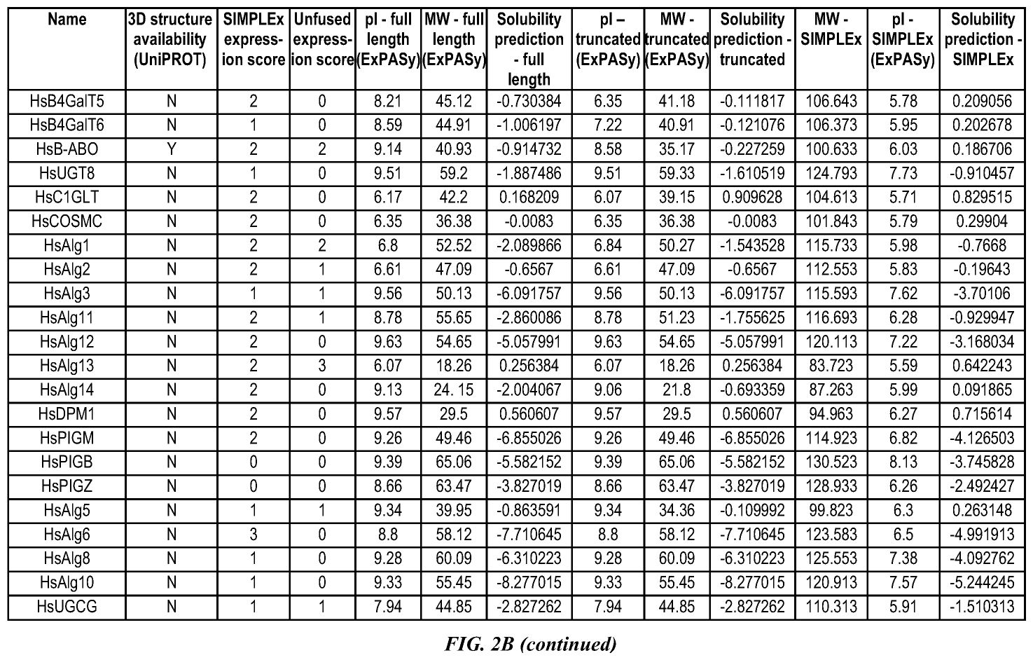

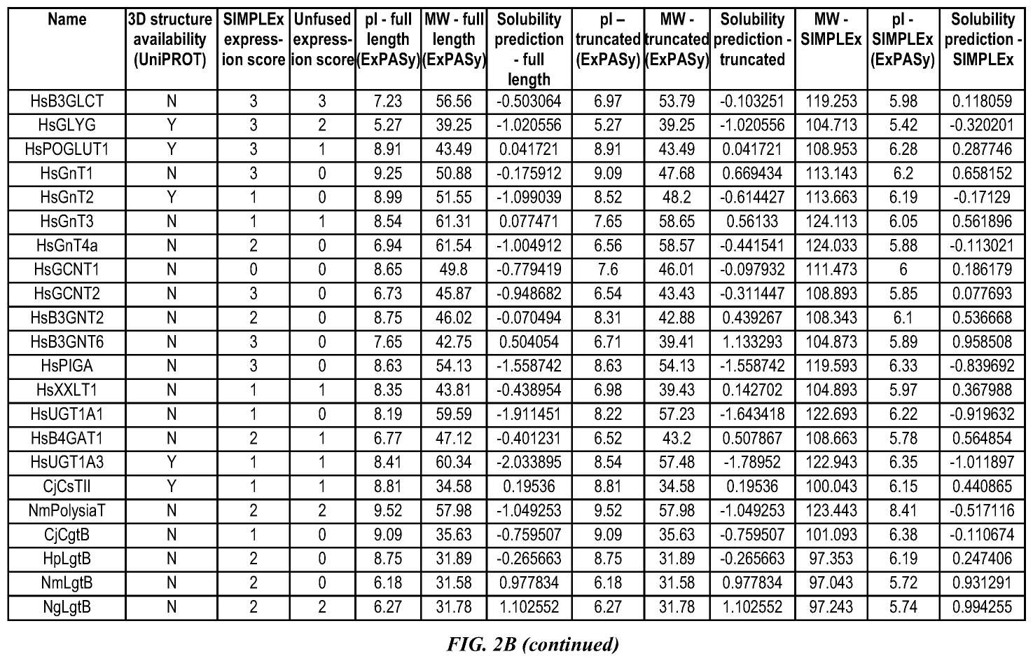

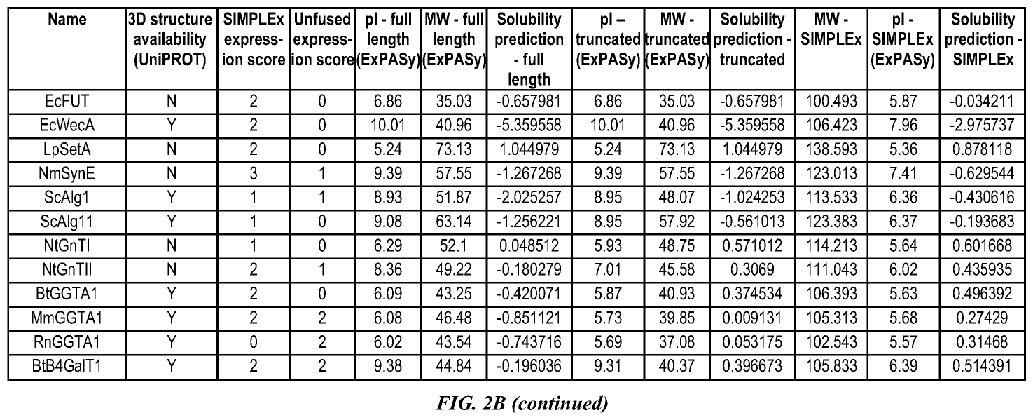

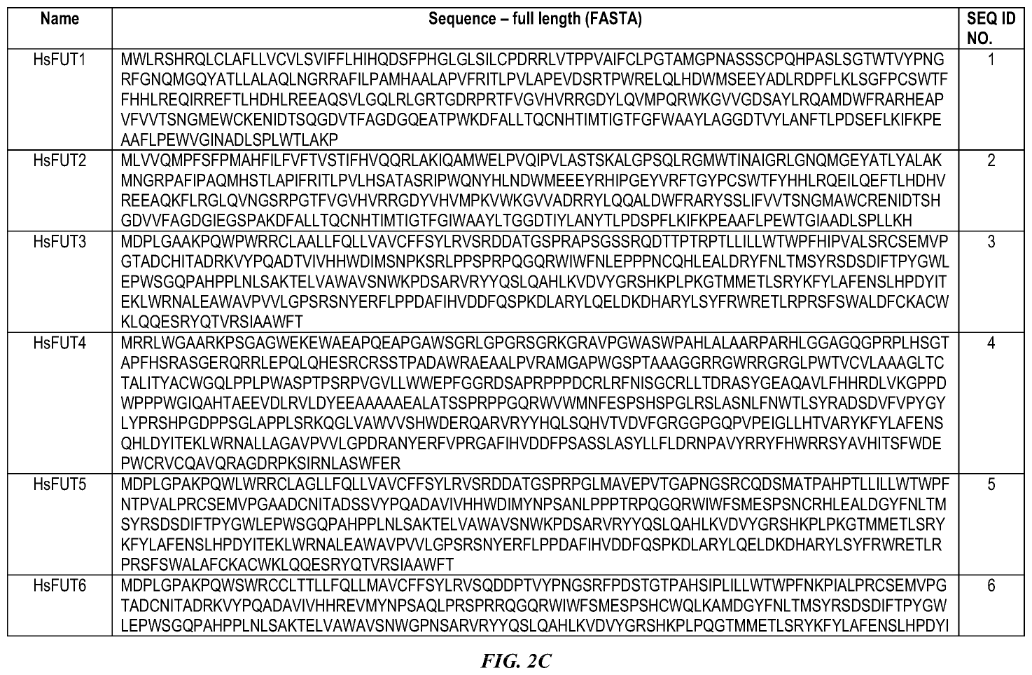

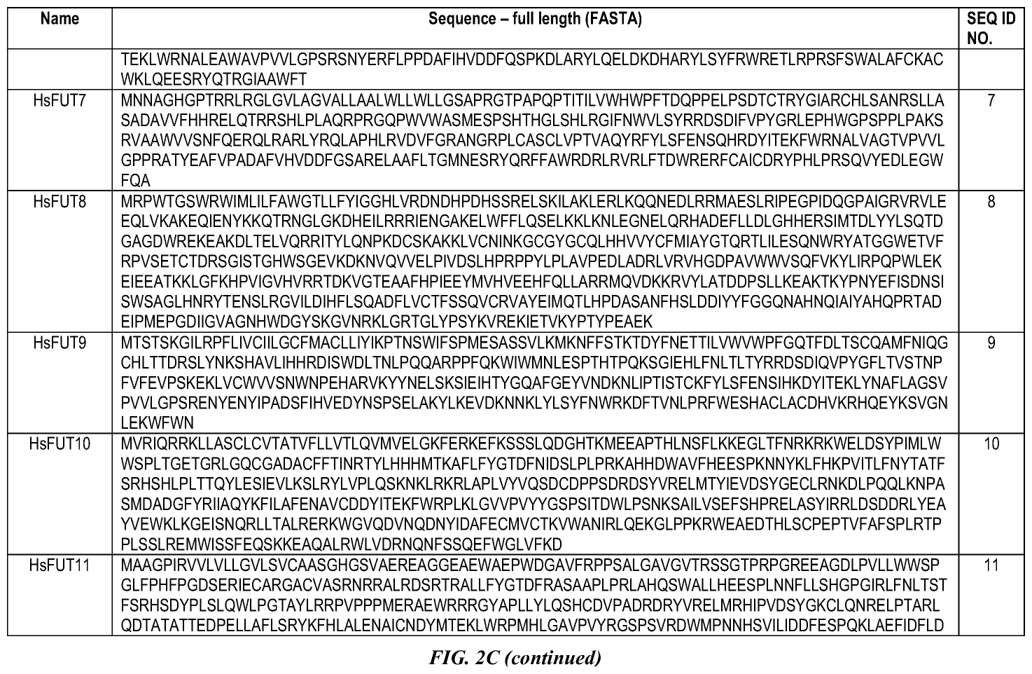

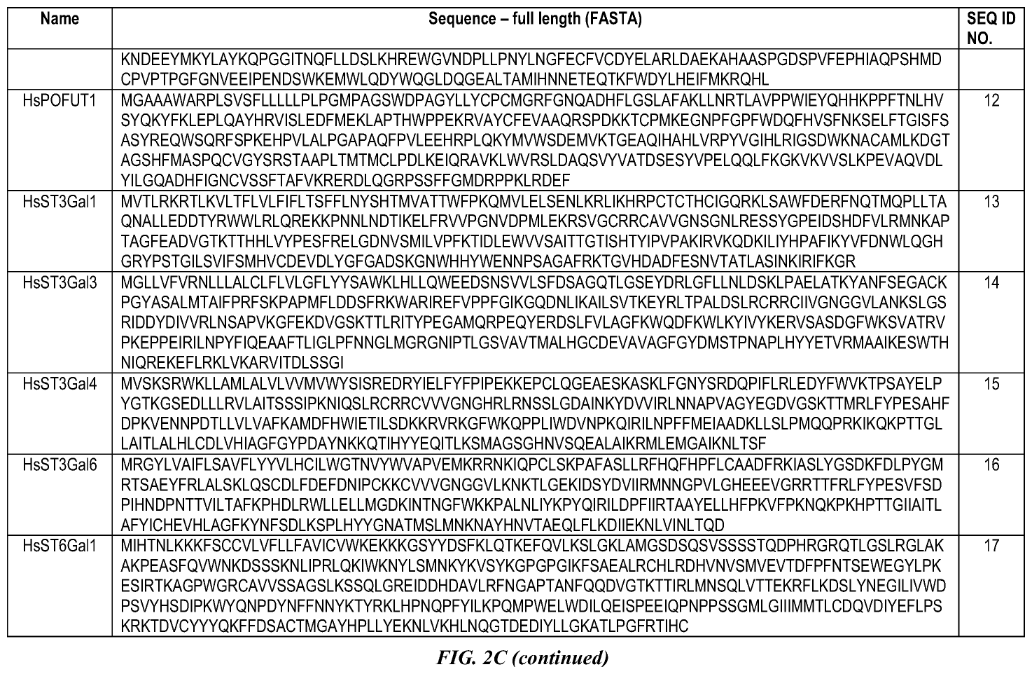

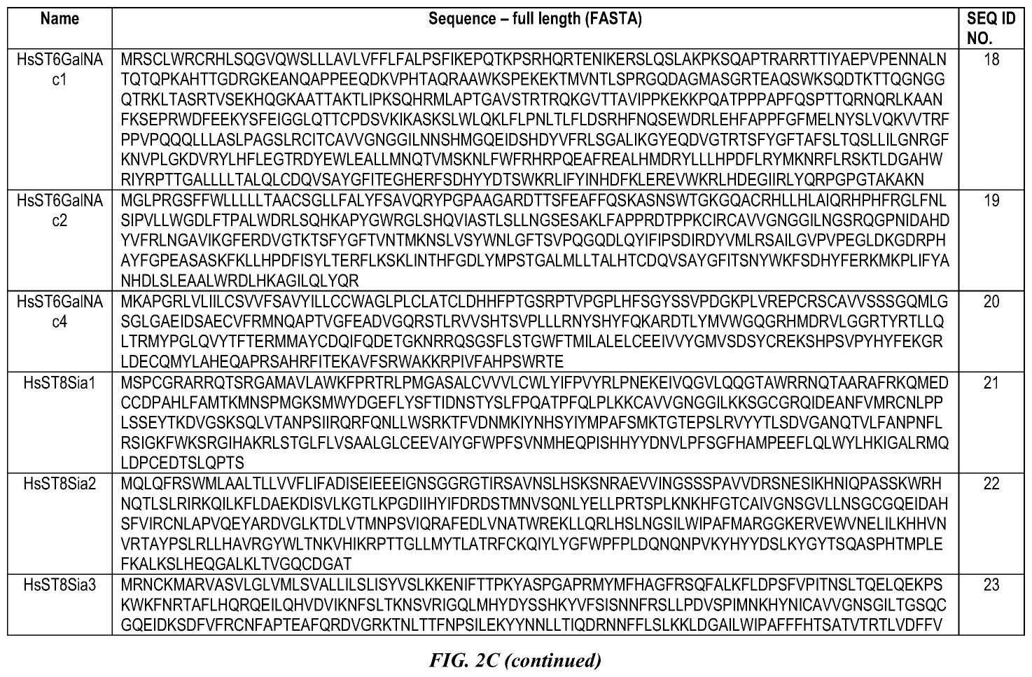

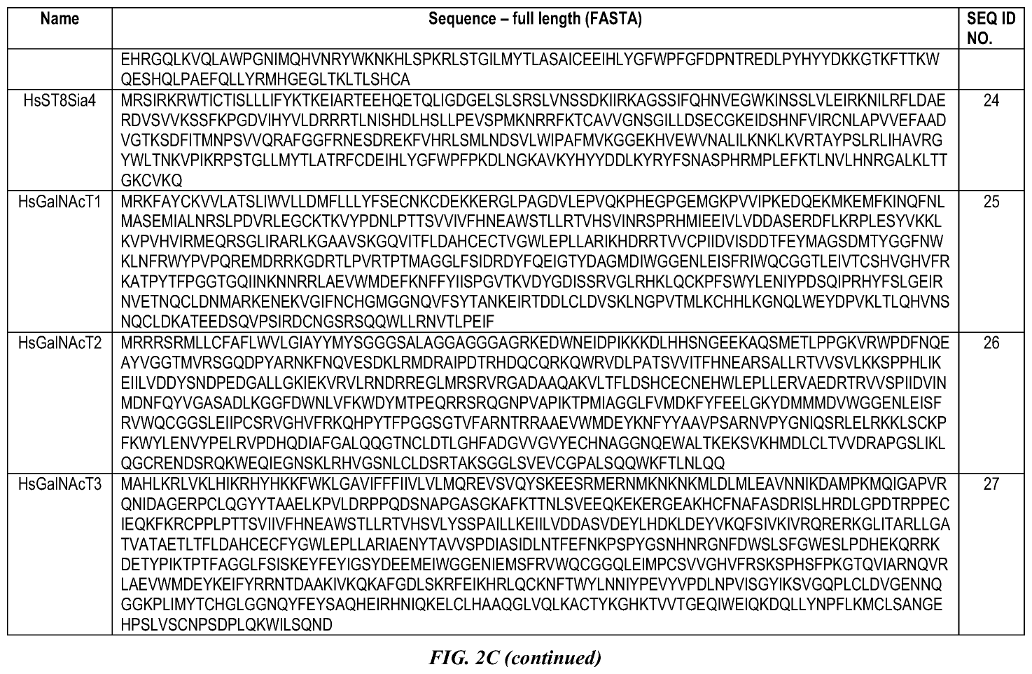

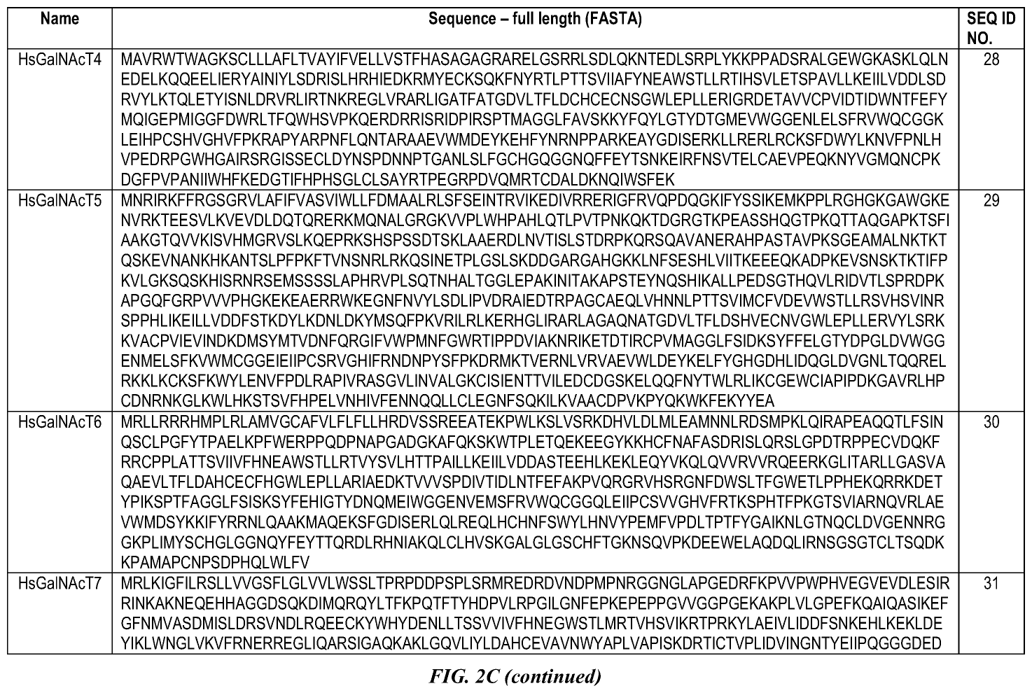

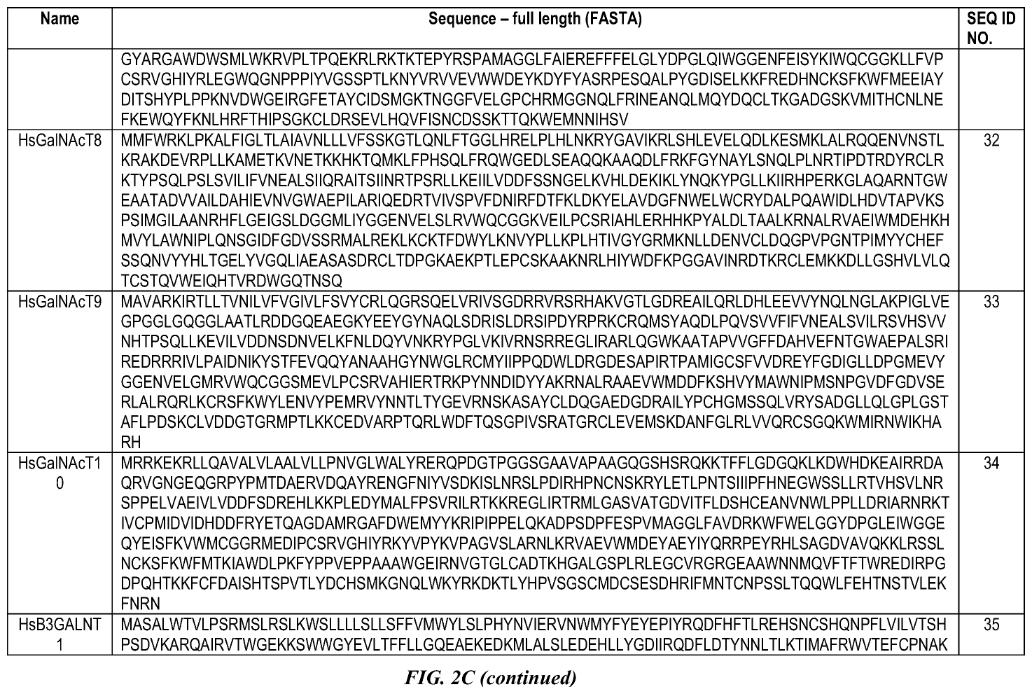

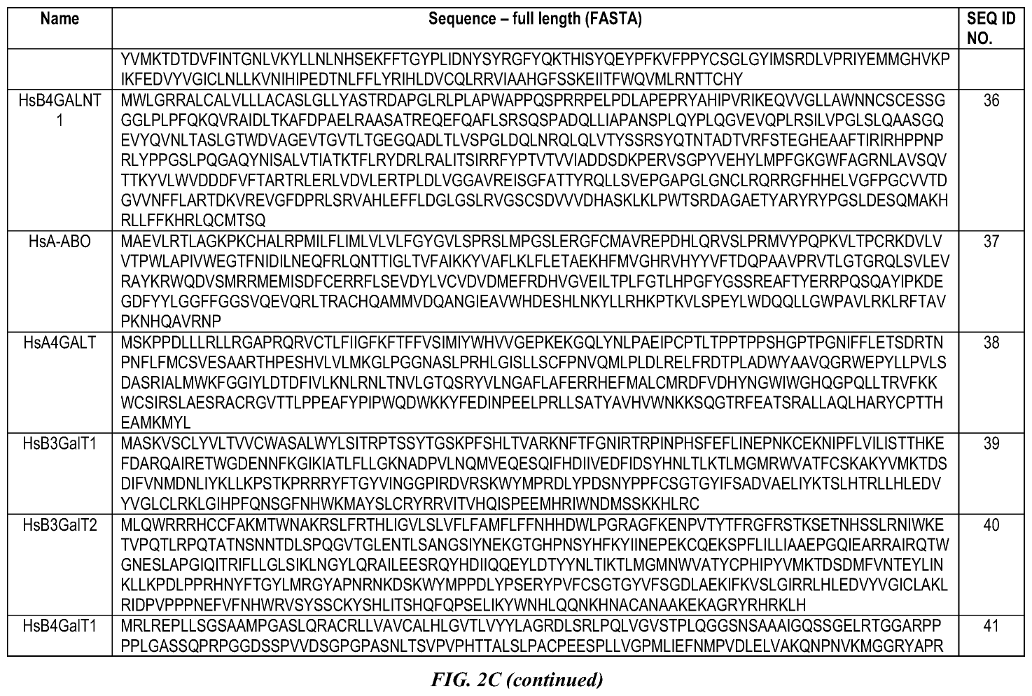

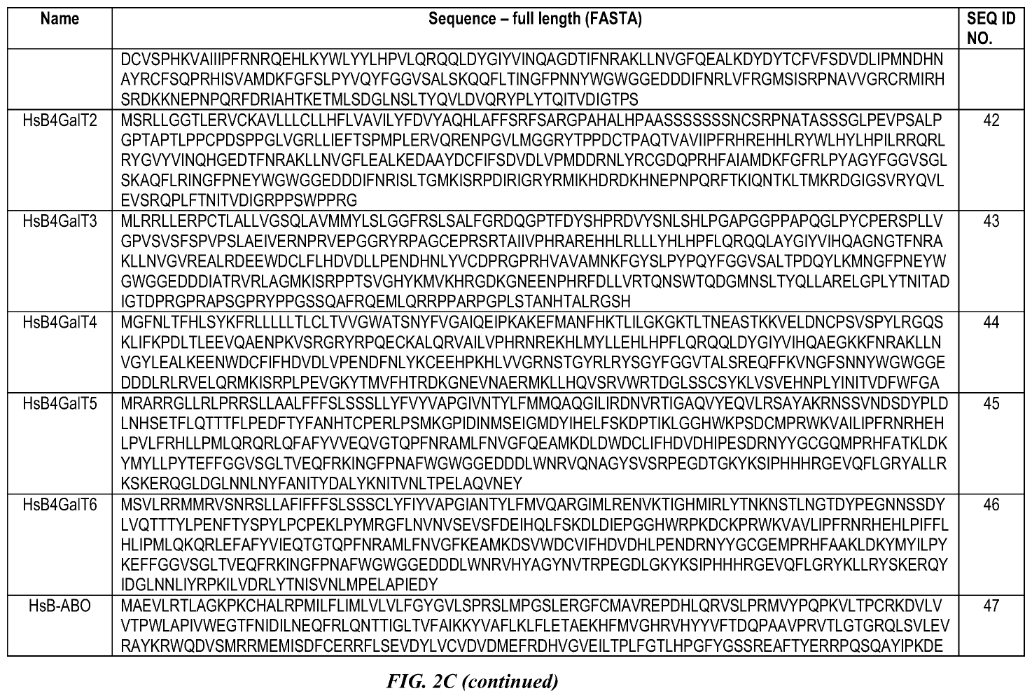

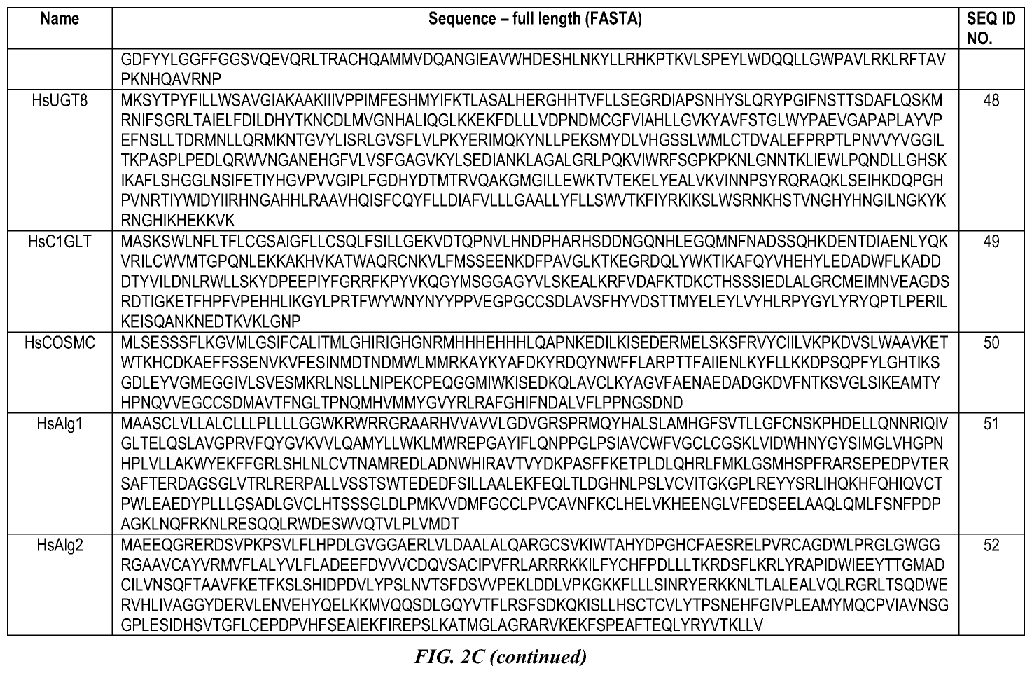

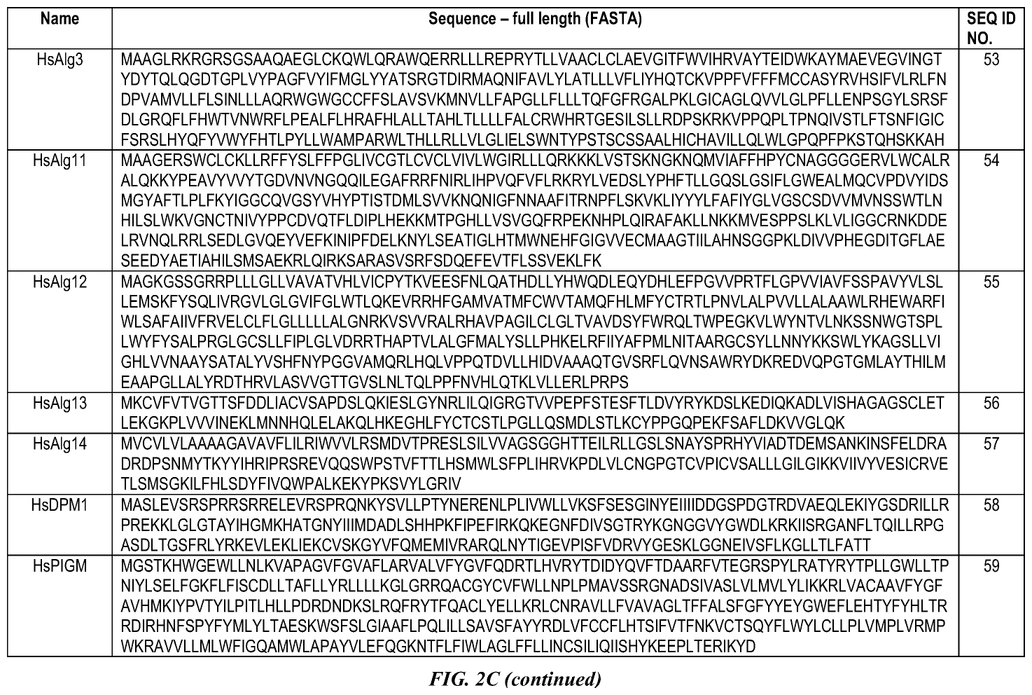

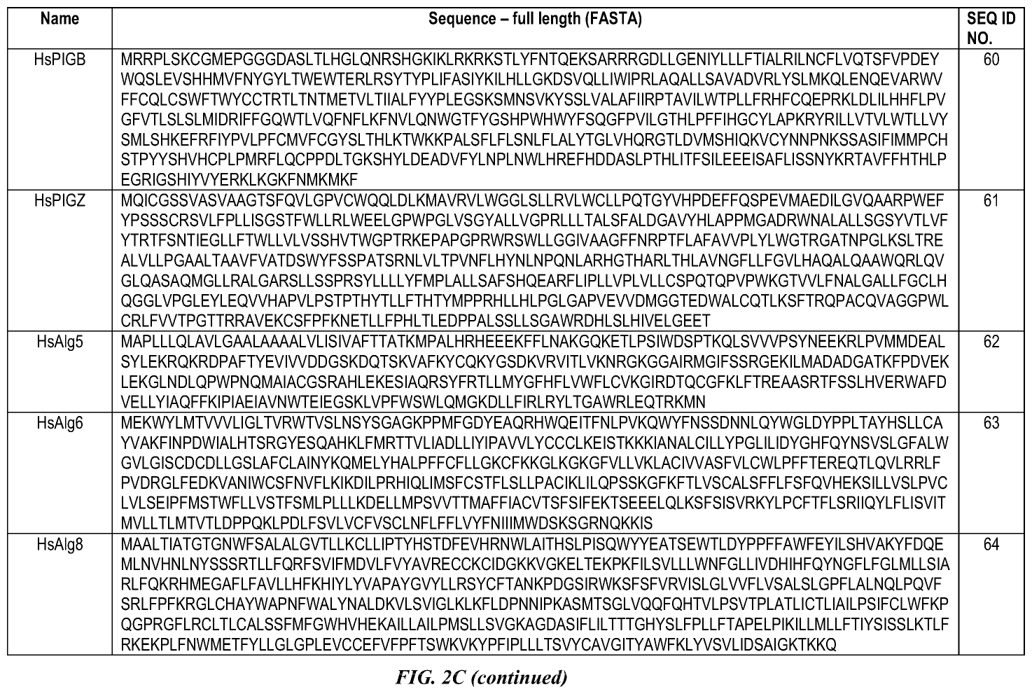

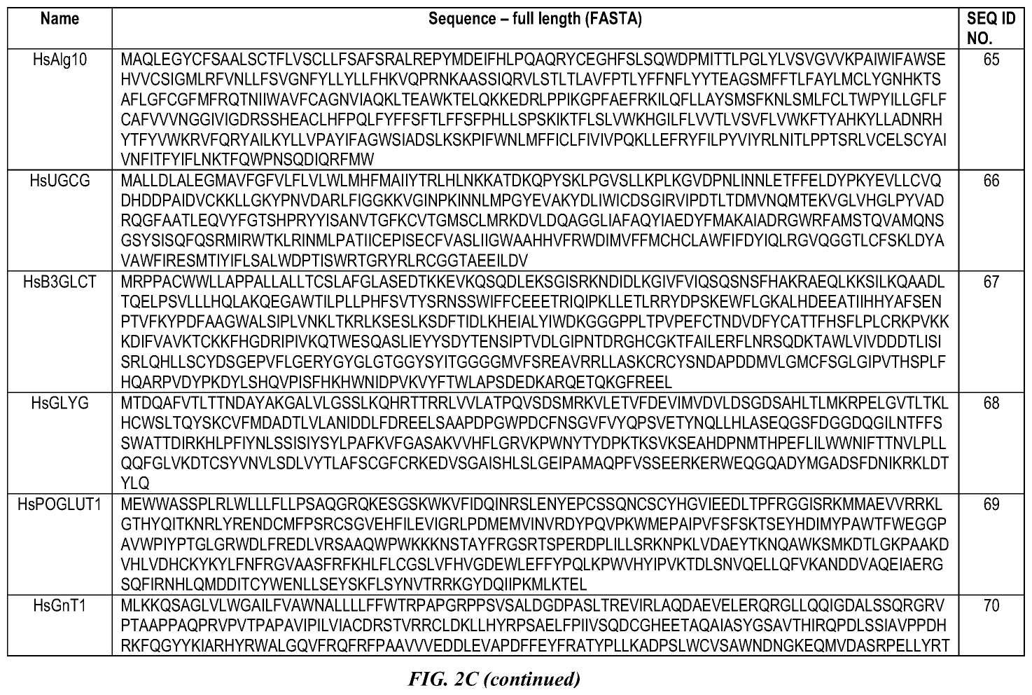

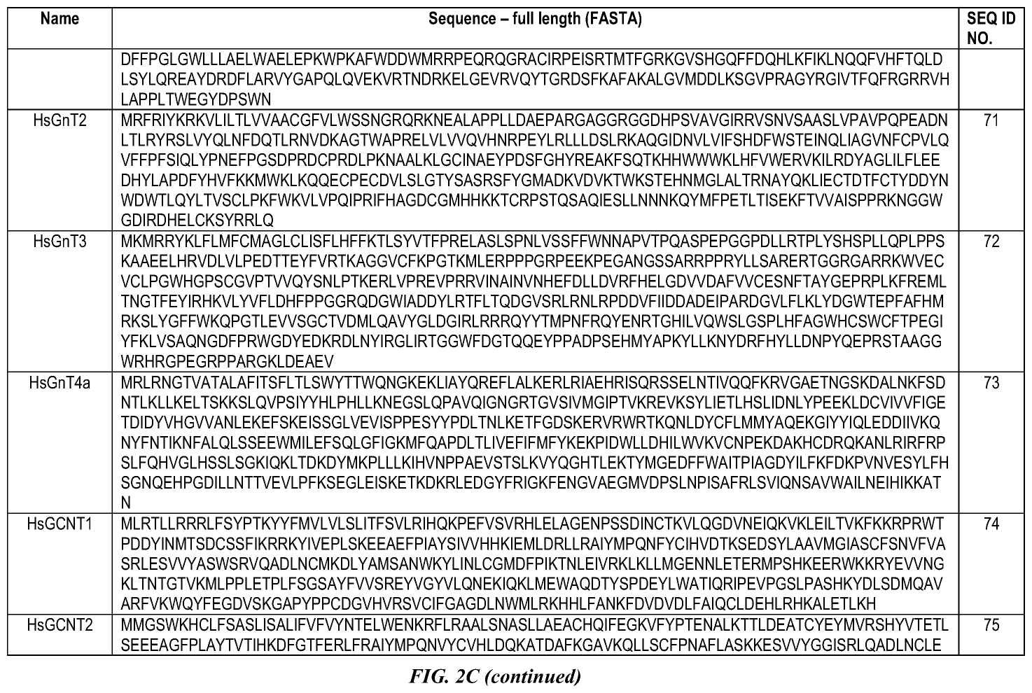

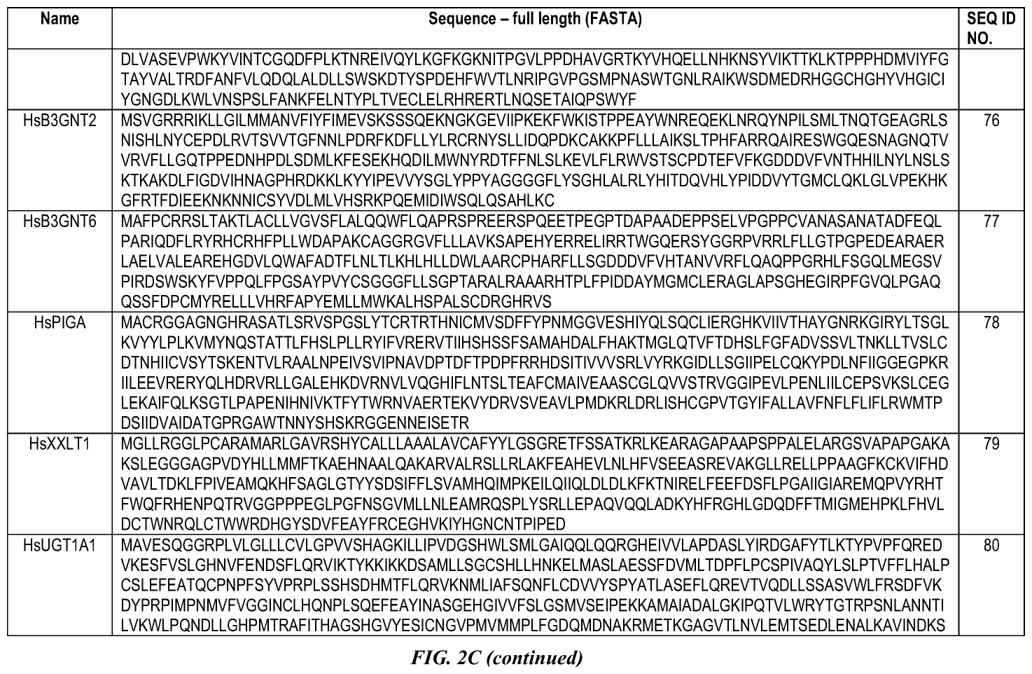

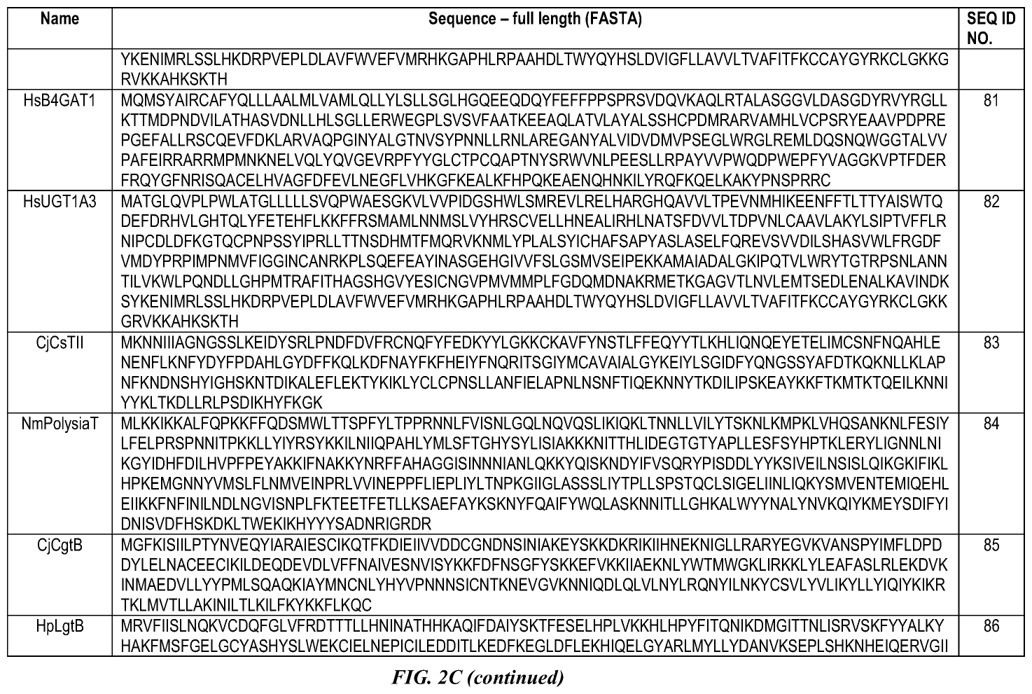

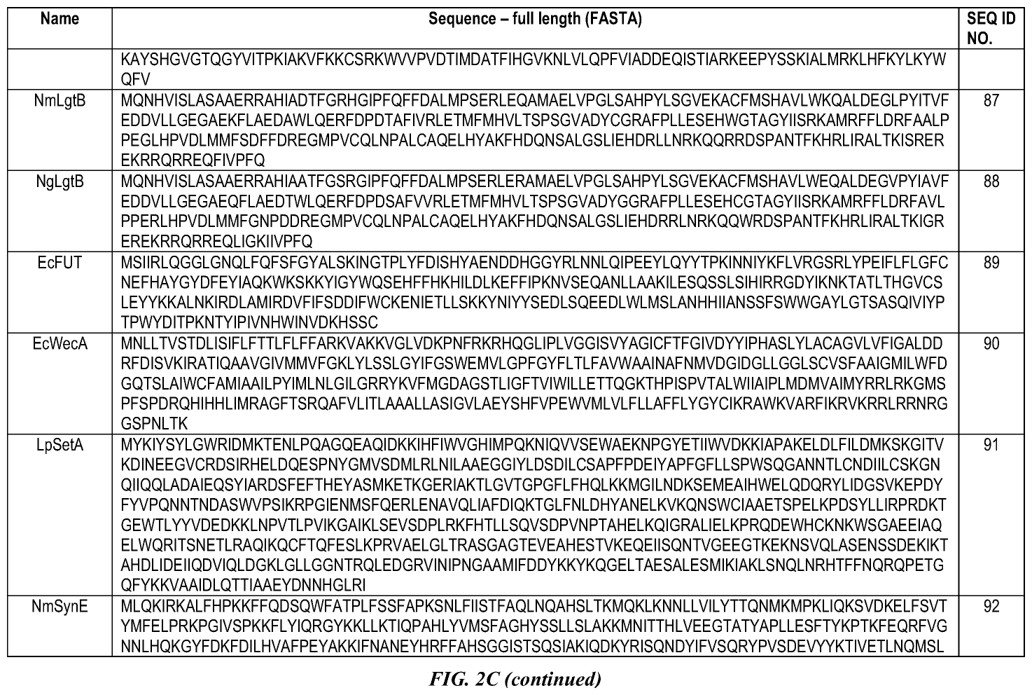

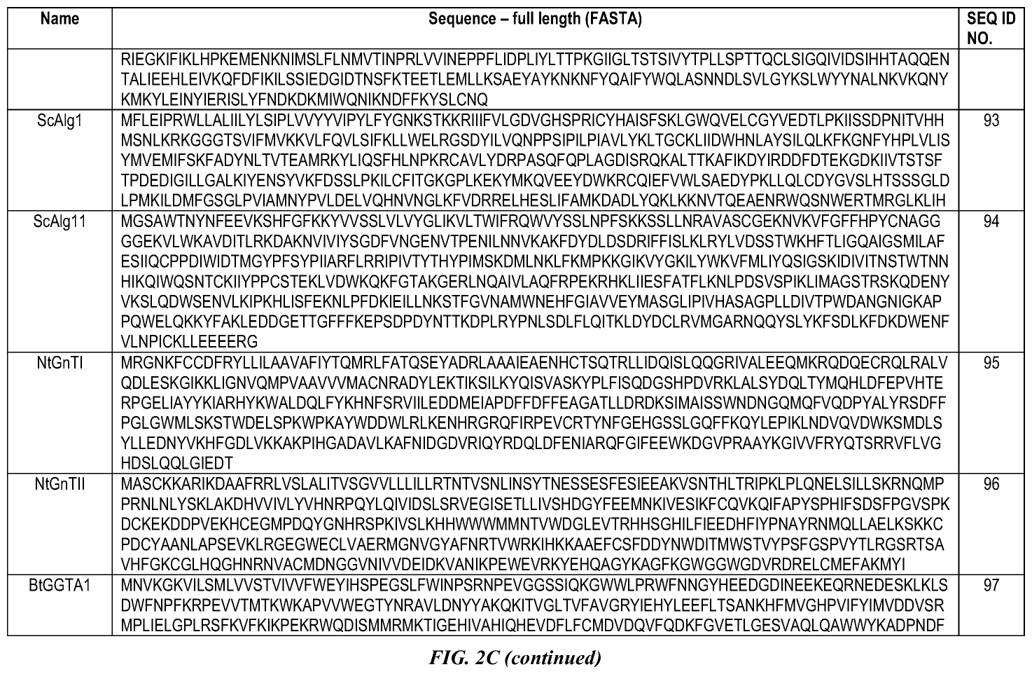

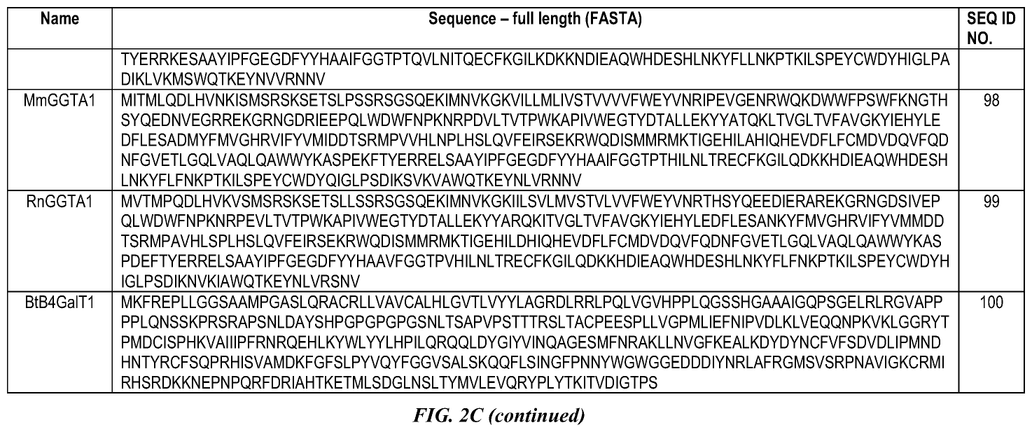

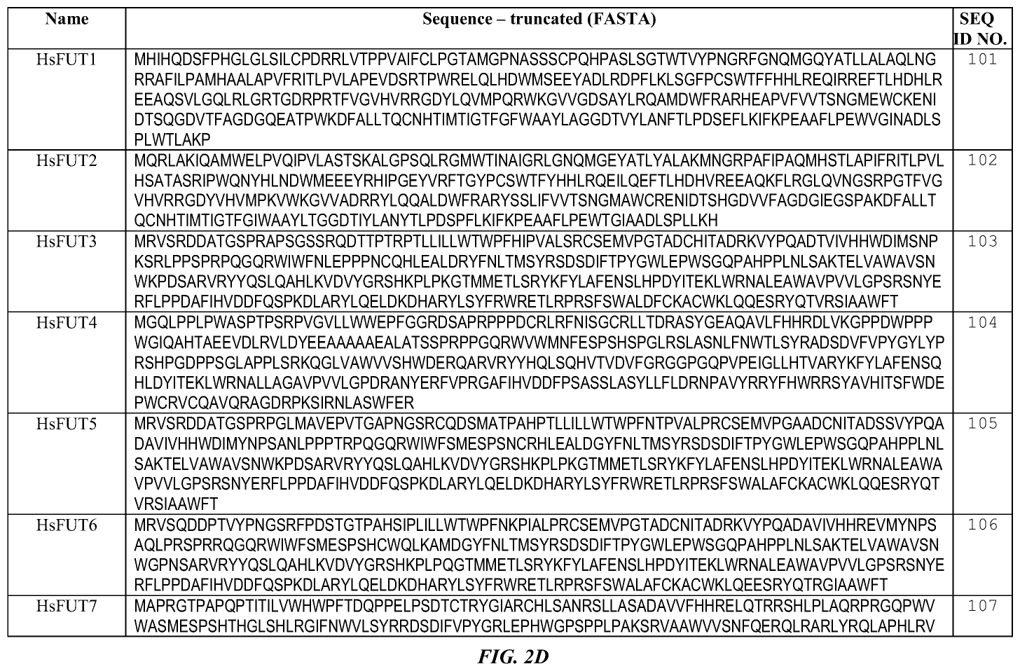

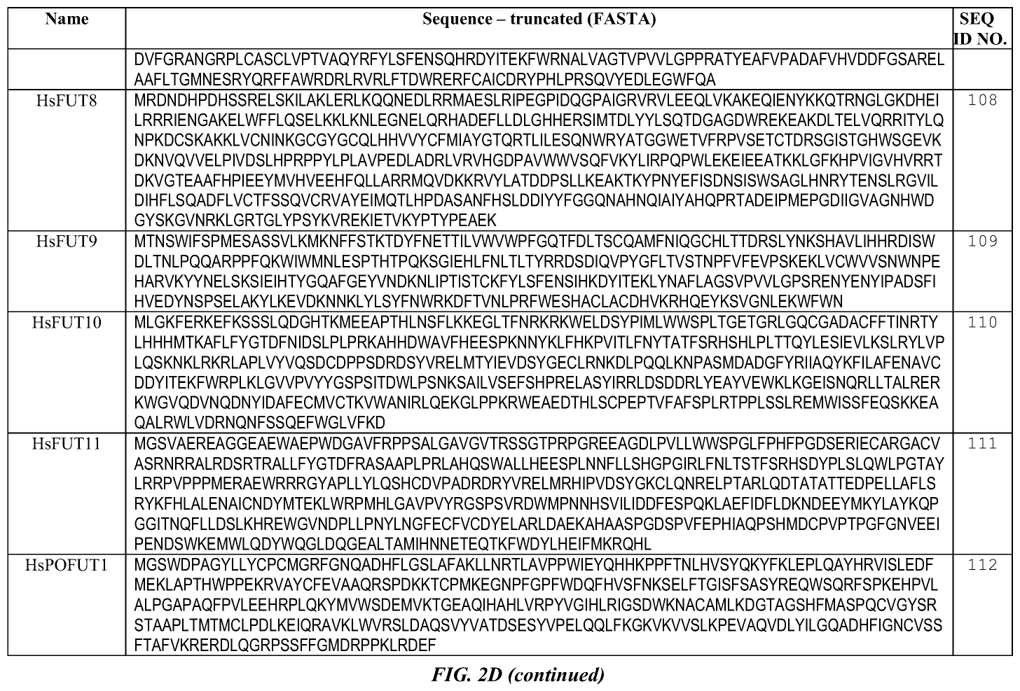

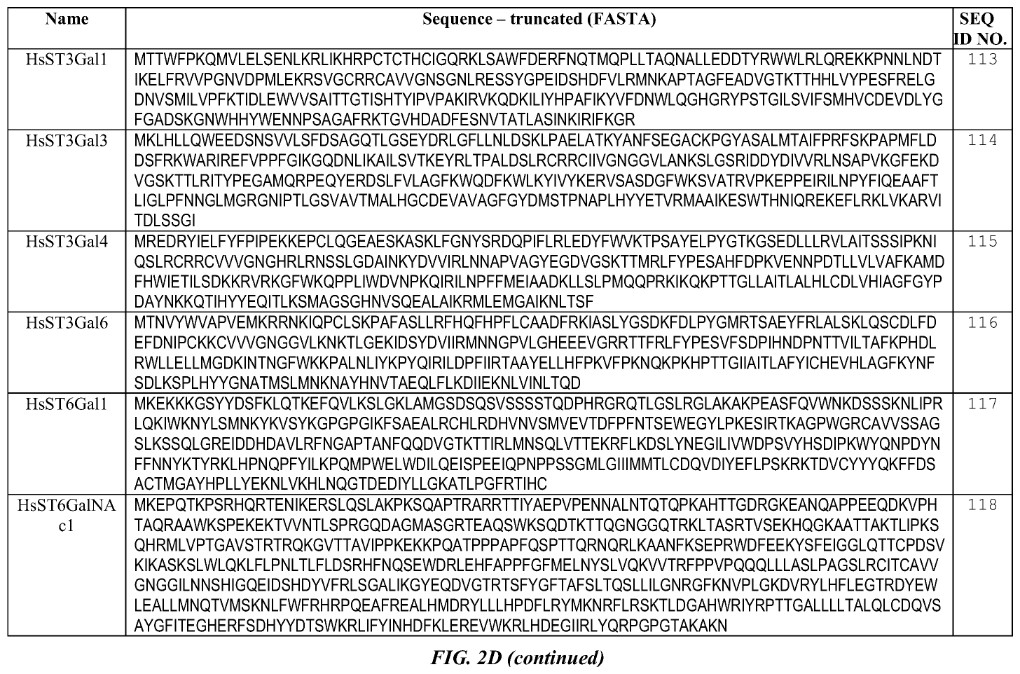

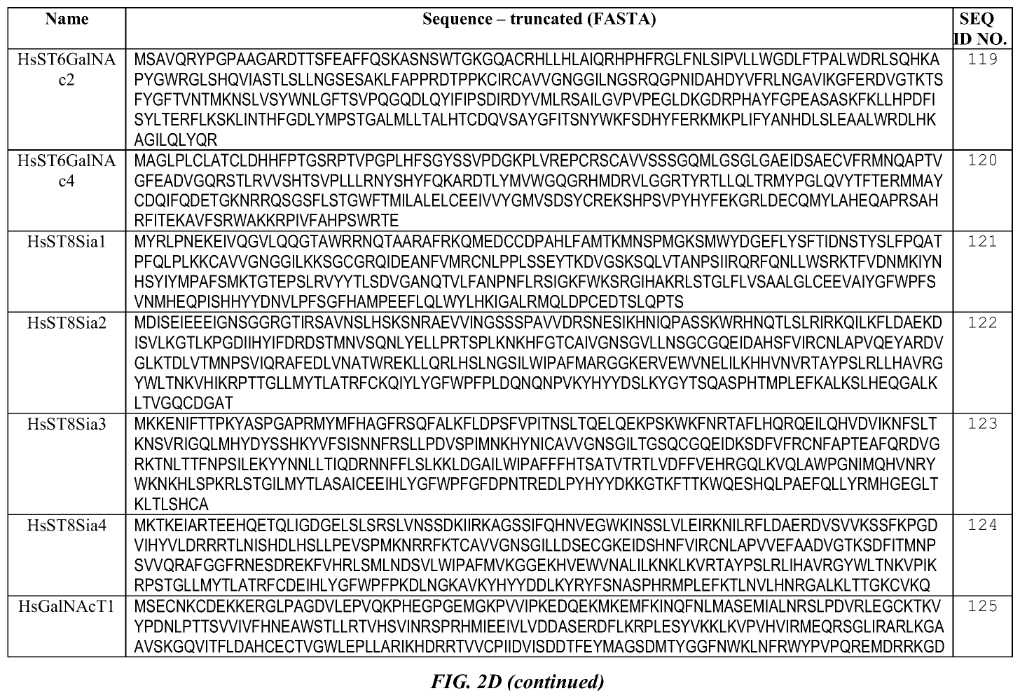

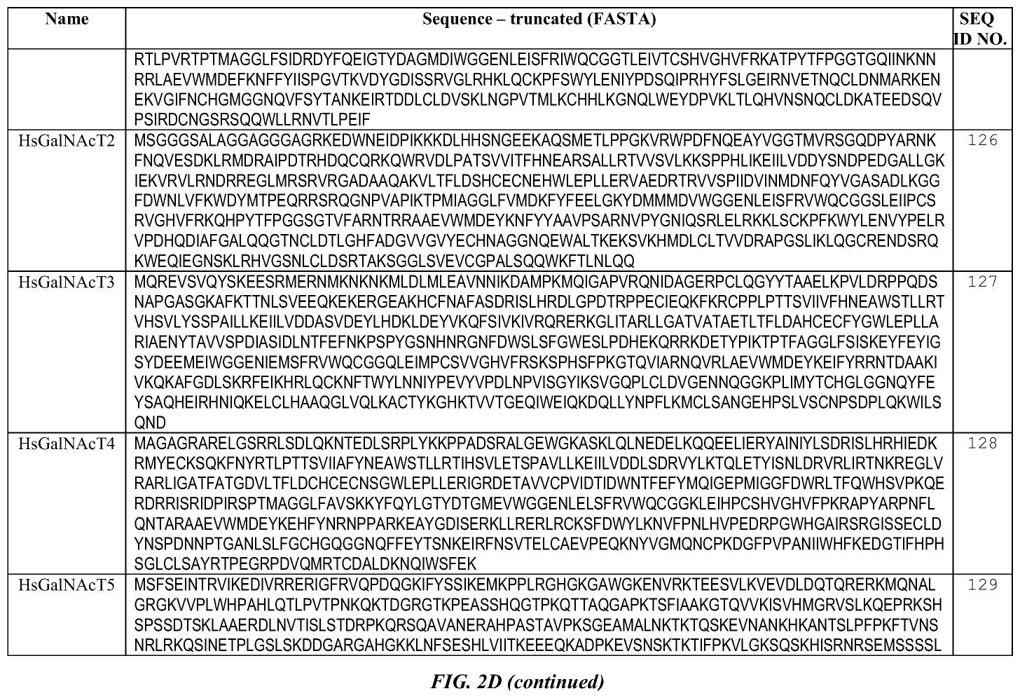

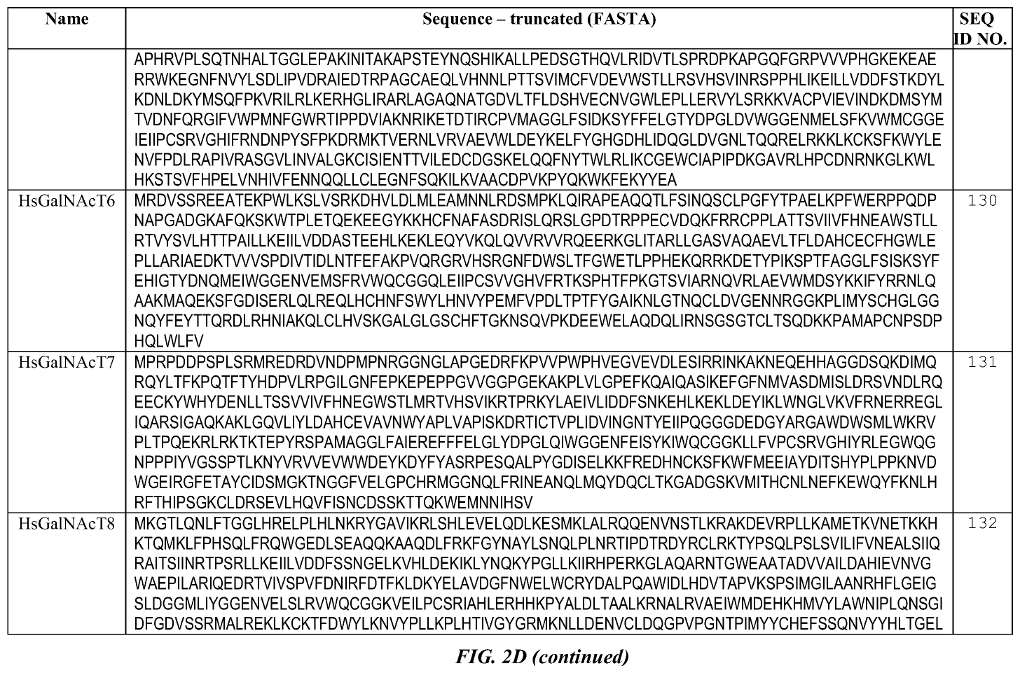

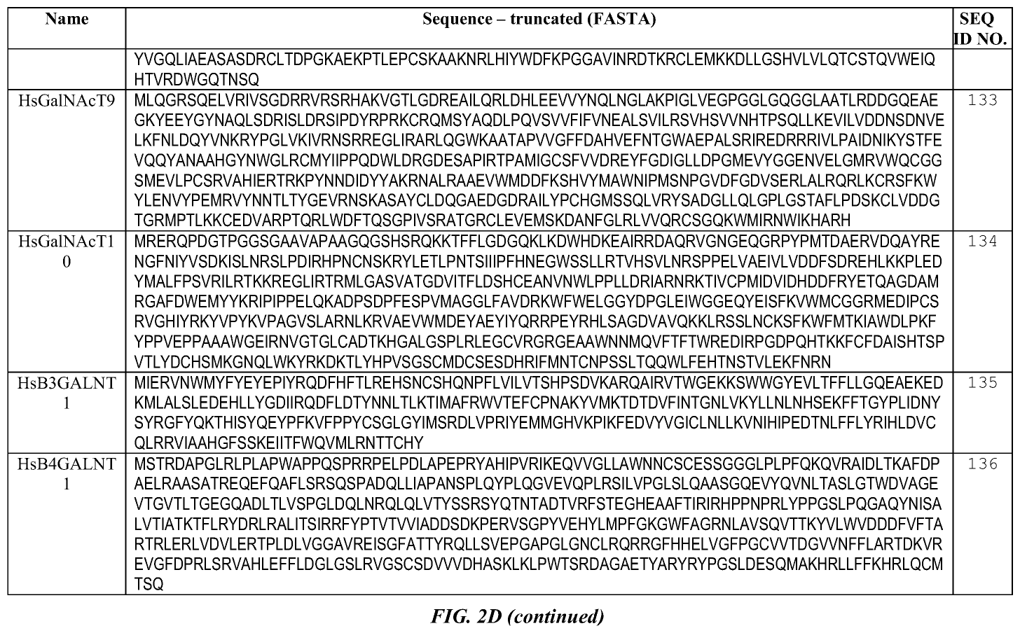

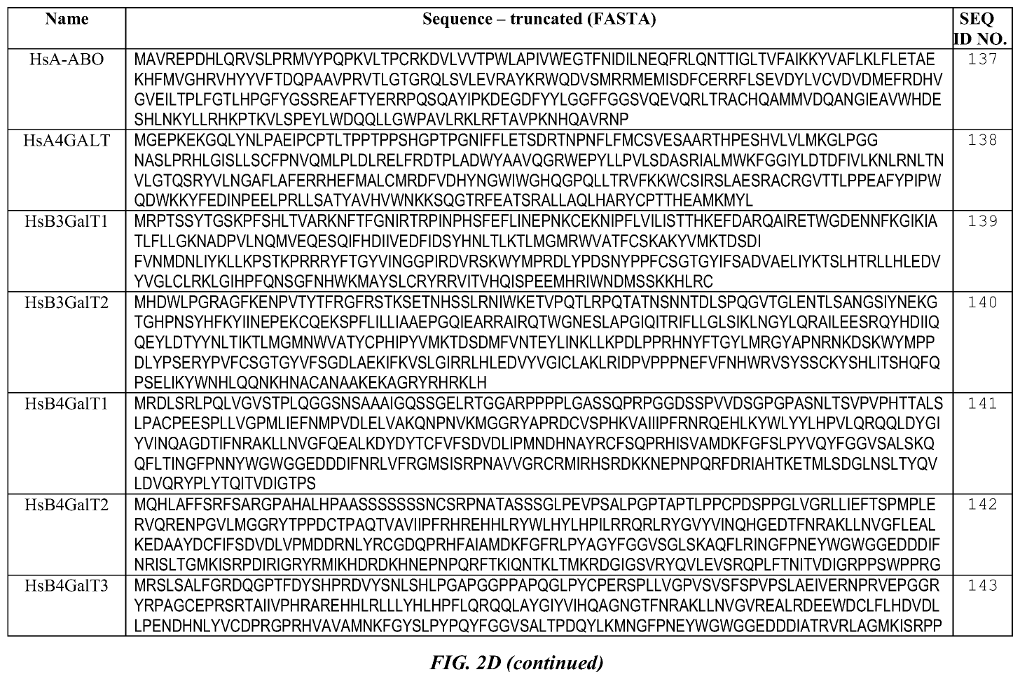

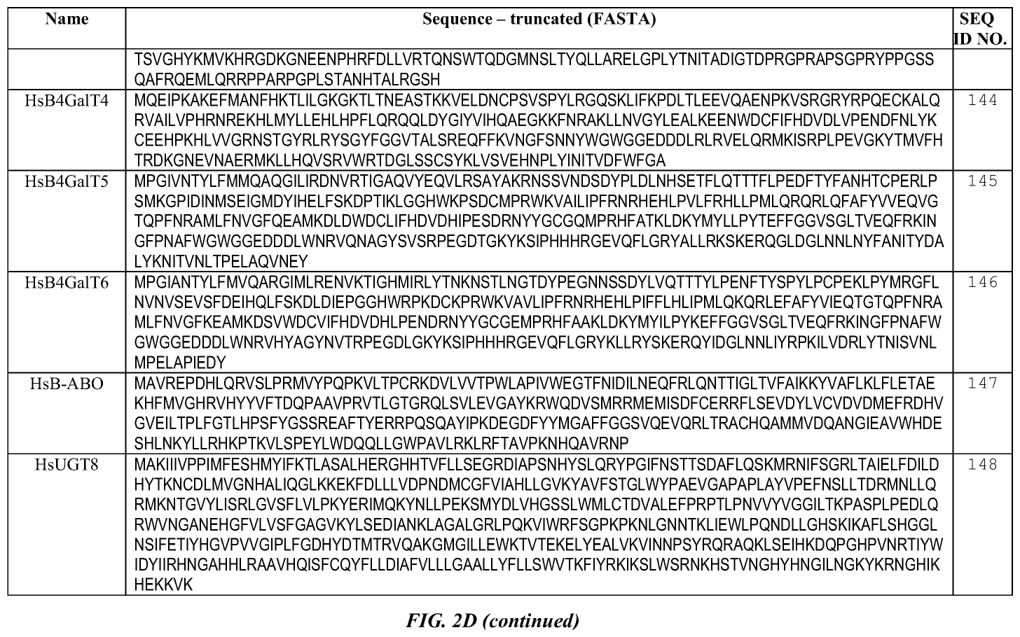

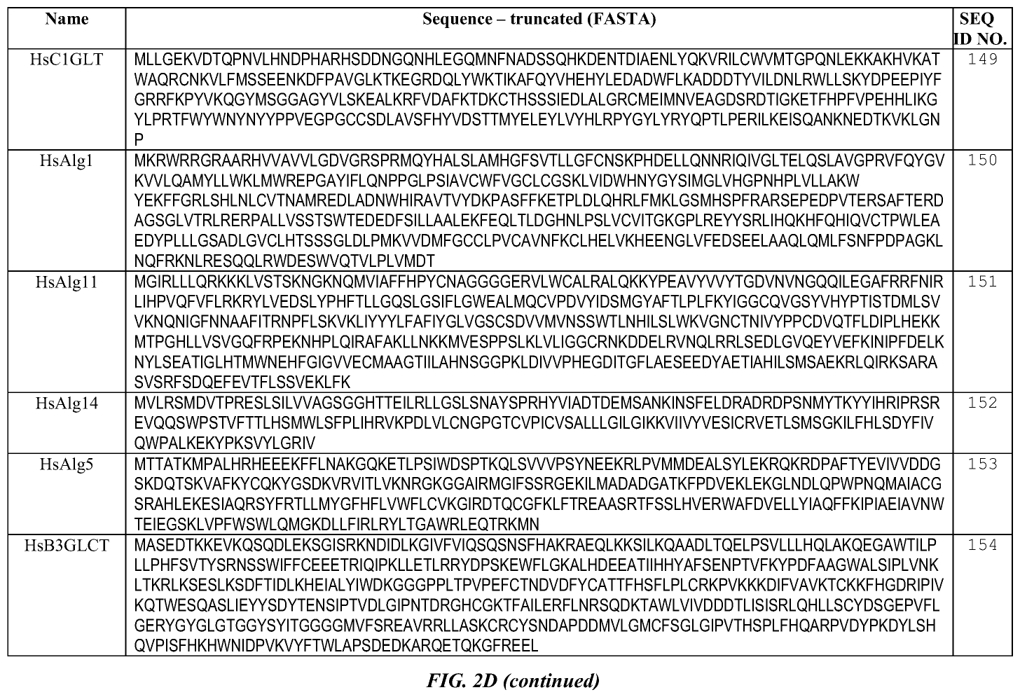

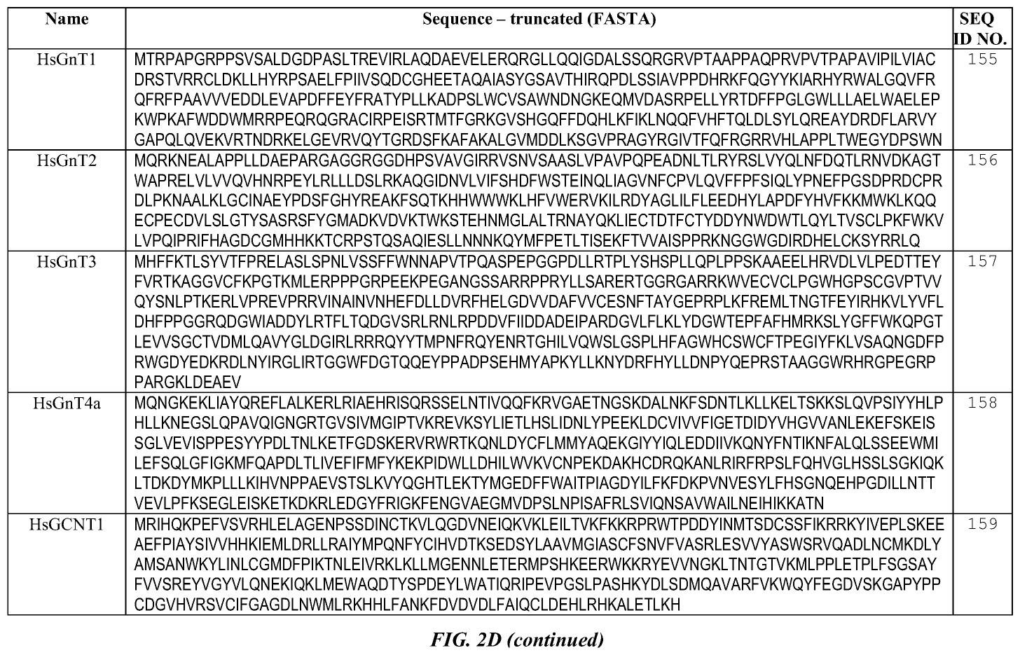

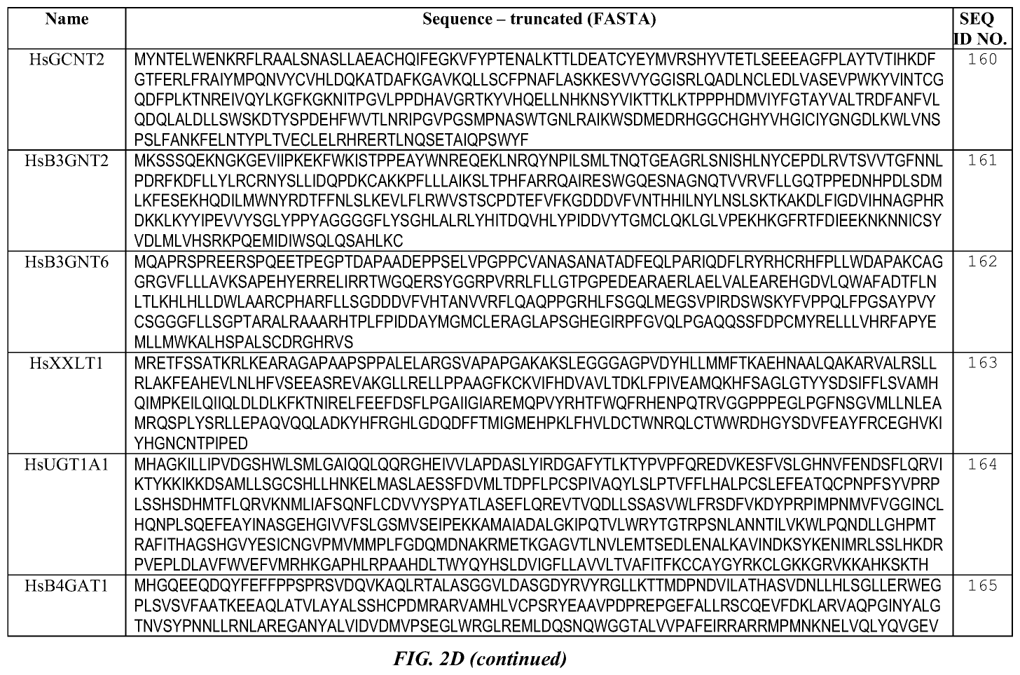

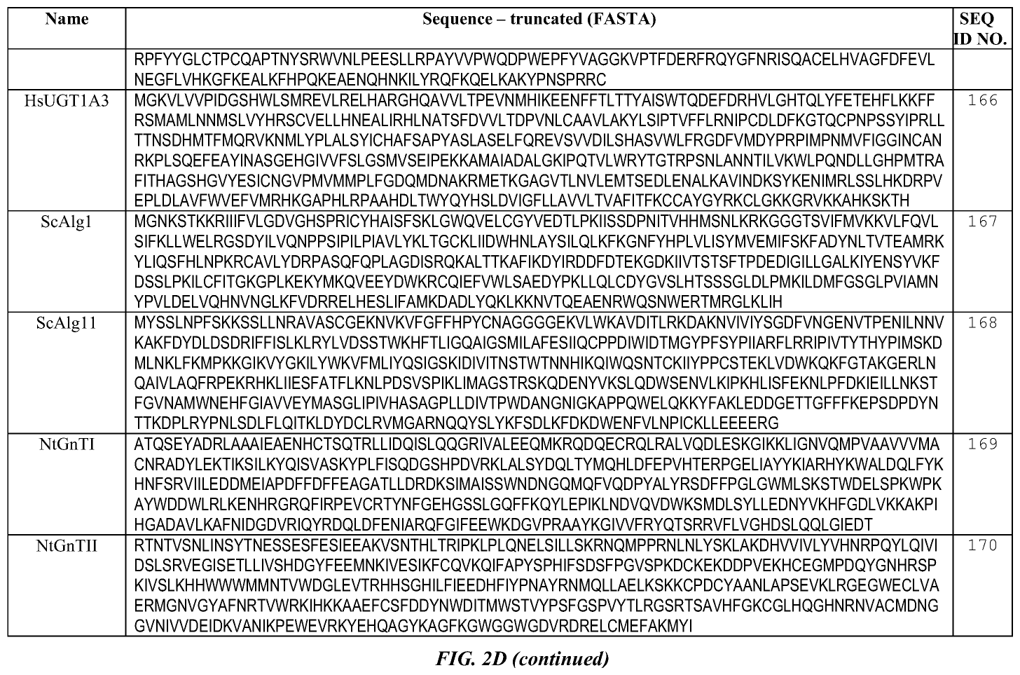

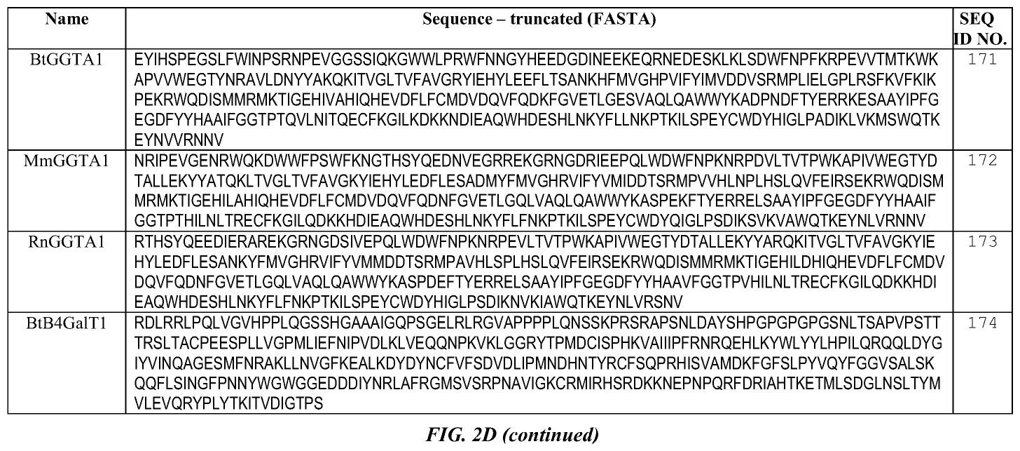

For example, the full-length GT may contain an internal single-pass or multi-pass TMD (e.g., human Dol-P-Man:Man(5)GlcNAc(2)-PP-Dol alpha-1,3-mannosyltransferase (HsAlg3), human Dol-P-Man:Man(7)GlcNAc(2)-PP-Dol alpha-1,6-mannosyltransferase (HsAlg12), human GPI mannosyltransferase 1 (HsPIGM), human GPI mannosyltransferase 3 (HsPIGB), human GPI mannosyltransferase 4 (HsPIGZ), human dolichyl pyrophosphate Man9GlcNAc2 alpha-1,3-glucosyltransferase (HsAlg6), human probable dolichyl pyrophosphate Glc1Man9GlcNAc2 alpha-1,3-glucosyltransferase (HsAlg8), human Dol-P-Glc:Glc(2)Man(9)GlcNAc(2)-PP-Dol alpha-1,2-glucosyltransferase (HsAlg10), human ceramide glucosyltransferase (HsUGCG), E. coli undecaprenyl-phosphate alpha-N-acetylglucosaminyl 1-phosphate transferase (EcWecA), yeast beta-1,4-mannosyltransferase OS=Saccharomyces cerevisiae (ScAlg1), and yeast GDP-Man:Man(3)GlcNAc(2)-PP-Dol alpha-1,2-mannosyltransferase (ScAlg11) (FIG. 2A)).

In other embodiments, the full-length GT may be a predicted cytosolic GT (e.g., human isoform 2 of putative UDP-N-acetylglucosamine transferase (HsAlg13), human dolichol-phosphate mannosyltransferase subunit 1 (HsDPM1), human glycogenin-1 (HsGLYG), Campylobacter jejuni CsTII (CjCsTII), Neisseria meningitidis polysialic acid O-acetyltransferase (NmPolysiaT), Campylobacter jejuni beta-1,3-galactosyltransferase (CjCgtB), Helicobacter pylori (strain 51) beta-4-galactosyltransferase (HpLgtB), Neisseria meningitidis serogroup B (strain MC58) lacto-N-neotetraose biosynthesis glycosyltransferase LgtB (NmLgtB), Neisseria gonorrhoeae lacto-N-neotetraose biosynthesis glycosyltransferase (NgLgtB), E. coli galactoside 2-alpha-L-fucosyltransferase WbgL (EcFUT), Legionella pneumophila subsp. Pneumophila subversion of eukaryotic traffic protein A (LpSetA), and Neisseria meningitidis Alpha-2,9-polysialyltransferase (NmSynE) (FIG. 2A)).

As described infra N-/C-terminal transmembrane domains (TMDs) as well as C-terminal ER retention domains in mammalian GTs are used as membrane anchors and are dispensable for catalytic activity (Harduin-Lepers et al., “The Human Sialyltransferase Family,” Biochimie 83727-83737 (2001), which is hereby incorporated by reference in its entirety). Thus, in some embodiments, the glycosyltransferase is a truncated glycosyltransferase. The truncated glycosyltransferase may exclude a GT C-terminal ER retention domain, a terminal TMD anchor, or both a C-terminal ER retention domain and a terminal TMD anchor. In some embodiments, the truncated glycosyltransferase excludes an N-terminal signal peptides. Various exemplary truncated GTs are provided in FIG. 2D.

Glycosyltransferases play vital roles in glycosylation and glycan remodeling. The tripartite glycosyltransferase fusion proteins according to the present disclosure are water soluble following extraction from their native environment (e.g., a cellular membrane) without the use of detergents and/or detergent-like amphiphiles, overproduction using recombinant systems, protein engineering, and/or mutations to the GT itself, thereby allowing for improved functional and structural studies of GTs as well as in vitro reconstitution of enzymatic activity or in vitro reconstitution of a biological pathway involving water soluble GT enzymes and engineering of biological/metabolic pathways involving the water soluble GTs.

The GTs according to the present disclosure may be prokaryotic glycosyltransferases or eukaryotic glycosyltransferase (e.g., human glycosyltransferases, rodent glycosyltransferases, yeast glycosyltransferases). Suitable exemplary prokaryotic and eukaryotic glycosyltransferases are identified in FIG. 2A.

The glycosyltransferase may be selected from the group consisting of fucosyltransferases (FucTs), galactosyltransferases (Gals), glucosyltransferases (GlcTs), mannosyltransferases (ManTs), N-acetylgalactosyltransferases (GalNAcTs), N-acetylglucosaminyltransferases (GlcNAcTs), and sialyltransferases (SiaTs).

Fucosyltransferases (FucTs) catalyze the transfer a fucose sugar from a donor substrate to an acceptor substrate. Suitable FucTs include, without limitation, human galactoside 2-alpha-L-fucosyltransferase 1 (HsFUT1), human galactoside 2-alpha-L-fucosyltransferase 2 (HsFUT2), HUMAN Galactoside 3(4)-L-fucosyltransferase (HsFUT3), human alpha-(1,3)-fucosyltransferase 4 (HsFUT4), human alpha-(1,3)-fucosyltransferase 5 (HsFUT5), human alpha-(1,3)-fucosyltransferase 6 (HsFUT6), human alpha-(1,3)-fucosyltransferase 7 (HsFUT7), human alpha-(1,6)-fucosyltransferase (HsFUT8), human alpha-(1,3)-fucosyltransferase 9 (HsFUT9), human alpha-(1,3)-fucosyltransferase 10 (HsFUT10), human alpha-(1,3)-fucosyltransferase 11 (HsFUT11), and human GDP-fucose protein O-fucosyltransferase 1 (HsPOFUT1) (see, e.g., FIG. 2A).

Galactosyltransferases (Gals) catalyze the transfer of a galactose sugar from a donor substrate to an acceptor substrate. Suitable Gals include, without limitation, human beta-1,3-galactosyltransferase 1 (HsB3GalT1), human beta-1,3-galactosyltransferase 2 (HsB3GalT2), human beta-1,4-galactosyltransferase 1 (HsB4GalT1), human beta-1,4-galactosyltransferase 2 (HsB4GalT2), human beta-1,4-galactosyltransferase 3 (HsB4GalT3), human beta-1,4-galactosyltransferase 4 (HsB4GalT4), human beta-1,4-galactosyltransferase 5 (HsB4GalT5), and human beta-1,4-galactosyltransferase 6 (HsB4GalT6) (see, e.g., FIG. 2A).

Glucosyltransferases (GlcTs) catalyze the transfer of a glucose sugar from a donor substrate to an acceptor substrate. Suitable GlcTs include, without limitation, human dolichyl-phosphate beta-glucosyltransferase (HsAlg5), human dolichyl pyrophosphate man₉GlcNAc₂ alpha-1,3-glucosyltransferase (HsAlg6), human probable dolichyl pyrophosphate Glc1Man₉GlcNAc₂ alpha-1,3-glucosyltransferase (HsAlg8), human Dol-P-Glc:Glc₂Man₉GlcNAc₂—PP-Dol alpha-1,2-glucosyltransferase (HsAlg10), human ceramide glucosyltransferase (HsUGCG), human beta-1,3-glucosyltransferase (HsB3GLCT), and human protein O-glucosyltransferase 1 (HsPOGLUT1) (see, e.g., FIG. 2A).

Mannosyltransferases (ManTs) catalyze the transfer of a mannose sugar from a donor substrate to an acceptor substrate. Suitable ManTs include, without limitation, human chitobiosyldiphosphodolichol beta-mannosyltransferase (HsAlg1), human alpha-1,3/1,6-mannosyltransferase (HsAlg2), human Dol-P-Man:Man(5)GlcNAc(2)-PP-Dol alpha-1,3-mannosyltransferase (HsAlg3), human GDP-man:man(3)GlcNAc(2)-PP-Dol alpha-1,2-mannosyltransferase (HsAlg11), human dol-p-man:man(7)GlcNAc(2)-PP-Dol alpha-1,6-mannosyltransferase (HsAlg12), human dolichol-phosphate mannosyltransferase subunit 1 (HsDPM1), human GPI mannosyltransferase 1 (HsPIGM), human GPI mannosyltransferase 3 (HsPIGB), human GPI mannosyltransferase 4 (HsPIGZ), yeast beta-1,4-mannosyltransferase OS=Saccharomyces cerevisiae (ScAlg1), and yeast GDP-Man:Man(3)GlcNAc(2)-PP-Dol alpha-1,2-mannosyltransferase (ScAlg11) (see, e.g., FIG. 2A).

N-acetylgalactosyltransferases (GalNAcTs) catalyze the transfer of an N-acetylgalactosamine to an acceptor substrate. Suitable GalNAcTs include, without limitation, human alpha-N-acetylgalactosaminide alpha-2,6-sialyltransferase 1 (HsST6GalNAc1), human alpha-N-acetylgalactosaminide alpha-2,6-sialyltransferase 2 (HsST6GalNAc2), human alpha-N-acetyl-neuraminyl-2,3-beta-galactosyl-1,3-N-acetyl-galactosaminide alpha-2,6-sialyltransferase (HsST6GalNAc4), human polypeptide N-acetylgalactosaminyltransferase 1 (HsppGalNAcT1), human polypeptide N-acetylgalactosaminyltransferase 2 (HsppGalNAcT2), human polypeptide N-acetylgalactosaminyltransferase 3 (HsppGalNAcT3), human polypeptide N-acetylgalactosaminyltransferase 4 (HsppGalNAcT4), human polypeptide N-acetylgalactosaminyltransferase 5 (HsppGalNAcT5), human polypeptide N-acetylgalactosaminyltransferase 6 (HsppGalNAcT6), human N-acetylgalactosaminyltransferase 7 (HsppGalNAcT7), human probable polypeptide N-acetylgalactosaminyltransferase 8 (HsppGalNAcT8), human polypeptide N-acetylgalactosaminyltransferase 9 (HsppGalNAcT9), human polypeptide N-acetylgalactosaminyltransferase 10 (HsppGalNAcT10), and human UDP-GalNAc:beta-1,3-N-acetylgalactosaminyltransferase 1 (HsB3GALNT1) (see, e.g., FIG. 2A).

N-acetylglucosaminyltransferases (GlcNAcTs) catalyze the transfer of an N-acetylglucosamine to an acceptor substrate. Suitable GlcNAcTs include, without limitation, human alpha-1,3-mannosyl-glycoprotein 2-beta-N-acetylglucosaminyltransferase (HsGnTI/MGAT1), human alpha-1,6-mannosyl-glycoprotein 2-beta-N-acetylglucosaminyltransferase (HsGnTII/MGAT2), human beta-1,4-mannosyl-glycoprotein 4-beta-N-acetylglucosaminyltransferase (HsGnTIII/MGAT3), human alpha-1,3-mannosyl-glycoprotein 4-beta-N-acetylglucosaminyltransferase a (HsGnTIV/MGAT4), human beta-1,3-galactosyl-O-glycosyl-glycoprotein beta-1,6-N-acetylglucosaminyltransferase (HsGCNT1), human N-acetyllactosaminide beta-1,6-N-acetylglucosaminyltransferase (HsGCNT2), human N-acetyllactosaminide beta-1,3-N-acetylglucosaminyltransferase 2 (HsB3GNT2), human acetylgalactosaminyl-O-glycosyl-glycoprotein beta-1,3-N-acetylglucosaminyltransferase (HsB3GNT6), human phosphatidylinositol N-acetylglucosaminyltransferase subunit A (HsPIGA), Nicotiana tabacum alpha-1,3-mannosyl-glycoprotein 2-beta-N-acetylglucosaminyltransferase (NtGnTI), and Nicotiana tabacum alpha-1,6-mannosyl-glycoprotein 2-beta-N-acetylglucosaminyltransferase-like (NtGnTII) (see, e.g., FIG. 2A).

Sialyltransferases (SiaTs) catalyze the transfer of sialic acid to an acceptor substrate. Suitable SiaTs include, without limitation, human CMP-N-acetylneuraminate-beta-galactosamide-alpha-2,3-sialyltransferase 1 (HsST3Gal1), human CMP-N-acetylneuraminate-beta-1,4-galactoside alpha-2,3-sialyltransferase (HsST3Gal3), human CMP-N-acetylneuraminate-beta-galactosamide-alpha-2,3-sialyltransferase 4 (HsST3Gal4), human type 2 lactosamine alpha-2,3-sialyltransferase (HsST3Gal6), human beta-galactoside alpha-2,6-sialyltransferase 1 (HsST6Gal1), human alpha-N-acetylgalactosaminide alpha-2,6-sialyltransferase 1 (HsST6GalNAc1), human alpha-N-acetylgalactosaminide alpha-2,6-sialyltransferase 2 (HsST6GalNAc2), human alpha-N-acetyl-neuraminyl-2,3-beta-galactosyl-1,3-N-acetyl-galactosaminide alpha-2,6-sialyltransferase (HsST6GalNAc4), human alpha-N-acetylneuraminide alpha-2,8-sialyltransferase (HsST8Sia1), human alpha-2,8-sialyltransferase 8b (HsST8Sia2), human sia-alpha-2,3-gal-beta-1,4-GlcNAc-R:alpha 2,8-sialyltransferase (HsST8Sia3), human CMP-N-acetylneuraminate-poly-alpha-2,8-sialyltransferase (HsST8Sia4), and Neisseria meningitidis alpha-2,9-polysialyltransferase (NmSynE) (see, e.g., FIG. 2A).

In some embodiments, the glycosyltransferase is selected from the group consisting of human galactoside 2-alpha-L-fucosyltransferase 1 (HsFUT1), human galactoside 2-alpha-L-fucosyltransferase 2 (HsFUT2), HUMAN Galactoside 3(4)-L-fucosyltransferase (HsFUT3), human alpha-(1,3)-fucosyltransferase 4 (HsFUT4), human alpha-(1,3)-fucosyltransferase 5 (HsFUT5), human alpha-(1,3)-fucosyltransferase 6 (HsFUT6), human alpha-(1,3)-fucosyltransferase 7 (HsFUT7), human alpha-(1,6)-fucosyltransferase (HsFUT8), human alpha-(1,3)-fucosyltransferase 9 (HsFUT9), human alpha-(1,3)-fucosyltransferase 10 (HsFUT10), human alpha-(1,3)-fucosyltransferase 11 (HsFUT11), human GDP-fucose protein O-fucosyltransferase 1 (HsPOFUT1), human CMP-N-acetylneuraminate-beta-galactosamide-alpha-2,3-sialyltransferase 1 (HsST3Gal1), human CMP-N-acetylneuraminate-beta-1,4-galactoside alpha-2,3-sialyltransferase (HsST3Gal3), human CMP-N-acetylneuraminate-beta-galactosamide-alpha-2,3-sialyltransferase 4 (HsST3Gal4), human type 2 lactosamine alpha-2,3-sialyltransferase (HsST3Gal6), human beta-galactoside alpha-2,6-sialyltransferase 1 (HsST6Gal1), human alpha-N-acetylgalactosaminide alpha-2,6-sialyltransferase 1 (HsST6GalNAc1), human alpha-N-acetylgalactosaminide alpha-2,6-sialyltransferase 2 (HsST6GalNAc2), human alpha-N-acetyl-neuraminyl-2,3-beta-galactosyl-1,3-N-acetyl-galactosaminide alpha-2,6-sialyltransferase (HsST6GalNAc4), human alpha-N-acetylneuraminide alpha-2,8-sialyltransferase (HsST8Sia1), human alpha-2,8-sialyltransferase 8b (HsST8Sia2), human sia-alpha-2,3-gal-beta-1,4-GlcNAc-R:alpha 2,8-sialyltransferase (HsST8Sia3), human CMP-N-acetylneuraminate-poly-alpha-2,8-sialyltransferase (HsST8Sia4), human polypeptide N-acetylgalactosaminyltransferase 1 (HsppGalNAcT1), human polypeptide N-acetylgalactosaminyltransferase 2 (HsppGalNAcT2), human polypeptide N-acetylgalactosaminyltransferase 3 (HsppGalNAcT3), human polypeptide N-acetylgalactosaminyltransferase 4 (HsppGalNAcT4), human polypeptide N-acetylgalactosaminyltransferase 5 (HsppGalNAcT5), human polypeptide N-acetylgalactosaminyltransferase 6 (HsppGalNAcT6), human N-acetylgalactosaminyltransferase 7 (HsppGalNAcT7), human probable polypeptide N-acetylgalactosaminyltransferase 8 (HsppGalNAcT8), human polypeptide N-acetylgalactosaminyltransferase 9 (HsppGalNAcT9), human polypeptide N-acetylgalactosaminyltransferase 10 (HsppGalNAcT10), human UDP-GalNAc:beta-1,3-N-acetylgalactosaminyltransferase 1 (HsB3GALNT1), human beta-1,4 N-acetylgalactosaminyltransferase 1 (HsB4GALNT1), human histo-blood group ABO system transferase (Hs-A-group), human lactosylceramide 4-alpha-galactosyltransferase (HsA4GalT), human beta-1,3-galactosyltransferase 1 (HsB3GalT1), human beta-1,3-galactosyltransferase 2 (HsB3GalT2), human beta-1,4-galactosyltransferase 1 (HsB4GalT1), human beta-1,4-galactosyltransferase 2 (HsB4GalT2), human beta-1,4-galactosyltransferase 3 (HsB4GalT3), human beta-1,4-galactosyltransferase 4 (HsB4GalT4), human beta-1,4-galactosyltransferase 5 (HsB4GalT5), human beta-1,4-galactosyltransferase 6 (HsB4GalT6), human histo-blood group ABO system transferase (Hs-B-group), human 2-hydroxyacylsphingosine 1-beta-galactosyltransferase (HsUGT8), human glycoprotein-N-acetylgalactosamine 3-beta-galactosyltransferase 1 (HsC1GLT), human C1GALT1-specific chaperone 1 (HsCOSMC), human chitobiosyldiphosphodolichol beta-mannosyltransferase (HsAlg1), human alpha-1,3/1,6-mannosyltransferase (HsAlg2), human Dol-P-Man:Man(5)GlcNAc(2)-PP-Dol alpha-1,3-mannosyltransferase (HsAlg3), human GDP-man:man(3)GlcNAc(2)-PP-Dol alpha-1,2-mannosyltransferase (HsAlg11), human dol-p-man:man(7)GlcNAc(2)-PP-Dol alpha-1,6-mannosyltransferase (HsAlg12), human isoform 2 of putative UDP-N-acetylglucosamine transferase (HsAlg13), human UDP-N-acetylglucosamine transferase subunit alg14 homolog (HsAlg14), human dolichol-phosphate mannosyltransferase subunit 1 (HsDPM1), human GPI mannosyltransferase 1 (HsPIGM), human GPI mannosyltransferase 3 (HsPIGB), human GPI mannosyltransferase 4 (HsPIGZ), human dolichyl-phosphate beta-glucosyltransferase (HsAlg5), human dolichyl pyrophosphate man₉GlcNAc₂ alpha-1,3-glucosyltransferase (HsAlg6), human probable dolichyl pyrophosphate Glc1Man₉GlcNAc₂ alpha-1,3-glucosyltransferase (HsAlg8), human Dol-P-Glc:Glc₂Man₉GlcNAc₂-PP-Dol alpha-1,2-glucosyltransferase (HsAlg10), human ceramide glucosyltransferase (HsUGCG), human beta-1,3-glucosyltransferase (HsB3GLCT), human glycogenin-1 (HsGLYG), human protein O-glucosyltransferase 1 (HsPOGLUT1), human alpha-1,3-mannosyl-glycoprotein 2-beta-N-acetylglucosaminyltransferase (HsGnTI/MGAT1), human alpha-1,6-mannosyl-glycoprotein 2-beta-N-acetylglucosaminyltransferase (HsGnTII/MGAT2), human beta-1,4-mannosyl-glycoprotein 4-beta-N-acetylglucosaminyltransferase (HsGnTIII/MGAT3), human alpha-1,3-mannosyl-glycoprotein 4-beta-N-acetylglucosaminyltransferase a (HsGnTIV/MGAT4), human beta-1,3-galactosyl-O-glycosyl-glycoprotein beta-1,6-N-acetylglucosaminyltransferase (HsGCNT1), human N-acetyllactosaminide beta-1,6-N-acetylglucosaminyl-transferase (HsGCNT2), human N-acetyllactosaminide beta-1,3-N-acetylglucosaminyltransferase 2 (HsB3GNT2), human acetylgalactosaminyl-O-glycosyl-glycoprotein beta-1,3-N-acetylglucosaminyltransferase (HsB3GNT6), human phosphatidylinositol N-acetylglucosaminyltransferase subunit A (HsPIGA), human xyloside xylosyltransferase 1 (HsXXLT1), human UDP-glucuronosyltransferase 1-1 (HsUGT1A1), human beta-1,4-glucuronyltransferase 1 (HsB4GAT1), human UDP-glucuronosyltransferase 1-3 (HsUGT1A3), Campylobacter jejuni CsTII (CjCstII), Neisseria meningitidis polysialic acid O-acetyltransferase (NmPst), Campylobacter jejuni beta-1,3-galactosyltransferase (CjCgtB), Helicobacter pylori (strain 51) beta-4-galactosyltransferase (HpLgtB), Neisseria meningitidis serogroup B (strain MC58) lacto-N-neotetraose biosynthesis glycosyltransferase LgtB (NmLgtB), Neisseria gonorrhoeae lacto-N-neotetraose biosynthesis glycosyltransferase (NgLgtB), E. coli galactoside 2-alpha-L-fucosyltransferase WbgL (EcWbgL), E. coli undecaprenyl-phosphate alpha-N-acetylglucosaminyl 1-phosphate transferase (EcWecA), Legionella pneumophila subsp. Pneumophila Subversion of eukaryotic traffic protein A (LpSetA), Neisseria meningitidis alpha-2,9-polysialyltransferase (NmSynE), yeast beta-1,4-mannosyltransferase OS=Saccharomyces cerevisiae (ScAlg1), yeast GDP-Man:Man(3)GlcNAc(2)-PP-Dol alpha-1,2-mannosyltransferase (ScAlg11), Nicotiana tabacum alpha-1,3-mannosyl-glycoprotein 2-beta-N-acetylglucosaminyltransferase (NtGnTI), Nicotiana tabacum alpha-1,6-mannosyl-glycoprotein 2-beta-N-acetylglucosaminyltransferase-like (NtGnTII), Bos taurus n-acetyllactosaminide alpha-1,3-galactosyltransferase (BtGGTA1), mouse n-acetyllactosaminide alpha-1,3-galactosyltransferase (MmGGTA1), rat n-acetyllactosaminide alpha-1,3-galactosyltransferase (RnGGTA1), and Bos taurus beta-1,4-galactosyltransferase 1 (BtB4GalT1) (see, e.g., FIG. 2A).

In some embodiments, the nucleic acid molecule encodes a second nucleic acid moiety encoding a glycosyltransferase having the amino acid sequence of any one of SEQ ID NOs: 1-174 (see FIGS. 2C-2D).

The Examples of the present disclosure demonstrate the use of tripartite glycosyltransferase fusion proteins (e.g., Sx-CjCstII, Sx-Δ36HsFucT7, Sx-Δ34HsST3Gal1, Sx-Δ29HsGnTI, Sx-Δ29HsGnTII, Sx-Δ44Hsβ4GalT1, Sx-Δ26HsST6Gal1, Sx-Δ44HsFucT8) to catalyze the formation of a spectrum of homogenous N-glycan structures on intact glycoproteins. Thus, in some embodiments, the glycosyltransferase is selected from the group consisting of Campylobacter jejuni CsTII (CjCstII), human alpha-(1,3)-fucosyltransferase 7 (HsFUT7), human CMP-N-acetylneuraminate-beta-galactosamide-alpha-2,3-sialyltransferase 1 (HsST3Gal1), human alpha-1,3-mannosyl-glycoprotein 2-beta-N-acetylglucosaminyltransferase (HsGnTI/MGAT1), human alpha-1,6-mannosyl-glycoprotein 2-beta-N-acetylglucosaminyltransferase (HsGnTII/MGAT2), human beta-1,4-galactosyltransferase 1 (Hsβ4GalT1), human β-galactoside-α2,6-sialyltransferase 1 (HsST6Gal1), and human alpha-(1,6)-fucosyltransferase (HsFUT8).

As used herein, the term “amphipathic shield domain protein” includes any protein that displays both hydrophilic and hydrophobic surfaces and is often associated with lipids as membrane anchors or involved in their transport as soluble particles. The amphipathic shield domain protein, in one embodiment, serves as a molecular shield to sequester large lipophilic surfaces of the glycosyltransferase from water.

In various other embodiments, the amphipathic shield domain protein is selected from the group consisting of apolipoprotein A (ApoA), apolipoprotein B (ApoB), apolipoprotein C (ApoC), apolipoprotein D (ApoD), apolipoprotein E (ApoE), apolipoprotein H (ApoH), truncated human apolipoprotein A1 lacking its 43-residue globular N-terminal domain (ApoAI*), and a peptide self-assembly mimic (PSAM). In particular, the amphipathic shield domain protein may be apolipoprotein A1 (ApoAI). As used herein, ApoAI avidly binds phospholipid molecules and organizes them into soluble bilayer structures or discs that readily accept cholesterol. ApoAI contains a globular amino-terminal (N-terminal) domain (residues 1-43) and a lipid-binding carboxyl-terminal (C-terminal) domain (residues 44-243). In some embodiments, the amphipathic shield domain protein is human apolipoprotein A1. The apolipoprotein A1 may be a truncated human apolipoprotein A1. Truncated variants of ApoA1 include, but are not limited to, human ApoAI lacking its 43-residue globular N-terminal domain (ApoA1*).

As used herein, ApoA1 exhibits remarkable structural flexibility, and may adopt a molten globular-like state for lipid-free ApoAI under conditions that may allow it to adapt to the significant geometry changes of the lipids with which it interacts. The present disclosure provides tripartite fusion proteins in which, for example, ApoAI* may be genetically fused to the carboxyl terminus of a glycosyltransferase (or truncated glycosyltransferase). As described herein, expression of such tripartite glycosyltransferase fusion proteins may yield appreciable amounts of globular, water-soluble tripartite glycosyltransferase fusion proteins that are stabilized in a hydrophobic environment and retain structurally relevant conformations. The approach provides, inter alia, a facile method for efficiently solubilizing structurally diverse glycosyltransferases, for example in both prokaryotic and eukaryotic cells, without the need for detergents or lipid reconstitutions.

As used herein, the term “water soluble expression decoy protein” includes any protein which serves to direct an glycosyltransferase into cellular cytoplasm. The water soluble expression decoy protein may assist in “tricking” a hydrophobic glycosyltransferase into thinking that it is not hydrophobic. The water soluble expression decoy protein may be selected from the group consisting of outer surface protein (OspA) lacking its native export signal peptide, DnaB lacking its native export signal peptide, and maltose-binding protein (MBP) lacking its N-terminal signal peptide. In some embodiments, the water soluble expression decoy protein is maltose-binding protein (MBP) lacking its N-terminal signal peptide.

In some embodiments of the nucleic acid constructs and the tripartite glycosyltransferase fusion proteins according to the present disclosure, the amphipathic shield domain protein is truncated human apolipoprotein A1 lacking its 43-residue globular N-terminal domain (ApoAI*) and the water soluble expression decoy protein is maltose-binding protein (MBP) lacking its N-terminal signal peptide. For example, the nucleic acid construct may comprise a chimeric nucleic acid molecule comprising a first nucleic acid moiety encoding truncated human apolipoprotein A1 lacking its 43-residue globular N-terminal domain (ApoAI*), a second nucleic acid moiety encoding human β-galactoside-α2,6-sialyltransferase 1 (HsST6Gal1) or a truncated HsST6Gal1 variant in which 26 amino acids from the N-terminus of HsST6Gal1 comprising its CT and TMD were deleted (Δ26HsST6Gal1), and a third nucleic acid moiety encoding maltose-binding protein (MBP) lacking its N-terminal signal peptide (ΔspMBP). Such embodiments are described in Example 1, where ΔspMBP-HsST6Gal1-ApoAI* (abbreviated as Sx-HsST6Gal1) and ΔspMBP-Δ26HsST6Gal1-ApoAI* (abbreviated as Sx-Δ26HsST6Gal1) are shown to accumulate almost exclusively in the soluble cytoplasmic fraction of E. coli cells. The importance of the amphipathic shield domain and water soluble expression decoy proteins is evidenced by expression of unfused HsST6Gal1 and Δ26HsST6Gal1, which were not observed to accumulate in the soluble fraction and were only observed in minimal amounts in the insoluble and detergent-solubilized fractions.

In some embodiments, the construct further includes a promoter and a termination sequence, where the promoter and the termination sequence are operatively coupled to the chimeric nucleic acid molecule.

The chimeric nucleic acid molecules of the present disclosure include DNA molecules (e.g., linear, circular, cDNA, chromosomal, genomic, or synthetic, double stranded, single stranded, triple-stranded, quadruplexed, partially double-stranded, branched, hair-pinned, circular, or in a padlocked conformation) and RNA molecules (e.g., tRNA, rRNA, mRNA, genomic, or synthetic) and analogs of the DNA or RNA molecules of the described as well as analogs of DNA or RNA containing non-natural nucleotide analogs, non-native inter-nucleoside bonds, or both.

In some embodiments, the first nucleic acid moiety, the second nucleic acid moiety, and/or the third nucleic acid moiety may be free of naturally flanking sequences (i.e., sequences located at the 5′ and 3′ ends of the first nucleic acid moiety, the second nucleic acid moiety, and/or the third nucleic acid moiety) in the chromosomal DNA of the organism from which the first nucleic acid moiety, the second nucleic acid moiety, and/or the third nucleic acid moiety was derived, respectively.

In various embodiments, the first nucleic acid moiety, the second nucleic acid moiety, and/or the third nucleic acid moiety may contain less than about 10 kb, 5 kb, 4 kb, 3 kb, 2 kb, 1 kb, 0.5 kb, 0.1 kb, 50 bp, 25 bp or 10 bp of naturally flanking nucleotide chromosomal DNA sequences of the microorganism from which the first nucleic acid moiety, the second nucleic acid moiety, and/or the third nucleic acid moiety was derived, respectively.

The chimeric nucleic acid molecules may further include one or more linker nucleic acid moieties coupling the first, second, and/or third nucleic acid moieties together.

The tripartite glycosyltransferase fusion proteins according to the present disclosure include a continuous polymer of amino acids which comprise the full or partial sequence of three or more distinct proteins. The construction of fusion proteins is well-known in the art. Two or more amino acids sequences may be joined chemically, for instance, through the intermediacy of a crosslinking agent. For example, a fusion protein may be generated by expression of a nucleic acid construct comprising a chimeric nucleic acid molecule according to the present disclosure in a host cell. Such nucleic acid constructs may generally also contain replication origins active in host cells and one or more selectable markers encoding, for example, drug or antibiotic resistance.

The tripartite glycosyltransferase fusion proteins of the present disclosure can be generated as described herein or using any other standard technique known in the art. For example, the tripartite glycosyltransferase fusion proteins can be prepared by translation of a chimeric nucleic acid molecule encoding a tripartite glycosyltransferase fusion protein according to the present disclosure. The chimeric nucleic acid molecule encoding a tripartite glycosyltransferase fusion protein is inserted into an expression vector which is used to transform or transfect a host cell.

Different chimeric nucleic acid molecules encoding unique tripartite glycosyltransferase fusion proteins may be present on separate nucleic acid constructs or on the same nucleic acid construct. Inclusion of different chimeric nucleic acid molecules encoding unique tripartite glycosyltransferase fusion proteins on the same nucleic acid molecule is advantageous, in that uptake of only a single species of nucleic acid by a host cell is sufficient to introduce sequences encoding the tripartite glycosystransferase(s) into the host cell. By contrast, when different chimeric nucleic acid molecules encoding unique tripartite glycosyltransferase fusion proteins are present on different nucleic acid constructs, both nucleic acid molecules are taken up by a particular host cell for the assay to be functional.

A nucleic acid construct comprising a chimeric nucleic acid molecule encoding a tripartite glycosyltransferase fusion proteins may be inserted into an expression system to which the nucleic acid construct is heterologous. The heterologous nucleic acid construct may be inserted into the expression system or vector in proper sense (5′-3′) orientation relative to the promoter and any other 5′ regulatory molecules, and correct reading frame. The preparation of the nucleic acid constructs can be carried out using standard cloning methods well known in the art as described by Sambrook et al., Molecular Cloning: A Laboratory Manual, Cold Springs Laboratory Press, Cold Springs Harbor, New York (1989), which is hereby incorporated by reference in its entirety. U.S. Pat. No. 4,237,224 to Cohen and Boyer, which is hereby incorporated by reference in its entirety, also describes the production of expression systems in the form of recombinant plasmids using restriction enzyme cleavage and ligation with DNA ligase.

Another aspect of the present disclosure is directed to tripartite glycosyltransferase fusion proteins produced by the host cells described herein.

As described herein, a variety of prokaryotic expression systems can be used to express the tripartite glycosyltransferase fusion proteins of the present disclosure. Expression vectors can be constructed which contain a promoter to direct transcription, a ribosome binding site, and a transcriptional terminator. Examples of regulatory regions suitable for this purpose in E. coli are the promoter and operator region of the E. coli tryptophan biosynthetic pathway (Yanofsky et al., “Repression is Relieved Before Attenuation in the trp Operon of Escherichia coli as Tryptophan Starvation Becomes Increasingly Severe,” J. Bacteria. 158:1018-1024 (1984), which is hereby incorporated by reference in its entirety) and the leftward promoter of phage lambda (N) (Herskowitz et al., “The Lysis-lysogeny Decision of Phage Lambda: Explicit Programming and Responsiveness,” Ann. Rev. Genet., 14:399-445 (1980), which is incorporated by reference in its entirety). Vectors used for expressing foreign genes in bacterial hosts generally will contain a sequence for a promoter which functions in the host cell. Plasmids useful for transforming bacteria include pBR322 (Bolivar et al., “Construction and Characterization of New Cloning Vehicles II. A Multipurpose Cloning System,” Gene 2:95-113 (1977), which is hereby incorporated by reference in its entirety), the pUC plasmids (Messing, “New M13 Vectors for Cloning,” Meth. Enzymol. 101:20-77 (1983), Vieira et al., “New pUC-derived Cloning Vectors with Different Selectable Markers and DNA Replication Origins,” Gene 19:259-268 (1982) which are hereby incorporated by reference in their entirety), and derivatives thereof. Plasmids may contain both viral and bacterial elements. Methods for the recovery of the proteins in biologically active form are discussed in U.S. Pat. No. 4,966,963 to Patroni and 4,999,422 to Galliher, which are incorporated herein by reference in their entirety. Suitable expression vectors include those which contain replicon and control sequences that are derived from species compatible with the host cell. For example, if E. coli is used as a host cell, plasmids such as pUC19, pUC18 or pBR322 may be used. Alternatively, plasmids such as pET28a and pMALc2× may be used. Other suitable expression vectors are described in Molecular Cloning: a Laboratory Manual: 3rd edition, Sambrook and Russell, 2001, Cold Spring Harbor Laboratory Press, which is hereby incorporated by reference in its entirety. Many known techniques and protocols for manipulation of nucleic acids, for example in preparation of nucleic acid constructs, mutagenesis, sequencing, introduction of DNA into cells and gene expression, and analysis of proteins, are described in detail in Current Protocols in Molecular Biology, Ausubel et al. eds., (1992), which is hereby incorporated by reference in its entirety.

Different genetic signals and processing events control many levels of gene expression (e.g., DNA transcription and messenger RNA (“mRNA”) translation) and subsequently the amount of fusion protein that is displayed on the ribosome surface. Transcription of DNA is dependent upon the presence of a promoter, which is a DNA sequence that directs the binding of RNA polymerase, and thereby promotes mRNA synthesis. Promoters vary in their “strength” (i.e., their ability to promote transcription). For the purposes of expressing a cloned gene, it is desirable to use strong promoters to obtain a high level of transcription and, hence, expression and surface display. Therefore, depending upon the host system utilized, any one of a number of suitable promoters may also be incorporated into the expression vector carrying the deoxyribonucleic acid molecule encoding the protein of interest coupled to a stall sequence. For instance, when using E. coli, its bacteriophages, or plasmids, promoters such as the T7 phage promoter, lac promoter, trp promoter, recA promoter, ribosomal RNA promoter, the P_{R} and P_{L} promoters of coliphage lambda and others, including but not limited, to lacUV5, ompF, bla, lpp, and the like, may be used to direct high levels of transcription of adjacent DNA segments. Additionally, a hybrid trp-lacUV5 (tac) promoter or other E. coli promoters produced by recombinant DNA or other synthetic DNA techniques may be used to provide for transcription of the inserted gene.

Translation of mRNA in prokaryotes depends upon the presence of the proper prokaryotic signals, which differ from those of eukaryotes. Efficient translation of mRNA in prokaryotes requires a ribosome binding site called the Shine-Dalgarno (“SD”) sequence on the mRNA. This sequence is a short nucleotide sequence of mRNA that is located before the start codon, usually AUG, which encodes the amino-terminal methionine of the protein. The SD sequences are complementary to the 3′-end of the 16S rRNA (ribosomal RNA) and probably promote binding of mRNA to ribosomes by duplexing with the rRNA to allow correct positioning of the ribosome. For a review on maximizing gene expression, see Roberts and Lauer, “Maximizing Gene Expression on a Plasmid Using Recombination In Vitro,” Methods in Enzymology 68:473-82 (1979), which is hereby incorporated by reference in its entirety.

In accordance with this and other aspects of the present disclosure, the amphipathic shield domain protein, glycosyltransferase, and/or water soluble expression decoy proteins are linked either directly or via a linker located adjacent to each other within the construct, coupled to each other in tandem or separated by at least one linker. In one embodiment, the chimeric nucleic acid molecule includes a linker coupling the nucleic acid moieties together. Likewise, the tripartite glycosyltransferase fusion proteins may include a linker coupling the amphipathic shield domain protein, the glycosyltransferase (or truncated glycosyltransferase), and the water soluble expression decoy protein together. The amphipathic shield domain protein, the glycosyltransferase (or truncated glycosyltransferase), and the water soluble expression decoy protein may be linked by a covalent linkage or may be linked by methods known in the art for linking peptides.

Linkers may include synthetic sequences of amino acids that are commonly used to physically connect polypeptide domains to each other or to biologically relevant moieties. Most linker peptides are composed of repetitive modules of one or more of the amino acids glycine and serine. Peptide linkers have been well-characterized and shown to adopt unstructured, flexible conformations. For example, linkers comprised of Gly and Ser amino acids have been found to not interfere with assembly and binding activity of the domains it connects. Freund et al., “Characterization of the Linker Peptide of the Single-chain Fv Fragment of an Antibody by NMR Spectroscopy,” FEBS 320:97 (1993), which is hereby incorporated by reference in its entirety.

The nucleic acid constructs and tripartite glycosyltransferase fusion proteins of the present disclosure may include a flexible polypeptide linker separating the amphipathic shield domain protein, glycosyltransferase (or truncated glycosyltransferase), and/or water soluble expression decoy proteins and allowing for their independent folding. The linker is optimally 15 amino acids or 60 Å in length (˜4 Å per residue) but may be as long as 30 amino acids but preferably not more than 20 amino acids in length. It may be as short as 3 amino acids in length, but more preferably is at least 6 amino acids in length. To ensure flexibility and to avoid introducing steric hindrance that may interfere with the independent folding of the fragment domain of reporter protein and the members of the putative binding pair, the linker should be comprised of small, preferably neutral residues such as Gly, Ala, and Val, but also may include polar residues that have heteroatoms such as Ser and Met, and may also contain charged residues. The first, second, and third proteins may be linked via a short polypeptide linker sequence. Suitable linkers include peptides of between about 2 and about 40 amino acids in length and may include, for example, glycine residues Gly185 and Gly186. Preferred linker sequences include glycine-rich (e.g. G₃0.5), serine-rich (e.g., GSG, GSGS, GSGSG, GS_{N}G), or alanine rich (e.g., TSAAA) linker sequences. Other exemplary linker sequences have a combination of glycine, alanine, proline and methionine residues such as AAAGGM; AAAGGMPPAAAGGM (SEQ ID NO: 175); AAAGGM; and PPAAAGGMM. Linkers may have virtually any sequence that results in a generally flexible chimeric protein.

Another aspect of the present disclosure relates to an expression vector including the nucleic acid construct of the present disclosure. Suitable nucleic acid vectors include, without limitation, plasmids, baculovirus vectors, bacteriophage vectors, phagemids, cosmids, fosmids, bacterial artificial chromosomes, viral vectors (for example, viral vectors based on vaccinia virus, poliovirus, adenovirus, adeno-associated virus, SV40, herpes simplex virus, and the like), P1-based artificial chromosomes, yeast plasmids, yeast artificial chromosomes, and other vectors. In some embodiments of the present disclosure, vectors suitable for use in prokaryotic host cells. Accordingly, exemplary vectors for use in prokaryotes such as Escherichia coli include, but are not limited to, pACYC184, pBeloBac11, pBR332, pBAD33, pBBR1MCS and its derivatives, pSC101, SuperCos (cosmid), pWE15 (cosmid), pTrc99A, pBAD24, vectors containing a ColE1 origin of replication and its derivatives, pUC, pBluescript, pGEM, and pTZ vectors.

Another aspect of the present disclosure relates to a host cell comprising the nucleic acid construct of the present disclosure. In accordance with this and other aspects of the present disclosure, suitable host cells include both eukaryotic and prokaryotic cells.

In some embodiments, the host cell is eukaryotic. Eukaryotic host cells, include without limitation, animal cells, fungal cells, insect cells, plant cells, and algal cells. In some embodiments, the eukaryotic host cells are selected from the group consisting of human cells, yeast, cells, and cell lines. Suitable eukaryotic host cells include, but are not limited to, Pichia pastoris, Pichia finlandica, Pichia trehalophila, Pichia koclamae, Pichia membranaefaciens, Pichia opuntiae, Pichia thennotolerans, Pichia salictaria, Pichia guercuum, Pichia pijperi, Pichia stiptis, Pichia methanolica, Pichia sp., Saccharomyces cerevisiae, Saccharomyces sp., Hansenula polymorpha, Kluyveromyces sp., Kluyveromyces lactis, Candida albicans, Aspergillus nidulans, Aspergillus niger, Aspergillus oryzae, Trichoderma reesei, Chrysosporium lucknowense, Fusarium sp., Fusarium gramineum, Fusarium venenatum, Neurospora crassa, Chlamydomonas reinhardtii, and the like. In some embodiments, the eukaryotic host cell is a yeast cell and the yeast cell strain is SBY49. In some embodiments, the eukaryotic host cell is a human cell. Exemplary human cells lines include, without limitation, HEK293T (ATCC), FreeStyle™ 293-F (Thermo Fisher), and Expi293F™ GnTI- (Thermo Fisher).

In accordance with the present disclosure, the host cell may be prokaryotic, such as a bacterial cell. Such cells serve as a host for expression of recombinant proteins for production of recombinant therapeutic proteins of interest. Suitable microorganisms include Pseudomonas sp. such as Pseudomonas aeruginosa, Escherichia sp., Escherichia coli and other Enterobacteriaceae, Salmonella sp. such as Salmonella gastroenteritis (typhimirium), S. typhi, S. enteriditis, Shigella sp. such as Shigella flexneri, S. sonnie, S dysenteriae, Neisseria sp. such as Neisseria gonorrhoeae, N. meningitides, Haemophilus sp. including Haemophilus influenzae H. pleuropneumoniae, Pasteurella sp. including Pasteurella haemolytica, P. multilocida, Legionella sp. such as Legionella pneumophila, Treponema pallidum, T. denticola, T. orales, Borrelia burgdorferi, Borrelia spp. Leptospira interrogans, Klebsiella sp. such as Klebsiella pneumoniae, Proteus vulgaris, P. morganii, P. mirabilis, Rickettsia prowazeki, R. typhi, R. richettsii, Porphyromonas (Bacteroides) gingivalis, Chlamydia psittaci, C. pneumoniae, C. trachomatis, Campylobacter sp. such as Campylobacter jejuni, C. intermedis, C. fetus, Helicobacter sp. such as Helicobacter pylori, Francisella sp. such as Francisella tularenisis, Vibrio cholerae, Vibrio parahaemolyticus, Bordetella sp. including Bordetella pertussis, Burkholderia sp. such as Burkholderie pseudomallei, Brucella sp. including Brucella abortus, B. susi, B. melitens is, B. canis, Spirillum minus, Pseudomonas mallei, Aeromonas sp. such as Aeromonas hydrophila, A salmonicida, and Yersinia sp. such as Yersinia pestis. Additional microorganisms include Wolinella sp., Desulfovibrio sp. Vibrio sp., Bacillus sp., Listeria sp., Staphylococcus sp., Streptococcus sp., Peptostreptococcus sp., Megasphaera sp., Pectinatus sp., Selenomonas sp., Zymophilus sp., Actinomyces sp., Arthrobacter sp., Frankia sp., Micromonospora sp., Nocardia sp., Propionibacterium sp., Streptomyces sp., Lactobacillus sp., Lactococcus sp., Leuconostoc sp., Pediococcus sp., Acetobacterium sp., Eubacterium sp., Heliobacterium sp., Heliospirillum sp., Sporomusa sp., Spiroplasma sp., Ureaplasma sp., Erysipelothrix, sp., Corynebacterium sp. Enterococcus sp., Clostridium sp., Mycoplasma sp., Mycobacterium sp., Actinobacteria sp., Moraxella sp., Stenotrophomonas sp., Micrococcus sp., Bdellovibrio sp., Hemophilus sp., Proteus mirabilis, Enterobacter cloacae, Serratia sp., Citrobacter sp., Proteus sp., Acinetobacter sp., Actinobacillus sp., Capnocytophaga sp., Cardiobacterium sp., Eikenella sp., Kingella sp., Flavobacterium sp. Xanthomonas sp., Plesiomonas sp., and alpha-proteobacteria such as Wolbachia sp., cyanobacteria, spirochaetes, green sulfur and green non-sulfur bacteria, Gram-negative cocci, Gram negative bacilli which are fastidious, Enterobacteriaceae-glucose-fermenting gram-negative bacilli, Gram negative bacilli—non-glucose fermenters, Gram negative bacilli—glucose fermenting, oxidase positive. In some embodiments, the prokaryotic host cells is an E. coli cells such as DH5a, BL21 (DE3), SHuffle® T7 Express lysY, and Origami2(DE3) gmd::kan ΔwaaL.

Methods for transforming/transfecting host cells with expression vectors are well-known in the art and depend on the host system selected as described in Sambrook et al., Molecular Cloning: A Laboratory Manual, Cold Springs Laboratory Press, Cold Springs Harbor, New York (1989), which is hereby incorporated by reference in its entirety.

The present disclosure is also directed to tripartite glycosyltransferase fusion proteins produced by the host cells of the present disclosure.

When the nucleic acid construct is assembled in a host cell, the host cell may be cultured in a suitable culture medium optionally supplemented with one or more additional agents, such as an inducer (e.g., where a nucleotide sequence encoding a chimeric protein is under the control of an inducible promoter). The inducer may be, for example, isopropyl-β-D-thiogalactoside. In one embodiment of the present disclosure, a substrate is endogenous to the host cell and upon assembly of the nucleic acid construct in the host cell, the substrate is readily converted. In another embodiment, a substrate is exogenous to the host cell. In accordance with this embodiment, the culture medium is supplemented with a substrate or substrate precursor that can be readily taken up by the host cell and converted. Suitable substrates include, without limitation, proteins, nucleic acid molecules, organic compounds, lipids, and glycans.

In some embodiments of the present disclosure, the tripartite glycosyltransferase fusion protein is separated from other products, macromolecules, etc., which may be present in the cell culture medium, the cell lysate, or the organic layer. Separation of the tripartite glycosyltransferase fusion protein from other products that may be present in the cell culture medium, cell lysate, or organic layer is readily achieved using standard methods known in the art, e.g., standard chromatographic techniques. Several methods are readily known in the art, including ion exchange chromatography, high performance liquid chromatography, hydrophobic interaction chromatography, affinity chromatography (e.g., Ni²⁺ affinity chromatography), size exclusion chromatography, gel filtration, and reverse phase chromatography. The tripartite glycosyltransferase fusion protein is preferably produced in purified form (at least about 40% pure, at least about 50% pure, at least about 60% pure, at least about 70% pure, at least about 80% pure, at least about 90% pure, at least about 95% pure, at least about 98%, or more than 98% pure) by conventional techniques. Depending on whether the host cell is made to secrete the protein into growth medium (see U.S. Pat. No. 6,596,509 to Bauer et al., which is hereby incorporated by reference in its entirety), the protein can be isolated and purified by centrifugation (to separate cellular components from supernatant containing the secreted protein) followed by sequential ammonium sulfate precipitation of the supernatant. The fraction containing the protein can be subjected to gel filtration in an appropriately sized dextran or polyacrylamide column to separate the protein from other cellular components and proteins. If necessary, the protein fraction may be further purified by HPLC. Accordingly, the tripartite glycosyltransferase fusion protein produced by the present disclosure can be used to isolate and solubilize a glycosyltransferase in a purified form, e.g., “pure” in the context of a tripartite glycosyltransferase fusion protein that is free from other intermediate or precursor products, macromolecules, contaminants, etc.

Expression of soluble tripartite glycosyltransferase fusion proteins may be increased by at least about 10%, at least about 20%, at least about 25%, at least about 30%, at least about 40%, at least about 50%, at least about 60%, at least about 70%, at least about 80%, at least about 90%, at least about 100% (or two-fold), as compared to when the corresponding glycosyltransferase is expressed in the absence of the amphipathic shield domain protein and/or water soluble expression decoy protein. In other embodiments of the present disclosure, the expression of tripartite glycosyltransferase fusion proteins from the nucleic acid constructs disclosed herein is at least about 2.5-fold, at least about 3-fold, at least about 5-fold, at least about 7-fold, at least about 10-fold, at least about 15-fold, at least about 20-fold, at least about 50-fold, at least about 100-fold, or more, higher compared to the expression level of the glycosyltransferase from nucleic acid constructs lacking the first nucleic acid moiety encoding an amphipathic shield domain protein and/or the third nucleic acid moiety encoding a water soluble expression decoy protein. Likewise, the expression of tripartite glycosyltransferase fusion proteins from the nucleic acid constructs disclosed herein may be at least about 2.5-fold, at least about 3-fold, at least about 5-fold, at least about 7-fold, at least about 10-fold, at least about 15-fold, at least about 20-fold, at least about 50-fold, at least about 100-fold, or more, higher compared to the expression of a corresponding wild type glycosyltransferase protein, which is not fused to a heterologous amphipathic shield domain protein and/or a water soluble expression decoy protein.

Methods for transforming/transfecting host cells with expression vectors are well-known in the art and depend on the host system selected, as described in Sambrook et al., Molecular Cloning: A Laboratory Manual, Cold Springs Laboratory Press, Cold Springs Harbor, New York (1989), which is hereby incorporated by reference in its entirety. For eukaryotic cells, suitable techniques may include calcium phosphate transfection, DEAE-Dextran, electroporation, liposome-mediated transfection and transduction using retrovirus or other virus, e.g. vaccinia or, for insect cells, baculovirus. For bacterial cells, suitable techniques may include calcium chloride transformation, electroporation, and transfection using bacteriophage.

The simplest single-celled organisms are composed of central regions filled with an aqueous material and a variety of soluble small molecules and macromolecules. Enclosing this central region is a membrane which is composed of phospholipids arranged in a bilayer structure. In more complex living cells, there are internal compartments and structures that are also enclosed by membranes. There are many protein molecules embedded or associated within these membrane structures, and these membrane proteins are often the most important to determining cell functions including communication and processing of information and energy. The largest problem in studying membrane proteins is that the inside of the phospholipid bilayer is hydrophobic and the embedded or anchored part of the membrane protein is itself also hydrophobic. In isolating these membrane proteins from their native membrane environments, the present disclosure overcomes the difficult task of preventing recombinant glycosyltransferases from forming inactive aggregates while remaining in a native configuration. In one embodiment of the present disclosure, a tripartite glycosyltransferase fusion protein is encoded by the nucleic acid construct of the present disclosure and, preferably, the tripartite glycosyltransferase fusion protein is in water soluble form. The term “solubilizing” according to the present disclosure includes dissolving a molecule in a solution. This aspect of the disclosure is carried out in substantially the same way as described above.

The present disclosure is also directed to tripartite glycosyltransferase fusion proteins produced by the host cells of the present disclosure.

In addition to cell-based expression hosts/systems of the present disclosure, the tripartite glycosyltransferase fusion proteins may also be expressed using cell-free expression platforms. Thus, another aspect of the present disclosure relates to a cell-free protein expression system. The cell-free protein expression system comprises a cell lysate or extract and a nucleic acid construct according to the present disclosure. The cell lysate or extract may include a heterologous and/or recombinant RNA polymerase. In some embodiments, the cell lysate or extract is capable of (i) transcribing the nucleic acid construct or the vector to form a translation template and (ii) translating the translation template. In some embodiments, the cell lysate or extract is an E. coli lysate or extract. Examples of cell-free expression platforms include, but are not limited to, the PURExpress kit from NEB and S30 lysate high-expression kit from Promega, among others.

The present disclosure is also directed to tripartite glycosyltransferase fusion proteins produced by the cell-free protein expression systems of the present disclosure.

Another aspect of the present disclosure relates to a method of recombinantly producing a tripartite glycosyltransferase fusion protein in water soluble form. This method involves providing a host cell according to the present disclosure or a cell-free expression system according to the present disclosure. The method further involves culturing the host cell or using the cell-free expression system under conditions effective to express the tripartite glycosyltransferase fusion protein in a water soluble form within the host cell cytoplasm or the cell-free expression system.

In some embodiments, the method further includes recovering the tripartite glycosyltransferase fusion protein from the host cell or the cell-free expression system following the culturing or the using, respectively. The tripartite glycosyltransferase fusion protein may be recovered from the cell's cytoplasm. The recovery of the tripartite glycosyltransferase fusion protein from the host cell is consistent with the recovery of proteins discussed supra.

In some embodiments where the host cell is provided, the recovering involves lysing the cell to form a cell lysate comprising a water soluble fraction and subjecting the water soluble fraction of the cell lysate to chromatography to isolate the tripartite glycosyltransferase fusion protein.

In some embodiments where the cell-free expression system is provided, the recovering involves subjecting the water soluble fraction of the cell lysate to chromatography to isolate the tripartite glycosyltransferase fusion protein.

In one embodiment of this aspect of the present disclosure, the tripartite glycosyltransferase fusion proteins are provided in a purified isolated form.

The tripartite glycosyltransferase fusion protein can be synthesized using standard methods of protein/peptide synthesis known in the art, including solid phase synthesis or solution phase synthesis. Alternatively, the tripartite glycosyltransferase fusion proteins can be generated using recombinant expression systems and purified using any method readily known in the art, including ion exchange chromatography, hydrophobic interaction chromatography, affinity chromatography, gel filtration, and reverse phase chromatography.

Nucleotide sequences encoding the tripartite glycosyltransferase fusion proteins may be modified such that the nucleotide sequence reflects the codon preference for a particular host cell. For example, when yeast host cells are utilized, the nucleotide sequences encoding the chimeric proteins can be modified for yeast codon preference (see, e.g., Bennetzen and Hall, “Codon Selection in Yeast,” J. Biol. Chem. 257(6):3026-3031 (1982), which is hereby incorporated by reference in its entirety). Likewise, when bacterial host cells are utilized, e.g., E. coli cells, the nucleotide sequences encoding the chimeric biological pathway proteins can be modified for E. coli codon preference (see e.g., Gouy and Gautier, “Codon Usage in Bacteria: Correlation With Gene Expressivity,” Nucleic Acids Res. 10(22):7055-7074 (1982); Eyre-Walker et al., “Synonymous Codon Bias is Related to Gene Length in Escherichia coli: Selection for Translational Accuracy?,” Mol. Biol. Evol. 13(6):864-872 (1996) and Nakamura et al., “Codon Usage Tabulated From International DNA Sequence Databases: Status for the year 2000,” Nucleic Acids Res. 28(1):292 (2000), which are hereby incorporated by reference in their entirety).

A variety of genetic signals and processing events that control many levels of gene expression (e.g., DNA transcription and messenger RNA (“mRNA”) translation) can be incorporated into the nucleic acid construct encoding the chimeric proteins to maximize protein production. For the purpose of expressing a cloned nucleic acid sequence encoding the desired tripartite glycosyltransferase fusion protein, it is advantageous to use strong promoters to obtain a high level of transcription. Depending upon the host system utilized, any one of a number of suitable promoters may be used. For instance, when cloning in E. coli, its bacteriophages, or plasmids, promoters such as the T7 phage promoter, lac promoter, trp promoter, recA promoter, ribosomal RNA promoter, the P_{R} and P_{L} promoters of coliphage lambda and others, including but not limited, to lacUV5, ompF, bla, lpp, and the like, may be used to direct high levels of transcription of adjacent DNA segments. Additionally, a hybrid trp-lacUV5 (tac) promoter or other E. coli promoters produced by recombinant DNA or other synthetic DNA techniques may be used to provide for transcription of the inserted chimeric genetic construct. Common promoters suitable for directing expression in mammalian cells include, without limitation, SV40, MMTV, metallothionein-1, adenovirus Ela, CMV, immediate early, immunoglobulin heavy chain promoter and enhancer, and RSV-LTR. Common promoters suitable for directing expression in a yeast cell include constitutive promoters such as an ADH1 promoter, a PGK1 promoter, an ENO promoter, a PYK1 promoter and the like; or a regulatable promoter such as a GAL1 promoter, a GAL10 promoter, an ADH2 promoter, a PHO5 promoter, a CUP1 promoter, a GAL7 promoter, a MET25 promoter, a MET3 promoter, a CYC1 promoter, a HIS3 promoter, a PGK promoter, a GAPDH promoter, an ADC1 promoter, a TRP1 promoter, a URA3 promoter, a LEU2 promoter, an ENO promoter, a TP1 promoter, and a AOX1 promoter.

There are other specific initiation signals required for efficient gene transcription and translation in eukaryotic and prokaryotic cells that can be included in the nucleic acid construct to maximize chimeric protein production. Depending on the vector system and host utilized, any number of suitable transcription and/or translation elements, including constitutive, inducible, and repressible promoters, as well as minimal 5′ promoter elements, enhancers, or leader sequences may be used. For a review on maximizing gene expression see Roberts and Lauer, “Maximizing Gene Expression On a Plasmid Using Recombination In Vitro,” Methods in Enzymology 68:473-82 (1979), which is hereby incorporated by reference in its entirety.

A nucleic acid molecule encoding a tripartite glycosyltransferase fusion protein of the present disclosure, a promoter molecule of choice, including, without limitation, enhancers, and leader sequences; a suitable 3′ regulatory region to allow transcription in the host, and any additional desired components, such as reporter or marker genes, are cloned into a vector of choice using standard cloning procedures in the art, such as described in Sambrook et al., MOLECULAR CLONING: A LABORATORY MANUAL (Cold Springs Harbor 1989); Ausubel, SHORT PROTOCOLS IN MOLECULAR BIOLOGY (Wiley 1999), and U.S. Pat. No. 4,237,224 to Cohen and Boyer, which are hereby incorporated by reference in their entirety. Suitable expression vectors include those described supra. Two or more nucleic acid constructs encoding two or more tripartite glycosyltransferase fusion proteins can be housed in the same or different expression vectors. In one embodiment of the present disclosure, two or more nucleic acid molecules encoding two or more tripartite glycosyltransferase fusion proteins are present in the same nucleic acid vector.

In some embodiments, the recovered tripartite glycosyltransferase fusion protein is conformationally correct.

Another aspect of the present disclosure relates to a tripartite glycosyltransferase fusion protein produced by the methods of recombinantly producing a tripartite glycosyltransferase fusion protein according to the present disclosure.

As will be apparent to one of skill in the art, the present disclosure allows for a broad range of in vivo or in vitro glycan remodeling. The constructs of the present disclosure allow for solubilized tripartite glycosyltransferase fusion proteins for use in methods of in vivo or in vitro glycan remodeling. Accordingly, another aspect of the present disclosure relates to a method of cell-free glycan remodeling. This method involves providing a glycan primer; providing one or more tripartite glycosyltransferase fusion protein(s) according to the present disclosure; and incubating the glycan primer with the one or more tripartite glycosyltransferase fusion protein(s) under conditions effective to transfer a glycosyl group to the glycan primer to produce a modified glycan structure.

The glycan primer may be a monosaccharide or an oligosaccharide. For example, the glycan primer may comprise Man₃GlcNAc₂ or Man₅GlcNAc₂.

In some embodiments, the glycan primer is attached to an amino acid residue such as an asparagine residue. In some embodiments, the glycan primer is attached to a protein. Accordingly, the glycan primer may be attached to a glycoprotein. The glycoprotein may comprise an N-glycosidic linkage. For example, the glycoprotein may comprises an N-acetylglucosamine (GlcNAc) linkage to asparagine.

The glycoprotein may be selected from the group consisting of an antibody or a hormone.

In some embodiments, the glycoprotein comprises an O-glycosidic linkage.

Suitably tripartite glycosyltransferase fusion proteins are described in detail supra. In some embodiments, the glycosyltransferase fusion protein is selected from the group consisting of Sx-Δ29HsGnTI, Sx-Δ29HsGnTII, Sx-Δ30HsFucT8, Sx-Δ44Hsβ4GalT1, Sx-Δ26HsST6Gal1, and combinations thereof.

In some embodiments, when the incubating step is carried out with a plurality of different tripartite glycosyltransferase fusion proteins, at least some of the different tripartite glycosyltransferase proteins being used sequentially during said incubating. In accordance with such embodiments, the incubating step produces a modified glycan primer. In some embodiments, the method may further involve incubating a modified glycan primer with one or more glycosyl hydrolases. In accordance with such embodiments, the one or more hydrolases may be used sequentially during said further incubating.

In some embodiments, when the incubating step is carried out with a plurality of different tripartite glycosyltransferase fusion proteins, at least some of the different tripartite glycosyltransferase proteins being used simultaneously during said incubating.

The above disclosure generally describes the present disclosure. A more specific description is provided below in the following examples. The examples are described solely for the purpose of illustration and are not intended to limit the scope of the present disclosure. Changes in form and substitution of equivalents are contemplated as circumstances suggest or render expedient. Although specific terms have been employed herein, such terms are intended in a descriptive sense and not for purposes of limitation.

### EXAMPLES

### Example 1—Materials and Methods

### Strains and Cell Lines

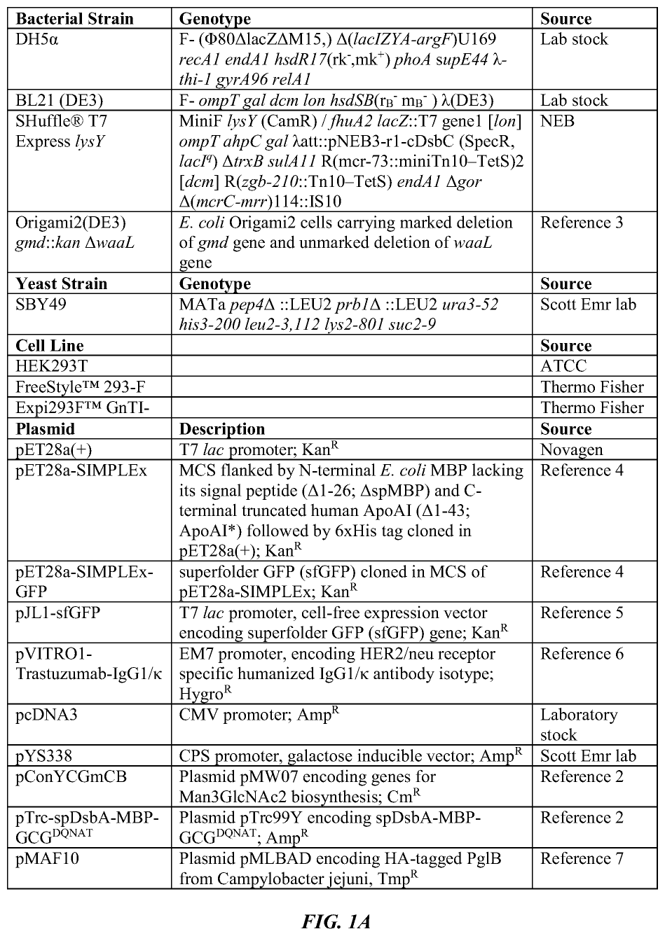

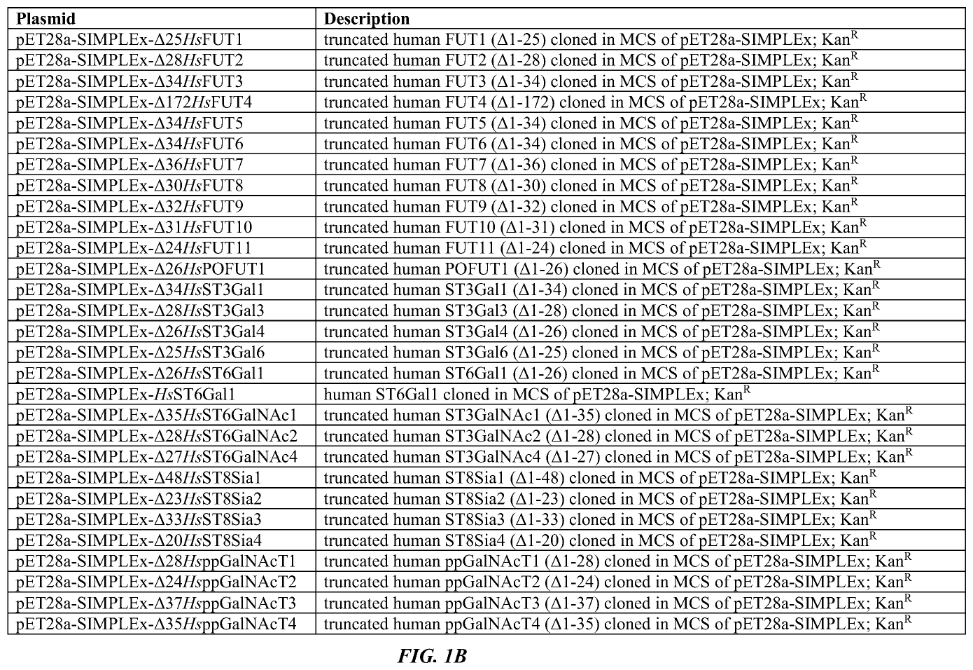

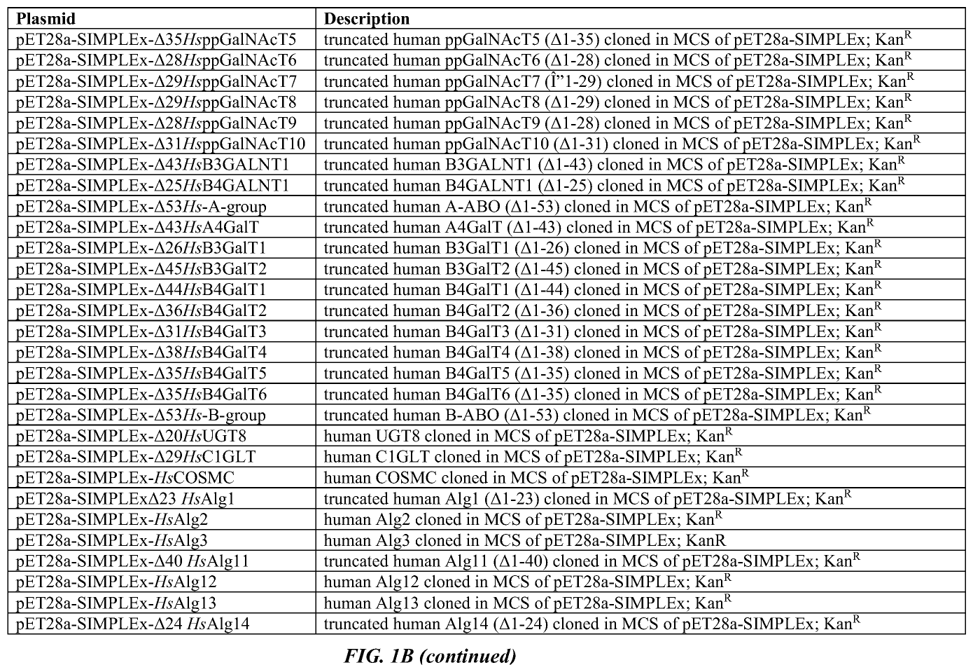

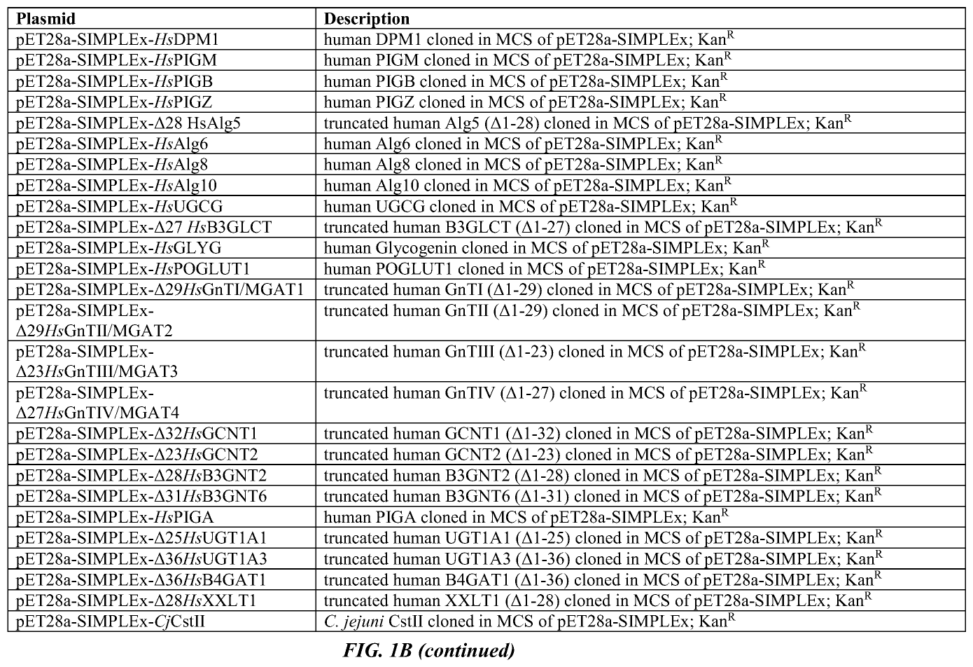

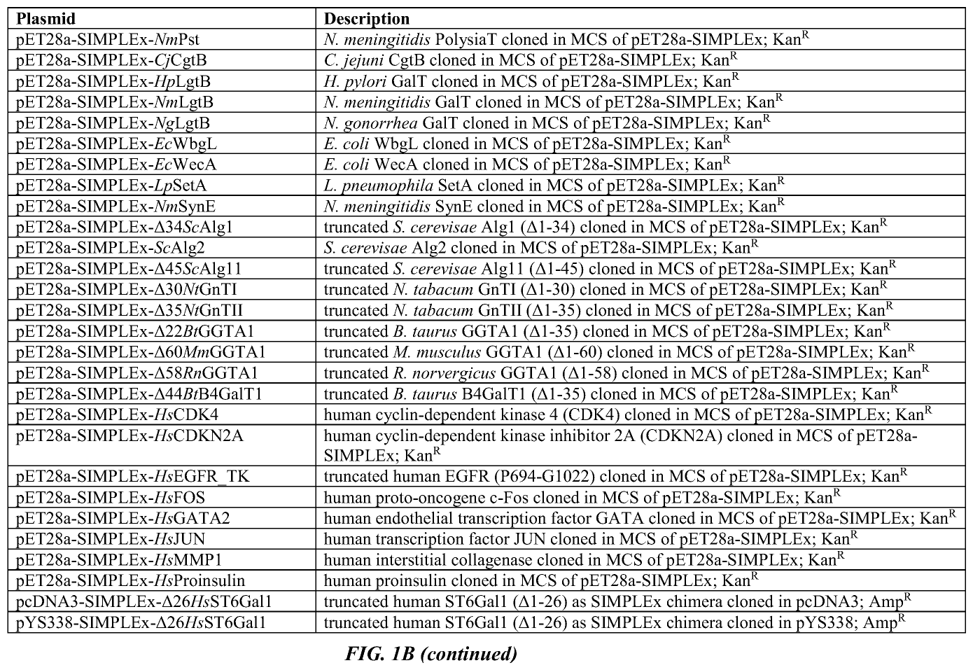

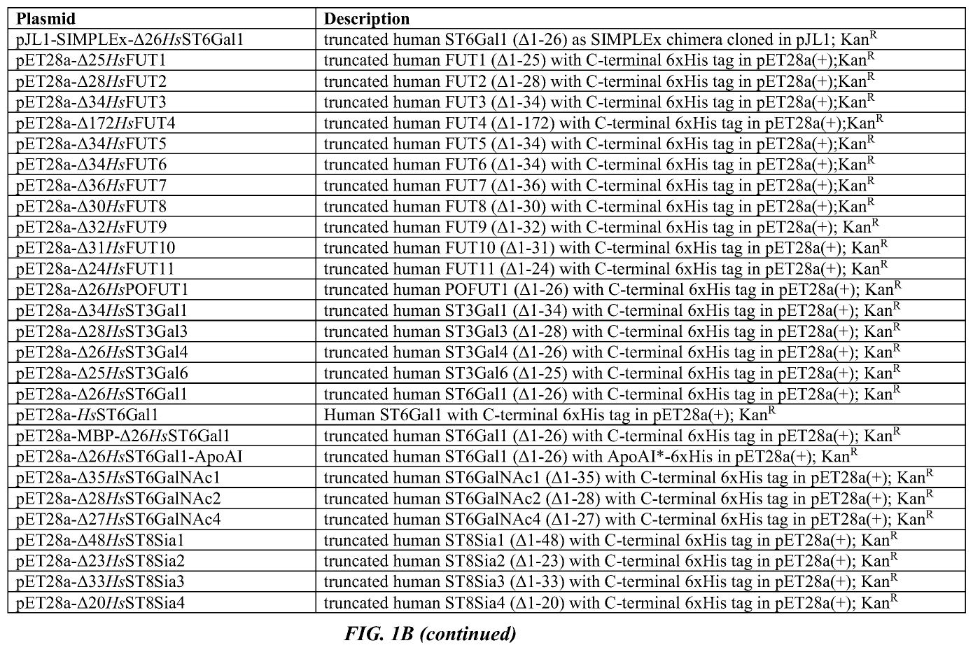

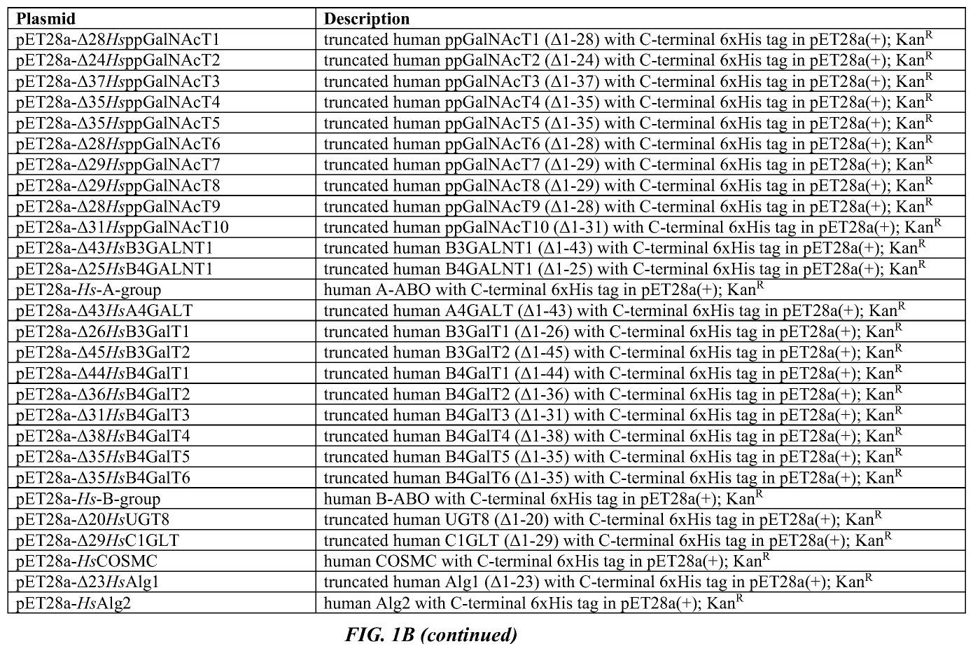

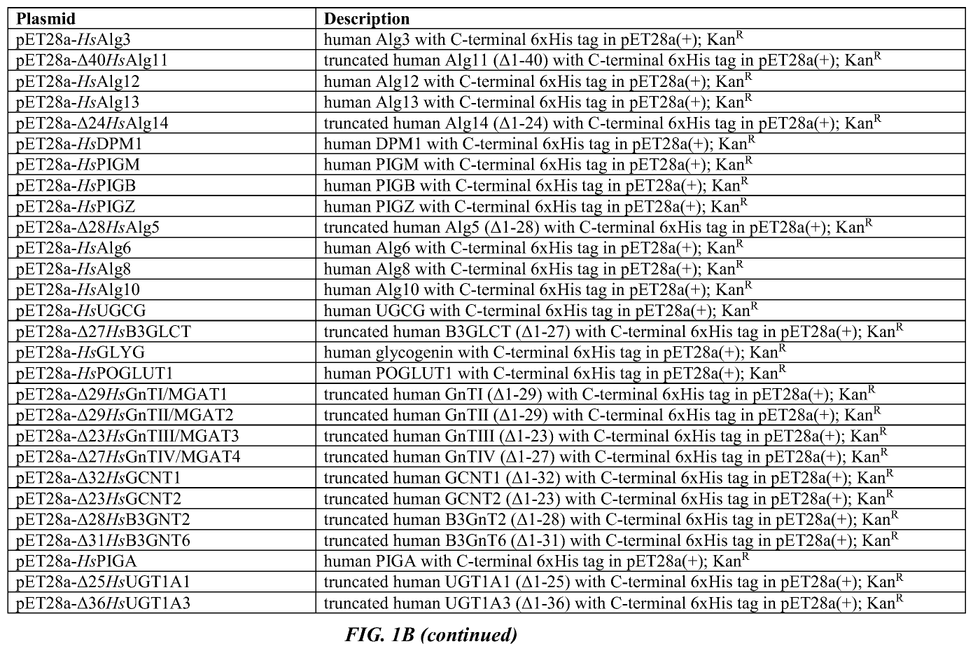

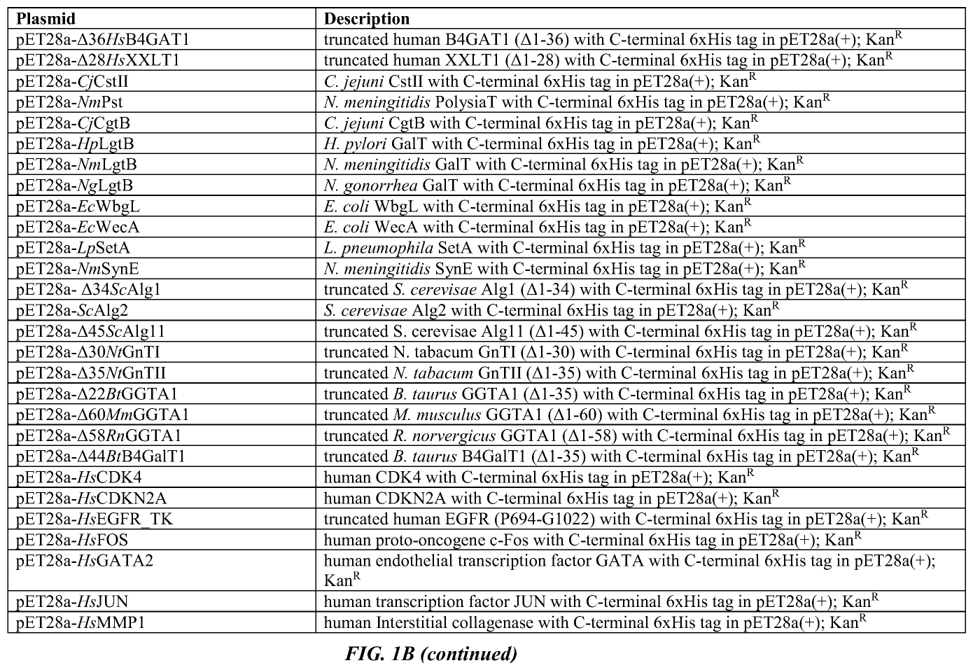

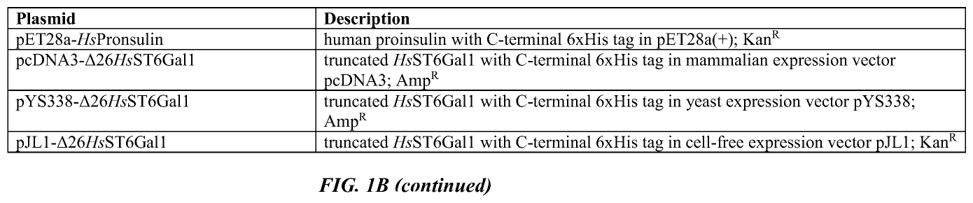

The bacterial, yeast, and mammalian cells used in Examples 1-9 are listed in FIGS. 1A-1B. E. coli strain DH5a was used for all molecular cloning and plasmid storage. E. coli strain BL21(DE3) and its derivative SHuffle T7 Express lysY (New England Biolabs) were used for all protein expression and purification. Luria-Bertani medium (LB) was used to culture E. coli in all experiments and was supplemented with appropriate antibiotics for plasmid maintenance. The final concentration for each antibiotic used was: 50 g/mL kanamycin, 20 g/mL chloramphenicol, and 100 g/mL ampicillin. Yeast strain SBY49 was grown in complex yeast extract peptone dextrose (YPD) medium or yeast nitrogen base (YNB) medium without amino acids supplemented with uracil dropout amino acids (-URA media) for plasmid maintenance. HEK293T cells were obtained from ATCC (CRL-3216) and cultured in DMEM supplemented with 10% FetalClone (VWR), 4.5 g/L glucose and L-glutamine, and 1% (w/v) penicillin-streptomycin-amphotericin B (Thermo Fisher Scientific). FreeStyle™ 293-F cells (HEK293F) were obtained from Thermo Fisher Scientific (Cat #R79007). Expi293-F™ GnTI-cells (HEK293F GnTI-) were obtained from Thermo Fisher Scientific (Cat #A39240) and were cultured in Expi293™ Expression Medium supplemented with 1% (w/v) penicillin-streptomycin-amphotericin B (Thermo Fisher Scientific). All cells were maintained in a 37° C. incubator with 5% CO₂ and 90% relative humidity. Authentication of each cell line used in this study included morphology analysis, PCR assays with species-specific primers, and STR profiling, the latter of which was performed using ATCC's human cell STR profiling service.

### Cell Growth Analysis

To facilitate high-throughput cell growth measurements, three individual colonies corresponding to each construct were seeded into 96-deep well plates (Eppendorf) where each well contained 100 μL LB media. Culture plates were then sealed using plate sealer and placed in an incubator shaker at 37° C. for 16 hours. Then, 5 μL of the overnight culture was subcultured into fresh 100 μL LB media and incubated for 8 hours, after which IPTG was supplemented to a final concentration of 0.1 mM. Protein expression proceeded at 16° C. for 18 hours. To measure OD₆₀₀, 10 μL of each sample was mixed with 90 μL DI water in a Costar 96-well assay plate (Corning) and OD₆₀₀ of all samples was measured in an Infinite M1000Pro spectrophotometer (Tecan).

### Plasmid Construction.

All plasmids used in this study are listed in FIGS. 1A-1B. The collection of prokaryotic and eukaryotic glycoenzymes was selected from the CAZy database (Lombard et al., “The Carbohydrate-active Enzymes Database (CAZy) in 2013,” Nucleic Acids Res 42:D490-5 (2014), which is hereby incorporated by reference in its entirety). Amino acid sequences were all extracted from the UniProt database (UniProt, C., “UniProt: A Worldwide Hub of Protein Knowledge,” Nucleic Acids Res 47:D506-D515 (2019), which is hereby incorporated by reference in its entirety). Each GT coding region was examined for membrane domains using the UniProt database to determine the TMD topology. GTs with internal or multi-pass TMDs were expressed as full-length proteins. For type II transmembrane proteins, N- and C-terminal TMD segments were truncated while stem regions were generally retained. For other classes, N-terminal signal peptides and C-terminal ER retention signals were generally removed. Amino acid sequences of full-length and truncated variants of all GTs in this study are provided in FIGS. 2C-2D. All GT genes were codon-optimized for expression in E. coli using GeneArt software (Thermo Fisher Scientific). These genes were then synthesized and ligated into the previously described SIMPLEx plasmid (Mizrachi et al., “Making Water-soluble Integral Membrane Proteins In Vivo Using an Amphipathic Protein Fusion Strategy,” Nat Commun. 6:6826 (2015), which is hereby incorporated by reference in its entirety) to generate plasmids encoding SIMPLEx-reformatted GTs having the form pET28a(+)-MBP-(NdeI)-GT-(EcoRI)-ApoAI*-6×His. PCR was used to amplify each GT gene with flanking NcoI and NotI restriction sites, and then ligated into pET28a(+) vector to create plasmids for expression of unfused GT constructs having the form pET28a(+)-(NcoI)-GT-(NotI)-6×His. All PCR reactions were performed using 0.1 μM gene-specific primers, 50 ng DNA template, and Phusion® High-Fidelity DNA Polymerase (New England Biolabs). Ligation products were used to chemically transform E. coli DH5a, and the transformation cultures were plated on LB-agar plates containing kanamycin. Clones were selected and screened by colony PCR using 2×-OneTaq Quickload master mix (New England Biolabs). Successful clones were confirmed by Sanger sequencing at the Cornell Biotechnology Resource Center. Due to incompatibility of DNA restriction sites, plasmids used for expression in yeast and mammalian cells were constructed using Gibson assembly. Briefly, standard PCR was used to amplify target genes containing 20-25 bp homologous regions with vector at both ends. 50 ng of linearized vector and 150 ng of amplified insert were then combined in a Gibson Assembly Master Mix (New England Biolabs) and incubated for 1 hour. Assembly reactions were then used to transform E. coli DH5a, after which clones were screened and confirmed according to a similar procedure as described above.

### Small-Scale Expression and Subcellular Fractionation

Plasmids encoding Sx-GT and unfused GT constructs were used to transform either E. coli strain BL21(DE3) for GTs containing no disulfide bonds or SHuffle T7 Express lysY for GTs contain predicted or confirmed to contain disulfide bonds. Small 5-mL LB cultures of E. coli harboring either a Sx-GT or GT plasmid were grown to an optical density at 600 nm (OD₆₀₀) of approximately 0.6-0.8 and then induced with IPTG to a final concentration of 0.1 mM. Protein expression proceeded for 18 hours at 16° C., after which culture volumes equivalent to OD₆₀₀ of 2.0 were harvested. Media was removed by centrifugation and the resulting cell pellet was resuspended in 1 mL phosphate buffer saline (PBS). Cells were lysed using a Q125 Sonicator (Qsonica) with a 3.175-mm diameter probe at a frequency of 20 kHz and 40% amplitude. Lysate was first centrifuged at 15,000×g for 30 minutes at 4° C. Supernatant was collected and centrifuged at 100,000×g for 1 hour at 4° C. The supernatant from this ultracentrifugation step was collected as the soluble fraction. Pellet was then resuspended in 1 mL PBS containing 1% (v/v) Triton X-100. The suspension was incubated for 1 hour at 4° C. to allow partitioning of membrane proteins into Triton X-containing buffer. Following ultracentrifugation at 100,000×g for 1 hour at 4° C., supernatant was collected as the detergent-solubilized fraction, while the pellet was taken as the insoluble fraction.

### Protein Purification and Yield Determination

A single colony of E. coli harboring plasmid DNA encoding a specific glycoenyzme was selected from a transformation plate and grown overnight in LB media at 37° C. The next day, cells were subcultured 5% into 1 L of fresh LB media. Cells were grown at 37° C. until OD₆₀₀ reached approximately 0.6-0.8, after which IPTG was supplemented into culture at 0.1 mM final concentration. Protein expression proceeded at 16° C. for 18 hours. Unless otherwise noted, all purification procedures were performed at 4° C. Cells were harvested, resuspended in PBS supplemented with 10% (v/v) glycerol, and lysed by passing the cell suspension through an Emulsiflex C5 homogenizer (Avestin) twice at 15,000 psi maximum pressure. Supernatant was collected following centrifugation at 15,000×g for 30 minutes and then incubated with 300 μL pre-washed HisPur™ Ni-NTA resin (Thermo Fisher Scientific) at 4° C. for 1 hour. The suspension was loaded onto an Econo-Pac© gravity flow chromatography column (Bio-Rad) and resin was washed with 6 column volumes HisPur wash buffer (50 mM NaH₂PO₄, 300 mM NaCl, 10 mM imidazole, pH 8.0). The target protein was eluted with HisPur elusion buffer (50 mM NaH₂PO₄, 300 mM NaCl, 300 mM imidazole, pH 8.0). Sample was then buffer exchanged into PBS using Zeba spin desalting columns, 7K MWCO (Thermo Fisher Scientific). Protein concentration was determined using Bradford assay (Bio-Rad). Purified protein fractions were subjected to standard Coomassie-blue staining of SDS-PAGE gels and purity of each was determined by densitometry analysis using BioRad Image Lab software (version 6.1.0 build 7), whereby the intensity of the band corresponding to the full-length Sx-GT construct was normalized to the intensity of all bands that appeared in the same lane of the gel. In general, purity of isolated Sx-GTs was approximately 50-80% following just a single-step Ni-NTA purification. Final yield values were tabulated based on both total protein concentration and purity, and were representative of three biological replicates starting from freshly transformed cells.

All other purification was performed as described above but with amylose resin (NEB) instead of Ni-NTA resin. Clarified lysate was incubated with 300 μL pre-washed amylose resin with rotation for 2 hours at 4° C. The suspension was loaded onto an Econo-Pac© gravity column (Bio-Rad) and resin was washed with 6 column volumes of amylose column buffer (20 mM Tris-HCl, 200 mM NaCl, 1 mM EDTA, pH 7.4). The target protein was eluted with amylose elusion buffer (10 mM maltose in column buffer). Protein purity and concentration were determined by Coomassie staining and Bradford assay (both from Bio-Rad), respectively. Proteins were kept at 4° C. for 2 weeks. For longer term storage at −80° C., protein solution was supplemented with 10% (v/v) glycerol and 0.02% (w/v) sodium azide as a cryogenic agent and bacteriostat, respectively.

For human MAN2A1 expression and purification, an expression construct encoding the truncated catalytic domain of human MAN2A1 (UniProt Q16706, residues 27-1144) was used (Moremen Et Al., “Expression System For Structural And Functional studies of Human Glycosylation Enzymes,” Nat. Chem. Biol. 14:156-162 (2018), which is hereby incorporated by reference in its entirety). This recombinant human MAN2A1 construct was expressed by transient transfection of suspension culture HEK293F cells, with soluble recombinant human MAN2A1 expressed as a soluble secreted product that was purified as described (Kadirvelraj et al., “Human N-acetylglucosaminyltransferase II Substrate Recognition Uses a Modular Architecture That Includes a Convergent Exosite,” Proc. Natl. Acad. Sci. USA 115:4637-4642 (2018), which is hereby incorporated by reference in its entirety). Briefly, the conditioned culture medium was loaded on a Ni²⁺-NTA Superflow column (Qiagen) equilibrated with 20 mM HEPES, 300 mM NaCl, 20 mM imidazole, pH 7.4, washed with column buffer, and eluted successively with column buffers containing stepwise increasing imidazole concentrations (40-300 mM). The eluted fusion protein was pooled, concentrated, and concurrently mixed with recombinant TEV protease and EndoF1 at ratios of 1:10 relative to the GFP-MGAT2 for each enzyme, respectively, and incubated at 4° C. for 36 hours to cleave the tag and glycans. Dilution to lower the imidazole concentration was followed by passing the sample through a Ni²⁺-NTA column to remove the fusion tag and His-tagged TEV protease and EndoF1. The protein was further purified on a Superdex 75 gel filtration column (GE Healthcare) and peak fractions of MGAT2 were collected. The protein buffer was exchanged by ultrafiltration and adjusted to 1 mg/mL with buffer containing 20 mM HEPES, 100 mM NaCl, pH 7.0, 0.05% sodium azide, and 10% glycerol and stored at −80° C. until use.

For antibody expression and purification, glycoengineered HEK293F GnTI⁻ cells were used as follows. After at least three passages, cells were washed and resuspended at 3 million cells per mL concentration. Plasmid pVITRO1-Trastuzumab-IgG1/κ (Addgene #61883) was prepared from E. coli culture and the purified plasmid was flowed through an endotoxin removal column to remove contaminating endotoxin. Plasmid DNA-cationic lipid complex was then generated using Lipofectamine™ Transfection Reagent (Thermo Fisher Scientific) and was slowly added into the culture media with gentle mixing. The amount of DNA, cationic-lipid reagents, and cells were scaled linearly according to the manufacturer's protocol. Cells were maintained in a 37° C. incubator shaker for 24 hours prior to being supplemented with Expression Enhancer Reagents (Thermo Fisher Scientific). Cell cultures were maintained at the same condition for another 5 days to allow antibody accumulation in the culture supernatant. Cells were then removed by centrifugation at 1,000×g for 5 minutes and supernatant was filtered through a 0.2-micron bottle-top filter. Supernatant was then mixed with 1×PBS at a 1:1 (v/v) ratio. This solution was flowed through MabSelect SuRe resin (Sigma-Aldrich) twice to allow antibody capture on protein A/G beads. Following extensive washing with 1×PBS, captured antibodies were eluded using glycine solution (pH 2.0) directly into neutralizing buffer (Tris-HCl pH 8.5). The antibody product was then buffer exchanged into 1×PBS supplemented with 0.01% sodium azide. Antibody was stored at 4° C. and was stable at the described conditions for at least a month.

### Immunoblot Analysis

Prior to electrophoretic separation, samples were combined with NuPAGE™ 4× LDS Sample Buffer (Invitrogen) supplemented with 2.5% β-mercaptoethanol and then boiling at 100° C. for 10 minutes. Samples equivalent to OD₆₀₀ of 0.375 for small-scale expression or 15 μL of CFPS reaction were loaded into each well of Bolt™ 8% Bis-Tris Plus Gels (Thermo Fisher Scientific). Following electrophoretic separation and transfer to Immobilon-P polyvinylidene difluoride (PVDF) membranes (0.45 m), blots were washed with TBS buffer (80 g/L NaCl, 20 g/L KCl, and 30 g/L Tris-base) followed by a 1-hour incubation in blocking solution (50 g/L non-fat milk in TBS supplemented with 0.05% (v/v %) Tween-20; TBST). Blots were then washed 4 times with TBST in 10-minute intervals and probed with primary antibodies including rabbit polyclonal antibody to 6×His epitope tag (Thermo Fisher Scientific; Cat #PA1-983B; 1:5,000 dilution), mouse monoclonal anti-GAPDH clone 6C5 (Calbiochem; Cat #CB1001; 1:10,000 dilution), rabbit polyclonal anti-GroEL (Sigma-Aldrich; Cat #G6532; 1:20,000 dilution), and rabbit anti-alpha tubulin clone EPR13799 (Abcam; Cat #ab184970; 1:10,000 dilution). Secondary antibodies were used as needed and these include goat anti-rabbit IgG H&L (HRP) (Abcam; Cat #ab6721; 1:5,000 dilution), rabbit anti-mouse IgG H&L (HRP) (Abcam; Cat #ab6728; 1:5,000 dilution), and ExtrAvidin®-Peroxidase (Sigma-Aldrich; Cat #E2886; 1:4,000 dilution). Blots were then washed as above. Imaging of blots was performed using a ChemiDoc™ XRS⁺ System following a brief incubation with Western ECL substrate (Bio-Rad).

### Sialyltransferase Activity Assay

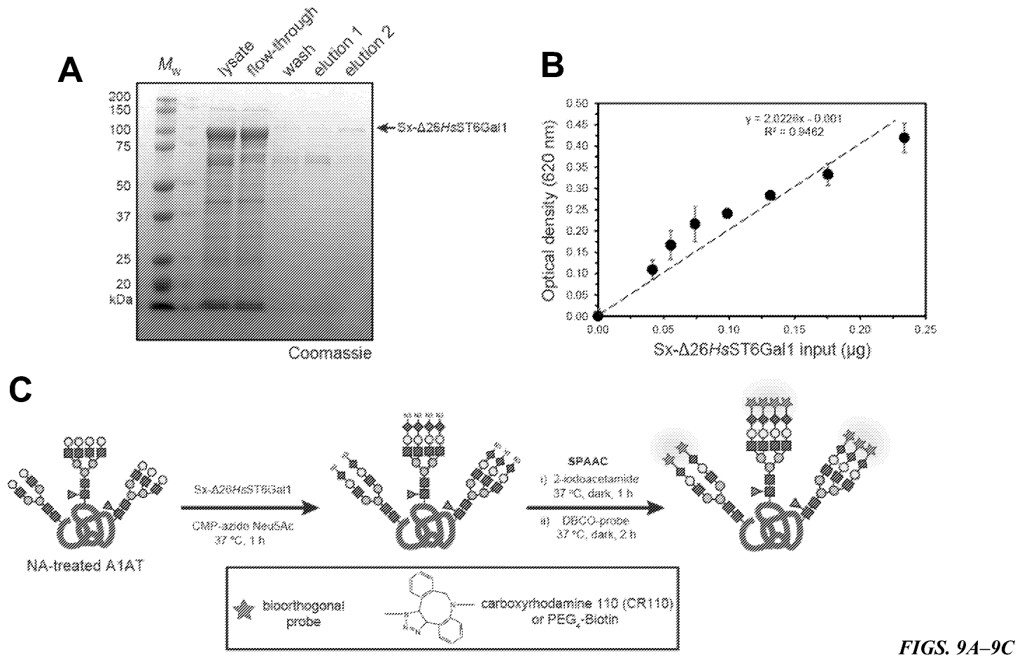

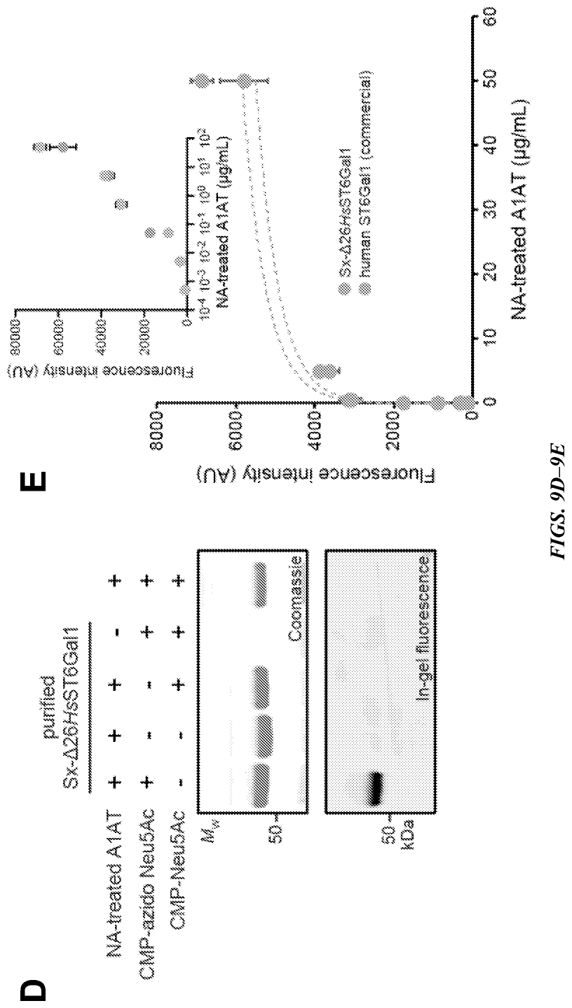

Kinetic analysis of sialytransferases was performed using a commercial sialytransferase activity kit (R&D Systems, Cat #EA002) according to manufacturer's protocols. Briefly, assays used 2 g/mL of purified Sx-Δ26HsST6Gal1 or commercial human ST6Gal1 (amino acids 44-406) (R&D Systems; Cat #7620-GT-010), 1.0 mg/mL of asialofetuin (Sigma-Aldrich; Cat #A4781-50MG) as acceptor substrate, and 0.02-0.8 mM of CMP-Neu5Ac as donor substrate. All reactions were incubated for 15 minutes at 37° C. Values for Vₘₐₓ and Kₘ were determined using Prism 9 for MacOS version 9.2.0. A conversion factor used for calculating the amount of enzymatically released inorganic phosphate from CMP-Neu5Ac was determined to be 3,833.5 pmol/OD₆₂₀ using the phosphate standards included in the kit and was used for all data analysis. Specific activity was calculated using 0.1 mM of CMP-Neu5Ac, 1.0 mg/mL of asialofetuin, and 0.04-0.23 g of Sx-Δ26HsST6Gal1. A linear plot of absorbance (OD₆₂₀) versus amount of Sx-Δ26HsST6Gal1 was generated (FIG. 9B). The slope of this plot was transformed using the conversion factor and divided by the reaction time to calculate the specific activity in units of pmol/min/g.

### Bioorthogonal Click Chemistry-Based Chemoenzymatic Remodeling

Strain-promoted alkyne-azide cycloaddition was used to assess the ability of Sx-GTs to chemoenzymatically remodel glycoprotein substrates. In a typical reaction, a 1.5-mL microcentrifuge tube was charged with 20 μL of reaction mixture consisting of 1 μg purified Sx-GT or 50 μg cell lysate, 3 μg purified acceptor glycoprotein substrate, and 10 mM nucleotide-activated monosaccharide donor modified with an azide functional group. Depending on the GT reactions, the nucleotide-activated monosaccharide donors included UDP-GlcNAz, UDP-GalNAz, GDP-AzFuc, and CMP-AzNeu5Ac (all from R&D Systems). Following an incubation in a 37° C. water bath for 1 hour, reaction mixtures were supplemented with 2-iodoacetamide (Sigma-Aldrich) at 100 mM final concentration and incubated in the dark at room temperature for 1 hour. Then, 100 mM final concentration of carboxyrhodamine 110 or biotin(PEG)₄ conjugated dibenzocyclooctyne-amines (Click Chemistry Tools) in N,N-dimethylformamide (DMF) was supplemented into the reaction mixture. Strain-promoted click reactions were carried out at 37° C. for 2 hours. Samples were then combined with 4×LDS Sample Buffer (Invitrogen) supplemented with 2.5% β-mercaptoethanol and heated at 65° C. for 5 minutes. Following SDS-PAGE analysis, in-gel fluorescence from carboxyrhodamine110-linked glycans on glycoproteins was measured using a ChemiDoc™ MP Imaging System (Bio-Rad) with 501/523 nm λₑₓ/λₑₘ. Biotin-linked glycans on glycoproteins were analyzed following immunoblot analysis using horseradish peroxidase conjugated streptavidin (Sigma-Aldrich) in a similar manner as described above for immunoblot analysis.

### Cell-Free Protein Synthesis

E. coli lysate was prepared according to an established protocol (Kwon and Jewett, “High-throughput Preparation Methods of Crude Extract for Robust Cell-free Protein Synthesis,” Scientific Reports 5:8663 (2015), which is hereby incorporated by reference in its entirety). Briefly, E. coli strain BL21(DE3) was cultured in 2×YTPG media (16 g/L tryptone, 10 g/L yeast extract, 5 g/L NaCl, 7 g/L potassium phosphate monobasic, 3 g/L potassium phosphate dibasic and 18 g/L glucose) at 37° C. with 0.5 mM IPTG until OD₆₀₀ reached approximately 1.0. Cells were then harvested and washed twice with cold S30 buffer (10 mM tris-acetate pH 8.2, 14 mM magnesium acetate and 60 mM potassium acetate). The resulting pellet was stored at −80° C. until used. To prepare crude extract, pellets were thawed on ice and resuspended with S30 buffer (1 mL per gram cell pellet). Cells were lysed using a Q125 Sonicator with a 3.175-mm diameter probe at a frequency of 20 kHz and 40% amplitude until the total energy input reached 1500 J. Lysate was then centrifuged twice at 30,000×g at 4° C. for 30 minutes. Supernatant was then collected, aliquoted, and stored at −80° C. until used. Cell-free synthesis of Sx-GT and unfused GT constructs was performed using the modified PANOx-SP system (Jewett and Swartz, “Mimicking the Escherichia coli Cytoplasmic Environment Activates Long-lived and Efficient Cell-free Protein Synthesis,” Biotechnology and Bioengineering 86:19-26 (2004), which is hereby incorporated by reference in its entirety). Specifically, S30 lysate was pre-conditioned with 750 M iodoacetamide in the dark at room temperature for 30 minutes and then lysate was supplemented with 200 mM glutathione at a 3:1 ratio between oxidized and reduced forms. Then, 200 ng plasmid DNA was introduced into cell-free protein synthesis reaction containing 30% (v/v) S30 lysate and the following: 12 mM magnesium glutamate, 10 mM ammonium glutamate, 130 mM potassium glutamate, 1.2 mM adenosine triphosphate (ATP), 0.85 mM guanosine triphosphate (GTP), 0.85 mM uridine triphosphate (UTP), 0.85 mM cytidine triphosphate (CTP), 0.034 mg/mL folinic acid, 0.171 mg/mL E. coli tRNA (Roche), 2 mM each of 20 amino acids, 30 mM phosphoenolpyruvate (PEP, Roche), 0.33 mM nicotinamide adenine dinucleotide (NAD), 0.27 mM coenzyme-A (CoA), 4 mM oxalic acid, 1 mM putrescine, 1.5 mM spermidine, and 57 mM HEPES. The synthesis reaction was carried out at 30° C. for 6 hours, after which the sample was centrifuged at 15,000×g for 30 minutes at 4° C. Supernatant was collected and stored at −20° C. until further analysis.

### Yeast and Mammalian Cell Expression

Yeast cells were transformed with plasmid pYS338 encoding Δ26HsST6Gal1 using the LiAc/single stranded carrier DNA/PEG method (Gietz and Schiestl, “High-efficiency Yeast Transformation Using the LiAc/SS Carrier DNA/PEG Method,” Nat. Protoc. 2:31-4 (2007), which is hereby incorporated by reference in its entirety). For yeast expression, SBY49 cells were grown in-URA media at 30° C. until OD₆₀₀ reached approximately 0.6-0.8, after which protein expression was induced with galactose to a final concentration of 2% (w/v). Protein expression was performed for 22 hours at 30° C. Yeast cells were lysed by vortexing the cell suspension with glass beads in PBS containing zymolyase enzyme. For mammalian cell expression, 2.0 mL of HEK293T cells at approximately 80% confluency in a 6-well plate were transfected with 2 g plasmid DNA using jetPRIME® transfection reagent (Polyplus Transfection). After transfection, cells were maintained in an incubator at 37° C. with 5% CO₂ and 90% relative humidity for 36 hours, after which they were harvested. HEK293T cells were lysed by tip sonication. Subcellular fractionation analysis for yeast and HEK293T cells was performed similarly as described above. All samples were stored at −20° C. until further analysis.

### Cell-Free Bioenzymatic Glycan Synthesis

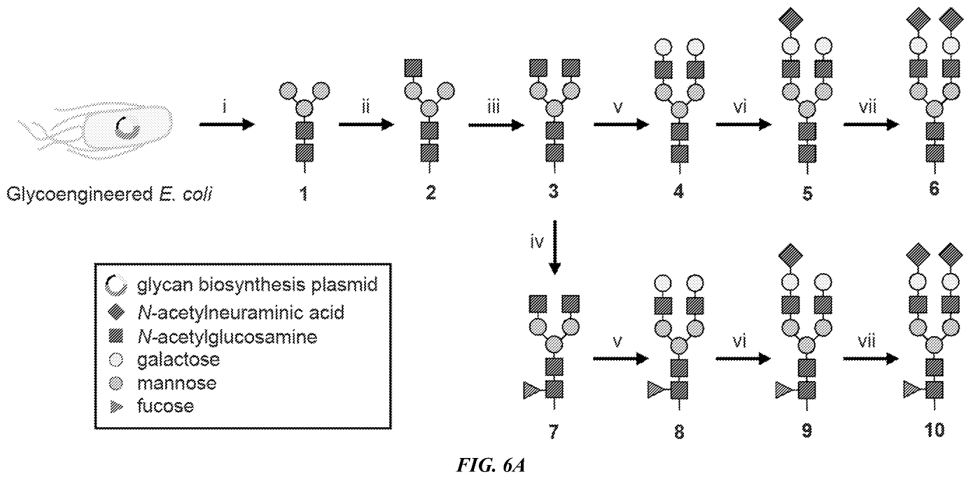

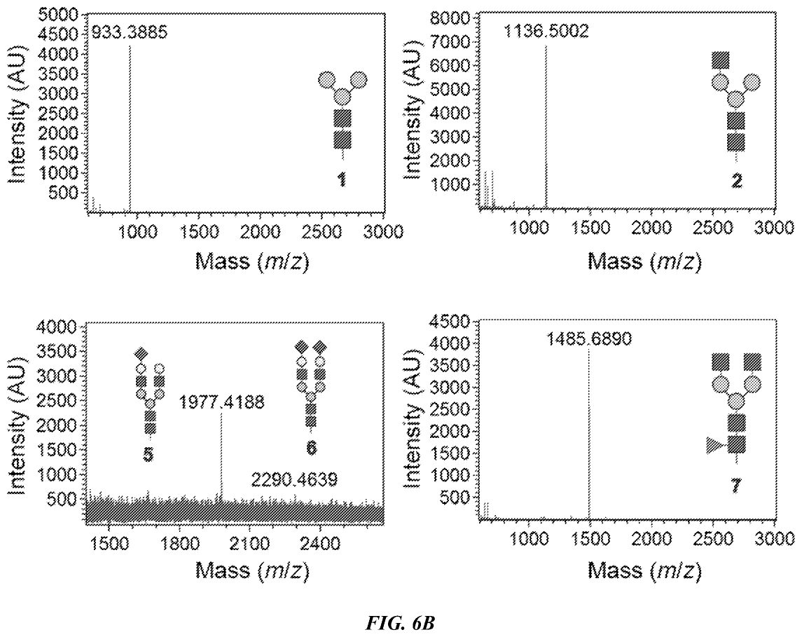

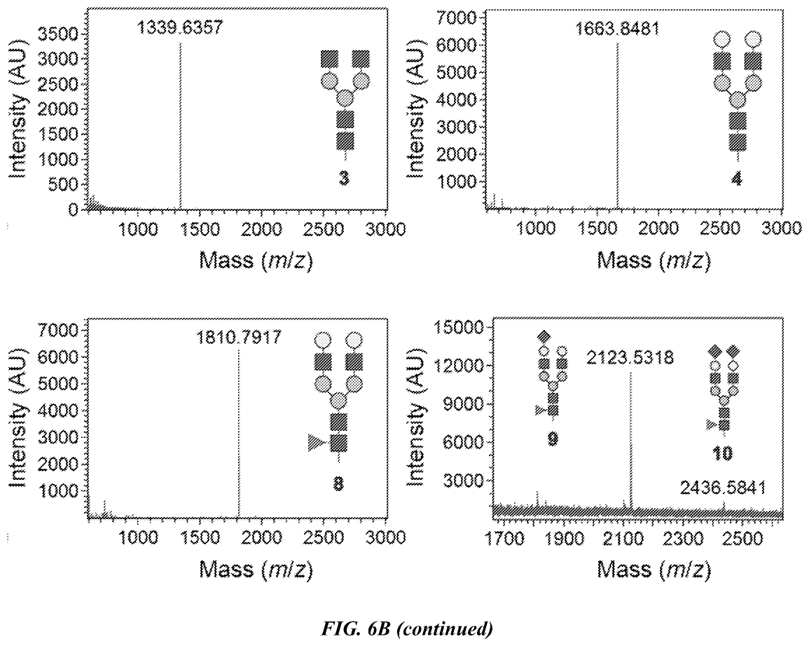

All glycans and nucleotide-activated sugar substrate solutions were prepared in sterile DI water and stored at −20° C. Glycan 1 was prepared as described (Hamilton et al., “A Library of Chemically Defined Human N-glycans Synthesized From Microbial Oligosaccharide Precursors,” Sci. Rep. 7:15907 (2017), which is hereby incorporated by reference in its entirety). Briefly, dried cell pellets from a 250-mL culture of E. coli Origami2(DE3) gmd::kan ΔwaaL cells carrying plasmid pConYCGmCB (Glasscock et al., “A Flow Cytometric Approach to Engineering Escherichia coli for Improved Eukaryotic Protein Glycosylation,” Metab. Eng. 47:488-495 (2018), which is hereby incorporated by reference in its entirety) were resuspended in 2:1 chloroform: methanol, sonicated, and the remaining solids collected by centrifugation. This pellet was sonicated in water and collected by centrifugation. The resulting pellet was sonicated in 10:10:3 chloroform: methanol:water to isolate the lipid-linked oligosaccharides (LLOs) from the inner membrane. The LLOs were purified using acetate-converted DEAE anion exchange chromatography as they bind to the anion exchange resin via the phosphates that link the lipid and glycan. The resulting compound was dried and treated by mild acid hydrolysis to release glycans from the lipids. The released glycans were then separated from the lipid by a 1:1 butanol:water extraction, wherein the water layer contains the glycans. The glycans were then further purified with a graphitized carbon column using a 0-50% water: acetonitrile gradient. Following this procedure, approximately 750 g of glycan 1 that was well resolved from contaminant peaks was reproducibly obtained (FIG. 6B). To synthesize glycan 2, 5 μg of glycan 1 was incubated with 20 g/mL Sx-Δ29HsGnTI and 10 mM UDP-GlcNAc (Sigma-Aldrich) in GnT buffer (20 mM HEPES, 50 mM NaCl, 10 mM MnCl₂, pH 7.2) at 37° C. for 16 hours. To synthesize glycan 3, glycan 2 was incubated with 80 g/mL Sx-Δ29HsGnTII and 20 mM UDP-GlcNAc in GnT buffer at 37° C. for 36 hours. Glycan 3 was then incubated with 20 g/mL Sx-Δ44Hsβ4GalT1 and 10 mM UDP-Gal (Sigma-Aldrich) in GalT buffer (20 mM HEPES, 150 mM NaCl, 10 mM MnCl₂, pH 7.5) at 37° C. for 16 hours to produce glycan 4. Sialic acid terminal glycans 5 and 6 were synthesized by incubating glycan 4 with 20 g/mL Sx-Δ26HsST6Gal1 and 20 mM CMP-Neu5Ac (Sigma-Aldrich) in SiaT buffer (50 mM sodium phosphate, 150 mM NaCl, 10 mM MgCl₂, pH 8.0) at 37° C. for 16 hours. Glycan 7 was synthesized by incubating glycan 4 with 20 g/mL Sx-Δ30HsFucT8 and 10 mM GDP-fucose (Sigma-Aldrich) in FucT buffer (100 mM MES, 10 mM MgCl₂, pH 7.0) at 37° C. for 16 hours. Glycans 8, 9, and 10 were synthesized sequentially from glycan 7 using Sx-Δ44Hsβ4GalT1 and Sx-Δ26HsST6Gal1 as described above for glycans 4, 5, and 6. Following reaction clean-up and glycan purification, reaction progress was monitored by MALDI-TOF MS. Briefly, 1 μL (˜25 ng) of partially purified glycan was co-crystalized with 1 μl matrix consists of 2,5-dihydroxybenzoic acid (10 mg/ml) in 70% (v/v) acetonitrile. The sample was analyzed in positive mode MALDI-TOF (SCIEX TOF/TOF 5800) operated in linear mode with data acquisition at 2000 shots/spot in the 5-100-kDa mass range. Because sialic acid is subject to MS-induced in-source and metastable decay, successful biosynthesis of glycans 5, 6, 9, and 10 was verified by nano LC-MS/MS analysis as described below.

### Cell-Free Bioenzymatic Glycan Remodeling on Glycoproteins

Unless noted otherwise, all glycoprotein remodeling reactions were performed at 37° C. for 1 hour prior to bioorthogonal labeling reaction as described above. The sialytransferase activity of Sx-CjCstII was assessed using human A1AT as glycoprotein acceptor substrate. A total of 3 g of recombinant A1AT (R&D Systems) was treated with 20 U/L a2-3,6,8,9 neuraminidase A (NEB) in a 10-μL reaction at 37° C. for 2 hours to remove terminal sialic acid residues on A1AT glycans. Reaction mixtures were then heated at 85° C. for 15 minutes to inactivate neuraminidase A. Neuraminidase A-treated A1AT was then incubated with Sx-CjCstII and CMP-AzNec5Ac in SiaT buffer in a 37° C. water bath for 1 hour. Sialyltransferase activity of Sx-Δ34HsST3Gal1 was evaluated in a similar manner but neuraminidase-treated bovine submaxillary glands mucin (Sigma-Aldrich) was used as the glycoprotein substrate. N-acetylglucosaminyltransferase activity of Sx-Δ29HsGnTI was assessed using MBP-GCG^{DQNAT} a fusion between E. coli MBP and human glucagon (residues 1-29) followed by a C-terminal DQNAT glycosylation tag (Glasscock et al., “A Flow Cytometric Approach to Engineering Escherichia coli for Improved Eukaryotic Protein Glycosylation,” Metab. Eng. 47:488-495 (2018), which is hereby incorporated by reference in its entirety). The MBP-GCG^{DQNAT} construct was glycosylated with Man₃GlcNAc₂ using glycoengineered E. coli as described (Glasscock et al., “A Flow Cytometric Approach to Engineering Escherichia coli for Improved Eukaryotic Protein Glycosylation,” Metab. Eng. 47:488-495 (2018), which is hereby incorporated by reference in its entirety). Briefly, Origami2(DE3) gmd::kan ΔwaaL cells carrying plasmid pConYCGmCB along with plasmid pMAF10 (Feldman et al., “Engineering N-linked Protein Glycosylation With Diverse O Antigen Lipopolysaccharide Structures in Escherichia coli,” Proc Natl Acad Sci USA 102:3016-21 (2005), which is hereby incorporated by reference in its entirety) and pTrc-spDsbA-MBP-GCG^{DQNAT} (Glasscock et al., “A Flow Cytometric Approach to Engineering Escherichia coli for Improved Eukaryotic Protein Glycosylation,” Metab. Eng. 47:488-495 (2018), which is hereby incorporated by reference in its entirety) were grown in 100 mL of LB at 37° C. until OD₆₀₀ reached ≈1.5. Culture temperature was reduced to 30° C. and allowed to grow overnight at 30° C. The next day, cells were induced with 0.1 mM isopropyl β-D-1-thiogalactopyranoside (IPTG) to initiate synthesis of the MBP-GCG^{DQNAT} acceptor protein. Protein expression proceeded for 8 hours at 30° C. Cells were then harvested and subjected to subcellular fractionation. This involved pelleting and washing 100 mL of IPTG-induced culture with subcellular fractionation buffer (0.2 M Tris-Ac (pH 8.2), 0.25 mM EDTA, and 0.25 M sucrose, and 160 g/mL lysozyme). Cells were resuspended in 1.5 mL subcellular fractionation buffer and then incubated for 5 minutes on ice and spun down. After addition of 60 μL of 1M MgSO₄, cells were incubated for 10 minutes on ice. Cells were spun down, and the supernatant was taken as the periplasmic fraction. To isolate glycoproteins, periplasmic fractions were subjected to affinity chromatography using HisPur™ Ni-NTA resin (Thermo Fisher Scientific). Eluates were collected, solubilized in Laemmli sample buffer containing 5% β-mercaptoethanol, and resolved on SDS-polyacrylamide gels. Purified MBP-GCG^{DQNAT} was incubated with Sx-Δ29HsGnTI and UDP-GlcNAz in GnT buffer in a 37° C. water bath for 1 hour. Fucosyltransferase activity was evaluated by incubating A1AT or neuraminidase A-treated A1AT with Sx-Δ36HsFucT7 and GDP-AzFuc in FucT buffer in a 37° C. water bath for 1 hour.

### Endoglycosidase Sensitivity Assay

In a sterile Eppendorf microcentrifuge tube, 1 g of purified trastuzumab bearing Man₅GlcNAc₂ glycan was incubated with: (i) Streptococcus pyogenes Endo S2 (Genovis #AO-GL8-020) in Glycobuffer 1 (NEB #B1727SVIAL); (ii) Elizabethkingia meningosepticum Endo F1 (Sigma-Aldrich #324725) in GlycoBuffer 4 (NEB #B1703); (iii) Elizabethkingia miricola Endo F3 (NEB #P0771S) in GlycoBuffer 4; or (iv) PBS control. Reaction mixtures were incubated at 37° C. for 16 hours and the product was analyzed by LC-MS using intact protein MS mode.

### Cell-Free Bioenzymatic Glycan Remodeling on Trastuzumab

Glycan remodeling on full-length mAb was performed in an on-column mode. 50 g purified trastuzumab bearing Man₅GlcNAc₂ glycan was first incubated with MabSelect SuRe resin (Sigma-Aldrich) for 10 minutes to allow antibody capture on protein A/G beads. This mixture was then transferred to a spin column, followed by washing twice with PBS. The bottom of the spin column was then capped with rubber cap. In a separate tube, 50 μL of a specific glycan remodeling reaction mixture was prepared. For preparing N-acetylglucosaminyltransferase, galactosyltransferase, fucosyltransferase, and sialyltransferase reaction mix. UDP-GlcNAz substrate was used at the same concentration as UDP-GlcNAc. Reaction using β-N-acetylglucosaminidase S (NEB #P0744S) was performed in Glycobuffer 1 (NEB) at 37° C. for 4 hours. Reactions using human Man2A1 mannosidase were performed in 50 mM sodium acetate buffer (pH 5.5) 1 mM ZnCl₂ at 37° C. for 16 hours. Following each reaction step, the reaction mixture was removed by centrifugation at 300×g for 2 minutes. Resin was then washed twice with PBS using the same centrifugation setting. In general, approximately 80-90% recovery yield of IgG was observed following purification as determined by NanoDrop spectrophotometer. Subsequent reaction mixture was then added to the column and the clean-up process was repeated for each reaction step. Final IgG product was eluted using glycine solution (pH 2.0) and analyzed immediately by LC-MS.

### Chromatography and Mass Spectrometry

Hydrophilic interaction liquid chromatography (HILIC) was carried out using an Exion HPLC system with built-in autosampler (SCIEX). The free glycan samples were reconstituted in buffer A (80%: 20% acetonitrile: water), filtered with 0.22 μm spin filter (Corning) and loaded onto a Kinetex HILIC column (2.6 μm, 2.6×150 mm; Phenomenex) with 80% ACN/20% water as buffer A and 50 mM NH₄FA with pH 4.4 as buffer B. LC was performed using a 7-min gradient from 80 to 0% of buffer B at a flow rate of 400 μL/min.

All LC-MS/MS analysis was carried out using an X500B QTOF (SCIEX) mass spectrometer equipped with an electrospray ion source and coupled with an Exion HPLC system. Each reconstituted sample was injected onto a Kinetex HILIC column (2.6 μm, 2.6×150 mm; Phenomenex). The free glycans were eluted in a 9-min gradient of 80% to 0% (80% ACN/20% water) at 400 nL/min followed by a 3-minute hold at 80% (80% ACN/20% water) for re-equilibration. The instrument was operated in positive ion mode with ESI voltage set at 5.0 kV, ion source gas 1, gas 2=50 psi, curtain gas=35 and CAD gas=7, and source temperature of 350° C. Calibration was done using positive calibrant with CDS system. For free glycan analysis, the instrument was operated in MS full-scan mode from m/z range from 2,00-2,000 followed by multiple reaction monitoring high-resolution (MRM-HR) scan from 0-12 minutes at two different collision energies of 20 and 35 V with DP=20 V and accumulation time of 0.25 s. MS survey scans were performed for the mass range of m/z 2,00-2000 with DP=20 V, CE=7 V and accumulation time of 0.25 s and MS/MS MRM-HR scans were at the same DP voltage and CE=20 V and with Q1 unit resolution. All MS and MS/MS raw spectra from each sample obtained by MRM-HR scan were analyzed by SCIEX OS 1.4 data analysis system. XIC spectra were extracted from MS full-scan with each MRM transition. The glycan structure was annotated manually using GlycanMass-ExPAsy tool.

### Physicochemical Data Collection and Analysis

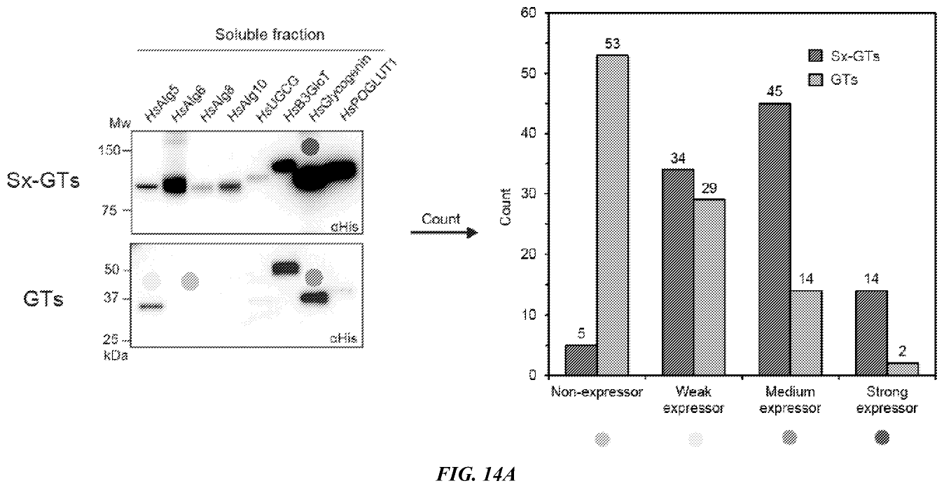

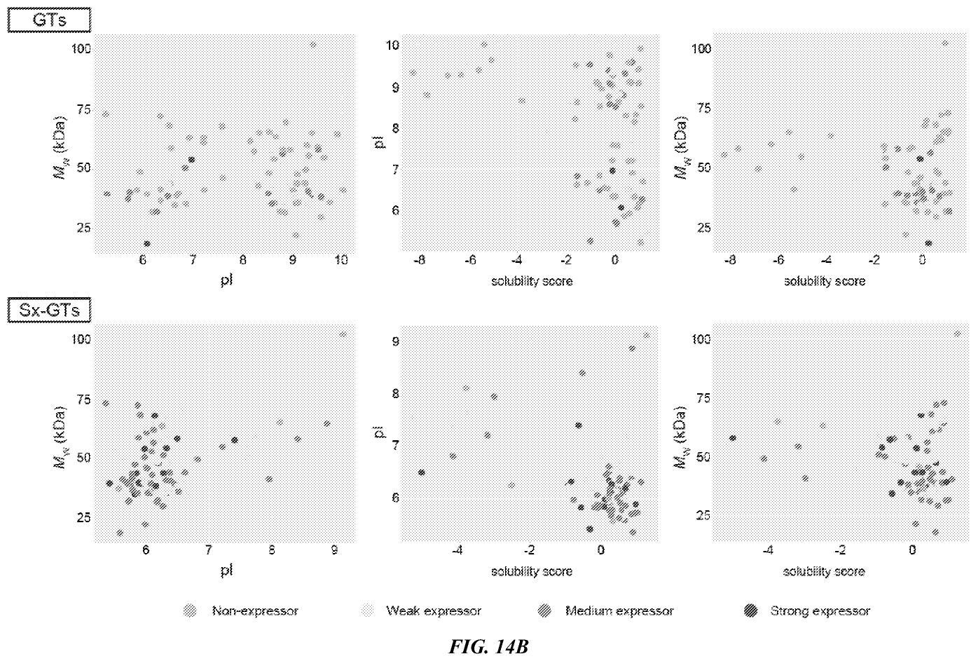

The name, amino acid sequence, structure availability (full-length or partial), and predicted post-translational modifications (i.e., disulfide bonds, glycosylation) for each GT enzyme were retrieved from the UniProt database (UniProt, C., “UniProt: A Worldwide Hub of Protein Knowledge,” Nucleic Acids Res 47:D506-D515 (2019), which is hereby incorporated by reference in its entirety). GT family members were annotated from the CAZy database (Lombard et al., “The Carbohydrate-active Enzymes Database (CAZy) in 2013,” Nucleic Acids Res 42:D490-5 (2014), which is hereby incorporated by reference in its entirety). Amino acid sequences of full length, truncated, and SIMPLEx-fused GTs were compiled in FASTA format. The M_{w} and pI were calculated using the ExPASy Bioinformatics resource portal in average resolution setting (Wilkins et al., “Protein Identification and Analysis Tools in the ExPASy Server,” Methods Mol. Biol. 112:531-52 (1999), which is hereby incorporated by reference in its entirety). Solubility prediction score was calculated using CamSol Intrinsic version 2.1 (Sormanni et al., “The CamSol Method of Rational Design of Protein Mutants With Enhanced Solubility,” J. Mol. Biol. 427:478-90 (2015), which is hereby incorporated by reference in its entirety). The expression scores for all constructs were annotated based on immunoblots in FIGS. 14A-14B. Correlation between protein properties (M_{w}, pI, solubility prediction score, and expression score) were analyzed using R software version 3.4.2. Specifically, scatter plots between protein properties, generated with data points colored according to expression score, were used to examine any possible correlations. For the correlation between expression score and pI, datasets were analyzed as a function of expression score movement. Scatter plots comparing the pI of Sx-GT versus GT constructs were created and the data were colored according to the change in expression score. A similar approach was used to analyze the correlation between expression score and solubility prediction score. Because no statistical significance was observed for the correlation between expression score and either pI or solubility prediction score, general observations from the plots were described instead. For the correlation between expression score and M_{w}, data were categorized into 3 groups: M_{w}<40 kDa, M_{w}=40-60 kDa, and MW>60 kDa, before average expression score for each group was calculated. Both Sx-GT and GT constructs were binned using the same criteria since the added mass from the SIMPLEx fusion was constant for all constructs. Welch's t-test was used to analyze statistical significance for categorical datasets.

### Statistical Analysis and Reproducibility

To ensure robust reproducibility of all results, experiments were performed with at least three biological replicates and at least three technical measurements. Sample sizes were not predetermined based on statistical methods but were chosen according to the standards of the field (at least three independent biological replicates for each condition), which gave sufficient statistics for the effect sizes of interest. All data were reported as average values with error bars representing standard error of the mean (SEM). Statistical significance was determined by Welch's t-test and p-values of <0.05 were considered significant. All graphs were generated using Microsoft Excel, Prism 9 for MacOS version 9.2.0, or R software version 3.4.2. No data were excluded from the analyses. The experiments were not randomized. The Investigators were not blinded to allocation during experiments and outcome assessment.

### Example 2—SIMPLEx Promotes Soluble Expression of Human ST6Gal1

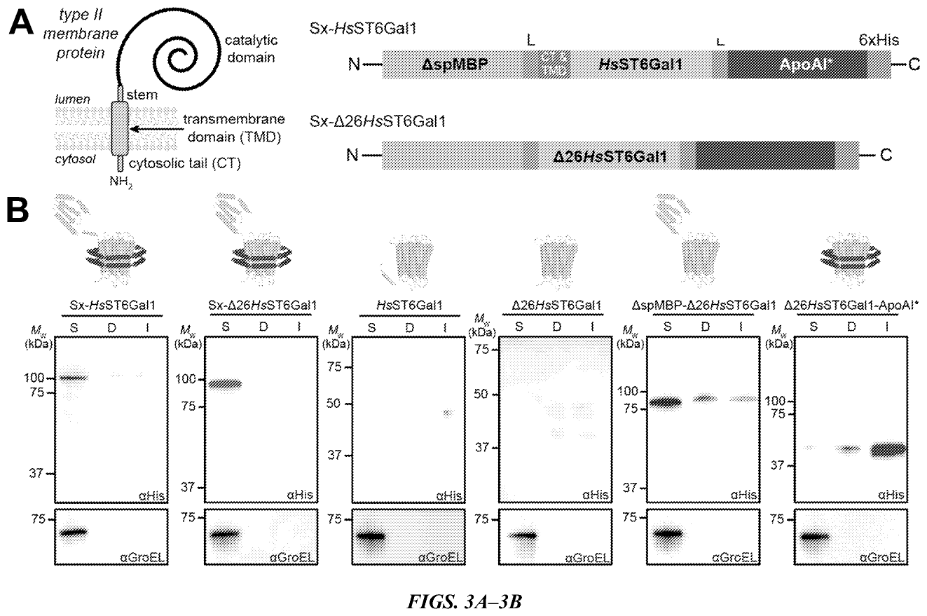

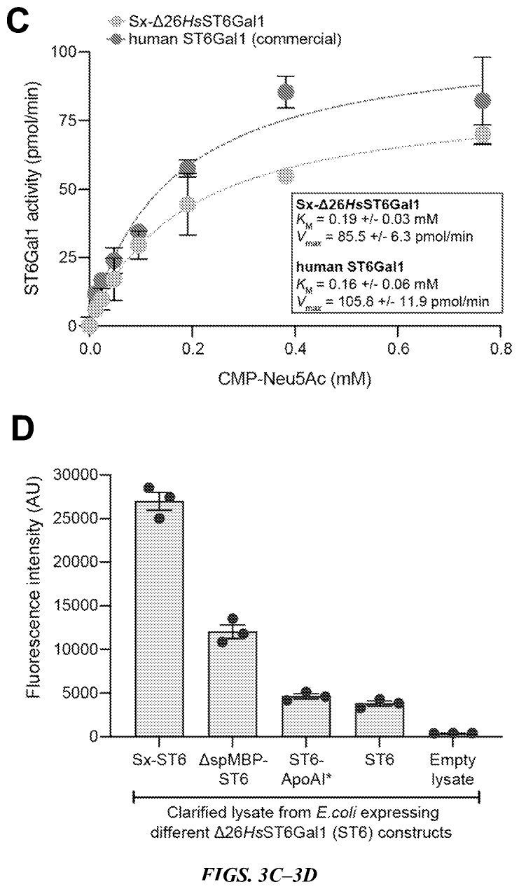

Towards the goal of developing a versatile and universal approach for large-scale GT production, it was hypothesized that SIMPLEx could relieve bottlenecks that have hampered GT expression in E. coli. The rationale for this hypothesis was based on two observations. First, the SIMPLEx strategy has previously been shown as a promising technique for converting IMPs into water-soluble proteins with retention of biological function (Mizrachi et al., “Making Water-soluble Integral Membrane Proteins In Vivo Using an Amphipathic Protein Fusion Strategy,” Nat. Commun. 6:6826 (2015) and Mizrachi et al., “A Water-soluble DsbB Variant That Catalyzes Disulfide-bond Formation In Vivo,” Nat. Chem. Biol. 13:1022-1028 (2017), which are hereby incorporated by reference in their entirety). Second, SIMPLEx was able to rescue soluble expression of a diverse panel of globular proteins that were previously reported to be recalcitrant to soluble expression in E. coli (Dyson et al., “Production of Soluble Mammalian Proteins in Escherichia coli: Identification of Protein Features That Correlate With Successful Expression,” BMC Biotechnol. 4:32 (2004), which is hereby incorporated by reference in its entirety) (FIG. 3A). Collectively, these results highlight the capacity of SIMPLEx to shield large amounts of protein hydrophobicity that drive misfolding and aggregation and promote soluble expression of membrane and non-membrane proteins alike.

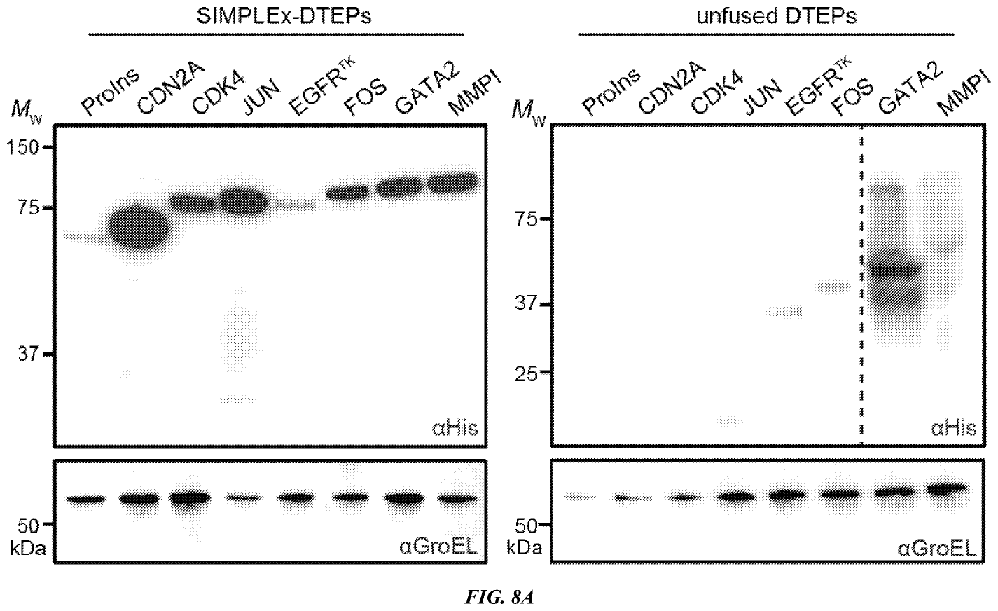

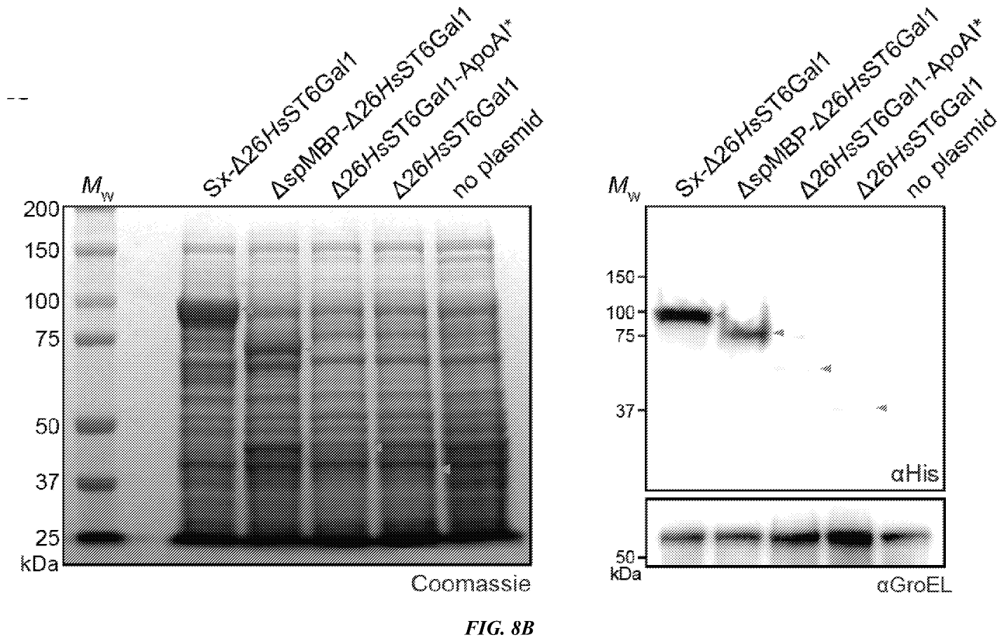

To see if the benefits of SIMPLEx could be leveraged for GT expression, the human β-galactoside-α2,6-sialyltransferase 1 (HsST6Gal1), a sialytransferase belonging to the GT29 family, was chosen as a model GT for proof-of-concept experiments. HsST6Gal1 consists of a short N-terminal cytoplasmic tail (CT), a transmembrane domain (TMD), a stem region that serves as a linker, and a large C-terminal catalytic domain that adopts a variant GT-A fold containing a seven-stranded central 3-sheet flanked by α-helices (FIG. 3A) (Kuhn et al., “The Structure of Human Alpha-2,6-sialyltransferase Reveals the Binding Mode of Complex Glycans,” Acta. Crystallogr. D. Biol. Crystallogr. 69:1826-38 (2013), which is hereby incorporated by reference in its entirety). Overexpression of HsST6Gal1 has been documented in several cancer cell types (Garnham et al., “ST6GAL1: A Key Player in Cancer,” Oncol Lett 18:983-989 (2019), which is hereby incorporated by reference in its entirety); hence, the ability to produce preparative amounts of HsST6Gal1 could help to understand its role in cancer biology and therapy. To express this enzyme in the SIMPLEx architecture, a tripartite HsST6Gal1 chimera in which its N-terminus was genetically fused to a water-soluble “decoy” protein, namely E. coli maltose-binding protein lacking its N-terminal signal peptide (ΔspMBP), while its C-terminus was fused to an amphipathic “shield” protein, namely truncated human apolipoprotein A1 lacking its 43-residue globular N-terminal domain (ApoAI*) was designed, yielding ΔspMBP-HsST6Gal1-ApoAI* (hereafter Sx-HsST6Gal1) (FIG. 3A). As removal of the transmembrane anchor segment is a common practice to improve expression and solubility of mammalian GTs (Moremen Et Al., “Expression System For Structural And Functional studies of Human Glycosylation Enzymes,” Nat. Chem. Biol. 14:156-162 (2018), which is hereby incorporated by reference in its entirety), the Sx-Δ26HsST6Gal1 variant in which 26 amino acids from the N-terminus of HsST6Gal1, comprising its CT and TMD, were genetically removed was also generated (FIG. 3A). The HsST6Gal1 enzyme contains 3 disulfide bonds in its native structure (Kuhn et al., “The Structure of Human Alpha-2,6-sialyltransferase Reveals the Binding Mode of Complex Glycans,” Acta. Crystallogr. D. Biol. Crystallogr. 69:1826-38 (2013), which is hereby incorporated by reference in its entirety). Therefore, the commercially available E. coli strain named SHuffle T7 Express (Lobstein et al., “SHuffle, a Novel Escherichia coli Protein Expression Strain capable of Correctly Folding Disulfide Bonded Proteins in its Cytoplasm,” Microb. Cell Fact 1156 (2012), which is hereby incorporated by reference in its entirety), which has been engineered with a more oxidizing cytoplasmic environment and expresses a cytoplasmic version of the disulfide bond isomerase DsbC (Lobstein et al., “SHuffle, a Novel Escherichia coli Protein Expression Strain capable of Correctly Folding Disulfide Bonded Proteins in its Cytoplasm,” Microb. Cell Fact 1156 (2012), which is hereby incorporated by reference in its entirety), was selected as an expression host to facilitate cytoplasmic disulfide bond formation. Following expression of the two engineered chimeras in SHuffle T7 Express cells, stable products corresponding to Sx-HsST6Gal1 and Sx-Δ26HsST6Gal1 that accumulated almost exclusively in the soluble cytoplasmic fraction were observed (FIG. 3B). In stark contrast, no detectable expression of unfused HsST6Gal1 or Δ26HsST6Gal1 was seen in the soluble fraction and only minimal amounts were observed in the insoluble and detergent-solubilized fractions (FIG. 3B), in agreement with previous findings that human sialyltransferases are poorly expressed in bacteria (Skretas et al., “Expression of Active Human Sialyltransferase ST6GalNAcI in Escherichia coli,” Microb. Cell Fact 8:50 (2009) and Ortiz-Soto and Seibel, “Expression of Functional Human Sialyltransferases ST3Gal1 and ST6Gal1 in Escherichia coli,” PLoS One 11:e0155410 (2016), which are hereby incorporated by reference in their entirety). The large expression difference seen for the Sx-Δ26HsST6Gal1 fusion relative to its unfused counterpart was also clearly observed in whole cell lysates (FIG. 8B).

To demonstrate the importance of the decoy and shield domains, chimeras lacking each of these elements were also expressed. When the decoy protein was omitted, Δ26HsST6Gal1-ApoAI* partitioned almost entirely in the insoluble fraction (FIG. 3B) and was undetectable in whole cell lysates (FIG. 8B). Omission of the shield protein resulted in accumulation of ΔspMBP-Δ26HsST6Gal1 primarily in the soluble fraction, but with significant amounts also detected in the detergent-solubilized and insoluble fractions (FIG. 3B). Consistent with earlier studies with integral membrane protein targets (Mizrachi et al., “Making Water-soluble Integral Membrane Proteins In Vivo Using an Amphipathic Protein Fusion Strategy,” Nat. Commun. 6:6826 (2015) and Mizrachi et al., “A Water-soluble DsbB Variant That Catalyzes Disulfide-bond Formation In Vivo,” Nat. Chem. Biol. 13:1022-1028 (2017), which are hereby incorporated by reference in their entirety), results confirmed the importance of the decoy and shield in directing folding away from the membrane and promoting water solubility. Moreover, SIMPLEx-based expression in a redox-engineered bacterial host sidestepped the need for chaperones that occur uniquely in the mammalian secretory pathway and for N-linked glycosylation of the GT that is not required for activity but needed for folding, stability, and solubility of the enzyme (Chen and Colley, “Minimal Structural and Glycosylation Requirements for ST6Gal I Activity and Trafficking,” Glycobiology 10:531-83 (2000) and Meng et al., “Enzymatic Basis for N-glycan Sialylation: Structure of rat Alpha2,6-sialyltransferase (ST6GAL1) Reveals Conserved and Unique Features for Glycan Sialylation,” J. Biol. Chem. 288:34680-98 (2013), which are hereby incorporated by reference in their entirety).

### Example 3—Soluble HsST6Gal1 in the SIMPLEx Framework Retains Biological Activity

To determine whether soluble Sx-Δ26HsST6Gal1 was biologically active, the enzyme was purified (FIG. 9A) and subjected to kinetic analysis using a commercial kit for quantifying release of nucleotide cytidine 5′-monophosphate (CMP) from the donor substrate CMP-N-acetylneuraminic acid (CMP-Neu5Ac). From this assay, the apparent K_{M} and Vₘₐₓvalues for Sx-Δ26HsST6Gal1 were determined as 0.19±0.03 mM and 85.5±6.3 pmol/min, respectively (FIG. 3C). These parameters were in reasonable agreement with the apparent kinetic parameters measured for the commercial human ST6Gal1 (produced recombinantly using N60 mouse myeloma cells) and that were measured previously (Mbua et al., “Selective Exo-enzymatic Labeling of N-glycans on the Surface of Living Cells by Recombinant ST6Gal I,” Angew Chem. Int. Ed. Engl. 52:13012-5 (2013), which is hereby incorporated by reference in its entirety). The specific activity of the soluble Sx-Δ26HsST6Gal1 enzyme was 516.9 pmol/min/g (FIG. 9B), which was also consistent with previously published data for human ST6Gal1 (Houeix and Cairns, “Engineering of CHO Cells for the Production of Vertebrate Recombinant Sialyltransferases,” Peer J 7:e5788 (2019), which is hereby incorporated by reference in its entirety).

Upon confirming that Sx-Δ26HsST6Gal1 was enzymatically active, it was next sought to demonstrate its practical utility for chemoenzymatic remodeling of N-linked glycans present on glycoprotein substrates. To this end, a bioorthogonal click chemistry-based assay for quantifying sialyltransferase-mediated chemoenzymatic modification was developed (FIG. 9C). Specifically, Sx-Δ26HsST6Gal1 enzyme preparations were evaluated for their ability to transfer azido-Neu5Ac from CMP-activated glycosyl donor onto terminal Gal residues of the alpha-1 antitrypsin (A1AT) serpin protein, which was first treated with neuraminidase to remove native sialic acids. The modified A1AT was then fluorescently labeled through a strain-promoted azide-alkyne cycloaddition (SPAAC) reaction using carboxyrhodamine 110 DBCO and separated by standard SDS-PAGE. Fluorescence intensity of the labeled A1AT proteins, which corresponded to the extent of chemoenzymatic remodeling by Sx-Δ26HsST6Gal1, was then directly visualized and quantified by in-gel fluorescence analysis.

Using clarified lysate generated from E. coli cells expressing Sx-Δ26HsST6Gal1 as a catalyst source, a strong fluorescence from the treated A1AT was detected (FIG. 3D). In contrast, clarified lysates containing either Δ26HsST6Gal1 or Δ26HsST6Gal1-ApoAI* yielded only a weak fluorescent signal (FIG. 3D), which was consistent with the barely detectable levels of soluble expression observed for these constructs that both lacked the ΔspMBP decoy (FIG. 3B). Interestingly, while addition of the ΔspMBP moiety alone was able to promote soluble expression of ΔspMBP-Δ26HsST6Gal1 (FIG. 3B and FIG. 8B), the clarified lysate containing this construct exhibited about 50% less activity than that measured for the Sx-Δ26HsST6Gal1 enzyme (FIG. 3D). A significant portion of the soluble ΔspMBP-Δ26HsST6Gal1 protein lacking ApoAI* was misfolded aggregates, consistent with previous findings (Mizrachi et al., “Making Water-soluble Integral Membrane Proteins In Vivo Using an Amphipathic Protein Fusion Strategy,” Nat Commun 6:6826 (2015), which is hereby incorporated by reference in its entirety) and indicative of the essential nature of both ΔspMBP and ApoAI* domains for producing this GT in a highly soluble, active conformation within the E. coli cytoplasm. Importantly, it was also confirmed that purified Sx-Δ26HsST6Gal1 catalyzed chemoenzymatic remodeling to an extent that was indistinguishable from that of commercial human ST6Gal1 (FIGS. 9D-9E). The fact that the fused ApoAI* domain did not measurably interfere with important C-terminal catalytic regions in Δ26HsST6Gal1 including sialyl motifs III (Tyr354-Gln357), S (Pro321-Phe343), and VS (His370-Glu375) suggests that its helical bundle structure is sufficiently flexible to promote solubility while still allowing proper protein-glycan interactions that are necessary for native-like enzyme function.

### Example 4—Large-Scale Soluble Expression of Diverse GTs Using SIMPLEx Platform

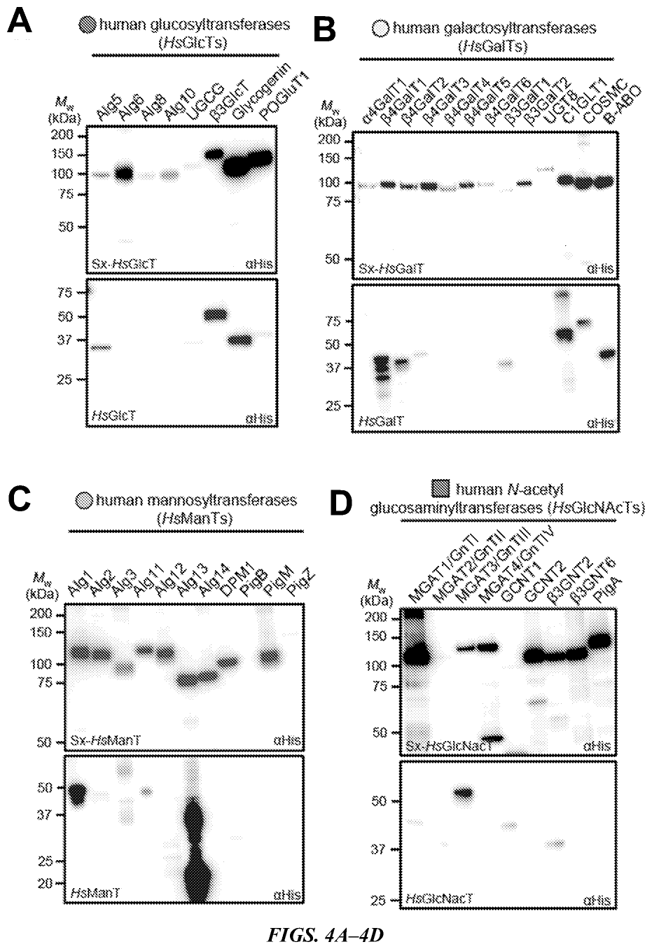

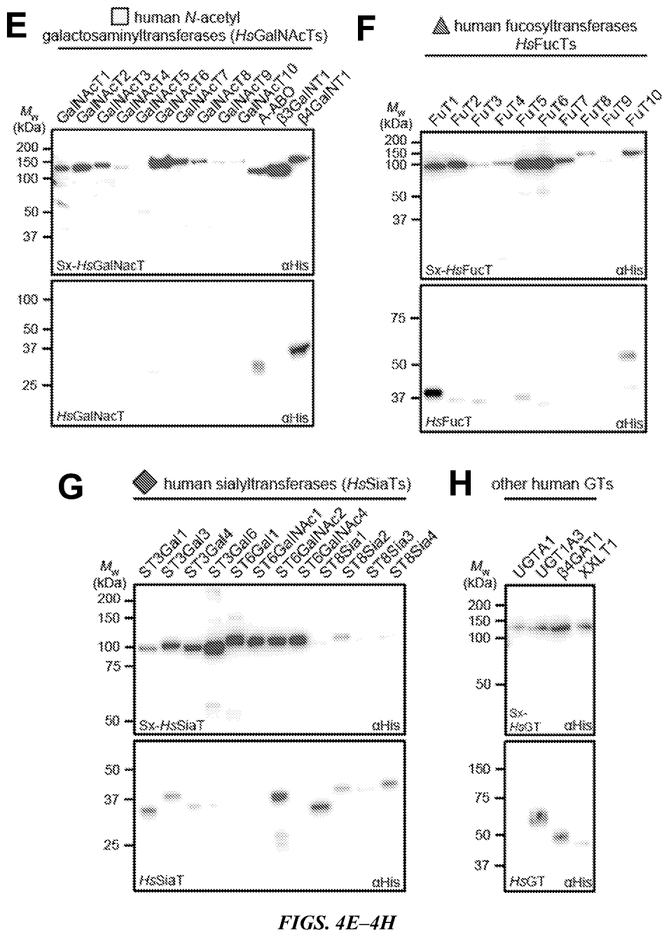

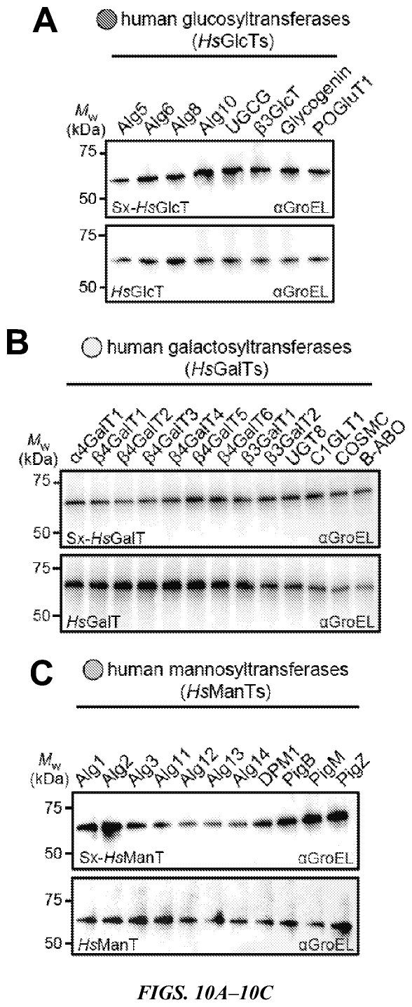

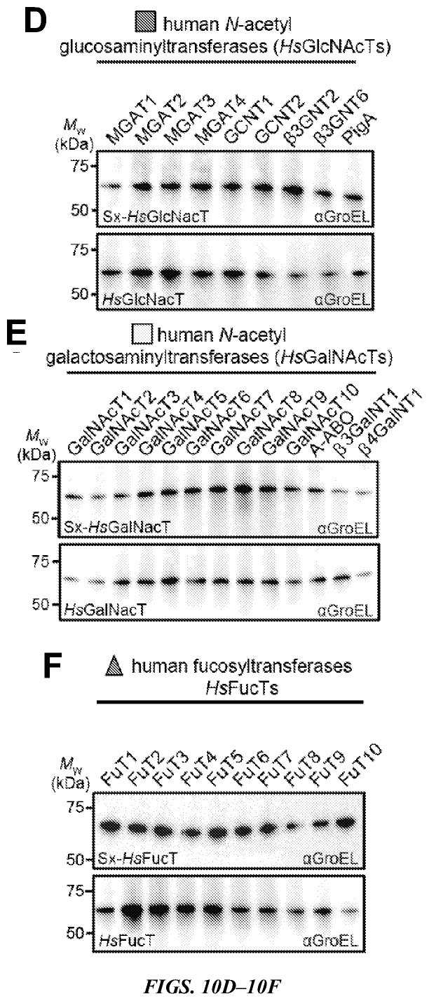

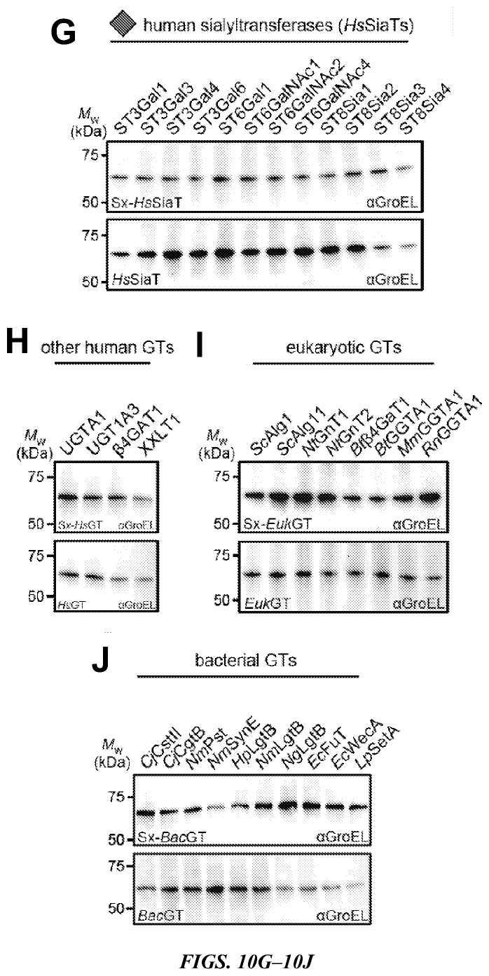

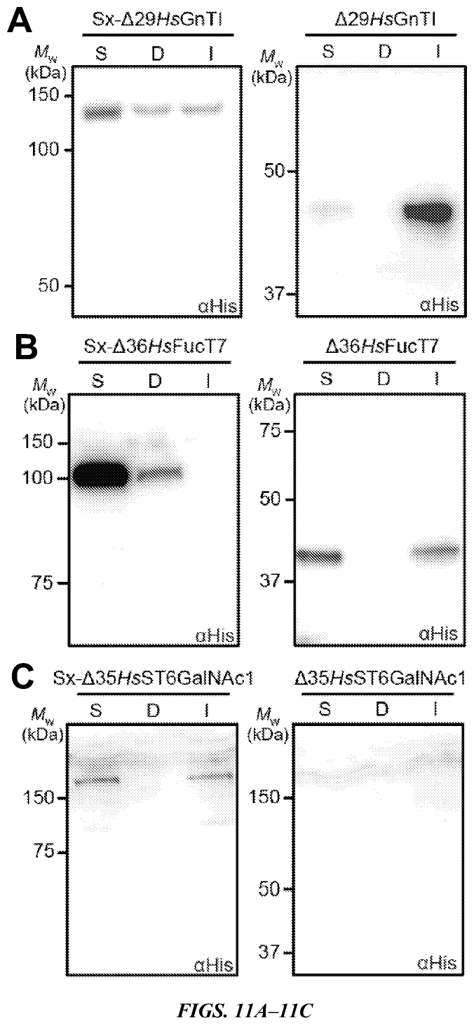

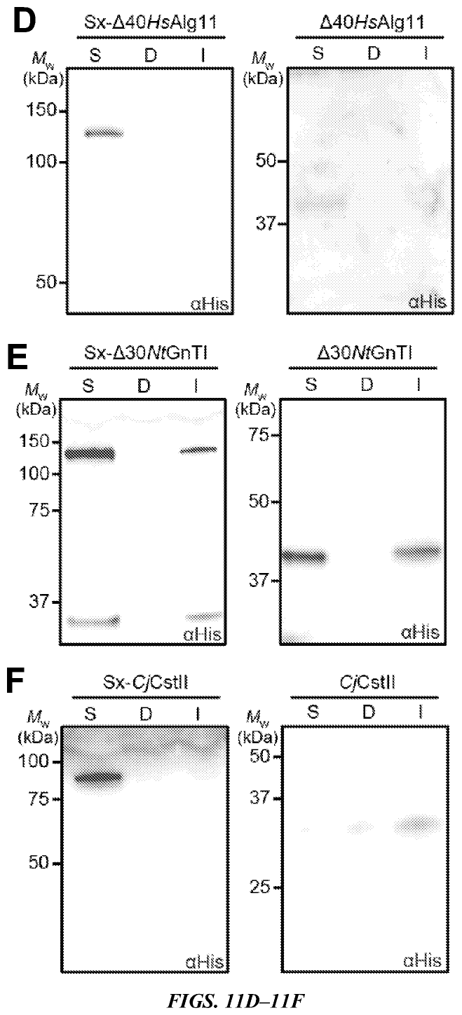

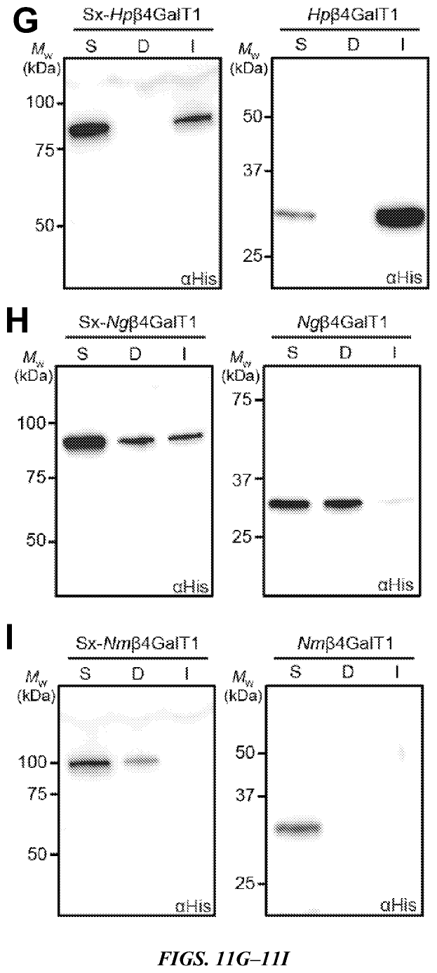

Encouraged by the ability of SIMPLEx to promote soluble expression of HsST6Gal1 in E. coli while preserving its biological activity, whether the strategy could be extended to a larger collection of structurally diverse GTs was next investigated. To this end, a library of 98 GT genes from diverse prokaryotic and eukaryotic organisms was compiled, with an emphasis placed on those of human origin (FIGS. 2A-2D). These genes were organized according to their species of origin and activity, and included the following: human fucosyltransferases (HsFucTs), human galactosyltransferases (HsGals), human glucosyltransferases (HsGlcTs), human mannosyltransferases (HsManTs), human N-acetylgalactosyltransferases (HsGalNAcTs), human N-acetylglucosaminyltransferases (HsGlcNAcTs), human sialyltransferases (HsSiaTs), and a collection of other human, eukaryotic, and prokaryotic GTs. Using the UniProt database (UniProt, C., “UniProt: The Universal Protein Knowledgebase in 2021,” Nucleic Acids Res. 49:D480-D489 (2021), which is hereby incorporated by reference in its entirety), these GTs were annotated based on the following characteristics: (i) single-pass transmembrane protein with C-terminus in cytoplasm (type I transmembrane protein); (ii) single-pass transmembrane protein with N-terminus in cytoplasm (type II transmembrane protein); (iii) multi-pass transmembrane protein; (iv) secretory protein with N-terminal signal peptide and C-terminal ER retention domain; and (v) cytosolic protein. It is known that N-/C-terminal TMDs as well as C-terminal ER retention domains in mammalian GTs are used as membrane anchors and are dispensable for catalytic activity (Harduin-Lepers et al., “The Human Sialyltransferase Family,” Biochimie 83727-83737 (2001), which is hereby incorporated by reference in its entirety), as was seen above for HsST6Gal1. Because SIMPLEx-mediated solubility enhancement of HsST6Gal1 was independent of whether the TMD was present or absent (FIG. 3B), these terminal TMD anchors were generally removed from the designed constructs. N-terminal signal peptides that natively route GTs to the secretory pathway were not necessary in the context of the disclosed bacterial cytoplasmic expression system and thus were also removed. GTs containing internal single-pass or multi-pass TMDs as well as predicted cytosolic GTs were designed as full-length genes. Each designed construct in the GT library (see FIGS. 2C-2D for amino acid sequences) was cloned into a T7 promoter-based expression vector as both a stand-alone GT (full-length or truncated) with C-terminal 6×His tag (hereafter GT) and a tripartite SIMPLEx fusion (hereafter Sx-GT). The expression of all Sx-GT constructs was tested in small-scale, batch-mode microbial cultures. SHuffle T7 Express cells were used to produce enzymes containing previously observed or predicted disulfide bonds while BL21(DE3) cells were used to express enzymes without such bonds (FIG. 2A). Cytoplasmic expression of the Sx-GTs was profiled by immunoblot analysis of clarified lysates derived from E. coli cells expressing the respective constructs. Importantly, 95 of the Sx-GT constructs showed clearly visible accumulation in the soluble cytoplasmic fractions, with most exhibiting moderate to strong expression and only a few that were faintly expressed (FIGS. 4A-4J and FIG. 10A-10J). It should be noted that this success rate was achieved under standard bacterial expression conditions (starting OD₆₀₀≈0.6, induction with 0.1 mM β-D-1-thiogalactopyranoside (IPTG) at 16° C. for 16-20 hours in LB medium) that were identical for each construct and did not require any of the lengthy optimization trials that are commonly associated with expression campaigns employing bacteria. Conversely, only ˜45% of the unfused GT constructs could be detected in the soluble fraction under the same conditions, and in most cases the level of soluble GT expression was visibly lower compared to its Sx-GT counterpart (FIGS. 4A-4J and FIGS. 10A-10J). Subcellular fractionation analysis of select 9 candidates revealed that all SIMPLEx constructs accumulated predominantly in the soluble fraction whereas unfused versions of the enzymes partitioned mostly in the insoluble or detergent-solubilized fractions (FIGS. 11A-11I), consistent with the solubility profiles observed above for Sx-HsST6Gal1 and Sx-Δ26HsST6Gal1. Additionally, aberrant expression products such as high-molecular-weight aggregates and proteolytic degradants were prevalently detected among the GT but not the Sx-GT constructs (FIGS. 4A-4J), highlighting the intrinsic ability of the SIMPLEx strategy to enhance intracellular stability and prevent off-pathway misfolding and aggregation of target enzymes.

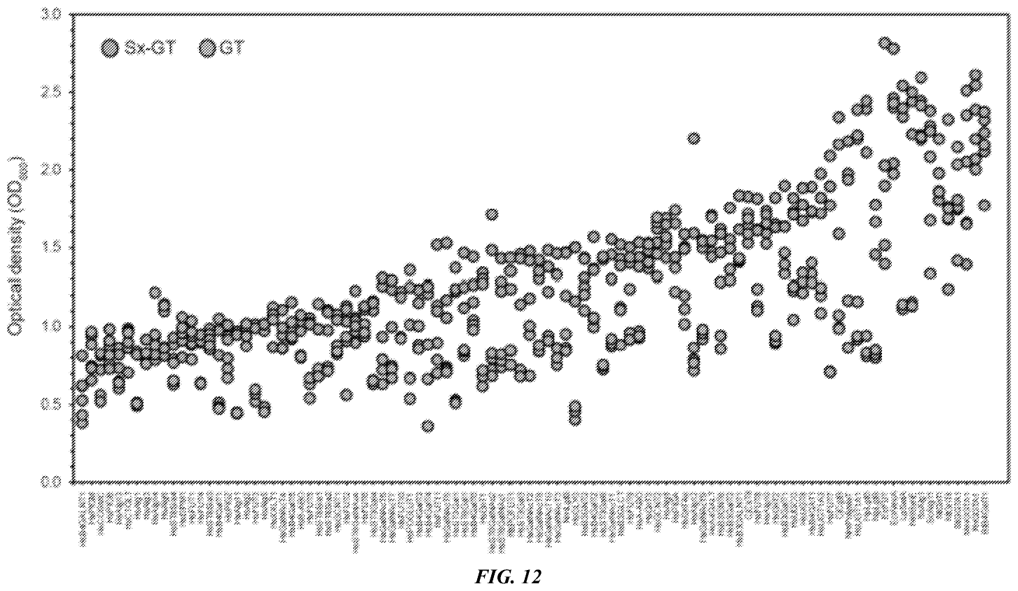

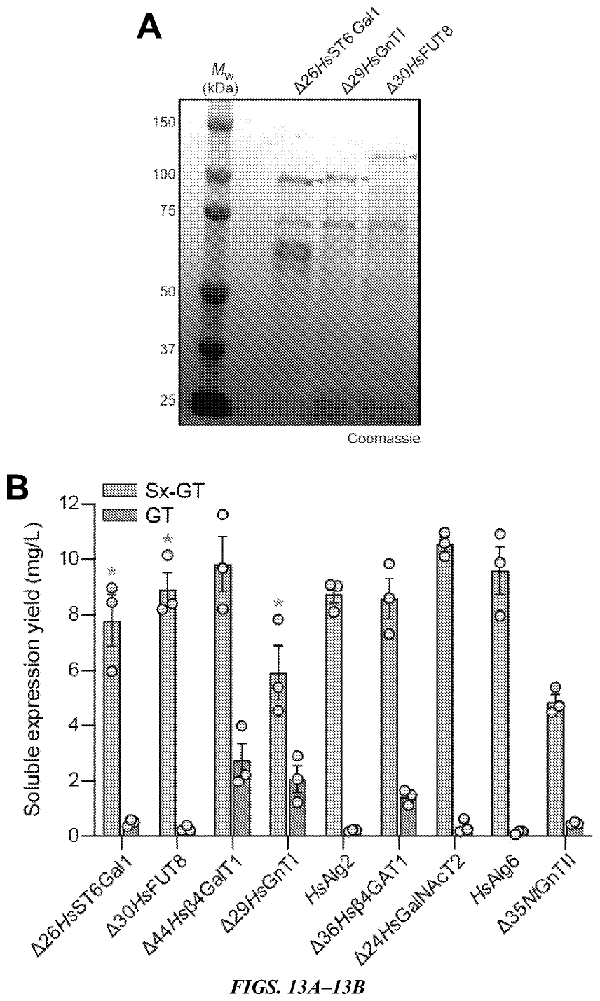

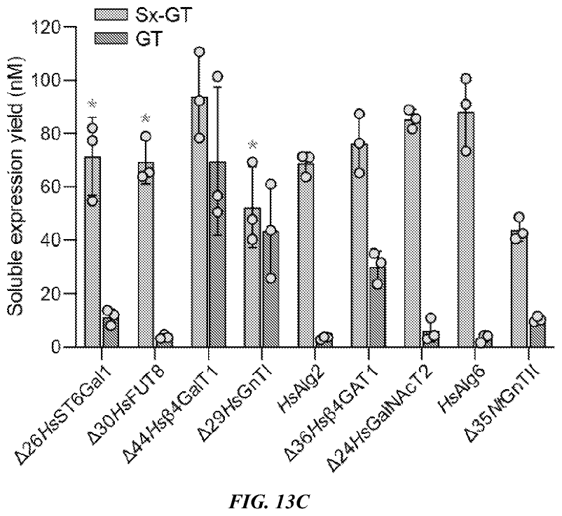

Another advantage of expressing GTs in the SIMPLEx framework is the potential to relieve cellular stress that arises from high-level accumulation of severely misfolded proteins (e.g., inclusion bodies) or destabilization of the cytoplasmic membrane caused by high-level expression of membrane proteins, phenomena that are both well-known to negatively impact cell growth and productivity. Indeed, cultures expressing Sx-GTs were consistently observed to reach higher final cell densities than those expressing unfused GTs (FIG. 12). Likewise, titers of selected Sx-GT candidates purified from 1-L cultures were also higher on both a mass and molar basis relative to unfused GTs, with all SIMPLEx constructs accumulating in the 5-10 mg/L range (FIGS. 13A-13C). Taken together, these results demonstrate (i) large-scale GT expression using E. coli as the host organism and (ii) SIMPLEx as a universal strategy for high-yield, soluble expression of GTs having diverse origins, structures, and activities.

### Example 5—Correlates of Successful GT Expression in E. coli

It was next sought to identify the protein features that correlated with soluble protein expression by comparing physicochemical properties of the proteins including molecular weight (M_{w}), isoelectric point (pI), and amino acid content. This involved assigning an expression score to each of the Sx-GT and GT constructs based on their soluble expression profiles (FIG. 14A). Next, expression scores were used to bin these proteins into four groups: non-expressor (score 0); weak expressor (score 1); medium expressor (score 2); and strong expressor (score 3). According to this classification, ˜95% of the Sx-GTs were identified as expressible (score ≥1), with more than 50% falling into the medium-to-high expressor groups. In stark contrast, over 50% of the GT constructs were identified as non expressors, with most of the others classifying as weak expressors (FIG. 14A). A scatter plot of protein M_{w}, excluding added mass from the ΔspMBP and ApoAI* domains, versus solubility score calculated by Protein-Sol, a web tool for predicting protein solubility from sequence (Hebditch et al., “Protein-Sol: A Web Tool for Predicting Protein Solubility From Sequence,” gBioinformatics 33:3098-3100 (2017), which is hereby incorporated by reference in its entirety), revealed that expressible Sx-GT constructs clustered within a 25-60 kDa range whereas expressible GT constructs were clustered in a narrower 25-40 kDa range that was skewed to smaller proteins (FIG. 14B).

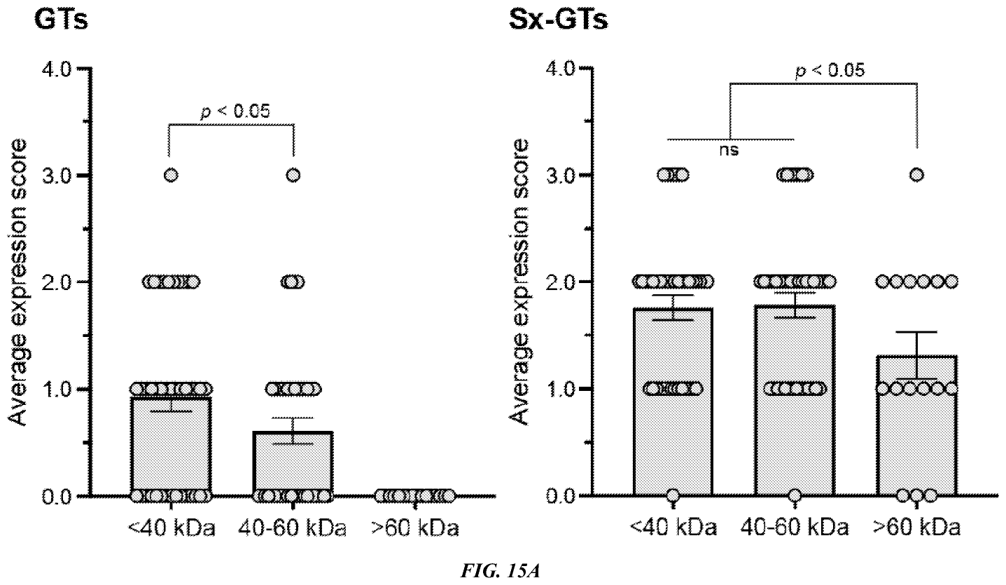

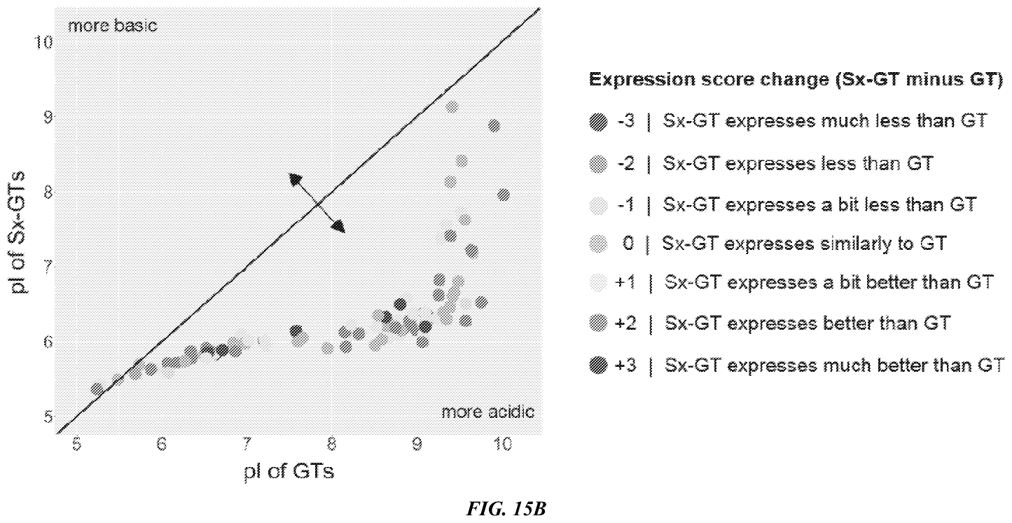

This observation prompted further investigation of the relationship between soluble expression of the protein and its M_{w}. To this end, all GTs were categorized into one of three size groups: small (M_{w}<40 kDa), medium (M_{w}=40-60 kDa), and large (M_{w}>60 kDa). The average expression score (E̅x̅) was then calculated for each size group within the Sx-GT or GT datasets. For GT constructs, a significant decrease in E̅x̅ was observed as protein M_{w} increased, with no soluble expression for large proteins (FIG. 15A), consistent with the observation that bacterial translation machinery has evolved to express shorter polypeptides (Netzer and Hartl, “Recombination of Protein Domains Facilitated by Co-translational Folding in Eukaryotes,” Nature 388:343-9 (1997), which is hereby incorporated by reference in its entirety) and that expression of larger eukaryotic proteins in bacteria frequently leads to misfolding and aggregation (Dyson et al., “Production of Soluble Mammalian Proteins in Escherichia coli: Identification of Protein Features That Correlate With Successful Expression,” BMC Biotechnol. 4:32 (2004) and Marston, F. A., “The Purification of Eukaryotic Polypeptides Synthesized in Escherichia coli,” Biochem. J. 240:1-12 (1986), which are hereby incorporated by reference in their entirety). On the contrary, E̅x̅ was high for all Sx-GT constructs, with no significant difference between small- and medium-sized proteins and only a small decrease in E̅x̅ for large-sized proteins (FIG. 15A). These results suggested that the SIMPLEx framework helps to overcome the protein size barrier that typically restricts successful expression in E. coli. Unfortunately, attempts to identify additional parameters such as protein pI and amino acid content that correlated with expressibility did not yield conclusive results (FIG. 14B and FIG. 15B). Nonetheless, the data presented here reveal important design parameters that could guide efforts to express even more GT enzymes in the future.

### Example 6—Efficient Production of Sx-GTs Across Diverse Expression Platforms

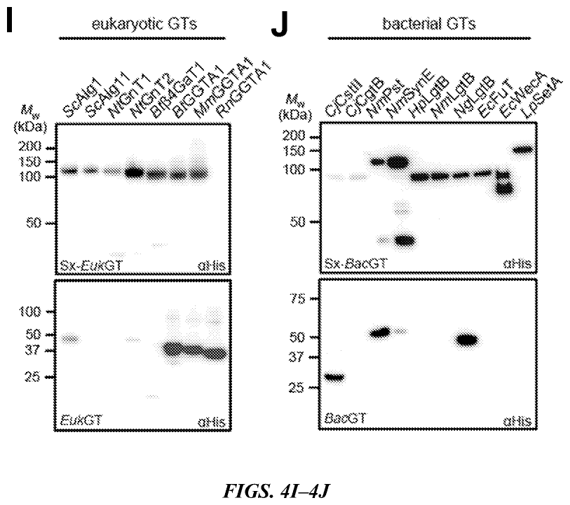

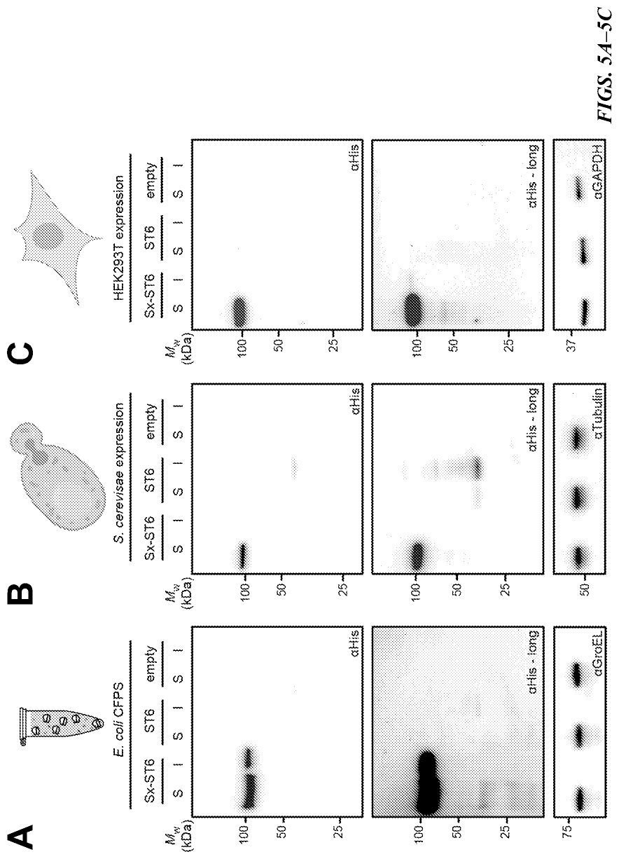

To further expand the utility of the platform and demonstrate its universality, SIMPLEx fusions in other popular expression platforms including: (i) E. coli-based cell-free protein synthesis (CFPS); (ii) Saccharomyces cerevisiae strain SBY49; and (iii) human embryonic kidney (HEK) 293T cells were produced. Using appropriate expression vectors for each system, significant accumulation of the Sx-Δ26HsST6Gal1 construct in the soluble fractions derived from each of these three systems was observed (FIGS. 5A-5C). In contrast, little to no soluble expression of the unfused Δ26HsST6Gal1 construct lacking the ΔspMBP and ApoAI* domains was detected in any of these systems (FIGS. 5A-5C). While Sx-Δ26HsST6Gal1 was also found in the insoluble fraction derived from the CFPS system, the amount of this construct that partitioned in the soluble fraction was significantly higher (FIG. 5A). For cell-based expression of Sx-Δ26HsST6Gal1, both yeast and human cells yielded products that accumulated almost exclusively in the soluble fractions (FIGS. 5B-5C), in line with the E. coli cell-based expression results observed above. Importantly, these results highlight the cross-platform compatibility of the SIMPLEx strategy and the ease with which it was adapted to these microbial, mammalian, and cell-free expression systems.

### Example 7—Cell-Free Construction of Free Human N-Glycans Using Sx-GTs

To date, a growing number of cell-free bio/chemoenzymatic synthesis strategies have been reported that provide access to large repertoires of pure and chemically-defined glycans, especially complex structures that are otherwise difficult to obtain by conventional chemical synthesis (Hamilton et al., “A Library of Chemically Defined Human N-glycans Synthesized From Microbial Oligosaccharide Precursors,” Sci. Rep. 7:15907 (2017); Li and Wang, “Chemoenzymatic Methods for the Synthesis of Glycoproteins,” Chem. Rev. 118:8359-8413 (2018); and Li et al., “Strategies for Chemoenzymatic Synthesis of Carbohydrates,” Carbohydr. Res. 472:86-97 (2019), which are hereby incorporated by reference in their entirety). Because these approaches generally depend on the availability of glycoenzymes, many of which cannot be recombinantly expressed or purified at scale, it was sought to demonstrate the practical utility of Sx-GTs as biocatalysts for constructing customized glycan structures via a previously described bioenzymatic synthesis approach (Hamilton et al., “A Library of Chemically Defined Human N-glycans Synthesized From Microbial Oligosaccharide Precursors,” Sci. Rep. 7:15907 (2017), which is hereby incorporated by reference in its entirety). To this end, two multi-GT enzyme pathways for de novo biosynthesis of a library of human hybrid- and complex-type N-glycans starting from a mannose₃-N-acetylglucosamine₂ (Man₃GlcNAc₂) primer were devised (FIG. 6A). To generate this primer, a glycoengineered E. coli strain carrying a heterologous biosynthesis pathway for producing undecaprenyl-linked Man₃GlcNAc₂ glycan was leveraged (Valderrama-Rincon et al., “An Engineered Eukaryotic Protein Glycosylation Pathway in Escherichia coli,” Nat. Chem. Biol. 8:434-6 (2012), which is hereby incorporated by reference in its entirety). Following glycolipid extraction from these cells, Man₃GlcNAc₂ (M3; glycan 1) was removed from undecaprenol by mild acid hydrolysis and purified to homogeneity as confirmed by matrix-assisted laser desorption/ionization-time of flight mass spectrometry (MALDI-TOF MS) analysis (FIG. 6B).

Using 1 as a primer, glycan elaboration with GlcNAc was carried out by sequential treatment with purified Sx-Δ29HsGnTI and Sx-Δ29HsGnTII, yielding hybrid-type glycan 2 (also known as G0-GlcNAc) and complex-type glycan 3 (G0), respectively, as evidenced by MALDI-TOF MS analysis of each reaction (FIG. 6B). Further elaboration of glycan 3 with galactose was achieved using Sx-Δ44Hsβ4GalT1 to generate glycan 4 (G2), which was subsequently elaborated using Sx-Δ26HsST6Gal1 to produce glycan 5 (G2S1) and glycan 6 (G2S2), the mono- and di-sialylated complex-type N-glycans, respectively (FIG. 6B). Alternatively, glycan 3 was first fucosylated using Sx-Δ30HsFucT8 to generate glycan 7 (G0F), which was then further elaborated to yield glycan 8 (G2F), glycan 9 (G2S1F), and glycan 10 (G2S2F) using a similar bioenzymatic strategy (FIG. 6B).

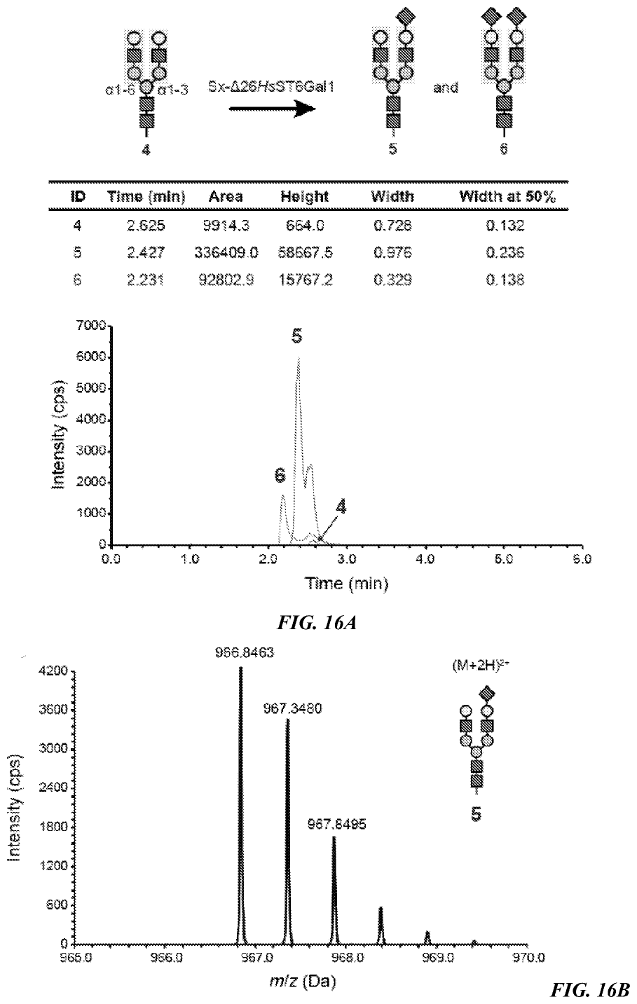

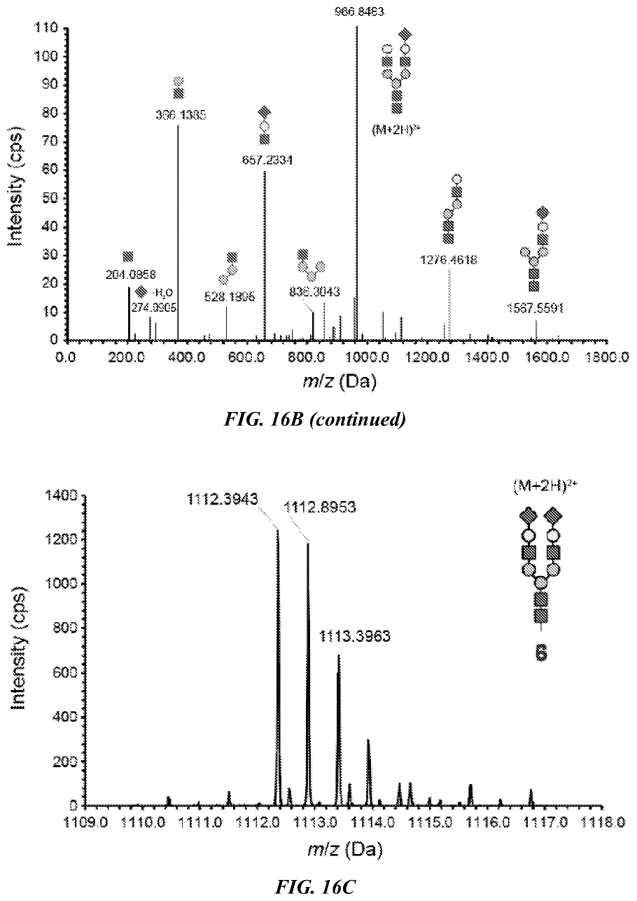

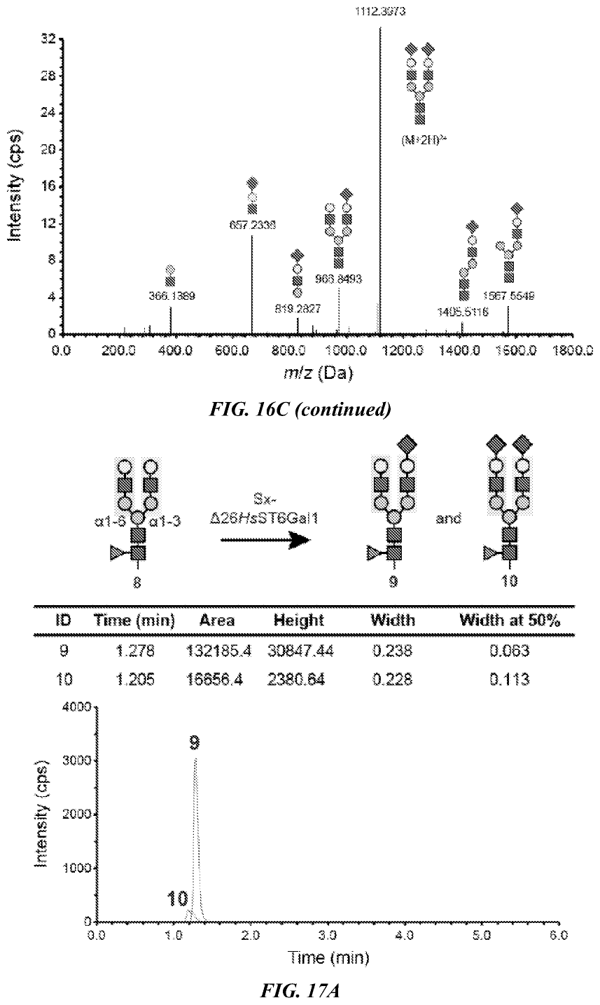

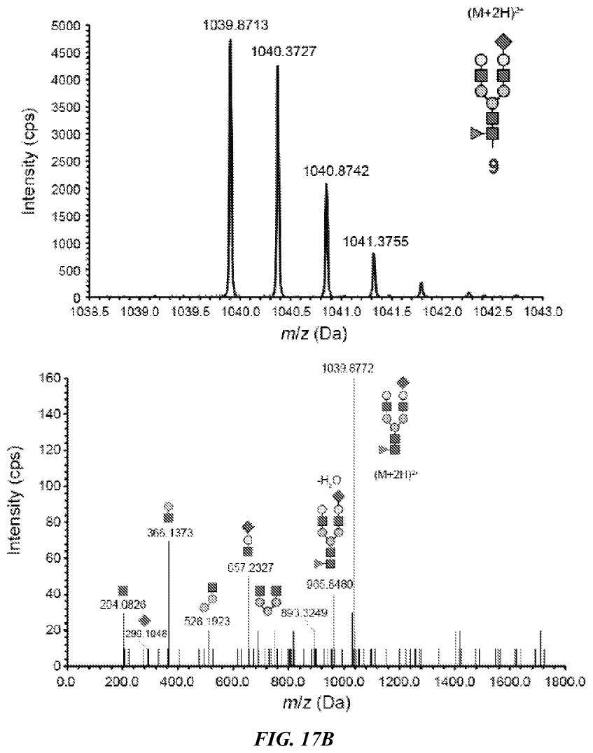

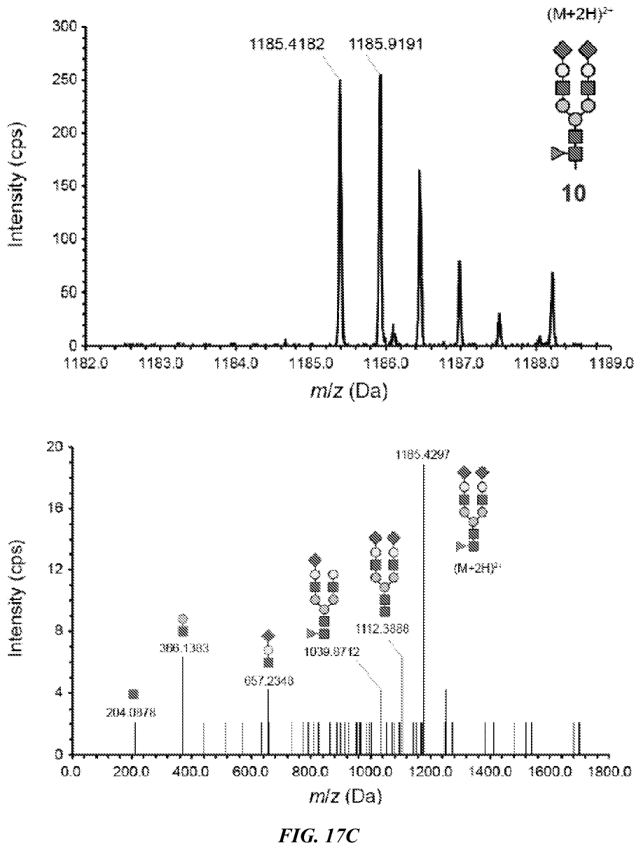

Overall, enzymatic conversion in each of these reactions was at or near 100% except in the cases involving the Sx-Δ26HsST6Gal1-catalyzed sialyation reactions. However, because the unstable nature of sialic acid-containing glycans in MALDI-TOF MS may have confounded the sialylation analysis, nano-scale reverse phase chromatography and tandem MS (nano LC-MS/MS) analysis were performed to confirm the abundance and identity of the sialylated glycans 5, 6, 9, and 10. While both mono- and di-sialylated products were clearly detected, this analysis revealed an approximate 5:1 ratio between the G2S1 and G2S2 glycans as well as the G2S1F and G2S2F glycans (FIGS. 16A-16C and FIGS. 17A-17C). It is worth pointing out that this phenomenon has been well documented (Barb et al., “Branch-specific Sialylation of IgG-Fc Glycans by ST6Gal-I,” Biochemistry 48:9705-7 (2009) and Tayi and Butler, “Solid-Phase Enzymatic Remodeling Produces High Yields of Single Glycoform Antibodies,” Biotechnol. J. 13:e1700381 (2018), which are hereby incorporated by reference in their entirety) and arises from the fact that human ST6Gal1 exhibits a preference for α1-3Man-β1,2-GlcNAc-β1,4-Gal (hereafter α1-3Man branch). As a result, ST6Gal1 readily installs Neu5Ac on this branch first, with subsequent sialyation of α1-6Man-β1,2-GlcNAc-β1,4-Gal (hereafter α1-6Man branch) known to be very slow (Barb et al., “Branch-specific Sialylation of IgG-Fc Glycans by ST6Gal-I,” Biochemistry 48:9705-7 (2009), which is hereby incorporated by reference in its entirety).

### Example 8—Cell-Free Remodeling of Protein-Linked N- and O-Glycans Using Sx-GTs

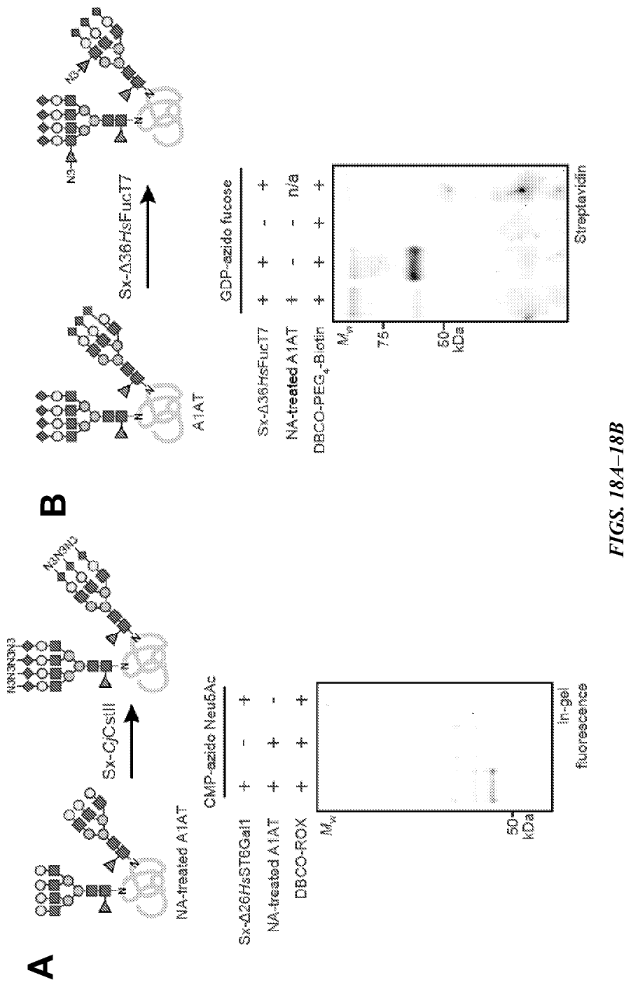

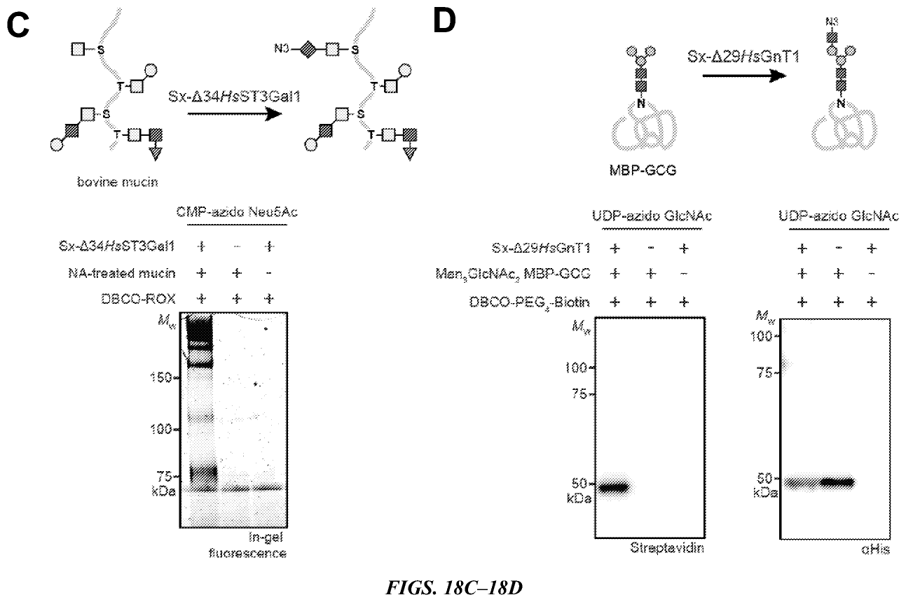

Glycoform manipulation is an emerging strategy for improving pharmacokinetics and pharmacodynamics of therapeutic glycoproteins (Wang et al., “Glycoengineering of Antibodies for Modulating Functions,” Annu. Rev. Biochem. 88:433-459 (2019) and Wang and Lomino, “Emerging Technologies for Making Glycan-defined Glycoproteins,” ACS Chem. Biol. 7:110-22 (2012), which are hereby incorporated by reference in their entirety). The remodeling of protein-linked glycans can be readily achieved using one or more GTs; however, the limited availability of requisite enzymes for customizing glycan structures represents a barrier to widespread adoption. To address this technology gap, members from the disclosed library of SIMPLEx-reformatted GTs were employed to alter the glycan profiles on several biomedically-relevant glycoproteins. Remodeling reactions included: (i) Sx-CjCstII-mediated α2,3-sialylation of the N-glycoforms on α₁-antitrypsin (A1AT), a serpin used in prophylactic treatment of the genetic disorder α₁-antitrypsin deficiency; (ii) Sx-Δ36HsFucT7-mediated fucosylation of the N-glycoforms on A1AT; (iii) Sx-Δ34HsST3Gal1-mediated α2,3-sialylation of the O-glycoforms on bovine submaxillary mucin (BSM), a glycoprotein with potential uses as a biocompatible material and drug delivery vehicle; and (iv) Sx-Δ29HsGnTI-catalyzed GlcNAc transfer onto Man₃GlcNAc₂ glycans present on a neoglycoprotein variant of human glucagon (GCG). In all cases, Sx-GTs readily remodeled their glycoprotein substrates, installing respective monosaccharides in 1-hour reactions that were monitored using bioorthogonal click chemistry-based assays with either a fluorophore or biotin reporter for glycan labeling (FIGS. 18A-18D). It should be noted that significantly decreased activity was observed for Sx-Δ36HsFucT7 when the N-glycans on A1AT were pre-treated with neuraminidase to remove native Neu5Ac residues. This observation was in line with earlier reports (Tu et al., “Development of Fucosyltransferase and Fucosidase Inhibitors,” Chem Soc Rev 42:4459-75 (2013), which is hereby incorporated by reference in its entirety) and highlights how subtle differences in substrate specificity can be directly investigated using GTs within the SIMPLEx framework.

### Example 9—Remodeling IgG N-Glycans Using Sx-GTs

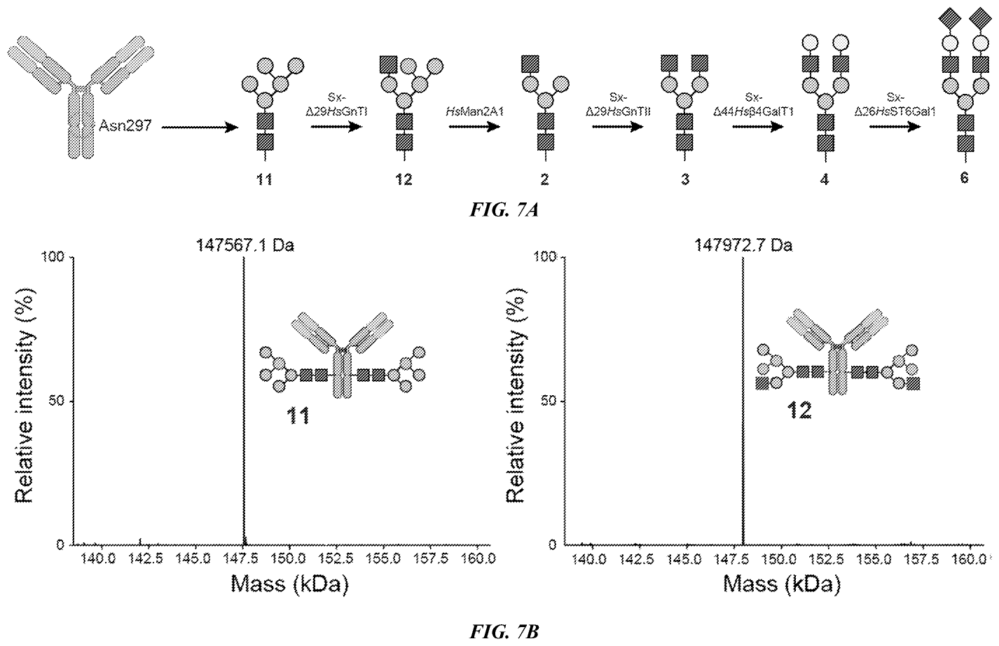

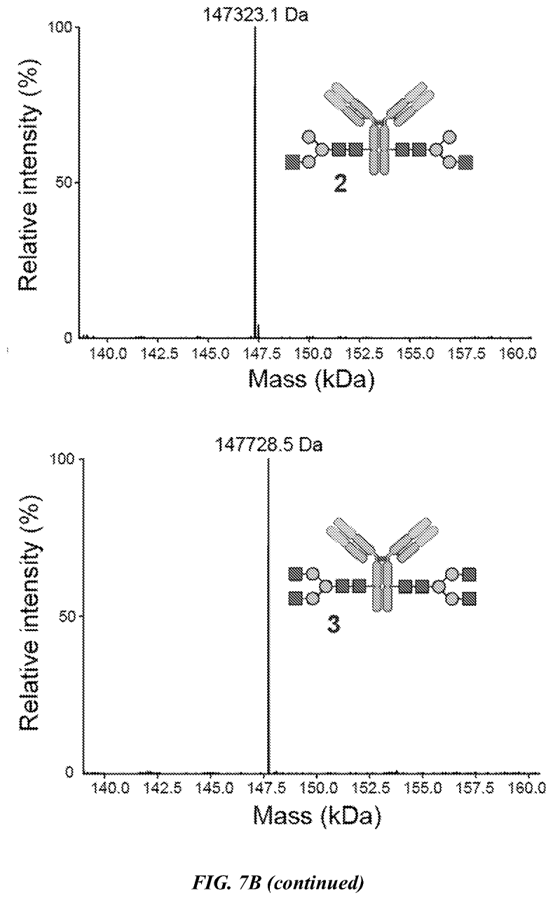

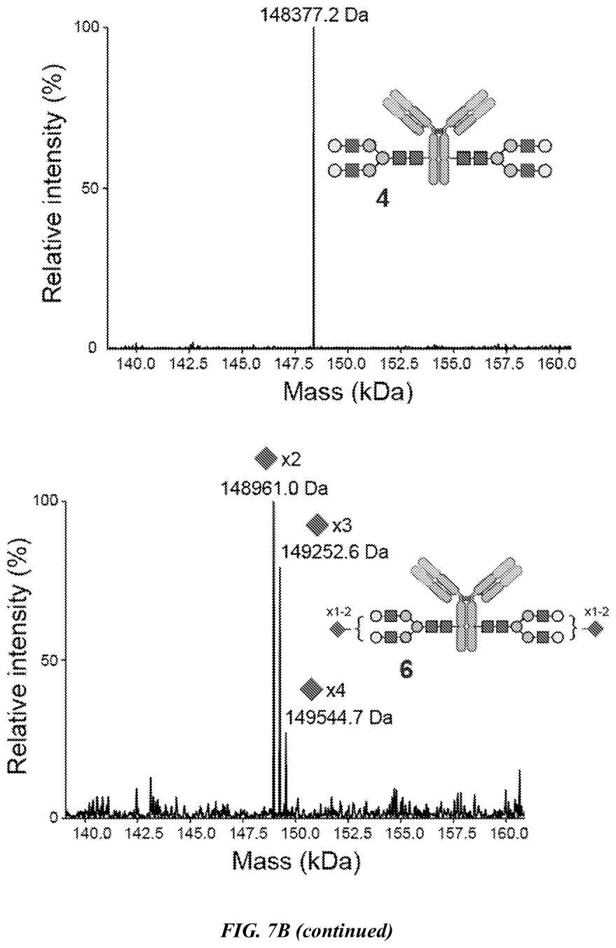

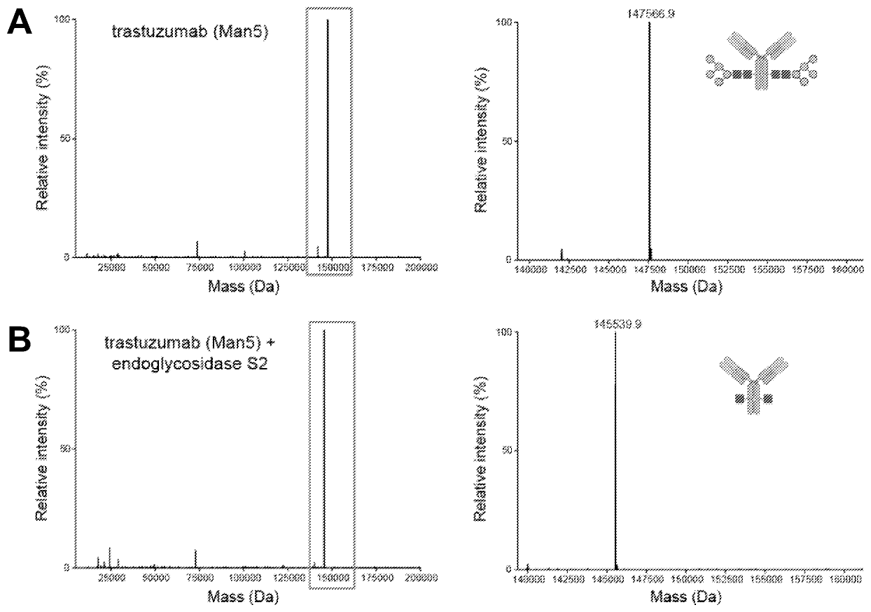

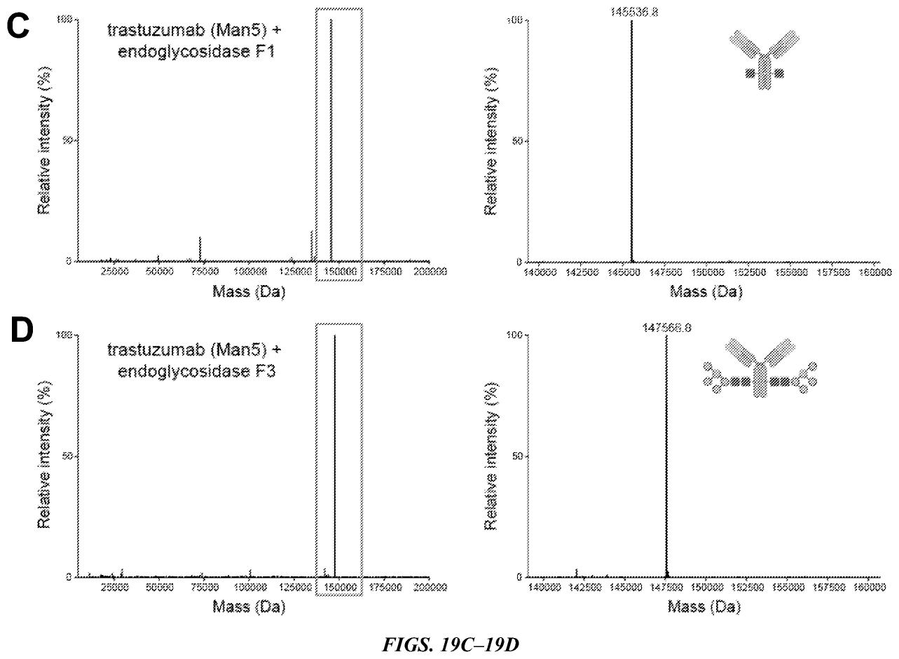

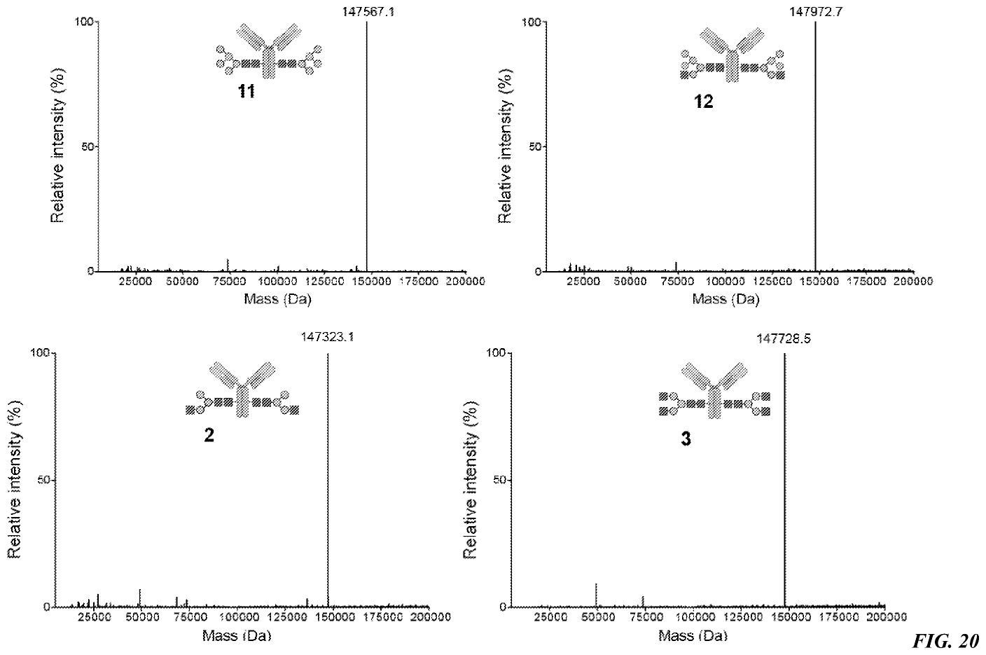

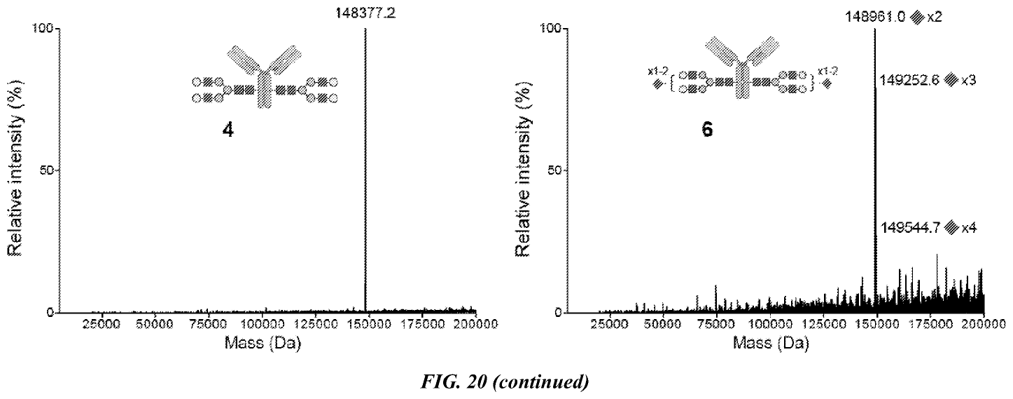

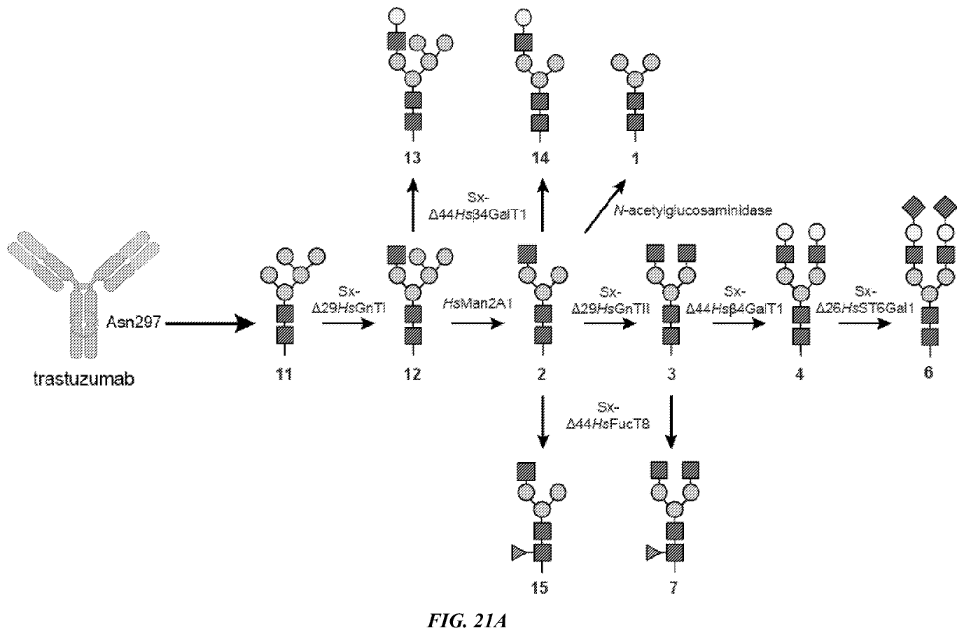

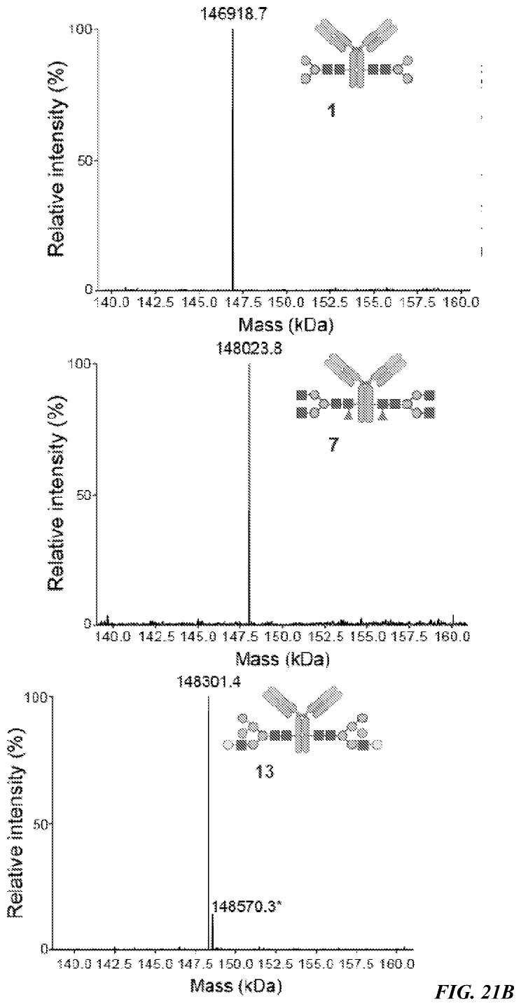

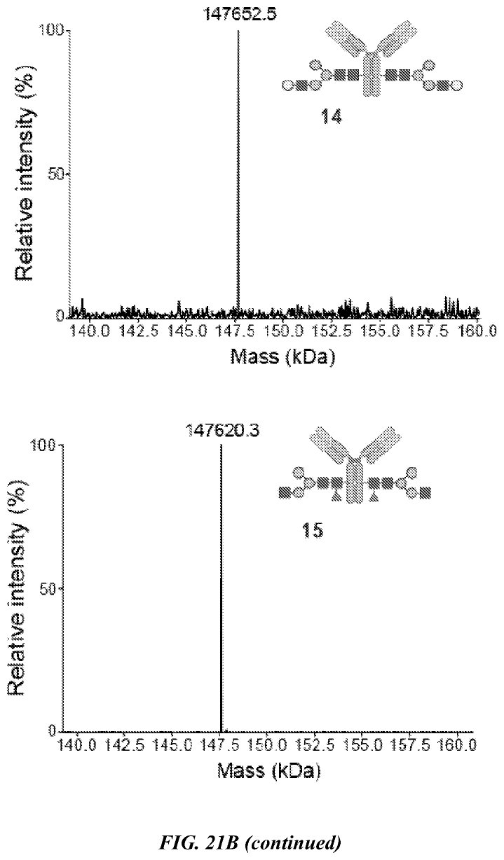

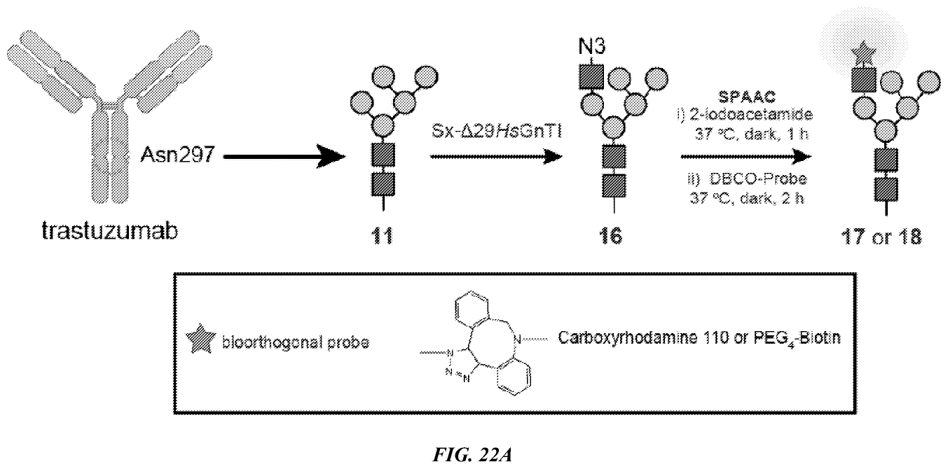

N-glycans present on the Fc domain of IgG antibodies play a critical role in the structure and function of these important proteins, but understanding of how discrete glycan structures affect IgG behavior remains limited due to naturally occurring microheterogeneity. Hence, strategies for generating structurally-defined N-glycans on IgG-Fc are expected to improve the understanding of the roles played by these structures in human immunity and to open the door to creating better medicines through glycoengineering. To this end, members from the disclosed library of Sx-GTs were leveraged to generate a homogenously glycosylated variant of trastuzumab (FIG. 7A), an anti-human epidermal growth factor receptor 2 (HER2) mAb used to treat HER2-positive breast, gastroesophageal, and gastric cancers. This involved first preparing trastuzumab using a glycoengineered cell line, Expi293F™ GnTI, that homogeneously produces N-glycoproteins bearing Man₅GlcNAc₂ glycans (FIG. 7A, glycan 11). Using a glycosidase sensitivity assay coupled with LC-MS analysis of the intact antibody, it was confirmed that the N-glycans on trastuzumab derived from Expi293F™ GnTI⁻ were indeed Man₅GlcNAc₂ glycans (FIGS. 19A-19D). Next, Sx-Δ29HsGnTI was used to install GlcNAc on the α1,3-man branch of 11 to generate GlcNAcMan₅GlcNAc₂ glycan (glycan 12) directly on trastuzumab (FIG. 7B). The two terminal Man residues on the α1,6-man branch of 12 were then removed using human Golgi Man2A1 (HsMan2A1), yielding trastuzumab bearing glycan 2. Subsequent cell-free glycan remodeling reactions using Sx-Δ29HsGnTII and Sx-Δ44Hsβ4GalT1 furnished trastuzumab with glycans 3 and 4, respectively. Finally, Sx-Δ26HsST6Gal1 was used to cap glycan 4 with Neu5Ac, efficiently generating glycans 5 and, to a lesser extent, glycan 6 (FIG. 7B). Additional N-glycan structures including paucimannose (glycan 1), hybrid (glycan 13, 14), and complex (glycan 7, 15) types were also prepared directly on trastuzumab IgG-Fc using a variety of Sx-GTs (FIGS. 21A-21B). In most cases, glycan remodeling efficiency was near 100% following incubation with Sx-GTs for 16-36 hours at 37° C. with approximately 80-90% recovery yield from purification between each step. Only the conversion to the disialylated N-glycan using Sx-Δ26HsST6Gal1 resulted in notably lower efficiency. While the reason for this inefficiency is unclear, it likely results from human ST6Gal1's known preference for the α1-3Man branch (Barb et al., “Branch-specific Sialylation of IgG-Fc Glycans by ST6Gal-I,” Biochemistry 48:9705-7 (2009), which is hereby incorporated by reference in its entirety).

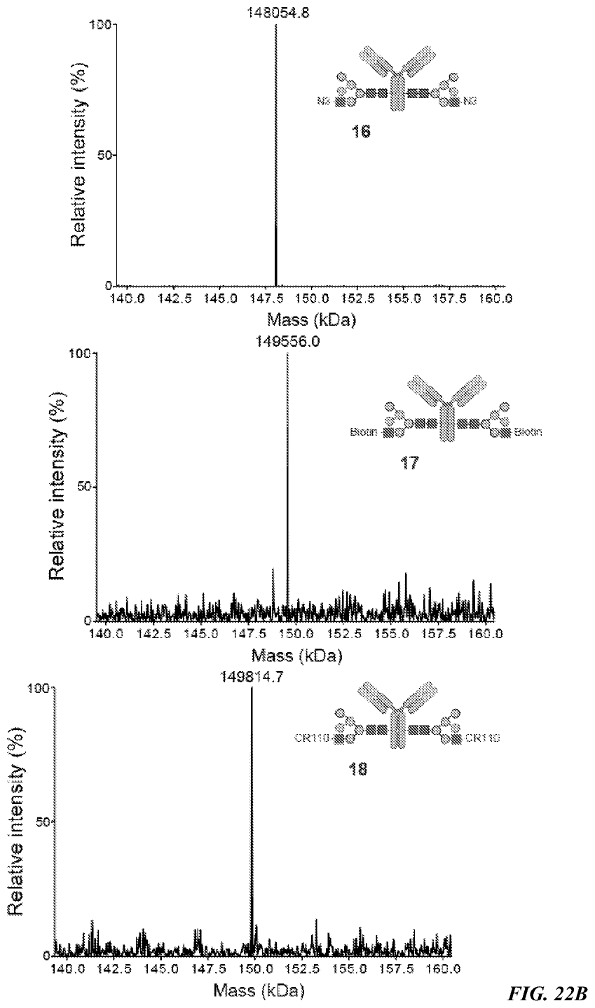

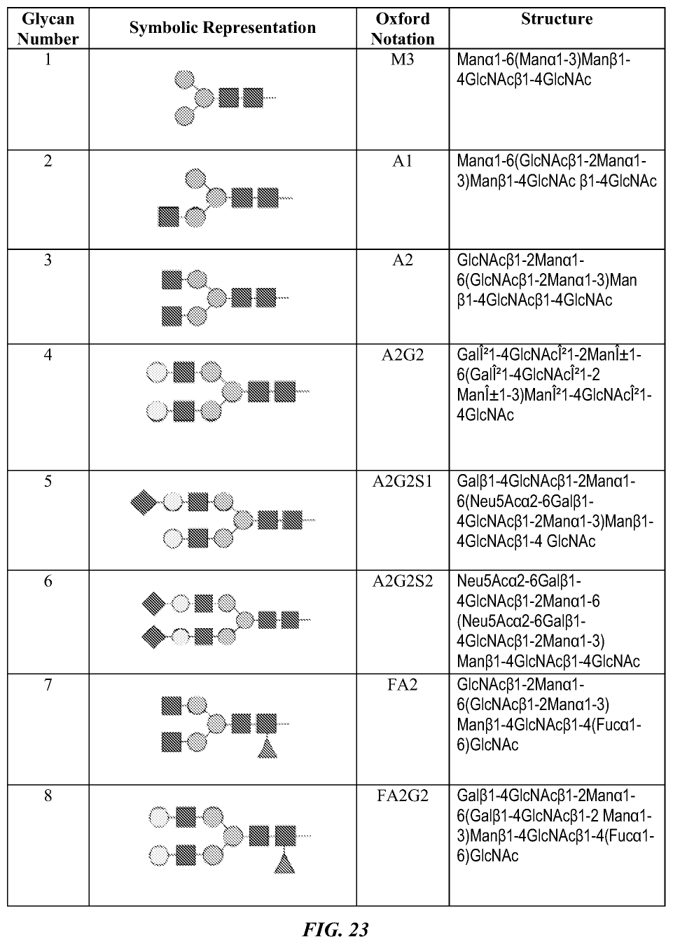

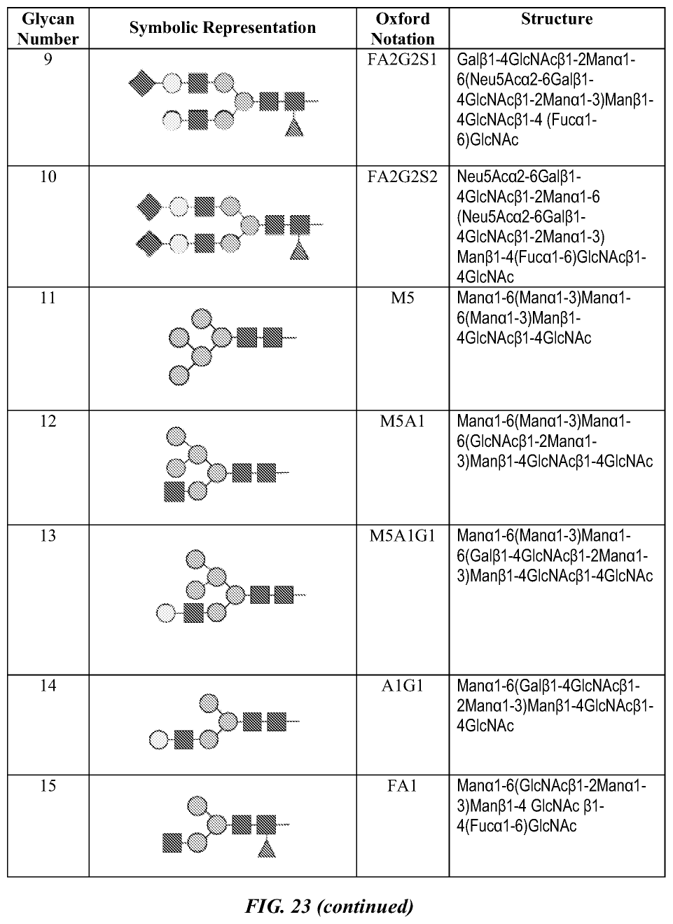

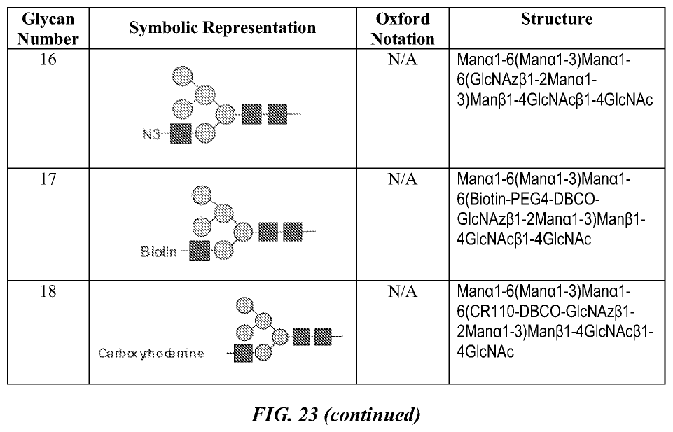

In addition to producing authentic, homogeneous human N-glycans, whether Sx-GTs could generate IgG-Fc bearing unnatural glycan structures was also investigated. To this end, Sx-Δ29HsGnTI was used to elaborate trastuzumab N-glycans with N-azidoacetylglucosamine (GlcNAz), a synthetic monosaccharide containing an azide moiety (FIGS. 22A-22B, glycan 13) that served as a versatile chemical handle for regiospecific conjugation via bioorthogonal click chemistry. Indeed, it was possible to site-specifically modify this handle on trastuzumab with either a biotin group (glycan 17) or a fluorescent reporter (glycan 18), thereby providing a convenient route for extending the functional utility of Fc domain-linked glycans. Collectively, these results highlight the biocatalytic potential of SIMPLEx glycoenzymes in the construction of homogeneous glycans as both free and protein-linked structures and effectively paves the way for accelerating protein glycosylation studies as well as tailoring the biological, biophysical, and biomedical properties of glycoproteins.

### Discussion of Examples 1-9

Examples 1-9 describe the creation of a universal expression platform for producing nearly 100 different GTs, predominantly of human origin, at relatively high titers (approximately 5-10 mg/L) using standard bacterial culture. This platform leverages SIMPLEx to engineer GT chimeras that are rendered highly soluble in the cytoplasm of E. coli cells. Consistent with earlier works (Mizrachi et al., “Making Water-soluble Integral Membrane Proteins In Vivo Using an Amphipathic Protein Fusion Strategy,” Nat. Commun. 6:6826 (2015) and Mizrachi et al., “A Water-soluble DsbB Variant That Catalyzes Disulfide-bond Formation In Vivo,” Nat. Chem. Biol. 13:1022-1028 (2017), which are hereby incorporated by reference in their entirety), SIMPLEx-reformatted GTs retained biological activity as exemplified by the human ST6Gal1 chimera that exhibited activity that was similar to a commercially sourced enzyme. The ability to solubilize such a large set of GTs without compromising function made it possible to remodel the structures of different free and protein-linked glycans including those found on the monoclonal antibody trastuzumab. Overall, the platform described infra represents a versatile addition to the synthetic glycobiology toolkit, providing easy access to a vast collection of transformative reagents that are expected to find use in structure-function studies of GTs and to fuel myriad applications where complex glycomolecules are featured.

Previous studies revealed the capacity of SIMPLEx to broadly transform all major classes of IMPs into water-soluble molecules (Mizrachi et al., “Making Water-soluble Integral Membrane Proteins In Vivo Using an Amphipathic Protein Fusion Strategy,” Nat. Commun. 6:6826 (2015) and Mizrachi et al., “A Water-soluble DsbB Variant That Catalyzes Disulfide-bond Formation In Vivo,” Nat. Chem. Biol. 13:1022-1028 (2017), which are hereby incorporated by reference in their entirety). These IMPs included proteins having both bitopic and polytopic α-helical structures such as glutamate receptor (GluA2) and bacteriorhodopsin (bR) as well as polytopic β-barrel structures such as voltage-dependent anion channel 1 (VDAC1). Here, this solubilization capacity was broadened to include polytopic α-helical GTs with multiple TMDs such as found in human mannosyltransferases Alg2, Alg3, and Alg12 and human glucosyltransferases Alg6, Alg8, and Alg10 as well as monotopic α-helical GTs with single-pass internal TMDs that could not be easily removed such as Alg2 and PigA. For these complex integral membrane proteins, introduction of an N-terminal decoy protein, MBP, prevented co-translational insertion of the polypeptide into the inner membrane through the signal recognition particle (SRP) pathway (Luirink and Sinning, “SRP-mediated Protein Targeting: Structure and Function Revisited,” Biochim. Biophys. Acta. 1694:17-35 (2004), which is hereby incorporated by reference in its entirety) while the amphipathic ApoAI* domain effectively shielded the hydrophobic TMDs from the aqueous environment.

It is noteworthy that most of the GTs investigated (72 out of 98 total) were simpler type II transmembrane proteins. Type II GTs such as HsST6Gal1 possess just a single-pass TMD at their N- or C-termini (FIG. 3A), which is generally not required for activity and is thus commonly removed during expression campaigns (Moremen Et Al., “Expression System For Structural And Functional studies of Human Glycosylation Enzymes,” Nat. Chem. Biol. 14:156-162 (2018), which is hereby incorporated by reference in its entirety), which is hereby incorporated by reference in its entirety). Hence, while the rationale for using SIMPLEx with full-length GTs including their TMDs was clear, it was not obvious that this solubilization method would benefit type II GTs lacking a TMD altogether. That said, removal of GTs from their transmembrane contexts and the lack of native interacting/folding partners can create difficulties in folding upon expression in heterologous hosts. Indeed, many N-terminally truncated GTs accumulated exclusively in the insoluble or detergent-solubilized fractions of E. coli cells, in agreement with numerous previous reports involving expression of truncated GTs in bacteria. One possible reason for this poor expression is that GTs contain several moderately hydrophobic segments including around the stem region, just after the TMD, that can trigger unwanted membrane targeting or otherwise drive misfolding and aggregation. To circumvent this issue, fusion of solubility-enhancing partners such as MBP to the N-termini of truncated GTs is often obligatory, even in mammalian cells where GTs fused with GFP expressed significantly better than unfused versions of the same glycoenzymes (Moremen Et Al., “Expression System For Structural And Functional studies of Human Glycosylation Enzymes,” Nat. Chem. Biol. 14:156-162 (2018), which is hereby incorporated by reference in its entirety). However, introduction of fusion tags does not always lead to immediate success in terms of soluble GT expression and thus often requires lengthy optimization of growth and induction conditions as well as trial-and-error evaluation of different host strains and accessory factor (e.g., molecular chaperone) co-expression strategies (Skretas et al., “Expression of Active Human Sialyltransferase ST6GalNAcI in Escherichia coli,” Microb Cell Fact 8:50 (2009); Ortiz-Soto and Seibel, “Expression of Functional Human Sialyltransferases ST3Gal1 and ST6Gal1 in Escherichia coli,” PLoS One 11:e0155410 (2016); and Hidari et al., “Purification and Characterization of a Soluble Recombinant Human ST6Gal I Functionally Expressed in Escherichia coli,” Glycoconj J 22:1-11 (2005), which are hereby incorporated by reference in their entirety). Even when appreciable solubility is achieved, it is quite common for the resulting MBP fusions to accumulate as soluble but heterogeneous multimeric aggregates in which only a small fraction of the fusion protein is properly folded and active (Skretas et al., “Expression of Active Human Sialyltransferase ST6GalNAcI in Escherichia coli,” Microb Cell Fact 8:50 (2009); Ortiz-Soto and Seibel, “Expression of Functional Human Sialyltransferases ST3Gal1 and ST6Gal1 in Escherichia coli,” PLoS One 11:e0155410 (2016); and Pasek et al., “Galectin-1 as a Fusion Partner for the Production of Soluble and Folded Human Beta-1,4-galactosyltransferase-T7 in E. coli,” Biochem Biophys Res Commun 394:679-84 (2010), which are hereby incorporated by reference in their entirety). Along these lines, >50% less activity was observed for MBP-tagged Δ26HsST6Gal1 (lacking the native TMD) compared to its SIMPLEx counterpart, underscoring the essential contribution made by the ApoAI* domain in promoting solubility of a type II GT in the E. coli cytoplasm. It is therefore hypothesized that the amphipathic nature of ApoAI*, when expressed in the proximity of exposed hydrophobic patches in type II GT proteins, provides a stabilizing effect via hydrophobic interaction, akin to how ApoAI-based nanodiscs solubilize membrane proteins in solution (Grinkova et al., “Engineering Extended Membrane Scaffold Proteins for Self-assembly of Soluble Nanoscale Lipid Bilayers,” Protein Eng. Des. Sel. 23:843-8 (2010), which is hereby incorporated by reference in its entirety).

Importantly, the SIMPLEx architecture enabled soluble expression for nearly 100 GTs (>95% “hit” rate) under standard, identically matched conditions without any optimization, thereby offering a universal solution to GT production in E. coli that has not been possible with stand-alone fusion tags such as MBP or other expression optimization techniques (Wagner et al., “Rationalizing Membrane Protein Overexpression,” Trends Biotechnol. 24:364-71 (2006), which is hereby incorporated by reference in its entirety). An additional layer of universality stems from the compatibility of SIMPLEx-mediated GT solubilization with other commonly used expression hosts such as yeast and HEK293 cells as well as with E. coli-based cell-free protein synthesis (CFPS). Such platform flexibility is significant for several reasons. For one, each of these platforms is amenable to high-throughput profiling of protein expression and production that can be scaled up to larger volumes (Subedi et al., “High Yield Expression of Recombinant Human Proteins with the Transient Transfection of HEK293 Cells in Suspension,” J. Vis. Exp. e53568 (2015) and Spirin, A. S., “High-throughput Cell-free Systems for Synthesis of Functionally Active Proteins,” Trends Biotechnol. 22:538-45 (2004), which are hereby incorporated by reference in their entirety). Moreover, in the case of yeast and HEK293, the compatibility of SIMPLEx-reformatted GTs in these well-established eukaryotic hosts may provide access to protein folding networks and post-translational modifications including N- and O-linked glycosylation that may be important for the biological function of a subset of GTs (Mikolajczyk et al., “How Glycosylation Affects Glycosylation: The Role of N-glycans in Glycosyltransferase Activity,” Glycobiology 30:941-969 (2020), which is hereby incorporated by reference in its entirety) but are natively lacking in standard E. coli strains. In the case of E. coli-based CFPS, the “open” nature and multiplexability of these systems, combined with their speed and simplicity, should provide opportunities for high-throughput screening of GT function (Kightlinger et al., “Design of Glycosylation Sites by Rapid Synthesis and Analysis of Glycosyltransferases,” Nat. Chem. Biol. 14(6):627-635 (2018), which is hereby incorporated by reference in its entirety) as well as rapid discovery, prototyping, and optimization of glycomolecule synthesis pathways (Karim and Jewett, “A Cell-free Framework for Rapid Biosynthetic Pathway Prototyping and Enzyme Discovery,” Metab. Eng. 36:116-126 (2016) and Kightlinger et al., “A Cell-free Biosynthesis Platform for Modular Construction of Protein Glycosylation Pathways,” Nat. Commun. 10:5404 (2019), which are hereby incorporated by reference in their entirety).

As proof of concept for the utility of the disclosed SIMPLEx pipeline, some of the solubilized products were used in coordinated cell-free reaction networks to catalyze the formation of chemically-defined N-glycans. In one instance, it was possible to transform quantitative amounts of a simple paucimannose precursor N-glycan, Man₃GlcNAc₂ derived from glycoengineered E. coli (Valderrama-Rincon et al., “An Engineered Eukaryotic Protein Glycosylation Pathway in Escherichia coli,” Nat. Chem. Biol. 8:434-6 (2012) and Glasscock et al., “A Flow Cytometric Approach to Engineering Escherichia coli for Improved Eukaryotic Protein Glycosylation,” Metab. Eng. 47:488-495 (2018), which are hereby incorporated by reference in their entirety), into complex biantennary N-glycans including those containing core-fucose and sialic acid caps using a set of SIMPLEx-reformatted GTs. This workflow to efficiently generate a library of complex N-glycans, starting from expression and purification and then finally utilization of SIMPLEx-reformatted GTs, could be completed in less than one week. Using an identical strategy, it was possible to generate a spectrum of homogenous N-glycan structures on intact glycoproteins including trastuzumab, a mAb therapy used to treat breast and stomach cancers. Akin to earlier engineering of an artificial cytoplasmic disulfide formation pathway involving a water-soluble SIMPLEx variant of DsbB (Mizrachi et al., “A Water-soluble DsbB Variant That Catalyzes Disulfide-bond Formation In Vivo,” Nat. Chem. Biol. 13:1022-1028 (2017), which is hereby incorporated by reference in its entirety), ensembles of SIMPLEx-reformatted GTs could similarly be assembled into designer pathways, either in vitro or in living cells, for the on-demand biosynthesis of important glycans and glycoconjugates. Looking forward, it is anticipated that the constructs, expression systems, and workflows for glycoenzyme production described herein will find widespread use by those seeking to push the boundaries of our knowledge of glycobiology and glycochemistry and its application in health, energy, and materials science.

Although preferred embodiments have been depicted and described in detail herein, it will be apparent to those skilled in the relevant art that various modifications, additions, substitutions, and the like can be made without departing from the spirit of the invention and these are therefore considered to be within the scope of the invention as defined in the claims which follow.

What is claimed:

1. A nucleic acid construct comprising:
a chimeric nucleic acid molecule encoding a tripartite glycosyltransferase fusion protein, said chimeric nucleic acid molecule comprising:
a first nucleic acid moiety encoding an amphipathic shield domain protein;
a second nucleic acid moiety encoding a glycosyltransferase; and
a third nucleic acid moiety encoding a water soluble expression decoy protein, wherein said first nucleic acid moiety is coupled to said second nucleic acid moiety's 3′ end and said third nucleic acid moiety is coupled to said second nucleic acid moiety's 5′ end, said coupling being direct or indirect.

2. The nucleic acid construct according to claim 1, wherein the amphipathic shield domain protein is selected from the group consisting of apolipoprotein A (ApoA), apolipoprotein B (ApoB), apolipoprotein C (ApoC), apolipoprotein D (ApoD), apolipoprotein E (ApoE), apolipoprotein H (ApoH), truncated human apolipoprotein A1 lacking its 43-residue globular N-terminal domain (ApoAI*), and a peptide self-assembly mimic (PSAM).

3. The nucleic acid construct according to claim 1, wherein the amphipathic shield domain protein is human apolipoprotein A1.

4. The nucleic acid construct according to claim 3, wherein the human apolipoprotein A1 is a truncated human apolipoprotein A1.

5. The nucleic acid construct according to claim 4, wherein the truncated human apolipoprotein A1 protein is truncated human apolipoprotein A1 lacking its 43-residue globular N-terminal domain (ApoAI*).

6. The nucleic acid construct according to any one of claim 1, wherein the glycosyltransferase is a truncated glycosyltransferase.

7. The nucleic acid construct according to any one of claims 1 to 6, wherein the glycosyltransferase is a prokaryotic glycosyltransferase.

8. The nucleic acid construct according to any one of claims 1 to 6, wherein the glycosyltransferase is a eukaryotic glycosyltransferase.

9. The nucleic acid construct according to claim 8, wherein the glycosyltransferase is a human glycosyltransferase.

10. The nucleic acid construct according to any of claims 1 to 9, wherein the glycosyltransferase is selected from the group consisting of (i) a single-pass transmembrane protein with C-terminus in cytoplasm (type I transmembrane protein); (ii) a single-pass transmembrane protein with N-terminus in cytoplasm (type II transmembrane protein); (iii) a multi-pass transmembrane protein; and (iv) a secretory protein with N-terminal signal peptide and C-terminal ER retention domain.

11. The nucleic acid construct according to any of claims 1 to 9, wherein the glycosyltransferase is selected from the group consisting of fucosyltransferases (FucTs), galactosyltransferases (Gals), glucosyltransferases (GlcTs), mannosyltransferases (ManTs), N-acetylgalactosyltransferases (GalNAcTs), N-acetylglucosaminyltransferases (GlcNAcTs), and sialyltransferases (SiaTs).

12. The nucleic acid construct according to any of claims 1 to 9, wherein the glycosyltransferase is selected from the group consisting of human galactoside 2-alpha-L-fucosyltransferase 1 (HsFUT1), human galactoside 2-alpha-L-fucosyltransferase 2 (HsFUT2), HUMAN Galactoside 3(4)-L-fucosyltransferase (HsFUT3), human alpha-(1,3)-fucosyltransferase 4 (HsFUT4), human alpha-(1,3)-fucosyltransferase 5 (HsFUT5), human alpha-(1,3)-fucosyltransferase 6 (HsFUT6), human alpha-(1,3)-fucosyltransferase 7 (HsFUT7), human alpha-(1,6)-fucosyltransferase (HsFUT8), human alpha-(1,3)-fucosyltransferase 9 (HsFUT9), human alpha-(1,3)-fucosyltransferase 10 (HsFUT10), human alpha-(1,3)-fucosyltransferase 11 (HsFUT11), human GDP-fucose protein O-fucosyltransferase 1 (HsPOFUT1), human CMP-N-acetylneuraminate-beta-galactosamide-alpha-2,3-sialyltransferase 1 (HsST3Gal1), human CMP-N-acetylneuraminate-beta-1,4-galactoside alpha-2,3-sialyltransferase (HsST3Gal3), human CMP-N-acetylneuraminate-beta-galactosamide-alpha-2,3-sialyltransferase 4 (HsST3Gal4), human type 2 lactosamine alpha-2,3-sialyltransferase (HsST3Gal6), human beta-galactoside alpha-2,6-sialyltransferase 1 (HsST6Gal1), human alpha-N-acetylgalactosaminide alpha-2,6-sialyltransferase 1 (HsST6GalNAc1), human alpha-N-acetylgalactosaminide alpha-2,6-sialyltransferase 2 (HsST6GalNAc2), human alpha-N-acetyl-neuraminyl-2,3-beta-galactosyl-1,3-N-acetyl-galactosaminide alpha-2,6-sialyltransferase (HsST6GalNAc4), human alpha-N-acetylneuraminide alpha-2,8-sialyltransferase (HsST8Sia1), human alpha-2,8-sialyltransferase 8b (HsST8Sia2), human sia-alpha-2,3-gal-beta-1,4-GlcNAc-R:alpha 2,8-sialyltransferase (HsST8Sia3), human CMP-N-acetylneuraminate-poly-alpha-2,8-sialyltransferase (HsST8Sia4), human polypeptide N-acetylgalactosaminyltransferase 1 (HsppGalNAcT1), human polypeptide N-acetylgalactosaminyltransferase 2 (HsppGalNAcT2), human polypeptide N-acetylgalactosaminyltransferase 3 (HsppGalNAcT3), human polypeptide N-acetylgalactosaminyltransferase 4 (HsppGalNAcT4), human polypeptide N-acetylgalactosaminyltransferase 5 (HsppGalNAcT5), human polypeptide N-acetylgalactosaminyltransferase 6 (HsppGalNAcT6), human N-acetylgalactosaminyltransferase 7 (HsppGalNAcT7), human probable polypeptide N-acetylgalactosaminyltransferase 8 (HsppGalNAcT8), human polypeptide N-acetylgalactosaminyltransferase 9 (HsppGalNAcT9), human polypeptide N-acetylgalactosaminyltransferase 10 (HsppGalNAcT10), human UDP-GalNAc:beta-1,3-N-acetylgalactosaminyltransferase 1 (HsB3GALNT1), human beta-1,4 N-acetylgalactosaminyltransferase 1 (HsB4GALNT1), human histo-blood group ABO system transferase (Hs-A-group), human lactosylceramide 4-alpha-galactosyltransferase (HsA4GalT), human beta-1,3-galactosyltransferase 1 (HsB3GalT1), human beta-1,3-galactosyltransferase 2 (HsB3GalT2), human beta-1,4-galactosyltransferase 1 (HsB4GalT1), human beta-1,4-galactosyltransferase 2 (HsB4GalT2), human beta-1,4-galactosyltransferase 3 (HsB4GalT3), human beta-1,4-galactosyltransferase 4 (HsB4GalT4), human beta-1,4-galactosyltransferase 5 (HsB4GalT5), human beta-1,4-galactosyltransferase 6 (HsB4GalT6), human histo-blood group ABO system transferase (Hs-B-group), human 2-hydroxyacylsphingosine 1-beta-galactosyltransferase (HsUGT8), human glycoprotein-N-acetylgalactosamine 3-beta-galactosyltransferase 1 (HsC1GLT), human C1GALT1-specific chaperone 1 (HsCOSMC), human chitobiosyldiphosphodolichol beta-mannosyltransferase (HsAlg1), human alpha-1,3/1,6-mannosyltransferase (HsAlg2), human Dol-P-Man:Man(5)GlcNAc(2)-PP-Dol alpha-1,3-mannosyltransferase (HsAlg3), human GDP-man:man(3)GlcNAc(2)-PP-Dol alpha-1,2-mannosyltransferase (HsAlg11), human dol-p-man:man(7)GlcNAc(2)-PP-Dol alpha-1,6-mannosyltransferase (HsAlg12), human isoform 2 of putative UDP-N-acetylglucosamine transferase (HsAlg13), human UDP-N-acetylglucosamine transferase subunit alg14 homolog (HsAlg14), human dolichol-phosphate mannosyltransferase subunit 1 (HsDPM1), human GPI mannosyltransferase 1 (HsPIGM), human GPI mannosyltransferase 3 (HsPIGB), human GPI mannosyltransferase 4 (HsPIGZ), human dolichyl-phosphate beta-glucosyltransferase (HsAlg5), human dolichyl pyrophosphate man₉GlcNAc₂ alpha-1,3-glucosyltransferase (HsAlg6), human probable dolichyl pyrophosphate Glc1Man₉GlcNAc₂ alpha-1,3-glucosyltransferase (HsAlg8), human Dol-P-Glc:Glc₂Man₉GlcNAc₂—PP-Dol alpha-1,2-glucosyltransferase (HsAlg10), human ceramide glucosyltransferase (HsUGCG), human beta-1,3-glucosyltransferase (HsB3GLCT), human glycogenin-1 (HsGLYG), human protein O-glucosyltransferase 1 (HsPOGLUT1), human alpha-1,3-mannosyl-glycoprotein 2-beta-N-acetylglucosaminyltransferase (HsGnTI/MGAT1), human alpha-1,6-mannosyl-glycoprotein 2-beta-N-acetylglucosaminyltransferase (HsGnTII/MGAT2), human beta-1,4-mannosyl-glycoprotein 4-beta-N-acetylglucosaminyltransferase (HsGnTIII/MGAT3), human alpha-1,3-mannosyl-glycoprotein 4-beta-N-acetylglucosaminyltransferase a (HsGnTIV/MGAT4), human beta-1,3-galactosyl-O-glycosyl-glycoprotein beta-1,6-N-acetylglucosaminyltransferase (HsGCNT1), human N-acetyllactosaminide beta-1,6-N-acetylglucosaminyl-transferase (HsGCNT2), human N-acetyllactosaminide beta-1,3-N-acetylglucosaminyltransferase 2 (HsB3GNT2), human acetylgalactosaminyl-O-glycosyl-glycoprotein beta-1,3-N-acetylglucosaminyltransferase (HsB3GNT6), human phosphatidylinositol N-acetylglucosaminyltransferase subunit A (HsPIGA), human xyloside xylosyltransferase 1 (HsXXLT1), human UDP-glucuronosyltransferase 1-1 (HsUGT1A1), human beta-1,4-glucuronyltransferase 1 (HsB4GAT1), human UDP-glucuronosyltransferase 1-3 (HsUGT1A3), Campylobacter jejuni CsTII (CjCstII), Neisseria meningitidis polysialic acid O-acetyltransferase (NmPst), Campylobacter jejuni beta-1,3-galactosyltransferase (CjCgtB), Helicobacter pylori (strain 51) beta-4-galactosyltransferase (HpLgtB), Neisseria meningitidis serogroup B (strain MC58) lacto-N-neotetraose biosynthesis glycosyltransferase LgtB (NmLgtB), Neisseria gonorrhoeae lacto-N-neotetraose biosynthesis glycosyltransferase (NgLgtB), E. coli galactoside 2-alpha-L-fucosyltransferase WbgL (EcWbgL), E. coli undecaprenyl-phosphate alpha-N-acetylglucosaminyl 1-phosphate transferase (EcWecA), Legionella pneumophila subsp. Pneumophila Subversion of eukaryotic traffic protein A (LpSetA), Neisseria meningitidis alpha-2,9-polysialyltransferase (NmSynE), yeast beta-1,4-mannosyltransferase OS=Saccharomyces cerevisiae (ScAlg1), yeast GDP-Man:Man(3)GlcNAc(2)-PP-Dol alpha-1,2-mannosyltransferase (ScAlg11), Nicotiana tabacum alpha-1,3-mannosyl-glycoprotein 2-beta-N-acetylglucosaminyltransferase (NtGnTI), Nicotiana tabacum alpha-1,6-mannosyl-glycoprotein 2-beta-N-acetylglucosaminyltransferase-like (NtGnTII), Bos taurus n-acetyllactosaminide alpha-1,3-galactosyltransferase (BtGGTA1), mouse n-acetyllactosaminide alpha-1,3-galactosyltransferase (MmGGTA1), rat n-acetyllactosaminide alpha-1,3-galactosyltransferase (RnGGTA1), and Bos taurus beta-1,4-galactosyltransferase 1 (BtB4GalT1).

13. The nucleic acid construct according to any one of claims 1 to 12, wherein the glycosyltransferase is selected from the group consisting of Campylobacter jejuni CsTII (CjCstII), human alpha-(1,3)-fucosyltransferase 7 (HsFUT7), human CMP-N-acetylneuraminate-beta-galactosamide-alpha-2,3-sialyltransferase 1 (HsST3Gal1), human alpha-1,3-mannosyl-glycoprotein 2-beta-N-acetylglucosaminyltransferase (HsGnTI/MGAT1), human alpha-1,6-mannosyl-glycoprotein 2-beta-N-acetylglucosaminyltransferase (HsGnTII/MGAT2), human beta-1,4-galactosyltransferase 1 (Hsβ4GalT1), human β-galactoside-α2,6-sialyltransferase 1 (HsST6Gal1), and human alpha-(1,6)-fucosyltransferase (HsFUT8).

14. The nucleic acid construct according to any one of claims 1 to 13, wherein the water soluble expression decoy protein is selected from the group consisting of outer surface protein (OspA) lacking its native export signal peptide, DnaB lacking its native export signal peptide, and maltose-binding protein (MBP) lacking its N-terminal signal peptide.

15. The nucleic acid construct according to any one of claims 1 to 14, wherein the water soluble expression decoy protein is maltose-binding protein (MBP) lacking its N-terminal signal peptide.

16. The nucleic acid construct according to any one of claims 1 to 15, wherein the amphipathic shield domain protein is truncated human apolipoprotein A1 lacking its 43-residue globular N-terminal domain (ApoAI*) and the water soluble expression decoy protein is maltose-binding protein (MBP) lacking its N-terminal signal peptide.

17. The nucleic acid construct according to any one of claims 1 to 16, wherein the nucleic acid construct further comprises:
a promoter and
a termination sequence, wherein said promoter and said termination sequence are operatively coupled to the chimeric nucleic acid molecule.

18. The nucleic acid construct according to any one of claims 1 to 17, wherein the chimeric nucleic acid molecule further comprises:
one or more linker nucleic acid moieties coupling said first, second, and/or third nucleic acid moieties together.

19. An expression vector comprising the nucleic acid construct of any one of claims 1 to 18.

20. A host cell comprising the nucleic acid construct of any one of claims 1 to 18 or the expression vector of claim 19.

21. The host cell according to claim 20, wherein the host cell is prokaryotic.

22. The host cell according to claim 21, wherein the prokaryotic cell is E. coli.

23. The host cell according to claim 20, where in the host cell is eukaryotic.

24. The host cell according to claim 23, wherein the eukaryotic cell is a yeast cell.

25. The host cell according to claim 23, wherein the eukaryotic cell is a human cell line.

26. A tripartite glycosyltransferase fusion protein produced by the host cell according to any one of claims 20-25.

27. A cell-free protein expression system comprising:
a cell lysate or extract and
the nucleic acid construct according to anyone of claims 1 to 18 or the expression vector according to claim 19.

28. The cell-free protein expression system according to claim 27, wherein the cell lysate or extract comprises a heterologous and/or recombinant RNA polymerase.

29. The cell-free protein expression system according to claim 27 or claim 28, wherein the cell lysate or extract is capable of (i) transcribing the nucleic acid construct or the vector to form a translation template and (ii) translating the translation template.

30. The cell-free protein expression system according to any of claim 27 to claim 29, wherein the cell lysate or extract is an E. coli lysate or extract.

31. A tripartite glycosyltransferase fusion protein produced by the cell-free expression system according to any one of claims 27-30.

32. A method of recombinantly producing a tripartite glycosyltransferase fusion protein in water soluble form, said method comprising:
providing the host cell of any one of claims 20 to 25 or the cell-free expression system of any one of claims 27 to 30 and
culturing the host cell or using the cell-free expression system under conditions effective to express the tripartite glycosyltransferase fusion protein in a water soluble form within the host cell cytoplasm or the cell-free expression system.

33. The method according to claim 32 further comprising:
recovering the tripartite glycosyltransferase fusion protein from the host cell or the cell-free expression system following said culturing or said using, respectively.

34. The method according to claim 33, wherein the host cell is provided and said recovering comprises:
lysing the host cell to form a cell lysate comprising a water soluble fraction; and
subjecting the water soluble fraction of the cell lysate to chromatography to isolate the tripartite glycosyltransferase fusion protein.

35. The method according to claim 33, wherein the cell-free expression system is provided and said recovering comprises:
subjecting the water soluble fraction of the cell lysate to chromatography to isolate the tripartite glycosyltransferase fusion protein.

36. The method according to any one of claim 33 to claim 35, wherein the recovered tripartite glycosyltransferase fusion protein is conformationally correct.

37. A tripartite glycosyltransferase fusion protein produced by the methods of any one of claims 32 to 36.

38. A tripartite glycosyltransferase fusion protein comprising:
an amino terminal water soluble expression decoy protein;
a glycosyltransferase; and
a carboxyl terminal amphipathic shield domain protein.

39. The tripartite glycosyltransferase fusion protein according to claim 38, wherein the amphipathic shield domain protein is selected from the group consisting of apolipoprotein A (ApoA), apolipoprotein B (ApoB), apolipoprotein C (ApoC), apolipoprotein D (ApoD), apolipoprotein E (ApoE), apolipoprotein H (ApoH), truncated human apolipoprotein A1 lacking its 43-residue globular N-terminal domain (ApoAI*), and a peptide self-assembly mimic (PSAM).

40. The tripartite glycosyltransferase fusion protein according to claim 38, wherein the amphipathic shield domain protein is human apolipoprotein A1.

41. The tripartite glycosyltransferase fusion protein according to claim 40, wherein the human apolipoprotein A1 is a truncated human apolipoprotein A1.

42. The tripartite glycosyltransferase fusion protein according to claim 41, wherein the truncated human apolipoprotein A1 protein is truncated human apolipoprotein A1 lacking its 43-residue globular N-terminal domain (ApoAI*).

43. The tripartite glycosyltransferase fusion protein according to any one of claims 38 to 42, wherein the glycosyltransferase is a truncated glycosyltransferase.

44. The tripartite glycosyltransferase fusion protein according to any one of claims 38 to 43, wherein the glycosyltransferase is a prokaryotic glycosyltransferase.

45. The tripartite glycosyltransferase fusion protein according to any one of claims 38 to 43, wherein the glycosyltransferase is a eukaryotic glycosyltransferase.

46. The tripartite glycosyltransferase fusion protein according to claim 45, wherein the glycosyltransferase is a human glycosyltransferase.

47. The tripartite glycosyltransferase fusion protein according to any of claims 38 to 46, wherein the glycosyltransferase is selected from the group consisting of (i) a single-pass transmembrane protein with C-terminus in cytoplasm (type I transmembrane protein); (ii) a single-pass transmembrane protein with N-terminus in cytoplasm (type II transmembrane protein); (iii) a multi-pass transmembrane protein; and (iv) a secretory protein with N-terminal signal peptide and C-terminal ER retention domain.

48. The tripartite glycosyltransferase fusion protein according to any of claims 38 to 46, wherein the glycosyltransferase is selected from the group consisting of fucosyltransferases (FucTs), galactosyltransferases (Gals), glucosyltransferases (GlcTs), mannosyltransferases (ManTs), N-acetylgalactosyltransferases (GalNAcTs), N-acetylglucosaminyltransferases (GlcNAcTs), and sialyltransferases (SiaTs).

49. The tripartite glycosyltransferase fusion protein according to any of claims 38 to 46, wherein the glycosyltransferase is selected from the group consisting of wherein the glycosyltransferase is selected from the group consisting of human galactoside 2-alpha-L-fucosyltransferase 1 (HsFUT1), human galactoside 2-alpha-L-fucosyltransferase 2 (HsFUT2), HUMAN Galactoside 3(4)-L-fucosyltransferase (HsFUT3), human alpha-(1,3)-fucosyltransferase 4 (HsFUT4), human alpha-(1,3)-fucosyltransferase 5 (HsFUT5), human alpha-(1,3)-fucosyltransferase 6 (HsFUT6), human alpha-(1,3)-fucosyltransferase 7 (HsFUT7), human alpha-(1,6)-fucosyltransferase (HsFUT8), human alpha-(1,3)-fucosyltransferase 9 (HsFUT9), human alpha-(1,3)-fucosyltransferase 10 (HsFUT10), human alpha-(1,3)-fucosyltransferase 11 (HsFUT11), human GDP-fucose protein O-fucosyltransferase 1 (HsPOFUT1), human CMP-N-acetylneuraminate-beta-galactosamide-alpha-2,3-sialyltransferase 1 (HsST3Gal1), human CMP-N-acetylneuraminate-beta-1,4-galactoside alpha-2,3-sialyltransferase (HsST3Gal3), human CMP-N-acetylneuraminate-beta-galactosamide-alpha-2,3-sialyltransferase 4 (HsST3Gal4), human type 2 lactosamine alpha-2,3-sialyltransferase (HsST3Gal6), human beta-galactoside alpha-2,6-sialyltransferase 1 (HsST6Gal1), human alpha-N-acetylgalactosaminide alpha-2,6-sialyltransferase 1 (HsST6GalNAc1), human alpha-N-acetylgalactosaminide alpha-2,6-sialyltransferase 2 (HsST6GalNAc2), human alpha-N-acetyl-neuraminyl-2,3-beta-galactosyl-1,3-N-acetyl-galactosaminide alpha-2,6-sialyltransferase (HsST6GalNAc4), human alpha-N-acetylneuraminide alpha-2,8-sialyltransferase (HsST8Sia1), human alpha-2,8-sialyltransferase 8b (HsST8Sia2), human sia-alpha-2,3-gal-beta-1,4-GlcNAc-R:alpha 2,8-sialyltransferase (HsST8Sia3), human CMP-N-acetylneuraminate-poly-alpha-2,8-sialyltransferase (HsST8Sia4), human polypeptide N-acetylgalactosaminyltransferase 1 (HsppGalNAcT1), human polypeptide N-acetylgalactosaminyltransferase 2 (HsppGalNAcT2), human polypeptide N-acetylgalactosaminyltransferase 3 (HsppGalNAcT3), human polypeptide N-acetylgalactosaminyltransferase 4 (HsppGalNAcT4), human polypeptide N-acetylgalactosaminyltransferase 5 (HsppGalNAcT5), human polypeptide N-acetylgalactosaminyltransferase 6 (HsppGalNAcT6), human N-acetylgalactosaminyltransferase 7 (HsppGalNAcT7), human probable polypeptide N-acetyl galactosaminyltransferase 8 (HsppGalNAcT8), human polypeptide N-acetylgalactosaminyltransferase 9 (HsppGalNAcT9), human polypeptide N-acetylgalactosaminyltransferase 10 (HsppGalNAcT10), human UDP-GalNAc:beta-1,3-N-acetylgalactosaminyltransferase 1 (HsB3GALNT1), human beta-1,4 N-acetylgalactosaminyltransferase 1 (HsB4GALNT1), human histo-blood group ABO system transferase (Hs-A-group), human lactosylceramide 4-alpha-galactosyltransferase (HsA4GalT), human beta-1,3-galactosyltransferase 1 (HsB3GalT1), human beta-1,3-galactosyltransferase 2 (HsB3GalT2), human beta-1,4-galactosyltransferase 1 (HsB4GalT1), human beta-1,4-galactosyltransferase 2 (HsB4GalT2), human beta-1,4-galactosyltransferase 3 (HsB4GalT3), human beta-1,4-galactosyltransferase 4 (HsB4GalT4), human beta-1,4-galactosyltransferase 5 (HsB4GalT5), human beta-1,4-galactosyltransferase 6 (HsB4GalT6), human histo-blood group ABO system transferase (Hs-B-group), human 2-hydroxyacylsphingosine 1-beta-galactosyltransferase (HsUGT8), human glycoprotein-N-acetylgalactosamine 3-beta-galactosyltransferase 1 (HsC1GLT), human C1GALT1-specific chaperone 1 (HsCOSMC), human chitobiosyldiphosphodolichol beta-mannosyltransferase (HsAlg1), human alpha-1,3/1,6-mannosyltransferase (HsAlg2), human Dol-P-Man:Man(5)GlcNAc(2)-PP-Dol alpha-1,3-mannosyltransferase (HsAlg3), human GDP-man:man(3)GlcNAc(2)-PP-Dol alpha-1,2-mannosyltransferase (HsAlg11), human dol-p-man:man(7)GlcNAc(2)-PP-Dol alpha-1,6-mannosyltransferase (HsAlg12), human isoform 2 of putative UDP-N-acetylglucosamine transferase (HsAlg13), human UDP-N-acetylglucosamine transferase subunit alg14 homolog (HsAlg14), human dolichol-phosphate mannosyltransferase subunit 1 (HsDPM1), human GPI mannosyltransferase 1 (HsPIGM), human GPI mannosyltransferase 3 (HsPIGB), human GPI mannosyltransferase 4 (HsPIGZ), human dolichyl-phosphate beta-glucosyltransferase (HsAlg5), human dolichyl pyrophosphate man₉GlcNAc₂ alpha-1,3-glucosyltransferase (HsAlg6), human probable dolichyl pyrophosphate Glc1Man₉GlcNAc₂ alpha-1,3-glucosyltransferase (HsAlg8), human Dol-P-Glc:Glc₂Man₉GlcNAc₂-PP-Dol alpha-1,2-glucosyltransferase (HsAlg10), human ceramide glucosyltransferase (HsUGCG), human beta-1,3-glucosyltransferase (HsB3GLCT), human glycogenin-1 (HsGLYG), human protein O-glucosyltransferase 1 (HsPOGLUT1), human alpha-1,3-mannosyl-glycoprotein 2-beta-N-acetylglucosaminyltransferase (HsGnTI/MGAT1), human alpha-1,6-mannosyl-glycoprotein 2-beta-N-acetylglucosaminyltransferase (HsGnTII/MGAT2), human beta-1,4-mannosyl-glycoprotein 4-beta-N-acetylglucosaminyltransferase (HsGnTIII/MGAT3), human alpha-1,3-mannosyl-glycoprotein 4-beta-N-acetylglucosaminyltransferase a (HsGnTIV/MGAT4), human beta-1,3-galactosyl-O-glycosyl-glycoprotein beta-1,6-N-acetylglucosaminyltransferase (HsGCNT1), human N-acetyllactosaminide beta-1,6-N-acetylglucosaminyl-transferase (HsGCNT2), human N-acetyllactosaminide beta-1,3-N-acetylglucosaminyltransferase 2 (HsB3GNT2), human acetylgalactosaminyl-O-glycosyl-glycoprotein beta-1,3-N-acetylglucosaminyltransferase (HsB3GNT6), human phosphatidylinositol N-acetylglucosaminyltransferase subunit A (HsPIGA), human xyloside xylosyltransferase 1 (HsXXLT1), human UDP-glucuronosyltransferase 1-1 (HsUGT1A1), human beta-1,4-glucuronyltransferase 1 (HsB4GAT1), human UDP-glucuronosyltransferase 1-3 (HsUGT1A3), Campylobacter jejuni CsTII (CjCstII), Neisseria meningitidis polysialic acid O-acetyltransferase (NmPst), Campylobacter jejuni beta-1,3-galactosyltransferase (CjCgtB), Helicobacter pylori (strain 51) beta-4-galactosyltransferase (HpLgtB), Neisseria meningitidis serogroup B (strain MC58) lacto-N-neotetraose biosynthesis glycosyltransferase LgtB (NmLgtB), Neisseria gonorrhoeae lacto-N-neotetraose biosynthesis glycosyltransferase (NgLgtB), E. coli galactoside 2-alpha-L-fucosyltransferase WbgL (EcWbgL), E. coli undecaprenyl-phosphate alpha-N-acetylglucosaminyl 1-phosphate transferase (EcWecA), Legionella pneumophila subsp. Pneumophila Subversion of eukaryotic traffic protein A (LpSetA), Neisseria meningitidis alpha-2,9-polysialyltransferase (NmSynE), yeast beta-1,4-mannosyltransferase OS=Saccharomyces cerevisiae (ScAlg1), yeast GDP-Man:Man(3)GlcNAc(2)-PP-Dol alpha-1,2-mannosyltransferase (ScAlg11), Nicotiana tabacum alpha-1,3-mannosyl-glycoprotein 2-beta-N-acetylglucosaminyltransferase (NtGnTI), Nicotiana tabacum alpha-1,6-mannosyl-glycoprotein 2-beta-N-acetylglucosaminyltransferase-like (NtGnTII), Bos taurus n-acetyllactosaminide alpha-1,3-galactosyltransferase (BtGGTA1), mouse n-acetyllactosaminide alpha-1,3-galactosyltransferase (MmGGTA1), rat n-acetyllactosaminide alpha-1,3-galactosyltransferase (RnGGTA1), and Bos taurus beta-1,4-galactosyltransferase 1 (BtB4GalT1).

50. The tripartite glycosyltransferase fusion protein according to any one of claims 38 to 49, wherein the glycosyltransferase is selected from the group consisting of Campylobacter jejuni CsTII (CjCstII), human alpha-(1,3)-fucosyltransferase 7 (HsFUT7), human CMP-N-acetylneuraminate-beta-galactosamide-alpha-2,3-sialyltransferase 1 (HsST3Gal1), human alpha-1,3-mannosyl-glycoprotein 2-beta-N-acetylglucosaminyltransferase (HsGnTI/MGAT1), human alpha-1,6-mannosyl-glycoprotein 2-beta-N-acetylglucosaminyltransferase (HsGnTII/MGAT2), human beta-1,4-galactosyltransferase 1 (Hsβ4GalT1), human β-galactoside-α2,6-sialyltransferase 1 (HsST6Gal1), and human alpha-(1,6)-fucosyltransferase (HsFUT8).

51. The tripartite glycosyltransferase fusion protein according to any one of claims 38 to 50, wherein the water soluble expression decoy protein is selected from the group consisting of outer surface protein (OspA) lacking its native export signal peptide, DnaB lacking its native export signal peptide, and maltose-binding protein (MBP) lacking its N-terminal signal peptide.

52. The tripartite glycosyltransferase fusion protein according to any one of claims 38 to 51, wherein the water soluble expression decoy protein is maltose-binding protein (MBP) lacking its N-terminal signal peptide.

53. The tripartite glycosyltransferase fusion protein according to claim 38, wherein the amphipathic shield domain protein is truncated human apolipoprotein A1 lacking its 43-residue globular N-terminal domain (ApoAI*) and the water soluble expression decoy protein is maltose-binding protein (MBP) lacking its N-terminal signal peptide.

54. A method of cell-free glycan remodeling, said method comprising:
providing a glycan primer;
providing one or more tripartite glycosyltransferase fusion protein(s) according to any one of claims 26, 31, 37, or claims 38 to 53; and
incubating the glycan primer with the one or more tripartite glycosyltransferase fusion protein(s) under conditions effective to transfer a glycosyl group to the glycan primer to produce a modified glycan structure.

55. The method according to claim 54, wherein the glycan primer is a monosaccharide.

56. The method according to claim 54, wherein the glycan primer is an oligosaccharide.

57. The method according to claim 56, wherein the glycan primer comprises Man₃GlcNAc₂ or Man₅GlcNAc₂.

58. The method according to any one of claims 54 to 57, wherein the glycan primer is attached to an amino acid residue.

59. The method according to claim 58, wherein the amino acid residue is an asparagine residue.

60. The method according to any one of claim 58 or claim 59, wherein the glycan primer is attached to a glycoprotein.

61. The method according to claim 60, wherein the glycoprotein is an antibody.

62. The method according to any one of claims claim 54 to 61, wherein the tripartite glycosyltransferase fusion protein is selected from the group consisting of Sx-Δ29HsGnTI, Sx-Δ29HsGnTII, Sx-Δ30HsFucT8, Sx-Δ44Hsβ4GalT1, Sx-Δ26HsST6Gal1, and combinations thereof.

63. The method according to any one of claims 54 to 62, wherein said incubating is carried out with a plurality of different tripartite glycosyltransferase fusion proteins, at least some of the different tripartite glycosyltransferase proteins being used sequentially during said incubating.

64. The method according to any one of claims 54 to 62, wherein said incubating is carried out with a plurality of different tripartite glycosyltransferase fusion proteins, at least some of the different tripartite glycosyltransferase proteins being used simultaneously during said incubating.
